(19) United States
(12) Patent Application Publication
(10) Pub. No.: US 2009/0275608 A1
(43) Pub. Date: Nov. 5, 2009

(54) Methods of diagnosing and treating parp-mediated diseases

(75) Inventors: Valeria S. Ossovskaya, San Francisco, CA (US); Barry M. Sherman, Hillsborough, CA (US)

Correspondence Address: BiPar Sciences Inc. c/o MoFo, 755 Page Mill Road, Palo Alto, CA, 94304, US

(73) Assignee: BiPar Sciences, Inc., Brisbane, CA (US)

(21) Appl. No.: 12/322,551

(22) Filed: Feb. 4, 2009

Related U.S. Application Data
(60) Provisional application No. 61/026,077, filed on Feb. 4, 2008.

(51) Int. Cl. A61K 31/47; C12Q 1/68; A61K 31/415; A61K 31/40; A61K 31/04; A61K 31/015

(52) U.S. Cl. 514/307; 435/6; 514/311; 514/396; 514/412; 514/741; 514/765

(57) ABSTRACT

Disclosed are methods of identifying a disease treatable with modulators of differentially expressed genes in a disease, including at least PARP modulators, by identifying the level of expression of differentially expressed genes, including at least PARP, in a plurality of samples from a population, making a decision regarding identifying the disease treatable by modulators to the differentially expressed genes wherein the decision is made based on the level of expression of the differentially expressed genes. The method can further comprise treating the disease in a subject population with modulators of identified differentially expressed genes. The methods relate to identifying up-regulated expression of identified differentially-expressed genes in a disease and making a decision regarding the treatment of the disease. The level of expression of the differentially expressed genes in a disease can also help in determining the efficacy of the treatment with modulators to the differentially expressed genes.

### CROSS REFERENCE TO RELATED APPLICATIONS

This application claims the benefit of U.S. Provisional Application No. 61/026,077, entitled, “Methods of Diagnosing and Treating PARP-Mediated Diseases,” filed Feb. 4, 2008, which is incorporated herein by reference in its entirety.

### BACKGROUND OF THE INVENTION

The etiology of cancer and other diseases involves complex interactions between cellular factors, including cellular enzymatic receptors and other downstream intracellular factors that relay signals through the intracellular signaling network. Growth factor receptors have been recognized as a key factor in cancer biology, playing a significant role in the progression and maintenance of the malignant phenotype (Jones et al., 2006, Endocrine-Rel. Cancer, 13:S45-S51). For example, the expression of Epidermal Growth Factor Receptor (EGFR), a tyrosine kinase receptor, has been implicated as necessary in the development of adenomas and carcinomas in intestinal tumors, and subsequent expansion of initiated tumors (Roberts et al., 2002, PNAS, 99:1521-1526). Overexpression of EGFR also plays a role in neoplasia, especially in tumors of epithelial origin (Kari et al., 2003, Cancer Res., 63:1-5). EGFR is a member of the ErbB family of receptors, which includes HER2c/neu, Her2 and Her3 receptor tyrosine kinases. The molecular signaling pathway of EGFR activation has been mapped through experimental and computer modeling, involving over 200 reactions and 300 chemical species interactions (see Oda et al., Epub 2005, Mol. Sys. Biol., 1:2005.0010).

Another critical cellular pathway that is overexpressed by tumors, including mediation of the proliferation of cancer cells, is the insulin-like growth factor (IGF) signaling pathway (Khandwala et al., 2000, Endo. Rev., 21:215-244; Moschos and Mantzoros, 2002, Oncology 63:317-332; Bohula et al., 2003, Anticancer Drugs, 14:669-682). The signaling involves the function of two ligands, IGF1 and IGF2, three cell surface receptors, at least six high affinity binding proteins and binding protein protease (Basearga et al., 2006, Endocrine-Rel. Cancer, 13:S33-S43; Pollak et al., 2004, Nature Rev. Cancer 4:505-518). The insulin-like growth factor receptor (IGF1R) is a transmembrane receptor tyrosine kinase that mediates IGF biological activity and signaling through several critical cellular molecular networks including RAS0RAF-ERK and PI3-AKT-mTOR pathways. A functional IGF1R is required for transformation, and has been shown to promote tumor cell growth and survival (Riedemann and Macaulay, 2006, Endocr. Relat. Cancer, 13:S33-43). Several genes that have been shown to promote cell proliferation in response to IGF-1/IGF-2 binding in the IGF1R pathway include Shc, IRS, Grb2, SOS, Ras, Raf, MEK and ERK. Genes that have been implicated in the cell proliferation, motility and survival functions of IGF1 R signaling include IRS, PI3-K, PIP2, PTEN, PTP-2, PDK and Akt.

The signaling interplay between IGF signaling, IGF1 receptor and EGFR is important in the regulation of EGFR-mediated-pathway, and can contribute to a resistance to EGFR antagonist therapy (Jones et al., 2006, Endocrine-Rel. Cancer, 13:S45-S51).

Another pathway that is of interest in the proliferation and control of cancer growth and development includes the Ets family of transcription factors. The Ets family domain proteins, which are defined on the basis of a conserved primary sequence of their DNA-binding domains, function as either transcriptional activators or repressors, and their activities are often regulated by signal transduction pathways, including MAP kinase pathways (Sharrocks, et al., 1997, Int. J. Biochem. Cell Biol. 29:1371-1387). ETS transcription factors, such as ETS1, regulate numerous genes and are involved in stem cell development, cell senescence and death, and tumorigenesis. The conserved ETS domain within these proteins is a winged helix-turn-helix DNA-binding domain that recognizes the core consensus DNA sequence GGAA/T of target genes (Dwyer et al., 2007, Ann. New York Acad. Sci. 1114:36-47). There is a growing body of evidence that Ets 1 protein has oncogenic potential by playing a key role in the acquisition of invasive behavior of a tumorigenic cell. Among the genes that belong to the Ets 1 pathway to carry out its tumorigenic functions include the matrix metalloproteases MMP-1, MMP-3, MMP-9, as well as urokinase type plasminogen activator (uPA) (Sementchenko and Watson, 2000, Oncogene, 19:6533-6548). These proteases are known to be involved in extracellular matrix (ECM) degradation, a key event in invasion. In angiosarcoma of the skin, Ets 1 is co-expressed with MMP-1 (Naito et al., 2000, Pathol. Res. Pract. 196:103-109). Ovarian carcinoma cells and stromal fibroblasts in breast and ovarian cancer produce MMP-1 and MMP-9 along with Ets 1 (Behrens et al., 2001, J. Pathol. 194:43-50; Behrens et al., 2001, Int. J. Mol. Med. 8:149-154). In lung and brain tumors, Ets expression correlates with uPA expression (Kitange et al., 1999, Lab. Invest. 79:407-416; Takanami et al., 2001, Tumour Biol. 22:205-210; Nakada et al., 1999, J. Neuropathol. Exp. Neurol. 58:329-334). When overexpressed in endothelial cells or hepatoma cells, Ets 1 was shown to induce the production of MMP-1, MMP-3 plus MMP-9, or MMP-1, MMP-9 plus uPA, respectively (Oda et al., 1999, J. Cell Physiol. 178:121-132; Sato et al., 2000, Adv. Exp. Med. Biol. 476:109-115; Jiang et al., 2001, Biochem. Biophys. Res. Commun. 286:1123-1130). Regulation of MMP1, MMP3, MMP9 and uPA, as well as VEGF and VEGF receptor gene expression has been ascribed to Ets 1. Moreover, Ets 1 expression in tumors is indicative of poor clinical prognosis. Table I summarizes expression patterns of Ets1 in tumors.

TABLE I
Ets 1 Expression in Different Tumor Types

| | | | Stromal(S)/Vascular (V) | |
|---|---|---|---|---|
| Tumor Tissue | Cancer Type | Tumoral Expression | Expression | Comments |
| Brain | astrocytoma | 0% (grade II), 25% | high expression in glioma | higher |
| | | (III), 65% (IV) | microvasculature | expression in |
| | | | | recurrent vs. |
| | | | | primary tumors; |
| | meningioma | benign (38%), | | invasive tumor: |
| | | invasive (86%) | | correlation with |
| | | | | uPA expression |
| Breast | invasive carcinoma, DCIS, | 62% | correlates with VEGF, MMP1 | prognostic |
| | LCIS invasive cell lines | | and MMP9 expression | marker for poor |
| | | | | prognosis |
| Cartilage/bone | chondro-sarcoma | 60% | | |
| (jaw) | | | | |
| | osteosarcoma | 0% | | |
| Cervix | cervical carcinoma | | correlates with TMD | correlates with |
| | | | | poor prognosis |
| colon/rectum | adenomas | 0-44% | | |
| | colon cancer | 48-84% | 65% (V) correlates with TMD, | vascular Ets1: |
| | | | 28% (S) correlated with lung | linked with |
| | | | metastasis | LNM and poor |
| | | | | prognosis |
| endometrium | endometrial carcinoma | | correlates with TMD | associates with |
| | | | | histological |
| | | | | grade, detected |
| | | | | in cytoplasm |
| esophagus | squamous carcinoma | | correlates with VEGF | heterogeneous |
| | | | | expression, |
| | | | | higher at |
| | | | | invasive sites |
| liver/biliary tract | hepatocellular carcinoma | 50-100% | | higher in poorly |
| | | | | differentiated |
| | | | | tumors |
| | Bile duct carcinoma | 61% | | higher in well- |
| | | | | differentiated |
| | | | | tumors |
| | cholangio-cellular | 22% | | |
| | carcinomas | | | |
| Lung | pulmonary adeno- | | | linked to LNM |
| | carcinoma | | | |
| lymphoid tissue | T-leukemic cells (T-ALL, | | | |
| | ATL) | | | |
| Mouth | squamous cell carcinoma | 58% | | correlates with |
| | | | | tumor stage and |
| | | | | LNM |
| Ovary | benign cystadenoma | 0% | | |
| | carcinoma | 42%, higher when | 33% (S), correlates with MMP1 | associated with |
| | | stroma is invaded | and MMP9 expression | poor prognosis |
| Pancreas | adeno-carcinoma | 81% | | lower in poorly |
| | | | | differentiated |
| | | | | carcinoma |
| Stomach | adenomas | 0% | | |
| | adeno-carcinoma | 64% | correlates with TMD | |
| | mucosal carcinoma | 12% | | |
| Thymus | thymoma | | | higher in higher |
| | | | | grade tumors |
| thyroid gland | thyroid carcinoma | 40% (adenomas), 50-98% | | |
| | | (carcinoma) | | |
| vascular system | haemangioma | Weak | | |
| (skin) | | | | |
| | granuloma pyogenicum | Weak | | |
| | angiosarcoma | strong expression | | correlates with |
| | | | | MMP1 |
| | | | | expression |

TMD = tumor microvessel density;
LNM = lymph node metastasis;
DCIS = ductal carcinoma in situ;
LCIS = lobular carcinoma in situ (Ditmmer, 2003, Mol. Cancer 2: 29)

Poly-ADP ribose polymerase (PARP1) has been implicated as a putative downstream signal molecule of EGFR activation or perturbation. EGFR, through its signaling cascade pathway, stimulates PARP activation to initiate downstream cellular events mediated through the PARP pathway (Hagan et al., 2007, J. Cell. Biochem., 101: 1384-1393. PARP1 signaling participates in a variety of DNA-related functions including cell proliferation, differentiation, apoptosis and DNA repair, and also affects telomere length and chromosome stability (d'Adda di Fagagna et al, 1999, Nature Gen., 23(1): 76-80). PARP has been implicated in the maintenance of genomic integrity—inhibition or depletion of PARP (in PARP −/− mice as compared to wild type littermates) increases genomic instability in cells exposed to genotoxic agents in oligonucleotide microarray analysis of gene expression between asynchronously dividing primary fibroblasts (Simbulan-Rosenthal et al., PNAS, 97(21): 11274-11279 (2000)). PARP deficient mice have also been shown to be protected against septic shock, diabetes type I, stroke and inflammation. The direct protein-protein interaction of PARP-1 with both subunits of NF-κB has been shown to be required for its co-activator function (Hassa et al., J. Biol. Chem., 276(49): 45588-45597 (2001)). Oxidative stress-induced over activation of PARP 1 consumes NAD+ and consequently ATP, culminating in cell dysfunction or necrosis. Vimentin expression in lung cancer cells has been shown to be regulated at the transcriptional level; PARP-1 binds and activates the vimentin promoter independent of its catalytic domain and may play a role in H₂O₂-induced inhibition of vimentin expression. (Chu et al., Am. J. Physiol. Lung Cell. Mol. Physiol., 293: L1127-L1134 (2007)).

This cellular suicide mechanism through PARP activation has been implicated in the pathomechanism of cancer, stroke, myocardial ischemia, diabetes, diabetes-associated cardiovascular dysfunction, shock, traumatic central nervous system injury, arthritis, colitis, allergic encephalomyelitis, and various other forms of inflammation. PARP1 has also been shown to associate with and regulate the function of several transcription factors. The multiple functions of PARP1 pathways make it a target for a variety of serious conditions including various types of cancer and neurodegenerative diseases.

As seen, there are numerous molecular targets for cancer therapy that, when perturbed, may inhibit the growth or proliferation of cancerous tissue. Treatment of cancerous states may involve therapies targeting the molecular cancer targets above, for example, EGFR, together with traditional chemotherapeutic or other cancer therapies (Rocha-Lima et al., 2007, Cancer Control, 14:295-304). EGFR overexpression has been implicated in colorectal cancer, pancreatic cancer, gliomal development, small-cell lung cancer, and other carcinomas (Karamouzis et al., 2007, JAMA 298:70-82; Toschi et al., 2007, Oncologist, 12:211-220; Sequist et al., 2007, Oncologist, 12:325-330; Hatake et al., 2007, Breast Cancer, 14:132-149). Ceuximab, panitunmumam, matuzuman, MDX-446, nimutozumab, mAb 806, erbitux (IMC-C2225), IRESSA® (ZD1839), erlotinib, gefitinib, EKB-569, lapatinib (GW572016), PKI-166 and canertinib are some of the EGFR inhibitors that have been tested in clinical settings (Rocha-Lima et al., 2007, Cancer Control, 14:295-304). The EGFR inhibitors have been tested alone, and in combination with chemotherapeutic agents.

Studies to date, however, have not shown success at detailing the interactions of different known molecular pathways in the development of cancer. Moreover, although there are enormous resources dedicated towards the development of monotherapy and other combination therapies directed towards a large variety of cancer targets, the rise incidence of resistance to these therapies, and the prevention thereof, has not been studied fully. For example, although EGFR inhibitors have shown efficacy in treating cancer patients, only a small cohort of patients have proven to be fully responsive to EGFR inhibitor therapy (Hutcheson et al., 2006, Endocrine-Rel. Cancer, 13:S89-S97). Instead, a large subset have shown either de novo or acquired resistance to EGFR inhibitors in recent studies. This resistance to anti-EGFR therapy is unknown, but may originate from the complex cellular signaling cascade pathway for EGFR, including co-signaling cross-talk between other surface receptors, such as IGR1-receptor therapy (Jones et al., 2006, Endocrine-Rel. Cancer, 13:S45-S51). Treatment protocols that reduce resistance to currently available cancer therapies, such as chemotherapeutic or chemotoxic agents, or reduce resistance to other targets, would be desirable as potential new therapeutic regimens.

In addition, cancer detection, prognosis and staging are viable with today's early detection strategies, when they are highly treatable. However, such screening procedures are not available for all cancers, including breast cancer. More efficient and robust strategies for early diagnosis of cancer can be extremely beneficial for prevention and more efficient treatment of cancers. Screening procedures may also afford expression information to a practicing physician that would be beneficial for effectively treating cancer patients.

### SUMMARY OF THE INVENTION

In one aspect, provided herein are methods of identifying a disease or disease state in a subject treatable by a combination of at least one PARP modulator and a modulator to at least one co-regulated (e.g. differentially co-expressed gene), by measuring the level of PARP expression and other genes in the subject, and if the level of PARP and at least one other gene is differentially expressed in the subject, treating said subject with a modulator to PARP and other differentially expressed gene(s).

In one embodiment, co-regulated expressed genes may be IGF1R, IGF2 or IGF1. In another embodiment, the co-regulated expressed gene may be EGFR. In yet another embodiment, the co-regulated expressed genes may be IGF1, IGF2, IGF1R, EGFR, mdm2 or Bcl2. In some embodiments, at least one co-regulated expressed gene may be chosen from the group consisting of IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, CDK1, CDK2, CDK9, farnesyl transferase, UBE2A, UBE2D2, UBE2G1, USP28 or UBE2S. In yet another embodiment, at least one co-regulated expressed gene may be chosen from the group consisting of IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGFR, VEGFR2, VEGF, UBE2S, ABCC1, ABCC5, ABCD4, ACADM, ACLSL1, ACSL3, ACY1L2, ADM, ADRM1, AGPAT5, AHCY, AK3L1, AK3L2, AKIIP, AKR1B1, AKR1C1, AKR1C2, AKR1C3, ALDH18A1, ALDOA, ALOX5, ALPL, ANP32E, AOF1, APG5L, ARFGEF1, ARL5, ARPP-19, ASPH, ATF5, ATF7IP, ATIC, ATP11A, ATP11C, ATP1A1, ATP1B1, ATP2A2, ATP5G3, ATP5J2, ATP6V0B, B3GNT1, B4GALT2, BACE2, BACH, BAG2, BASP1, BCAT1, BCL2L1, BCL6, BGN, BPNT1, C1QBP, CACNB3, CAMK2D, CAP2, CCAR1, CD109, CD24, CD44, CD47, CD58, CD74, CD83, CD9, CDC14B, CDC42EP4, CDC5L, CDK4, CDK6, CDS1, CDW92, CEACAM6, CELSR2, CFLAR, CGI-90, CHST6, CHSY1, CKLFSF4, CKLFSF6, CKS1B, CMKOR1, CNDP2, CPD, CPE, CPSF3, CPSF5, CPSF6, CPT1B, CRR9, CSH2, CSK, CSNK2A1, CSPG2, CTPSCTSB, CTSD, CXADR, CXCR4, CXXC5, CXXC6, DAAM1, DCK, DDAH1, DDIT4, DDR1, DDX21, DDX39, DHTKD1, DLAT, DNAJA1, DNAJB11, DNAJC1, DNAJC10, DNAJC9, DNAJD1, DUSP10, DUSP24, DUSP6, DVL3, ELOVL6, EME1, ENO1, ENPP4, EPS8, ETNK1, ETV6, F11R, FA2H, FABP5, FADS2, FAS, FBXO45, FBXO7, FLJ23091, FTL, FTLL1, FZD6, G1P2, GALNT2, GALNT4, GALNT7, GANAB, GART, GBAS, GCHFR, GCLC, GCLM, GCNT1, GFPT1, GGA2, GGH, GLUL, GMNN, GMPS, GPI, GPR56, GPR89, GPX1, GRB10, GRHPR, GSPT1, GSR, GTPBP4, HDAC1, HDGF, HIG2, HMGB3, HPRT1, HPS5, HRMT1L2, HS2ST1, HSPA4, HSPA8, HSPB1, HSPCA, HSPCAL3, HSPCB, HSPD1, HSPE1, HSPH1, HTATIP2, HYOU1, ICMT, IDE, IDH2, IFI27, IGFBP3, IGSF4, ILF2, INPP5F, INSIG1, KHSRP, KLF4, KMO, KPNA2, KTN1, LAP3, LASS2, LDHA, LDHB, LGR4, LPGAT1, LTB4DH, LYN, MAD2L1, MADP-1, MAGED1, MAK3, MALAT1, MAP2K3, MAP2K6, MAP3K13, MAP4K4, MAPK13, MARCKS, MBTPS2, MCM4, MCTS1, MDH1, MDH2, ME1, ME2, METAP2, METTL2, MGAT4B, MKNK2. MLPH, MOBK1B, MOBKL1A, MSH2, MTHFD2, MUC1, MX1, MYCBP, NAJD1, NAT1, NBS1, NDFIP2, NEK6, NET1, NME1, NNT, NQO1, NRAS, NSE2, NUCKS, NUSAP1, NY-RENA-41, ODC1, OLR1, P4HB, PAFAH1B1, PAICS, PANK1, PCIA1, PCNA, PCTK1, PDAP1, PDIA4, PDIA6, PDXK, PERP, PFKP, PFTK1, PGD, PGK1, PGM2L1, PHCA, PKIG, PKM2, PKP4, PLA2G4A, PLCB1, PLCG2, PLD3, PLOD1, PLOD2, PMS2L3, PNK1, PNPT1, PON2, PP, PPIF, PPP1CA, PPP2R4, PPP3CA, PRCC, PRKD3, PRKDC, PRPSAP2, PSAT1, PSENEN, PSMA2, PSMA5, PSMA7, PSMB3, PSMB4, PSMD14, PSMD2, PSMD3, PSMD4, PSMD8, PTGFRN, PTGS1, PTK9, PTPN12, PTPN18, PTS, PYGB, RAB10, RAB11FIP1, RAB14, RAB31, RAB3IP, RACGAP1, RAN, RANBP1, RAP2B, RBBP4, RBBP7, RBBP8, RDH10, RFC3, RFC4, RFC5, RGS19IP1, RHOBTB3, RNASEH2A, RNGTT, RNPEP, ROBO1, RRAS2, SART2, SAT, SCAP2, SCD4, SDC2, SDC4, SEMA3F, SERPINE2, SFI1, SGPL1, SGPP1, SGPP2, SH3GLB2, SHC1, SMARCC1, SMC4L1, SMC4L1, SMS, SNRPD1, SORD, SORL1, SPP1, SQLE, SRD5A1, SRD5A2L, SRM, SRPK1, SS18, SSBP1, SSR3, ST3GAL5, ST6GAL1, ST6GALNAC2, STX18, SULF2, SWAP70, TA-KRP, TALA, TBL1XR1, TFRC, TIAM1, TKT, TMPO, TNFAIP2, TNFSF9, TOX, TPD52, TPI1, TPP1, TRA1, TRIP13, TRPS1, TSPAN13, TSTA3, TXN, TXNL2, TXNL5, TXNRD1, UBAP2L, UBE2A, UBE2D2, UBE2G1, UBE2V1, UCHL5, UGDH, UNC5CL, USP28, USP47, UTP14A, VDAC1, WIG1, YWHAB, YWHAE and YWHAZ.

In one aspect, provided herein are methods to identify disease treatable by PARP inhibitor in combination with an inhibitor or activator to at least one co-regulated expressed gene, in a subject by measuring the level of PARP and other co-regulated expressed genes in the subject, and if the level of PARP and/or other co-regulated expressed gene is up-regulated in the subject, further providing treatment of the subject with PARP inhibitors itself in combination with inhibitors to the other co-regulated expressed gene or genes.

One aspect relates to a method of identifying a disease or a stage of a disease treatable by a modulator of PARP and other co-regulated expressed genes, comprising identifying a level of co-regulated expressed genes, including PARP, in a sample of a subject, making a decision regarding identifying the disease treatable by modulators of the co-regulated expressed genes, including at least PARP, wherein the decision is made based on the level of expression of the co-regulated expressed genes, including at least PARP. In some embodiments, the level of the co-regulated expressed genes, including at least PARP, is up-regulated.

In some embodiments, the disease is selected from the group consisting of cancer, inflammation, metabolic disease, CVS disease, CNS disease, disorder of hematolymphoid system, disorder of endocrine and neuroendocrine, disorder of urinary tract, disorder of respiratory system, disorder of female reproductive system, and disorder of male reproductive system. In some embodiments, the cancer is selected from the group consisting of colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, intraductal carcinoma, mucinous carcinoma, phyllodes tumor, ovarian adenocarcinoma, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung adenocarcinoma, kidney carcinoma, urinary bladder carcinoma, Wilm's tumor, and lymphoma.

In some embodiments, the inflammation is selected from the group consisting of Wegener's granulomatosis, Hashimoto's thyroiditis, hepatocellular carcinoma, chronic pancreatitis, rheumatoid arthritis, reactive lymphoid hyperplasia, osteoarthritis, ulcerative colitis, and papillary carcinoma. In other embodiments, the metabolic disease is diabetes or obesity. In yet other embodiments, the CVS disease is selected from the group consisting of atherosclerosis, coronary artery disease, granulomatous myocarditis, chronic myocarditis, myocardial infarction, and primary hypertrophic cardiomyopathy. In some embodiments, the CNS disease is selected from the group consisting of Alzheimer's disease, cocaine abuse, schizophrenia, and Parkinson's disease. In some embodiments, the disorder of hematolymphoid system is selected from the group consisting of Non-Hodgkin's lymphoma, chronic lymphocyte leukemia, and reactive lymphoid hyperplasia.

In some embodiments, the disorder of endocrine and neuroendocrine is selected from the group consisting of nodular hyperplasia, Hashimoto's thyroiditis, islet cell tumor, and papillary carcinoma. In some embodiments, the disorder of urinary tract is selected from the group consisting of renal cell carcinoma, transitional cell carcinoma, and Wilm's tumor. In some embodiments, the disorder of respiratory system is selected from the group consisting of adenocarcinoma, adenosquamous carcinoma, squamous cell carcinoma, and large cell carcinoma. In some embodiments, the disorder of female reproductive system is selected from the group consisting of adenocarcinoma, leiomyoma, mucinous cystadenocarcinoma, and serous cystadenocarcinoma. In some embodiments, the disorder of male reproductive system is selected from the group consisting of prostate cancer, benign nodular hyperplasia, and seminoma.

In some embodiments, the identification of the level of the co-regulated expressed genes, including at least PARP, comprises an assay technique. In some embodiments, the assay technique measures the level of expression of the co-regulated expressed genes, including at least PARP. In some embodiments, the sample is selected from the group consisting of human normal sample, tumor sample, hair, blood, cell, tissue, organ, brain tissue, blood, serum, sputum, saliva, plasma, nipple aspirant, synovial fluid, cerebrospinal fluid, sweat, urine, fecal matter, pancreatic fluid, trabecular fluid, cerebrospinal fluid, tears, bronchial lavage, swabbing, bronchial aspirant, semen, prostatic fluid, precervicular fluid, vaginal fluids, and pre-ejaculate. In some embodiments, the level of the co-regulated expressed genes, including at least PARP, is up-regulated. In some embodiments, the level of the co-regulated expressed genes, including at least PARP, is down-regulated. In some embodiments, the PARP modulator is a PARP inhibitor or antagonist. In some embodiments, the PARP inhibitor or antagonist is selected from the group consisting of benzamide, quinolone, isoquinolone, benzopyrone, cyclic benzamide, benzimidazole and indole, or metabolites of said PARP inhibitors or antagonists.

In some embodiments, the method further comprises providing a conclusion regarding the disease to a patient, a health care provider or a health care manager, the conclusion being based on the decision. In some embodiments, the treatment is selected from the group consisting of oral administration, transmucosal administration, buccal administration, nasal administration, inhalation, parental administration, intravenous, subcutaneous, intramuscular, sublingual, transdermal administration, and rectal administration.

Another aspect relates to a computer-readable medium suitable for transmission of a result of an analysis of a sample wherein the medium comprises information regarding a disease in a subject treatable by modulators to co-regulated expressed genes in said subject, the co-regulated expressed genes including at least PARP, the information being derived by identifying a level of expression of the co-regulated expressed genes, including at least PARP, in the sample of the subject, and making a decision based on the level of the co-regulated expressed genes, including at least PARP, regarding treating the disease by modulators of the co-regulated expressed genes. In some embodiments, at least one step in the methods is implemented with a computer.

Yet another aspect is a method of identifying genes useful in the treatment of a patient with a disease susceptible to PARP inhibitor treatment, the method comprising identifying a disease treatable with at least one PARP modulator, wherein the expression level of PARP in a plurality of samples from a population is regulated in comparison to a control sample; determining the expression level of a panel of genes in the plurality of samples; and identifying genes that are co-regulated with said PARP regulation, wherein the expression level of said co-regulated genes in the plurality of samples are increased or decreased in comparison to a control sample; wherein modulation of said genes that are co-regulated with PARP regulation is useful in the treatment of a disease susceptible to PARP modulator treatment.

One additional aspect includes a method of treating a patient with a disease susceptible to PARP modulator treatment, the method comprising identifying a disease treatable with at least one PARP modulator, wherein the expression level of PARP in a sample from a patient with said disease is regulated in comparison to a reference sample; identifying at least one co-regulated gene in said sample in comparison to a reference sample, and treating said patient with modulators to PARP and the co-regulated gene.

Another embodiment disclosed herein is a method of treating a disease, the method comprising providing a plurality of samples from patients afflicted with said disease; identifying at least one gene regulated in each sample as compared to a reference sample, and treating a patient with said disease with modulators to the identified regulated gene(s) and a PARP modulator.

Yet another aspect is a method of treating a disease susceptible to PARP modulator treatment, the method comprising identifying a disease treatable with at least one PARP modulator, wherein the expression level of PARP in a plurality of samples is regulated in comparison to a reference sample; identifying at least one co-regulated gene in said plurality of samples in comparison to a reference sample; and treating a patient with said disease with modulators to PARP and the co-regulated gene.

One additional aspect is a method of treating a cancer susceptible to PARP inhibitor treatment, the method comprising identifying a cancer treatable with at least one PARP inhibitor, wherein the expression level of PARP in a plurality of cancer samples is up-regulated, identifying at least one co-upregulated gene in said plurality of samples; and treating a patient with said cancer with inhibitors to PARP and the co-regulated gene.

Also disclosed is a method of treating a breast cancer susceptible to PARP inhibitor treatment, the method comprising identifying a breast cancer treatable with at least one PARP inhibitor, wherein the expression level of PARP in a plurality of breast cancer samples is up-regulated, identifying at least one co-upregulated gene in said plurality of samples, and treating a patient with said breast cancer with inhibitors to PARP and the co-regulated gene. One embodiment is the treatment of triple negative breast cancer.

Furthermore, a method of treating a lung cancer susceptible to PARP inhibitor treatment is disclosed herein, the method comprising, identifying a lung cancer treatable with at least one PARP inhibitor, wherein the expression level of PARP in a plurality of lung cancer samples is up-regulated, identifying at least one co-upregulated gene in said plurality of samples, and treating a patient with said lung cancer with inhibitors to PARP and the co-regulated gene.

Another embodiment disclosed herein is a method of treating an endometrial cancer susceptible to PARP inhibitor treatment, the method comprising identifying an endometrial cancer treatable with at least one PARP inhibitor, wherein the expression level of PARP in a plurality of endometrial cancer samples is up-regulated, identifying at least one co-upregulated gene in said plurality of samples, and treating said patient with inhibitors to PARP and the co-regulated gene. Furthermore, a method of treating an ovarian cancer susceptible to PARP inhibitor treatment, the method comprising identifying an ovarian cancer treatable with at least one PARP inhibitor, wherein the expression level of PARP in a plurality of ovarian cancer samples is up-regulated, identifying at least one co-upregulated gene in said plurality of samples and treating said patient with inhibitors to PARP and the co-regulated gene.

Also provided herein are kits for diagnosing or staging a disease, the kit comprising means for measuring expression level of PARP in a tissue sample, means for measuring expression level of genes previously identified as co-regulated with PARP; and comparing said expression levels of PARP and co-regulated genes to a reference sample, wherein the level of expression as compared to the reference sample is indicative of the presence of disease or the disease stage. Also included are kits for treatment of a disease susceptible to a PARP inhibitor, the kit comprising means for measuring expression level of PARP in a tissue sample, wherein an increase in expression level of PARP in comparison to a reference sample is indicative of a disease susceptible to a PARP inhibitor; means for measuring expression level of genes previously identified as co-regulated with PARP, wherein an increase in the expression of said co-regulated genes is indicative of a use of an inhibitor to said co-regulated gene in the treatment of said disease; and inhibitors to PARP and said co-regulated genes for treatment of said disease.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the embodiments are set forth in the appended claims. A better understanding of the features and advantages of the present embodiments may be obtained by reference to the following detailed description that sets forth illustrative embodiments, in which the principles of the embodiments are utilized, and the accompanying drawings of which:

FIG. 1 is a flow chart showing the steps of one embodiment of the methods disclosed herein.

FIG. 2 illustrates a computer for implementing selected operations associated with the methods disclosed herein.

FIG. 3 depicts PARP expression in human healthy tissues.

FIG. 4 depicts PARP expression in malignant and normal tissues.

FIG. 5 depicts PARP expression in human primary tumors.

FIG. 6 depicts correlation of high expression of PARP1 (FIG. 6A) with lower expression of BRCA1 (FIG. 6B) and 2 in primary ovarian tumors.

FIG. 7 depicts upregulation of PARP expression in an ER-, PR- and Her-2 negative tissue specimen. FIG. 7A provides normal breast tissue samples stained with hemolysin and eosin (H&E) or for the markers ER, PR, HER2 or PARP1. FIG. 7B provides breast adenocarcinoma tissue samples stained with H&E or for the markers ER, PR, HER2 or PARP1.

FIG. 8 illustrates a physical interaction network from genes selected with a 2-fold change cutoff and common in three tissues: ovary, endometrium and breast.

FIG. 9 depicts a regulatory interaction network from genes selected with a 2-fold change cutoff and common in three tissues: ovary, endometrium and breast tissue.

FIG. 10 depicts mRNA expression in lung normal and tumor tissues expression in a lung human normal and tumor tissues. FIG. 10A depicts Ki-67; FIG. 10B depicts PARP1; FIG. 10C depicts PARP2, and FIG. 10D depicts RAD51 mRNA expression.

FIG. 11 depicts PARP expression in a lung human normal and tumor syngeneic specimen.

FIG. 12 depicts PARP expression in lung human normal and tumor syngeneic specimens.

FIG. 13 depicts PARP expression in lung human normal and tumor syngeneic specimen.

FIG. 14 depicts PARP expression in a breast human normal and tumor tissues. FIG. 14A depicts Ki-67; FIG. 14B depicts PARP1, FIG. 14C depicts PARP2, and FIG. 14D depicts RAD51 mRNA expression.

FIG. 15 depicts PARP expression in a breast human normal and tumor syngeneic specimen.

FIG. 16 depicts PARP expression in a breast human normal and tumor syngeneic specimen.

FIG. 17 depicts PARP expression in a breast human normal and tumor syngeneic specimen.

FIG. 18 depicts PARP1 inhibition (Compound III) on tumor growth and improval of survival of mice in human ovarian adenocarcinoma OVCAR-3 xenograft model of cancer.

FIG. 19: Compound III potentiates the activity of IGF-1R inhibitor Picropodophyllin (PPP) in triple negative breast cancer cells MDA-MB-468.

FIG. 20: HCC827 NSCLC cell line is a well characterized model for analysis of EGFR inhibitors.

### DETAILED DESCRIPTION OF THE INVENTION

The term “inhibit” or its grammatical equivalent, such as “inhibitory,” is not intended to require complete reduction in PARP activity. Such reduction is may be by at least about 50%, at least about 75%, at least about 90%, or by at least about 95% of the activity of the molecule in the absence of the inhibitory effect, e.g., in the absence of an inhibitor, such as PARP inhibitors disclosed herein. The term refers to an observable or measurable reduction in activity. In treatment scenarios, inhibition may be sufficient to produce a therapeutic and/or prophylactic benefit in the condition being treated.

The terms “sample”, “biological sample” or its grammatical equivalents, as used herein mean a material known to or suspected of expressing a level of PARP. The test sample can be used directly as obtained from the source or following a pretreatment to modify the character of the sample. The sample can be derived from any biological source, such as tissues or extracts, including cells, and physiological fluids, such as, for example, whole blood, plasma, serum, saliva, ocular lens fluid, cerebrospinal fluid, sweat, urine, milk, ascites fluid, synovial fluid, peritoneal fluid and the like. The sample may be obtained from non-human animals or humans. In one embodiment, samples are obtained from humans. The sample can be treated as needed prior to use, such as preparing plasma from blood, diluting viscous fluids, and the like. Methods of treating a sample can involve filtration, distillation, extraction, concentration, inactivation of interfering components, the addition of reagents, and the like.

The term “subject,” “patient” or “individual” as used herein in reference to individuals suffering from a disorder, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the mammal is a human.

The term “treating” or its grammatical equivalents as used herein, means achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The term “level of expression” or its grammatical equivalent as used herein, means a measurement of the amount of nucleic acid, e.g. RNA or mRNA, or protein of a gene in a subject, or alternatively, the level of activity of a gene or protein in said subject.

The term “differentially expressed” or its grammatical equivalent as used herein, means a level of expression that varies or differs from a reference level, which may include a normal or average level of expression measured in a subject or group of subjects. The level of expression may either increase or decrease relative to the reference level of expression, and may be transient or long-term in effect. The related term “co-regulated” or its grammatical equivalents as used herein, means the level of expression is altered or changed along or in tandem with, another gene, here PARP1. In some embodiments, the level of expression of a gene, e.g., IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, CDK1, CDK2, CDK9, farnesyl transferase, UBE2A, UBE2D2, UBE2G1, USP28 or UBE2S., changes along with the level of expression of PARP1. In some embodiments, the co-regulated is at least one of the following genes: IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, CDK1, CDK2, CDK9, farnesyl transferase, UBE2A, UBE2D2, UBE2G1, USP28, UBE2S, ABCC1, ABCC5, ABCD4, ACADM, ACLSL1, ACSL3, ACY1L2, ADM, ADRM1, AGPAT5, AHCY, AK3L1, AK3L2, AKIIP, AKR1B1, AKR1C1, AKR1C2, AKR1C3, ALDH18A1, ALDOA, ALOX5, ALPL, ANP32E, AOF1, APG5L, ARFGEF1, ARL5, ARPP-19, ASPH, ATF5, ATF7IP, ATIC, ATP11A, ATP11C, ATP1A1, ATP1B1, ATP2A2, ATP5G3, ATP5J2, ATP6V0B, B3GNT1, B4GALT2, BACE2, BACH, BAG2, BASP1, BCAT1, BCL2L1, BCL6, BGN, BPNT1, C1QBP, CACNB3, CAMK2D, CAP2, CCAR1, CD109, CD24, CD44, CD47, CD58, CD74, CD83, CD9, CDC14B, CDC42EP4, CDC5L, CDK4, CDK6, CDS1, CDW92, CEACAM6, CELSR2, CFLAR, CGI-90, CHST6, CHSY1, CKLFSF4, CKLFSF6, CKS1B, CMKOR1, CNDP2, CPD, CPE, CPSF3, CPSF5, CPSF6, CPT1B, CRR9, CSH2, CSK, CSNK2A1, CSPG2, CTPSCTSB, CTSD, CXADR, CXCR4, CXXC5, CXXC6, DAAM1, DCK, DDAH1, DDIT4, DDR1, DDX21, DDX39, DHTKD1, DLAT, DNAJA1, DNAJB11, DNAJC1, DNAJC10, DNAJC9, DNAJD1, DUSP10, DUSP24, DUSP6, DVL3, ELOVL6, EME1, ENO1, ENPP4, EPS8, ETNK1, ETV6, F11R, FA2H, FABP5, FADS2, FAS, FBXO45, FBXO7, FLJ23091, FTL, FTLL1, FZD6, G1P2, GALNT2, GALNT4, GALNT7, GANAB, GART, GBAS, GCHFR, GCLC, GCLM, GCNT1, GFPT1, GGA2, GGH, GLUL, GMNN, GMPS, GPI, GPR56, GPR89, GPX1, GRB10, GRHPR, GSPT1, GSR, GTPBP4, HDAC1, HDGF, HIG2, HMGB3, HPRT1, HPS5, HRMT1L2, HS2ST1, HSPA4, HSPA8, HSPB1, HSPCA, HSPCAL3, HSPCB, HSPD1, HSPE1, HSPH1, HTATIP2, HYOU1, ICMT, IDE, IDH2, IFI27, IGFBP3, IGSF4, ILF2, INPP5F, INSIG1, KHSRP, KLF4, KMO, KPNA2, KTN1, LAP3, LASS2, LDHA, LDHB, LGR4, LPGAT1, LTB4DH, LYN, MAD2L1, MADP-1, MAGED1, MAK3, MALAT1, MAP2K3, MAP2K6, MAP3K13, MAP4K4, MAPK13, MARCKS, MBTPS2, MCM4, MCTS1, MDH1, MDH2, ME1, ME2, METAP2, METTL2, MGAT4B, MKNK2. MLPH, MOBK1B, MOBKL1A, MSH2, MTHFD2, MUC1, MX1, MYCBP, NAJD1, NAT1, NBS1, NDFIP2, NEK6, NET1, NME1, NNT, NQO1, NRAS, NSE2, NUCKS, NUSAP1, NY-REN-41, ODC1, OLR1, P4HB, PAFAH1B1, PAICS, PANK1, PCIA1, PCNA, PCTK1, PDAP1, PDIA4, PDIA6, PDXK, PERP, PFKP, PFTK1, PGD, PGK1, PGM2L1, PHCA, PKIG, PKM2, PKP4, PLA2G4A, PLCB1, PLCG2, PLD3, PLOD1, PLOD2, PMS2L3, PNK1, PNPT1, PON2, PP, PPIF, PPP1CA, PPP2R4, PPP3CA, PRCC, PRKD3, PRKDC, PRPSAP2, PSAT1, PSENEN, PSMA2, PSMA5, PSMA7, PSMB3, PSMB4, PSMD14, PSMD2, PSMD3, PSMD4, PSMD8, PTGFRN, PTGS1, PTK9, PTPN12, PTPN18, PTS, PYGB, RAB10, RAB11FIP1, RAB14, RAB31, RAB3IP, RACGAP1, RAN, RANBP1, RAP2B, RBBP4, RBBP7, RBBP8, RDH10, RFC3, RFC4, RFC5, RGS19IP1, RHOBTB3, RNASEH2A, RNGTT, RNPEP, ROBO1, RRAS2, SART2, SAT, SCAP2, SCD4, SDC2, SDC4, SEMA3F, SERPINE2, SFI1, SGPL1, SGPP1, SGPP2, SH3GLB2, SHC1, SMARCC1, SMC4L1, SMC4L1, SMS, SNRPD1, SORD, SORL1, SPP1, SQLE, SRD5A1, SRD5A2L, SRM, SRPK1, SS18, SSBP1, SSR3, ST3GAL5, ST6GAL1, ST6GALNAC2, STX18, SULF2, SWAP70, TA-KRP, TALA, TBL1XR1, TFRC, TIAM1, TKT, TMPO, TNFAIP2, TNFSF9, TOX, TPD52, TPI1, TPP1, TRA1, TRIP13, TRPS1, TSPAN13, TSTA3, TXN, TXNL2, TXNL5, TXNRD1, UBAP2L, UBE2A, UBE2D2, UBE2G1, UBE2V1, UCHL5, UGDH, UNC5CL, USP28, USP47, UTP14A, VDAC1, WIG1, YWHAB, YWHAE and YWHAZ.

### Method of Identifying a Disease or Stage of a Disease Treatable by Modulators of Differentially Expressed Genes, Including at Least PARP

In one aspect, the methods include identifying a disease treatable by modulators of regulated genes, including at least PARP, comprising identifying a level of expression of regulated genes in a sample of a subject, making a decision regarding identifying the disease treatable by the modulators of the regulated genes, including at least PARP, wherein the decision is made based on the level of expression of the regulated genes. In another aspect, the methods include treating a disease with modulators of the regulated genes in a subject comprising identifying a level of expression of the regulated genes in a sample of the subject, making a decision based on the level of expression of the regulated genes, including at least PARP, regarding identifying the disease treatable by modulators of the regulated genes, and treating the disease in the subject by modulators of the regulated genes. In yet another aspect, the methods include identifying the level of expression of regulated genes in a sample of a subject and treating a subject with modulators to the identified regulated genes and a PARP modulator. In another aspect, the method further includes providing a conclusion regarding the disease to a patient, a health care provider or a health care manager, where the conclusion is based on the decision. In some embodiments, disease is breast cancer. In some embodiments, the levels of the regulated genes, including at least PARP, are up-regulated. In some embodiments, the level of the regulated genes, including at least PARP, is down-regulated.

The present embodiments identify diseases such as, cancer, inflammation, metabolic disease, CVS disease, CNS disease, disorder of hematolymphoid system, disorder of endocrine and neuroendocrine, disorder of urinary tract, disorder of respiratory system, disorder of female reproductive system, and disorder of male reproductive system where the level of the regulated genes, including at least PARP, are up-regulated. Accordingly, the present embodiments identify these diseases to be treatable by modulators of the regulated genes identified. Modulation of PARP gene expression, at a minimum, together with other regulated genes identified by the methods described herein, will be useful in the treatment of these identified diseases. In some embodiments, the co-regulated genes, along with at least PARP, may be proteins expressed in the pathways of PARP, EGFR and/or IGF1R. In other embodiments, the co-regulated genes may include IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, CDK1, CDK2, CDK9, farnesyl transferase, UBE2A, UBE2D2, UBE2G1, USP28, UBE2S, ABCC1, ABCC5, ABCD4, ACADM, ACLSL1, ACSL3, ACY1L2, ADM, ADRM1, AGPAT5, AHCY, AK3L1, AK3L2, AKIIP, AKR1B1, AKR1C1, AKR1C2, AKR1C3, ALDH18A1, ALDOA, ALOX5, ALPL, ANP32E, AOF1, APG5L, ARFGEF1, ARL5, ARPP-19, ASPH, ATF5, ATF7IP, ATIC, ATP11A, ATP11C, ATP1A1, ATP1B1, ATP2A2, ATP5G3, ATP5J2, ATP6V0B, B3GNT1, B4GALT2, BACE2, BACH, BAG2, BASP1, BCAT1, BCL2L1, BCL6, BGN, BPNT1, C1QBP, CACNB3, CAMK2D, CAP2, CCAR1, CD109, CD24, CD44, CD47, CD58, CD74, CD83, CD9, CDC14B, CDC42EP4, CDC5L, CDK4, CDK6, CDS1, CDW92, CEACAM6, CELSR2, CFLAR, CGI-90, CHST6, CHSY1, CKLFSF4, CKLFSF6, CKS1B, CMKOR1, CNDP2, CPD, CPE, CPSF3, CPSF5, CPSF6, CPT1B, CRR9, CSH2, CSK, CSNK2A1, CSPG2, CTPSCTSB, CTSD, CXADR, CXCR4, CXXC5, CXXC6, DAAM1, DCK, DDAH1, DDIT4, DDR1, DDX21, DDX39, DHTKD1, DLAT, DNAJA1, DNAJB11, DNAJC1, DNAJC10, DNAJC9, DNAJD1, DUSP10, DUSP24, DUSP6, DVL3, ELOVL6, EME1, ENO1, ENPP4, EPS8, ETNK1, ETV6, F11R, FA2H, FABP5, FADS2, FAS, FBXO45, FBXO7, FLJ23091, FTL, FTLL1, FZD6, G1P2, GALNT2, GALNT4, GALNT7, GANAB, GART, GBAS, GCHFR, GCLC, GCLM, GCNT1, GFPT1, GGA2, GGH, GLUL, GMNN, GMPS, GPI, GPR56, GPR89, GPX1, GRB10, GRHPR, GSPT1, GSR, GTPBP4, HDAC1, HDGF, HIG2, HMGB3, HPRT1, HPS5, HRMT1L2, HS2ST1, HSPA4, HSPA8, HSPB1, HSPCA, HSPCAL3, HSPCB, HSPD1, HSPE1, HSPH1, HTATIP2, HYOU1, ICMT, IDE, IDH2, IFI27, IGFBP3, IGSF4, ILF2, INPP5F, INSIG1, KHSRP, KLF4, KMO, KPNA2, KTN1, LAP3, LASS2, LDHA, LDHB, LGR4, LPGAT1, LTB4DH, LYN, MAD2L1, MADP-1, MAGED1, MAK3, MALAT1, MAP2K3, MAP2K6, MAP3K13, MAP4K4, MAPK13, MARCKS, MBTPS2, MCM4, MCTS1, MDH1, MDH2, ME1, ME2, METAP2, METTL2, MGAT4B, MKNK2. MLPH, MOBK1B, MOBKL1A, MSH2, MTHFD2, MUC1, MX1, MYCBP, NAJD1, NAT1, NBS1, NDFIP2, NEK6, NET1, NME1, NNT, NQO1, NRAS, NSE2, NUCKS, NUSAP1, NY-REN-41, ODC1, OLR1, P4HB, PAFAH1B1, PAICS, PANK1, PCIA1, PCNA, PCTK1, PDAP1, PDIA4, PDIA6, PDXK, PERP, PFKP, PFTK1, PGD, PGK1, PGM2L1, PHCA, PKIG, PKM2, PKP4, PLA2G4A, PLCB1, PLCG2, PLD3, PLOD1, PLOD2, PMS2L3, PNK1, PNPT1, PON2, PP, PPIF, PPP1CA, PPP2R4, PPP3CA, PRCC, PRKD3, PRKDC, PRPSAP2, PSAT1, PSENEN, PSMA2, PSMA5, PSMA7, PSMB3, PSMB4, PSMD14, PSMD2, PSMD3, PSMD4, PSMD8, PTGFRN, PTGS1, PTK9, PTPN12, PTPN18, PTS, PYGB, RAB10, RAB11FIP1, RAB14, RAB31, RAB3IP, RACGAP1, RAN, RANBP1, RAP2B, RBBP4, RBBP7, RBBP8, RDH10, RFC3, RFC4, RFC5, RGS19IP1, RHOBTB3, RNASEH2A, RNGTT, RNPEP, ROBO1, RRAS2, SART2, SAT, SCAP2, SCD4, SDC2, SDC4, SEMA3F, SERPINE2, SFI1, SGPL1, SGPP1, SGPP2, SH3GLB2, SHC1, SMARCC1, SMC4L1, SMC4L1, SMS, SNRPD1, SORD, SORL1, SPP1, SQLE, SRD5A1, SRD5A2L, SRM, SRPK1, SS18, SSBP1, SSR3, ST3GAL5, ST6GAL1, ST6GALNAC2, STX18, SULF2, SWAP70, TA-KRP, TALA, TBL1XR1, TFRC, TIAM1, TKT, TMPO, TNFAIP2, TNFSF9, TOX, TPD52, TPI1, TPP1, TRA1, TRIP13, TRPS1, TSPAN13, TSTA3, TXN, TXNL2, TXNL5, TXNRD1, UBAP2L, UBE2A, UBE2D2, UBE2G1, UBE2V1, UCHL5, UGDH, UNC5CL, USP28, USP47, UTP14A, VDAC1, WIG1, YWHAB, YWHAE, YWHAZ, or a combination thereof. In yet other embodiments, the co-regulated genes may include IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, CDK1, CKD2, CDK9, farnesyl transferase, UBE2A, UBE2D2, UBE2G1, USP28, UBE2S, or a combination thereof.

In one embodiment, PARP inhibitors in combination with modulators of other regulated genes are PARP-1 inhibitors. The PARP inhibitors used in the methods described herein can act via a direct or indirect interaction with PARP, such as, for example, PARP-1. The PARP inhibitors used herein may modulate PARP or may modulate one or more entities in the PARP pathway. The PARP inhibitors can in some embodiments inhibit PARP activity.

The methods disclosed herein may be particularly useful in treating cancer of the female reproductive system. Breast tumors in women who inherit faults in either the BRCA1 or BRCA2 genes occur because the tumor cells have lost a specific mechanism that repair damaged DNA. BRCA1 and BRCA2 are important for DNA double-strand break repair by homologous recombination, and mutations in these genes predispose to breast and other cancers. PARP is involved in base excision repair, a pathway in the repair of DNA single-strand breaks. BRCA1 or BRCA2 dysfunction sensitizes cells to the inhibition of PARP enzymatic activity, resulting in chromosomal instability, cell cycle arrest and subsequent apoptosis.

PARP inhibitors, thus, may kill cells where this form of DNA repair is absent and so are effective in killing BRCA deficient tumor cells and other similar tumor cells. Normal cells may be unaffected by the drug as they may still possess this DNA repair mechanism. Accordingly, PARP inhibitors, in combination with modulators of other regulated genes identified through the methods described herein, may be useful in treating breast cancer patients with BRCA1 or BRCA2 deficiencies. This treatment might also be applicable to other forms of breast cancer that behave like BRCA deficient cancer. Typically, breast cancer patients are treated with drugs that kill tumor cells but also damage normal cells. It is damage to normal cells that can lead to distressing side effects, like nausea and hair loss. In some embodiments, an advantage of treating with PARP inhibitors is that it is targeted; tumor cells are killed while normal cells appear unaffected. This is because PARP inhibitors exploit the specific genetic make-up of some tumor cells.

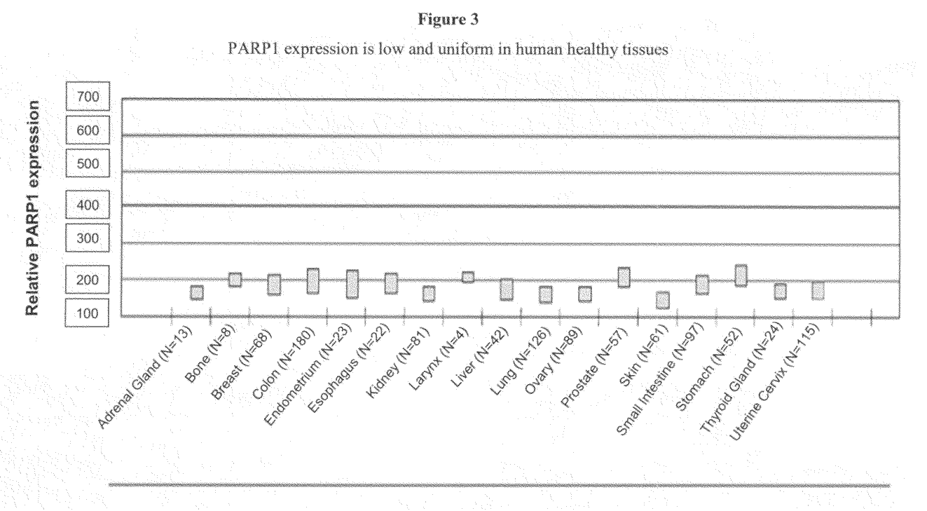

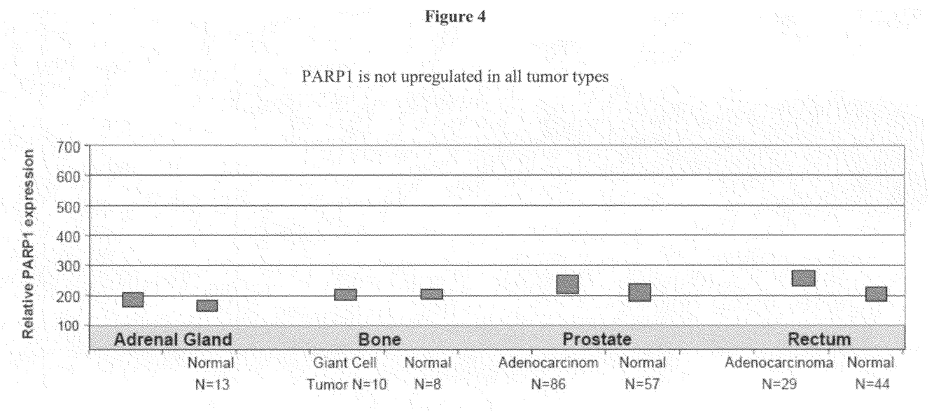

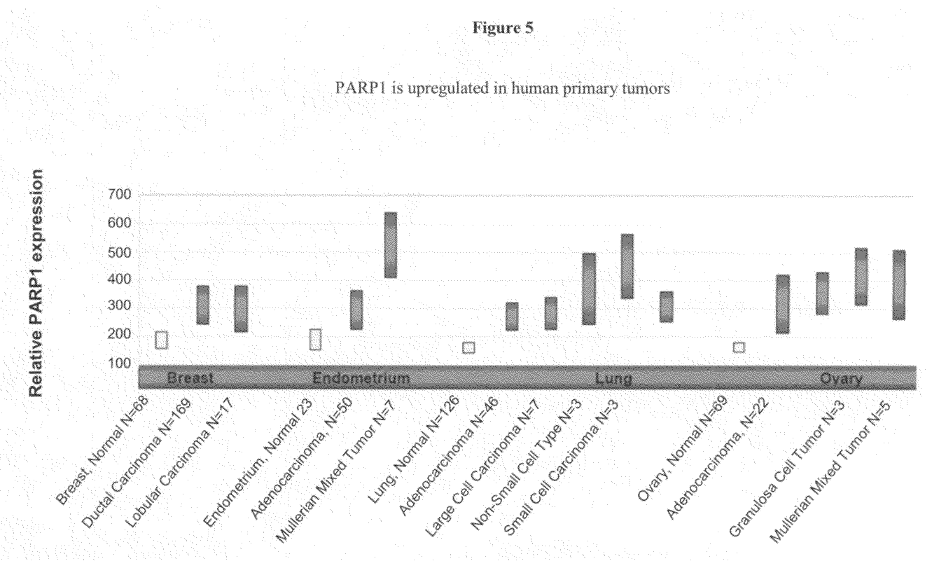

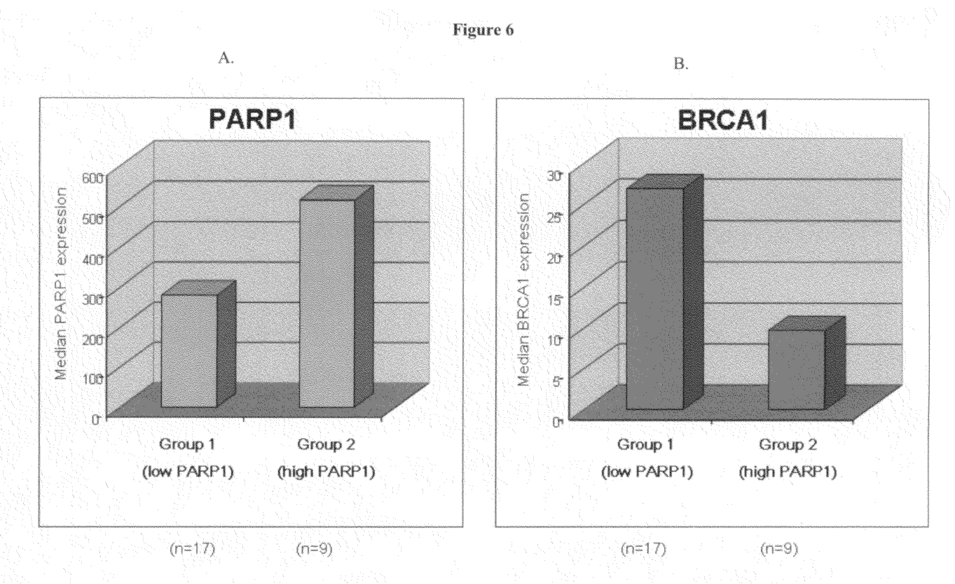

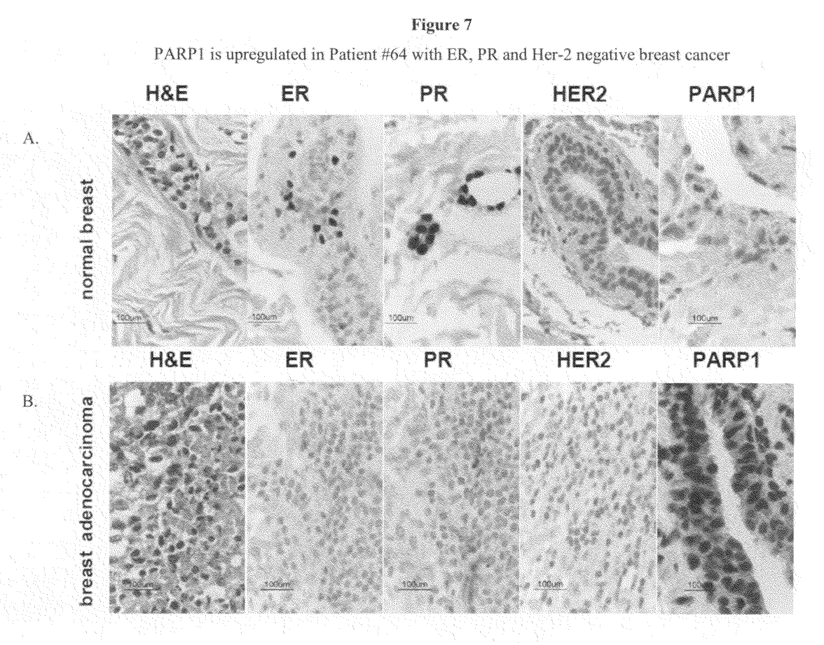

It has previously been shown that subjects deficient in BRCA genes have up-regulated levels of PARP. See, e.g., Example 2 and U.S. application Ser. No. 11/818,210, the entire contents of which are expressly incorporated by reference herein. FIGS. 3-5 depict the differential regulation of PARP in certain primary tumors as compared to reference normal samples. FIG. 6 depicts the correlation of high expression of PARP-1 (FIG. 6A) with lower expression of BRCA1 (FIG. 6B) in primary human ovarian tumors. Moreover, FIG. 7 depicts the upregulation of PARP expression in triple negative breast cancers (FIG. 7B) compared to normal breast tissue (FIG. 7A). PARP up-regulation may be an indicator of other defective DNA-repair pathways and unrecognized BRCA-like genetic defects. Assessment of PARP-1 gene expression is an indicator of tumor sensitivity to PARP inhibitor. The BRCA deficient patients treatable by PARP inhibitors can be identified if PARP is up-regulated. Further, such BRCA deficient patients can be treated with PARP inhibitors.

IGF1-R overexpression can be the result of loss of BRCA1 (Werner and Roberts, 2003, Genes, Chromo. Cancer 36:113-120; Riedemann and Macaulay, 2006, Endocr. Rel. Cancer, 13:Suppl 1:S33-S43). It was previously shown that BRCA1 can suppress IGF1-R promoter, and suggested that inactivation of BRCA1 can lead to activation of IGF1-R expression due to derepression of IGF1-R.

Activation of EGFR triggers mitotic signaling in gastrointestinal (G1) neoplasms, where prostaglandin E2 (PGE2) rapidly phosphorylates EGFR and triggers the extracellular signal-regulated kinase 2 (ERK2) mitogenic signaling in G1 cells and tumors. PARP1 can be activated via direct interaction with ERK2 that in turn can amplify ERK-signaling promoting growth, proliferation and differentiation regulated by the RAF-MEK-EREK signal transduction pathway (Cohen-Armon, 2007, Trends Pharmacol. Sci. 28:556-60 Epub).

Although IGF1-R overexpression and PARP1 upregulation are both seen in BRCA1 deficient breast cancers, previous studies have not shown or suggested any interrelationship between the two pathways in the treatment of breast cancer. The studies presented herein detail co-upregulation of PARP1 and IGFR-1 in a variety of tumors, including breast, endometrial mullerian mixed tumor, papillary serous type ovarian adenocarcinoma, ovarian mullerian mixed tumor and skin tumors (see Tables II-XVIII). Moreover, it has been previously shown that in the ovarian adenocarcinoma cell lines OVCAR-3 and OVCAR-4, the small molecule inhibitor NVP-AEW541 inhibited growth of the cells (Gotlieb et al., 2006, Gynecol. Oncol. 100:389-96). Accordingly, from the expression correlation tables as well as previous observations of IGF-1R's role in tumor growth and proliferation, treatment with PARP1 and IGF1R modulators may also increase sensitivity to chemotherapy of tumors treated by the combination of PARP and IGF1R inhibitors.

Similarly, PARP1 upregulation is also observed in the same subset of tumors where the upregulation of EGFR was also observed (see Tables II-XVIII, XXI). For example, co-upregulation of PARP1 and EGFR expression was seen in skin cancer, uterine cancer, breast and lung cancers, among others. (II-XVIII, XXI). Accordingly, treatment with PARP1 and EGFR may also increase sensitivity to chemotherapy of tumors treated by a combination of PARP1 and EGFR inhibitors.

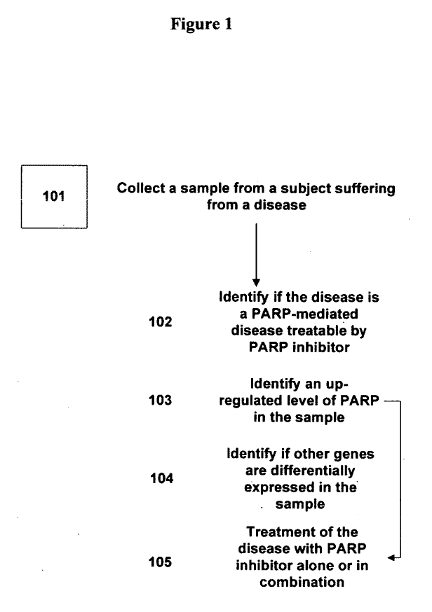

The steps to some embodiments are depicted in FIG. 1. Without limiting the scope of the present embodiments, the steps can be performed independent of each other or one after the other. One or more steps may be skipped in the methods described herein. A sample is collected from a subject suffering from a disease at step 101. In one embodiment, the sample is human normal and tumor samples, hair, blood, and other biofluids. A level of PARP is analyzed at step 102 by techniques well known in the art and based on the level of PARP such as, when PARP is up-regulated identifying the disease treatable by PARP inhibitors at step 103. Other co-regulated expressed genes are identified in step 104, where modulation of the identified co-regulated expressed genes may be used to treat the subject in step 105 suffering from the diseases identified with a combination of at least a PARP inhibitor and a modulator of the identified co-regulated expressed genes. It shall be understood that other methods contemplated not explicitly set forth herein. Without limiting the scope of the present embodiments, other techniques for collection of sample, analysis of PARP and co-regulated expressed genes in the sample and treatment of the disease with a combination of at least PARP inhibitors and modulators of the identified co-regulated expressed genes are known in the art and are within the scope of the present embodiments.

### Sample Collection, Preparation and Separation

Biological samples can be obtained from individuals with varying phenotypic states, such as various states of cancer or other diseases. Examples of phenotypic states also include phenotypes of normal subjects, which can be used for comparisons to diseased subjects. In some embodiments, subjects with disease are matched with control samples that are obtained from individuals who do not exhibit the disease. In yet other embodiments, subjects with disease may provide the control sample, for example, from a tissue or organ not affected by the disease.

Samples may be collected from a variety of sources from a mammal (e.g., a human), including a body fluid sample, or a tissue sample. Samples collected can be human normal and tumor samples, hair, blood, other biofluids, cells, tissues, organs or bodily fluids for example, but not limited to, brain tissue, blood, serum, sputum including saliva, plasma, nipple aspirants, synovial fluids, cerebrospinal fluids, sweat, urine, fecal matter, pancreatic fluid, trabecular fluid, cerebrospinal fluid, tears, bronchial lavage, swabbings, bronchial aspirants, semen, prostatic fluid, precervicular fluid, vaginal fluids, pre-ejaculate, etc. Suitable tissue samples include various types of tumor or cancer tissue, or organ tissue, such as those taken at biopsy.

The samples can be collected from individuals repeatedly over a longitudinal period of time (e.g., about once a day, once a week, once a month, biannually or annually). Obtaining numerous samples from an individual over a period of time can be used to verify results from earlier detections and/or to identify an alteration in biological pattern as a result of, for example, disease progression, drug treatment, etc.

Sample preparation and separation can involve any of the procedures, depending on the type of sample collected and/or analysis of the co-differentially expressed genes. Such procedures include, by way of example only, concentration, dilution, adjustment of pH, removal of high abundance polypeptides (e.g., albumin, gamma globulin, and transferin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g. aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation represents one method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermeable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermeable membrane based on size and charge, it renders electrodialysis useful for concentration, removal, or separation of electrolytes.

Separation and purification may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) represents one method for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) represents a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present embodiments include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

### Measuring Expression Levels of Regulated Genes

Levels of regulated expressed genes, including at least PARP, may be measured through assays detecting and quantitating nucleic acid, the expressed levels of protein in a subject's sample, or in the alternative, the level of activity of the co-regulated expressed genes or proteins in a subject's sample. For example, a practitioner may measure the expression levels of the regulated expressed genes through mRNA quantification. The most commonly used methods known in the art for the quantification of mRNA expression in a sample include northern blotting and in situ hybridization; RNAse protection assays; and PCR-based methods, such as reverse transcription polymerase chain reaction (RT-PCR). Alternatively, antibodies may be employed that can recognize specific duplexes, including DNA duplexes, RNA duplexes, and DNA-RNA hybrid duplexes or DNA-protein duplexes.

Representative methods for sequencing-based gene expression analysis include Serial Analysis of Gene Expression (SAGE), and gene expression analysis by massively parallel signature sequencing (MPSS), Comparative Genome Hybridization (CGH), Chromatin Immunoprecipitation (ChIP), Single nucleotide polymorphism (SNP) and SNP arrays, Fluorescent in situ Hybridization (FISH), Protein binding arrays, DNA microarray (also commonly known as gene or genome chip, DNA chip, or gene array), and RNA microarrays. As mentioned above, co-regulated levels of protein expression or protein activity may also be monitored and compared against reference levels.

In some embodiments, the level of regulated expressed genes, including at least PARP, in a sample from a patient is compared to a predetermined standard sample. The sample from the patient is typically from a diseased tissue, such as cancer cells or tissues. The standard sample can be from the same patient or from a different subject. The standard sample is typically a normal, non-diseased sample. However, in some embodiments, such as for staging of disease or for evaluating the efficacy of treatment, the standard sample is from a diseased tissue. The standard sample can be a combination of samples from several different subjects. In some embodiments, the level of co-regulated expressed genes, including at least PARP, from a patient is compared to a pre-determined level. This pre-determined level is typically obtained from normal samples. As described herein, a “pre-determined expression level” may be a level of expression of a panel of genes, including at least PARP, used to, by way of example only, evaluate a patient that may be selected for treatment, evaluate a response to a PARP inhibitor treatment, evaluate a response to a combination of a PARP inhibitor and a second therapeutic agent treatment, for example, modulators to co-regulated expressed genes, and/or diagnose a patient for cancer, inflammation, pain and/or related conditions. In other embodiments, a pre-determined level of expression for a panel of genes, including at least PARP, may be determined in populations of patients with or without cancer. The pre-determined expression levels for each identified gene, including at least PARP, can be a single number, equally applicable to every patient, or the pre-determined expression levels for each gene in a panel can vary according to specific subpopulations of patients. For example, men might have different pre-determined expression levels than women; non-smokers may have a different pre-determined expression level than smokers. Age, weight, and height of a patient may affect the pre-determined expression levels of the individual or of a designated patient population or sub-population. Furthermore, the pre-determined expression levels can be a level determined for each patient individually. The pre-determined expression level can be any suitable standard. For example, the pre-determined expression level can be obtained from the same or a different human for whom a patient selection is being assessed. In one embodiment, the pre-determined expression level can be obtained from a previous assessment of the same patient. In such a manner, the progress of the selection of the patient can be monitored over time. Similarly, the pre-determined expression levels of a panel of gene targets, including at least PARP, can be from a specific patient population or subpopulations. Accordingly, the standard can be obtained from an assessment of another human or multiple humans, e.g., selected groups of humans. In such a manner, the extent of the selection of the human for whom selection is being assessed can be compared to suitable other humans, e.g., other humans who are in a similar situation to the human of interest, such as those suffering from similar or the same condition(s).

In some embodiments the change of expression levels of each gene in a panel of gene targets identified from the pre-determined level is about 0.5 fold, about 1.0 fold, about 1.5 fold, about 2.0 fold, about 2.5 fold, about 3.0 fold, about 3.5 fold, about 4.0 fold, about 4.5 fold, or about 5.0 fold. In some embodiments the fold change is less than about 1, less than about 5, less than about 10, less than about 20, less than about 30, less than about 40, or less than about 50. In other embodiments, the changes in expression levels compared to a predetermined level is more than about 1, more than about 5, more than about 10, more than about 20, more than about 30, more than about 40, or more than about 50. Fold changes from a pre-determined level also include about 0.5, about 1.0, about 1.5, about 2.0, about 2.5, and about 3.0.

Tables I to XVII as shown below illustrate differential gene expression data, including PARP1 and other gene expression profiles, in subjects suffering from cancer, metabolic diseases, endocrine and neuroendocrine system disorders, cardiovascular diseases (CVS), central nervous system diseases (CNS), diseases of male reproductive system, diseases of female reproductive system, respiratory system, disorders of urinary tract, inflammation, hematolymphoid system, and disorders of digestive system. The minimum expression fold change for representation in tables I to XVII is at least a 2-fold change.

Provided herein is a monitoring method in which the expression level of each co-regulated identified gene, including at least PARP, in cancer patients or populations can be monitored during the course of cancer or anti-neoplastic treatment, and also in some cases, prior to and at the start of treatment. The determination of a decrease or increase in the expression levels of each identified gene target in a pre-determined panel of co-regulated genes in a cancer patient or population, compared to the expression levels of the same pre-determined panel of co-regulated genes in normal individuals without cancer allows the following evaluation related to patient progression and/or outcome: (i) a more severe stage or grade of the cancer; (ii) shorter time to disease progression, and/or (iii) lack of a positive, i.e., effective, response by the patient to the cancer treatment. For example, based on the monitoring of a patient's expression levels over time relative to normal levels of the same panel of gene targets, or in addition to or in the alternative, as to the patient's own prior-determined levels, a determination can be made as to whether a treatment regimen should be changed, i.e., to be more aggressive or less aggressive; to determine if the patient is responding favorably to his or her treatment; and/or to determine disease status, such as advanced stage or phase of the cancer, or a remission, reduction or regression of the cancer or neoplastic disease. The embodiments allow a determination of clinical benefit, time to progression (TTP), and length of survival time based upon the findings of up-regulated or down-regulated co-regulated gene expression levels in the predetermined panel compared to the levels in normal individuals.

The analysis of expression levels of genes and their pathways in individual patients or patient populations is particularly valuable and informative, as it allows a physician to more effectively select the best treatments, as well as to utilize more aggressive treatments and therapy regimens based on the up-regulated or down-regulated level of the identified co-regulated gene targets. More aggressive treatment, or combination treatments and regimens, can serve to counteract poor patient prognosis and overall survival time. Armed with this information, the medical practitioner can choose to provide certain types of treatment such as treatment with PARP inhibitors and/or modulators of other co-regulated expressed genes, and/or more aggressive therapy.

In monitoring an individual patient or patient population's co-regulated gene expression levels, including at least PARP, over a period of time, which may be minutes, hours, days, weeks, months, and in some cases, years, or various intervals thereof, the patient or patient population's body fluid samples, e.g., serum or plasma, can be collected at intervals, as determined by the practitioner, such as a physician or clinician, to determine the expression levels of each identified co-regulated target gene, including at least PARP, and compared to the levels in normal individuals or population over the course or treatment or disease. For example, patient samples can be taken and monitored every month, every two months, or combinations of one, two, or three month intervals. In addition, expression levels of each identified target co-regulated gene, including at least PARP, of the patient obtained over time can be conveniently compared with each other, as well as with the expression level values, of normal controls, during the monitoring period, thereby providing the patient's own level of expression values, as an internal, or personal, control for long-term expression monitoring. Similarly, expression levels from a patient population may also be compared with other populations, including a normal control population, providing a convenient means to compare the patient population results over the course of the monitoring period.

TABLE II
PARP1 Upregulated - Diff/X (Human); Name: Upreg Skin Basal Cell Carcinoma Primary (Minimum
Fold Change: 2.0); Experiment: Skin, Basal Cell Carcinoma, Primary; Control: normal skin.

| Fragment | | | | | | Fold | | |
|---|---|---|---|---|---|---|---|---|
| Name | Array | Pathway | Symbol | Description | Pres. Freq. | Change | t-Score | p-Value |
| 225431_x_at | hg133b | | ACY1L2 | aminoacylase 1-like 2 | 0.871158 | 2.291582 | 4.924157 | 9.99E−03 |
| 203044_at | hg133a | | CHSY1 | carbohydrate | 0.999101 | 2.309728 | 8.533783 | 2.10E−03 |
| | | | | (chondroitin) synthase 1 | | | | |
| 218062_x_at | hg133a | (Rho GTPase | CDC42EP4 | CDC42 effector protein | 0.931985 | 2.191675 | 5.744328 | 6.83E−03 |
| | | pathway | | (Rho GTPase binding) 4 | | | | |
| 224736_at | hg133b | | CCAR1 | cell division cycle and | 0.882365 | 2.692955 | 6.069085 | 5.27E−03 |
| | | | | apoptosis regulator 1 | | | | |
| 204620_s_at | hg133a | | CSPG2 | chondroitin sulfate | 0.893963 | 2.192021 | 4.798746 | 1.36E−02 |
| | | | | proteoglycan 2 | | | | |
| | | | | (versican) | | | | |
| 203917_at | hg133a | | CXADR | coxsackie virus and | 0.83738 | 2.493983 | 8.448274 | 1.66E−03 |
| | | | | adenovirus receptor | | | | |
| 228906_at | hg133b | | CXXC6 | CXXC finger 6 | 0.827674 | 2.252518 | 5.907488 | 6.59E−03 |
| 224847_at | hg133b | cyclin- | CDK6 | 0.741578 | Skin, Basal | 2.091715 | 5.103405 | 6.49E−03 |
| | | dependent | | | Cell | | | |
| | | kinase | | | Carcinoma, | | | |
| | | | | | Primary | | | |
| 202887_s_at | hg133a | DNA | DDIT4 | DNA-damage-inducible | 0.888889 | 2.653243 | 4.706684 | 5.16E−03 |
| | | damage | | transcript 4 | | | | |
| 212070_at | hg133a | GPCR | GPR56 | G protein-coupled | 0.797302 | 2.223807 | 5.063972 | 1.22E−02 |
| | | | | receptor 56 | | | | |
| 211969_at | hg133a | heat shock | HSPCA | heat shock 90 kDa | 0.997945 | 2.115775 | 6.139984 | 2.37E−03 |
| | | | | protein 1, alpha | | | | |
| 211969_at | hg133a | heat shock | HSPCAL3 | heat shock 90 kDa | 0.997945 | 2.115775 | 6.139984 | 2.37E−03 |
| | | | | protein 1, alpha-like 3 | | | | |
| 203284_s_at | hg133a | | HS2ST1 | heparan sulfate 2-O- | 0.889403 | 3.007556 | 7.610417 | 3.79E−03 |
| | | | | sulfotransferase 1 | | | | |
| 209031_at | hg133a | | IGSF4 | immunoglobulin | 0.762171 | 4.037252 | 7.950069 | 3.81E−03 |
| | | | | superfamily, member 4 | | | | |
| 200914_x_at | hg133a | | KTN1 | kinectin 1 (kinesin | 0.956262 | 3.367935 | 5.192132 | 1.22E−02 |
| | | | | receptor) | | | | |
| 226350_at | hg133b | | KMO | kynurenine 3- | 0.850758 | 2.753275 | 4.463178 | 1.34E−02 |
| | | | | monooxygenase | | | | |
| | | | | (kynurenine 3- | | | | |
| | | | | hydroxylase) | | | | |
| 225897_at | hg133b | myristoylated | myristoylated | 0.931217 | Skin, Basal | 2.388145 | 5.917078 | 7.77E−03 |
| | | alanine- | alanine- | | Cell | | | |
| | | rich protein | rich | | Carcinoma, | | | |
| | | kinase | protein | | Primary | | | |
| | | pathway | kinase C | | | | | |
| | | | substrate | | | | | |
| | | | (MARCKS) | | | | | |
| 202784_s_at | hg133a | | NNT | nicotinamide nucleotide | 0.745151 | 2.192563 | 4.837296 | 1.29E−02 |
| | | | | transhydrogenase | | | | |
| 222688_at | hg133b | | PHCA | phytoceramidase, | 0.961482 | 3.003031 | 6.463953 | 6.62E−03 |
| | | | | alkaline | | | | |
| 213655_at | hg133a | | PAFAH1B1 | platelet-activating factor | 0.999101 | 2.156965 | 5.4083 | 8.03E−03 |
| | | | | acetylhydrolase, isoform | | | | |
| | | | | Ib, alpha subunit 45 kDa | | | | |
| 221958_s_at | hg133a | NFkB | FLJ23091 | putative NFkB activating | 0.828838 | 2.299807 | 6.598901 | 5.26E−03 |
| | | pathway | | protein 373 | | | | |
| 204127_at | hg133a | DNA repair | RFC3 | replication factor C | 0.90578 | 2.108509 | 5.81375 | 5.66E−03 |
| | | | | (activator 1) 3, 38 kDa | | | | |
| 217301_x_at | hg133a | | RBBP4 | retinoblastoma binding | 0.982916 | 2.152989 | 6.361229 | 6.83E−03 |
| | | | | protein 4 | | | | |
| 212560_at | hg133a | | SORL1 | sortilin-related receptor, | 0.941169 | 6.344758 | 5.988721 | 9.04E−03 |
| | | | | L(DLR class) A repeats- | | | | |
| | | | | containing | | | | |
| 213655_at | hg133a | | YWHAE | tyrosine 3- | 0.999101 | 2.156965 | 5.4083 | 8.03E−03 |
| | | | | monooxygenase/tryptophan | | | | |
| | | | | 5-monooxygenase | | | | |
| | | | | activation protein, | | | | |
| | | | | epsilon polypeptide | | | | |
| 223701_s_at | hg133b | Ubiquitin | USP47 | ubiquitin specific | 0.816333 | 2.171685 | 6.634333 | 2.65E−03 |
| | | pathway | | protease 47 | | | | |
| 202779_s_at | hg133a | Ubiquitin | UBE2S | ubiquitin-conjugating | 0.743224 | 12.644663 | 4.360135 | 6.28E−03 |
| | | pathway | | enzyme E2S | | | | |

TABLE III
PARP1 Upregulated - Diff/X (Human); Name: Upreg Skin Malignant Melanoma Primary (Minimum
Fold Change: 2.0); Experiment: Skin, Malignant Melanoma, Primary; control: normal skin.

| Fragment | | | | | Pres. | Fold | | p- |
|---|---|---|---|---|---|---|---|---|
| Name | Array | Pathway | Symbol | Description | Freq. | Change | t-Score | Value |
| 234464_s_at | hg133b | | EME1 | essential meiotic | 0.916119 | 2.319178 | 3.522606 | 1.20E−02 |
| | | | | endonuclease 1 | | | | |
| | | | | homolog 1 (S. pombe) | | | | |
| 201178_at | hg133a | | FBXO7 | F-box protein 7 | 0.999486 | 2.095026 | 3.412365 | 1.40E−02 |
| 222140_s_at | hg133a | GPCR | GPR89 | G protein-coupled | 0.727232 | 2.148837 | 3.631779 | 1.01E−02 |
| | | | | receptor 89 | | | | |
| 211934_x_at | hg133a | | GANAB | glucosidase, alpha; | 0.804689 | 2.25914 | 3.381036 | 1.34E−02 |
| | | | | neutral AB | | | | |
| 200806_s_at | hg133a | Heat | HSPD1 | heat shock 60 kDa | 0.970328 | 3.17649 | 3.851348 | 7.87E−03 |
| | | Shock | | protein 1 (chaperonin) | | | | |
| 210338_s_at | hg133a | Heat | HSPA8 | heat shock 70 kDa | 0.987925 | 2.013615 | 5.278478 | 1.39E−03 |
| | | Shock | | protein 8 | | | | |
| 204544_at | hg133a | | HPS5 | Hermansky-Pudlak | 0.931214 | 2.249752 | 3.564928 | 1.14E−02 |
| | | | | syndrome 5 | | | | |
| 201030_x_at | hg133a | | LDHB | lactate dehydrogenase B | 0.986641 | 3.044588 | 3.83266 | 8.28E−03 |
| 203362_s_at | hg133a | | MAD2L1 | MAD2 mitotic arrest | 0.808863 | 3.707529 | 3.331468 | 1.39E−02 |
| | | | | deficient-like 1 (yeast) | | | | |
| 218211_s_at | hg133a | | MLPH | melanophilin | 0.982852 | 5.972088 | 3.659612 | 1.05E−02 |
| 202905_x_at | hg133a | | NBS1 | Nijmegen breakage | 0.886127 | 2.053746 | 3.993535 | 5.70E−03 |
| | | | | syndrome 1 (nibrin) | | | | |
| 223158_s_at | hg133b | Kinase | NEK6 | NIMA (never in | 0.817675 | 2.85397 | 3.884645 | 7.47E−03 |
| | | | | mitosis gene a)-related | | | | |
| | | | | kinase 6 | | | | |
| 201577_at | hg133a | | NME1 | non-metastatic cells 1, | 0.997559 | 2.20045 | 6.811967 | 2.53E−04 |
| | | | | protein (NM23A) | | | | |
| | | | | expressed in | | | | |
| 218039_at | hg133a | | NUSAP1 | nucleolar and spindle | 0.920938 | 2.689121 | 3.568467 | 9.76E−03 |
| | | | | associated protein 1 | | | | |
| 201013_s_at | hg133a | | PAICS | phosphoribosylaminoimidazole | 0.993706 | 3.31606 | 3.456283 | 1.34E−02 |
| | | | | carboxylase, | | | | |
| | | | | phosphoribosylaminoimidazole | | | | |
| | | | | succinocarboxamide | | | | |
| | | | | synthetase | | | | |
| 201274_at | hg133a | Proteosome | PSMA5 | proteasome (prosome, | 0.894348 | 2.021292 | 4.325387 | 4.64E−03 |
| | | | | macropain) subunit, | | | | |
| | | | | alpha type, 5 | | | | |
| 204127_at | hg133a | DNA | RFC3 | replication factor C | 0.90578 | 2.031709 | 6.485772 | 1.85E−04 |
| | | replication | | (activator 1) 3, 38 kDa | | | | |
| | | and repair | | | | | | |
| 200903_s_at | hg133a | | AHCY | S- | 0.994348 | 2.026971 | 4.353137 | 3.41E−03 |
| | | | | adenosylhomocysteine | | | | |
| | | | | hydrolase | | | | |
| 201664_at | hg133a | | SMC4L1 | SMC4 structural | 0.975915 | 2.251509 | 3.464165 | 1.26E−02 |
| | | | | maintenance of | | | | |
| | | | | chromosomes 4-like 1 | | | | |
| | | | | (yeast) | | | | |
| 230333_at | hg133b | | SAT | spermidine/spermine | 0.9796 | 3.405015 | 4.505068 | 3.38E−03 |
| | | | | N1-acetyltransferase | | | | |
| 202589_at | hg133a | | TYMS | thymidylate synthetase | 0.919332 | 4.582056 | 7.148353 | 3.31E−04 |
| | | | | Inhibitor: 5- | | | | |
| | | | | fluorouracil, 5-fluoro- | | | | |
| | | | | 2-prime-deoxyuridine, | | | | |
| | | | | and some folate | | | | |
| | | | | analogs | | | | |
| 208699_x_at | hg133a | | TKT | transketolase | 0.933398 | 2.009008 | 3.942088 | 5.05E−03 |
| | | | | (Wernicke-Korsakoff | | | | |
| | | | | syndrome) | | | | |
| 216449_x_at | hg133a | | TRA1 | tumor rejection | 0.761786 | 2.622949 | 4.141308 | 4.22E−03 |
| | | | | antigen (gp96) 1 | | | | |

TABLE IV
PARP1 Upregulated - Diff/X (Human); Name: Upregul Thyroid Gland Papillary Carcinoma Follicular
Variant Primary (Minimum Fold Change: 2.0); Experiment: Thyroid Gland, Papillary Carcinoma,
Follicular Variant, Primary; Control: normal thyroid gland.

| Fragment | | | | | Pres. | Fold | | |
|---|---|---|---|---|---|---|---|---|
| Name | Array | Pathway | Symbol | Description | Freq. | Change | t-Score | p-Value |
| 231793_s_at | hg133b | Kinase | CAMK2D | calcium/calmodulin- | 0.743122 | 2.1041 | 5.161011 | 5.67E−04 |
| | | | | dependent protein kinase | | | | |
| | | | | (CaM kinase) II delta | | | | |
| 213274_s_at | hg133a | | CTSB | cathepsin B | 0.999486 | 2.172113 | 4.678528 | 1.15E−03 |
| 208892_s_at | hg133a | epidermal | DUSP6 | dual specificity | 0.971098 | 2.055812 | 3.367706 | 9.13E−03 |
| | | growth | | phosphatase 6 | | | | |
| | | factor | | | | | | |
| | | receptor | | | | | | |
| | | pathway | | | | | | |
| 202609_at | hg133a | | EPS8 | epidermal growth factor | 0.884843 | 2.145576 | 2.983337 | 1.94E−02 |
| | | | | receptor pathway | | | | |
| | | | | substrate 8 | | | | |
| 215719_x_at | hg133a | | FAS | Fas (TNF receptor | 0.818176 | 2.05139 | 3.21089 | 1.26E−02 |
| | | | | superfamily, member 6) | | | | |
| 220189_s_at | hg133a | | MGAT4B | mannosyl (alpha-1,3-)- | 0.943353 | 2.020894 | 3.452749 | 9.43E−03 |
| | | | | glycoprotein beta-1,4-N- | | | | |
| | | | | acetylglucosaminyltransferase, | | | | |
| | | | | isoenzyme B | | | | |
| 219628_at | hg133a | | WIG1 | p53 target zinc finger | 0.916506 | 2.432043 | 3.727846 | 4.88E−03 |
| | | | | protein | | | | |
| 217744_s_at | hg133a | | PERP | PERP, TP53 apoptosis | 0.78754 | 2.141354 | 3.683438 | 6.76E−03 |
| | | | | effector | | | | |
| 201050_at | hg133a | | PLD3 | phospholipase D3 | 0.871933 | 2.033581 | 3.297074 | 1.15E−02 |
| 211503_s_at | hg133a | RAS | RAB14 | RAB14, member RAS | 0.97887 | 2.068063 | 3.147796 | 1.36E−02 |
| | | oncogene | | oncogene family | | | | |
| | | pathway | | | | | | |
| 222412_s_at | hg133b | | SSR3 | signal sequence receptor, | 0.818883 | 2.06322 | 2.94228 | 1.96E−02 |
| | | | | gamma (translocon- | | | | |
| | | | | associated protein | | | | |
| | | | | gamma) | | | | |
| 203217_s_at | hg133a | | ST3GAL5 | ST3 beta-galactoside | 0.816635 | 2.006942 | 4.210143 | 3.44E−03 |
| | | | | alpha-2,3-sialyltransferase 5 | | | | |
| 214196_s_at | hg133a | | TPP1 | tripeptidyl peptidase I | 0.837765 | 2.042539 | 3.456908 | 8.79E−03 |

TABLE V
PARP1 Upregulated - Diff/X (Human); Name: Upreg Testis Seminoma Primary
(Minimum Fold Change: 2.0); Experiment: Testis, Seminoma, Primary; Control: normal testis.

| Fragment | | | | | Pres. | Fold | | |
|---|---|---|---|---|---|---|---|---|
| Name | Array | Pathway | Symbol | Description | Freq. | Change | t-Score | p-Value |
| 226617_at | hg133b | ADP- | ARL5 | ADP-ribosylation factor- | 0.957187 | 2.24151 | 3.167936 | 7.52E−03 |
| | | ribosylation | | like 5 | | | | |
| 215783_s_at | hg133a | | ALPL | alkaline phosphatase, | 0.758574 | 3.643008 | 2.976099 | 1.32E−02 |
| | | | | liver/bone/kidney | | | | |
| 202511_s_at | hg133a | autophagy | APG5L | APG5 autophagy | 0.990751 | 2.059948 | 3.4546 | 4.27E−03 |
| | | | | 5-like (S. cerevisiae) | | | | |
| 208270_s_at | hg133a | | RNPEP | arginyl aminopeptidase | 0.847913 | 2.05514 | 3.701297 | 6.15E−03 |
| | | | | (aminopeptidase B) | | | | |
| 226785_at | hg133b | ATPase | ATP11C | ATPase, Class VI, type | 0.844987 | 2.64688 | 3.700799 | 3.91E−03 |
| | | | | 11C | | | | |
| 203981_s_at | hg133a | | ABCD4 | ATP-binding cassette, sub- | 0.74104 | 2.248088 | 3.09927 | 1.02E−02 |
| | | | | family D (ALD), member 4 | | | | |
| 34726_at | hg133a | | CACNB3 | calcium channel, voltage- | 0.736866 | 2.538816 | 3.12979 | 9.40E−03 |
| | | | | dependent, beta 3 subunit | | | | |
| 226545_at | hg133b | | CD109 | CD109 antigen (Gov | 0.790565 | 2.550122 | 3.275845 | 9.58E−03 |
| | | | | platelet alloantigens) | | | | |
| 221556_at | hg133a | | CDC14B | CDC14 cell division cycle | 0.893513 | 2.290001 | 3.966765 | 2.81E−03 |
| | | | | 14 homolog B (S. cerevisiae) | | | | |
| 228906_at | hg133b | | CXXC6 | CXXC finger 6 | 0.827674 | 4.3978 | 3.327614 | 6.85E−03 |
| 204256_at | hg133a | | ELOVL6 | ELOVL family member 6, | 0.937058 | 4.531694 | 3.633481 | 5.95E−03 |
| | | | | elongation of long chain | | | | |
| | | | | fatty acids (FEN1/Elo2, | | | | |
| | | | | SUR4/Elo3-like, yeast) | | | | |
| 209409_at | hg133a | | GRB10 | growth factor receptor- | 0.960244 | 2.158927 | 3.299707 | 6.07E−03 |
| | | | | bound protein 10 | | | | |
| 214359_s_at | hg133a | Heat | HSPCB | heat shock 90 kDa protein | 0.976814 | 2.560546 | 4.198958 | 1.31E−03 |
| | | shock | | 1, beta | | | | |
| 203607_at | hg133a | | INPP5F | inositol polyphosphate-5- | 0.876108 | 2.446044 | 3.404642 | 5.77E−03 |
| | | | | phosphatase F | | | | |
| 221841_s_at | hg133a | | KLF4 | Kruppel-like factor 4 (gut) | 0.880925 | 2.586 | 3.030116 | 9.93E−03 |
| 225997_at | hg133b | | MOBKL1A | MOB1, Mps One Binder | 0.961616 | 2.714915 | 3.42853 | 9.30E−03 |
| | | | | kinase activator-like 1A | | | | |
| | | | | (yeast) | | | | |
| 209421_at | hg133a | DNA | MSH2 | mutS homolog 2, colon | 0.807964 | 2.633251 | 3.433425 | 4.82E−03 |
| | | repair | | cancer, nonpolyposis type | | | | |
| | | | | 1 (E. coli) | | | | |
| 200827_at | hg133a | | PLOD1 | procollagen-lysine 1,2- | 0.856005 | 2.296081 | 4.426219 | 1.38E−03 |
| | | | | oxoglutarate 5- | | | | |
| | | | | dioxygenase 1 | | | | |
| 202006_at | hg133a | | PTPN12 | protein tyrosine | 0.885613 | 2.633664 | 3.085259 | 8.69E−03 |
| | | | | phosphatase, non-receptor | | | | |
| | | | | type 12 | | | | |
| 224603_at | hg133b | | ST6GALNAC2 | ST6 (alpha-N-acetyl- | 0.98517 | 2.332539 | 3.433029 | 7.12E−03 |
| | | | | neuraminyl-2,3-beta- | | | | |
| | | | | galactosyl-1,3)-N- | | | | |
| | | | | acetylgalactosaminide | | | | |
| | | | | alpha-2,6-sialyltransferase 2 | | | | |
| 212157_at | hg133a | | SDC2 | syndecan 2 (heparan | 0.962171 | 2.114359 | 4.043681 | 1.40E−03 |
| | | | | sulfate proteoglycan 1, cell | | | | |
| | | | | surface-associated, | | | | |
| | | | | fibroglycan) | | | | |
| 213135_at | hg133a | | TIAM1 | T-cell lymphoma invasion | 0.934297 | 2.424542 | 3.85277 | 2.37E−03 |
| | | | | and metastasis 1 | | | | |
| 217979_at | hg133a | | TSPAN13 | tetraspanin 13 | 0.973732 | 2.244878 | 3.049012 | 1.07E−02 |
| 202454_s_at | hg133a | HER3 | ERBB3 | v-erb-b2 erythroblastic | 0.861207 | 2.274337 | 3.104892 | 8.73E−03 |
| | | | | leukemia viral oncogene | | | | |
| | | | | homolog 3 (avian) | | | | |

TABLE VI
PARP1 Upregulated - Diff/X (Human); Name: Upregulated Lung Adenocarcinoma Primary
(Minimum Fold Change: 2.0); Experiment: Lung, Adenocarcinoma, Primary; Control: normal lung.

| Fragment | | | | | Pres. | Fold | | |
|---|---|---|---|---|---|---|---|---|
| Name | Array | Pathway | Symbol | Description | Freq. | Change | t-Score | p-Value |
| 222416_at | hg133b | | ALDH18A1 | aldehyde dehydrogenase | 0.73923 | 2.360222 | 9.196847 | 5.71E−13 |
| | | | | 18 family, member A1 | | | | |
| 216594_x_at | hg133a | | AKR1C1 | aldo-keto reductase family | 0.935517 | 4.329887 | 3.505597 | 1.04E−03 |
| | | | | 1, member C1 | | | | |
| | | | | (dihydrodiol | | | | |
| | | | | dehydrogenase 1; 20- | | | | |
| | | | | alpha (3-alpha)- | | | | |
| | | | | hydroxysteroid | | | | |
| | | | | dehydrogenase) | | | | |
| 216594_x_at | hg133a | | AKR1C2 | aldo-keto reductase family | 0.935517 | 4.329887 | 3.505597 | 1.04E−03 |
| | | | | 1, member C2 | | | | |
| | | | | (dihydrodiol | | | | |
| | | | | dehydrogenase 2; bile | | | | |
| | | | | acid binding protein; 3- | | | | |
| | | | | alpha hydroxysteroid | | | | |
| | | | | dehydrogenase, type III) | | | | |
| 209160_at | hg133a | | AKR1C3 | aldo-keto reductase family | 0.77386 | 2.942353 | 3.275998 | 2.01E−03 |
| | | | | 1, member C3 (3-alpha | | | | |
| | | | | hydroxysteroid | | | | |
| | | | | dehydrogenase, type II) | | | | |
| 209186_at | hg133a | | ATP2A2 | ATPase, Ca++ | 0.999294 | 2.100639 | 8.219938 | 3.19E−11 |
| | | | | transporting, cardiac | | | | |
| | | | | muscle, slow twitch 2 | | | | |
| 201242_s_at | hg133a | | ATP1B1 | ATPase, Na+/K+ | 0.975787 | 2.447242 | 4.588835 | 3.28E−05 |
| | | | | transporting, beta 1 | | | | |
| | | | | polypeptide | | | | |
| 201117_s_at | hg133a | | CPE | carboxypeptidase E | 0.77842 | 2.280145 | 3.410716 | 1.35E−03 |
| 266_s_at | hg133a | | CD24 | CD24 antigen (small cell | 0.724663 | 2.19691 | 3.862407 | 3.09E−04 |
| | | | | lung carcinoma cluster 4 | | | | |
| | | | | antigen) | | | | |
| 201897_s_at | hg133a | Kinase | CKS1B | CDC28 protein kinase | 0.761593 | 2.561978 | 5.329644 | 2.70E−06 |
| | | | | regulatory subunit 1B | | | | |
| 219429_at | hg133a | Fatty acid | FA2H | fatty acid 2-hydroxylase | 0.715414 | 2.605507 | 4.52537 | 3.94E−05 |
| | | pathway | | | | | | |
| 202923_s_at | hg133a | | GCLC | glutamate-cysteine ligase, | 0.796981 | 3.165989 | 3.762128 | 4.71E−04 |
| | | | | catalytic subunit | | | | |
| 202722_s_at | hg133a | | GFPT1 | glutamine-fructose-6- | 0.894541 | 2.217797 | 7.10894 | 2.94E−09 |
| | | | | phosphate transaminase 1 | | | | |
| 210095_s_at | hg133a | | IGFBP3 | insulin-like growth factor | 0.80501 | 3.165953 | 6.366044 | 6.07E−08 |
| | | | | binding protein 3 | | | | |
| 210046_s_at | hg133a | | IDH2 | isocitrate dehydrogenase 2 | 0.971034 | 2.306479 | 5.481501 | 1.46E−06 |
| | | | | (NADP+), mitochondrial | | | | |
| 226350_at | hg133b | | KMO | kynurenine 3- | 0.850758 | 2.651165 | 4.629216 | 2.83E−05 |
| | | | | monooxygenase | | | | |
| | | | | (kynurenine 3- | | | | |
| | | | | hydroxylase) | | | | |
| 218326_s_at | hg133a | | LGR4 | leucine-rich repeat- | 0.821451 | 3.055142 | 6.545887 | 3.21E−08 |
| | | | | containing G protein- | | | | |
| | | | | coupled receptor 4 | | | | |
| 217871_s_at | hg133a | | MIF | macrophage migration | 0.995633 | 2.174974 | 7.583763 | 2.79E−10 |
| | | | | inhibitory factor | | | | |
| | | | | (glycosylation-inhibiting | | | | |
| | | | | factor) | | | | |
| 222036_s_at | hg133a | DNA | MCM4 | MCM4 minichromosome | 0.878035 | 2.442457 | 5.757639 | 2.87E−07 |
| | | replication | | maintenance deficient 4 | | | | |
| | | | | (S. cerevisiae) | | | | |
| 201761_at | hg133a | | MTHFD2 | methylenetetrahydrofolate | 0.752922 | 2.054339 | 6.621109 | 1.60E−08 |
| | | | | dehydrogenase (NADP+ | | | | |
| | | | | dependent) 2, | | | | |
| | | | | methenyltetrahydrofolate | | | | |
| | | | | cyclohydrolase | | | | |
| 210519_s_at | hg133a | | NQO1 | NAD(P)H dehydrogenase, | 0.744894 | 5.024633 | 4.67042 | 2.67E−05 |
| | | | | quinone 1 | | | | |
| 200790_at | hg133a | | ODC1 | ornithine decarboxylase 1 | 0.934682 | 2.222311 | 3.499919 | 1.05E−03 |
| 201037_at | hg133a | | PFKP | phosphofructokinase, | 0.953565 | 2.939554 | 6.307969 | 9.17E−08 |
| | | | | platelet | | | | |
| 210145_at | hg133a | | PLA2G4A | phospholipase A2, group | 0.773796 | 4.288454 | 4.280026 | 9.58E−05 |
| | | | | IVA (cytosolic, calcium- | | | | |
| | | | | dependent) | | | | |
| 201013_s_at | hg133a | | PAICS | phosphoribosylaminoimid | 0.993706 | 2.573663 | 6.444726 | 3.79E−08 |
| | | | | azole carboxylase, | | | | |
| | | | | phosphoribosylaminoimid | | | | |
| | | | | azole succinocarboxamide | | | | |
| | | | | synthetase | | | | |
| 223062_s_at | hg133b | | PSAT1 | phosphoserine | 0.818749 | 3.26373 | 4.234361 | 5.73E−05 |
| | | | | aminotransferase 1 | | | | |
| 202619_s_at | hg133a | | PLOD2 | procollagen-lysine, 2- | 0.787219 | 2.482714 | 4.077228 | 1.64E−04 |
| | | | | oxoglutarate 5- | | | | |
| | | | | dioxygenase 2 | | | | |
| 211048_s_at | hg133a | | PDIA4 | protein disulfide | 0.803982 | 2.463043 | 7.209904 | 3.10E−09 |
| | | | | isomerase-associated 4 | | | | |
| 207668_x_at | hg133a | | PDIA6 | protein disulfide | 0.999936 | 2.068824 | 8.880199 | 3.92E−12 |
| | | | | isomerase-associated 6 | | | | |
| 226452_at | hg133b | | PDK1 | pyruvate dehydrogenase | 0.950745 | 2.576125 | 6.623535 | 1.59E−08 |
| | | | | kinase, isoenzyme 1 | | | | |
| 222750_s_at | hg133b | | SRD5A2L | steroid 5 alpha-reductase | 0.928332 | 2.329336 | 6.340054 | 3.85E−08 |
| | | | | 2-like | | | | |
| 204675_at | hg133a | | SRD5A1 | steroid-5-alpha-reductase, | 0.813809 | 3.255304 | 6.055318 | 2.12E−07 |
| | | | | alpha polypeptide 1 (3- | | | | |
| | | | | oxo-5 alpha-steroid delta | | | | |
| | | | | 4-dehydrogenase alpha 1) | | | | |
| 202589_at | hg133a | | TYMS | thymidylate synthetase; | 0.919332 | 2.654734 | 5.425874 | 8.53E−07 |
| | | | | Inhibitor: 5-fluorouracil, | | | | |
| | | | | 5-fluoro-2-prime- | | | | |
| | | | | deoxyuridine, and some | | | | |
| | | | | folate analogs | | | | |
| 202779_s_at | hg133a | Ubiquitin/ | UBE2S | ubiquitin-conjugating | 0.743224 | 2.133196 | 3.240654 | 1.76E−03 |
| | | proteosome | | enzyme E2S | | | | |
| 203343_at | hg133a | | UGDH | UDP-glucose | 0.808092 | 2.65764 | 4.497994 | 4.50E−05 |
| | | | | dehydrogenase | | | | |
| 218313_s_at | hg133a | | GALNT7 | UDP-N-acetyl-alpha-D- | 0.90578 | 2.355037 | 6.486027 | 7.40E−09 |
| | | | | galactosamine:polypeptide | | | | |
| | | | | N- | | | | |
| | | | | acetylgalactosaminyltransferase | | | | |
| | | | | 7 (GalNAc-T7) | | | | |
| 231008_at | hg133b | | UNC5CL | unc-5 homolog C (C. elegans)- | 0.870823 | 2.400073 | 7.275713 | 2.21E−09 |
| | | | | like | | | | |

TABLE VII
PARP1 Upregulated - Diff/X (Human); Name: Upregulated Lung Squamous Cell Carcinoma Primary
(Minimum Fold Change: 2.0); Experiment: Lung, Squamous Cell Carcinoma, Primary; Control: normal lung.

| | | | | | Pres. | Fold | | |
|---|---|---|---|---|---|---|---|---|
| Fragment Name | Array | Pathway | Symbol | Description | Freq. | Change | t-Score | p-Value |
| 209694_at | hg133a | | PTS | 6- | 0.951766 | 2.277376 | 8.469383 | 1.37E−10 |
| | | | | pyruvoyltetrahydropterin | | | | |
| | | | | synthase | | | | |
| 225342_at | hg133b | Kinase | AK3L2 | adenylate kinase 3-like 2 | 0.998591 | 2.450045 | 9.697055 | 3.57E−13 |
| 216594_x_at | hg133a | | AKR1C1 | aldo-keto reductase | 0.935517 | 7.001608 | 5.145986 | 8.26E−06 |
| | | | | family 1, member C1 | | | | |
| | | | | (dihydrodiol | | | | |
| | | | | dehydrogenase 1; 20- | | | | |
| | | | | alpha (3-alpha)- | | | | |
| | | | | hydroxysteroid | | | | |
| | | | | dehydrogenase) | | | | |
| 216594_x_at | hg133a | | AKR1C2 | aldo-keto reductase | 0.935517 | 7.001608 | 5.145986 | 8.26E−06 |
| | | | | family 1, member C2 | | | | |
| | | | | (dihydrodiol | | | | |
| | | | | dehydrogenase 2; bile | | | | |
| | | | | acid binding protein; 3- | | | | |
| | | | | alpha hydroxysteroid | | | | |
| | | | | dehydrogenase, type III) | | | | |
| 209160_at | hg133a | | AKR1C3 | aldo-keto reductase | 0.77386 | 5.470863 | 4.419198 | 7.93E−05 |
| | | | | family 1, member C3 (3- | | | | |
| | | | | alpha hydroxysteroid | | | | |
| | | | | dehydrogenase, type II) | | | | |
| 209186_at | hg133a | ATPase | ATP2A2 | ATPase, Ca++ | 0.999294 | 2.284561 | 11.71333 | 2.11E−16 |
| | | | | transporting, cardiac | | | | |
| | | | | muscle, slow twitch 2 | | | | |
| 202804_at | hg133a | | ABCC1 | ATP-binding cassette, | 0.997752 | 2.397733 | 4.661386 | 3.72E−05 |
| | | | | sub-family C | | | | |
| | | | | (CFTR/MRP), member 1 | | | | |
| 209380_s_at | hg133a | | ABCC5 | ATP-binding cassette, | 0.753565 | 3.135824 | 5.869529 | 8.32E−07 |
| | | | | sub-family C | | | | |
| | | | | (CFTR/MRP), member 5 | | | | |
| 212072_s_at | hg133a | Kinase | CSNK2A1 | casein kinase 2, alpha 1 | 0.938793 | 2.136742 | 10.1655 | 2.03E−13 |
| | | | | polypeptide | | | | |
| 201897_s_at | hg133a | | CKS1B | CDC28 protein kinase | 0.761593 | 3.029448 | 9.231723 | 1.30E−11 |
| | | | | regulatory subunit 1B | | | | |
| 224596_at | hg133b | | CDW92 | CDW92 antigen | 0.975372 | 2.134626 | 6.459808 | 7.99E−08 |
| 212977_at | hg133a | | CMKOR1 | chemokine orphan | 0.809891 | 2.184445 | 3.697104 | 6.40E−04 |
| | | | | receptor 1 | | | | |
| 221731_x_at | hg133a | | CSPG2 | chondroitin sulfate | 0.978613 | 2.141598 | 5.120157 | 6.41E−06 |
| | | | | proteoglycan 2 (versican) | | | | |
| 202246_s_at | hg133a | Kinase | CDK4 | cyclin-dependent kinase 4 | 0.924534 | 2.054219 | 9.603463 | 1.49E−12 |
| 201908_at | hg133a | Wnt/beta- | DVL3 | dishevelled, dsh homolog | 0.963198 | 2.179146 | 6.225294 | 2.42E−07 |
| | | catenin | | 3 (Drosophila) | | | | |
| | | pathway | | | | | | |
| 232353_s_at | hg133b | | DUSP24 | dual specificity | 0.744061 | 2.07963 | 7.11558 | 6.86E−09 |
| | | | | phosphatase 24 (putative) | | | | |
| 204256_at | hg133a | Fatty | ELOVL6 | ELOVL family member | 0.937058 | 2.255124 | 5.851689 | 4.64E−07 |
| | | acids | | 6, elongation of long | | | | |
| | | pathway | | chain fatty acids | | | | |
| | | | | (FEN1/Elo2, SUR4/Elo3- | | | | |
| | | | | like, yeast) | | | | |
| 203560_at | hg133a | | GGH | gamma-glutamyl | 0.901028 | 2.520354 | 5.072294 | 2.91E−06 |
| | | | | hydrolase (conjugase, | | | | |
| | | | | folylpolygammaglutamyl | | | | |
| | | | | hydrolase) | | | | |
| 208308_s_at | hg133a | | GPI | glucose phosphate | 0.998715 | 2.845709 | 6.923811 | 2.48E−08 |
| | | | | isomerase | | | | |
| 202923_s_at | hg133a | | GCLC | glutamate-cysteine ligase, | 0.796981 | 4.538398 | 6.330292 | 1.73E−07 |
| | | | | catalytic subunit | | | | |
| 225609_at | hg133b | | GSR | glutathione reductase | 0.942088 | 2.164992 | 4.87298 | 1.78E−05 |
| 214431_at | hg133a | | GMPS | guanine monophosphate | 0.921002 | 2.987449 | 7.966506 | 8.83E−10 |
| | | | | synthetase | | | | |
| 201841_s_at | hg133a | heat | HSPB1 | heat shock 27 kDa protein 1 | 0.923892 | 2.593016 | 6.693195 | 5.45E−08 |
| | | shock | | | | | | |
| 200807_s_at | hg133a | heat | HSPD1 | heat shock 60 kDa protein | 1 | 2.054097 | 8.947359 | 5.93E−12 |
| | | shock | | 1 (chaperonin) | | | | |
| 202854_at | hg133a | | HPRT1 | hypoxanthine | 0.998587 | 2.319045 | 8.797186 | 3.74E−11 |
| | | | | phosphoribosyltransferase | | | | |
| | | | | 1 (Lesch-Nyhan | | | | |
| | | | | syndrome) | | | | |
| 218507_at | hg133a | Hypoxia | HIG2 | hypoxia-inducible protein 2 | 0.854335 | 2.323142 | 3.692935 | 4.88E−04 |
| 210095_s_at | hg133a | | IGFBP3 | insulin-like growth factor | 0.80501 | 4.780732 | 5.783542 | 1.03E−06 |
| | | | | binding protein 3 | | | | |
| 210046_s_at | hg133a | | IDH2 | isocitrate dehydrogenase | 0.971034 | 2.417473 | 7.356119 | 3.81E−09 |
| | | | | 2 (NADP+), | | | | |
| | | | | mitochondrial | | | | |
| 217871_s_at | hg133a | NFkB; | MIF | macrophage migration | 0.995633 | 3.234484 | 10.54674 | 1.34E−13 |
| | | cell | | inhibitory factor | | | | |
| | | migration | | (glycosylation-inhibiting | | | | |
| | | | | factor) | | | | |
| 204059_s_at | hg133a | | ME1 | malic enzyme 1, | 0.869942 | 2.266546 | 4.373471 | 8.72E−05 |
| | | | | NADP(+)-dependent, | | | | |
| | | | | cytosolic | | | | |
| 203936_s_at | hg133a | NFkB; | matrix | 0.995247; Inhibitor: | Lung, | 2.293881 | 3.63562 | 4.31E−04 |
| | | cell | metalloproteinase 9 | MMP9 | Squamous | | | |
| | | migration; | (gelatinase | | Cell | | | |
| | | angiogenesis | B, | | Carcinoma, | | | |
| | | | 92 kDa | | Primary | | | |
| | | | gelatinase, | | | | | |
| | | | 92 kDa | | | | | |
| | | | type IV | | | | | |
| | | | collagenase) | | | | | |
| 222036_s_at | hg133a | DNA | MCM4 | MCM4 minichromosome | 0.878035 | 4.066684 | 7.487206 | 2.68E−09 |
| | | replication | | maintenance deficient 4 | | | | |
| | | | | (S. cerevisiae) | | | | |
| 201761_at | hg133a | | MTHFD2 | methylenetetrahydrofolate | 0.752922 | 2.477491 | 9.033929 | 9.65E−12 |
| | | | | dehydrogenase | | | | |
| | | | | (NADP+ dependent) 2, | | | | |
| | | | | methenyltetrahydrofolate | | | | |
| | | | | cyclohydrolase | | | | |
| 226556_at | hg133b | MAP | MAP3K13 | mitogen-activated protein | 0.961549 | 2.476006 | 6.941353 | 2.27E−08 |
| | | kinase | | kinase 13 | | | | |
| 210519_s_at | hg133a | | NQO1 | NAD(P)H | 0.744894 | 4.0396 | 5.13027 | 8.26E−06 |
| | | | | dehydrogenase, quinone 1 | | | | |
| 200790_at | hg133a | | ODC1 | ornithine decarboxylase 1 | 0.934682 | 2.165219 | 3.274084 | 2.23E−03 |
| 201489_at | hg133a | | PPIF | peptidylprolyl isomerase | 0.90668 | 2.846768 | 7.360372 | 2.85E−09 |
| | | | | F (cyclophilin F) | | | | |
| | | | | platelet | | | | |
| 201118_at | hg133a | | PGD | phosphogluconate | 0.835902 | 2.278703 | 4.190583 | 1.53E−04 |
| | | | | dehydrogenase | | | | |
| 201013_s_at | hg133a | | PAICS | phosphoribosylaminoimidazole | 0.993706 | 2.892041 | 8.70292 | 2.93E−11 |
| | | | | carboxylase, | | | | |
| | | | | phosphoribosylaminoimidazole | | | | |
| | | | | succinocarboxamide | | | | |
| | | | | synthetase | | | | |
| 223062_s_at | hg133b | | PSAT1 | phosphoserine | 0.818749 | 6.94196 | 6.302909 | 9.88E−08 |
| | | | | aminotransferase 1 | | | | |
| 225291_at | hg133b | | PNPT1 | polyribonucleotide | 0.996443 | 2.301183 | 6.863399 | 1.51E−08 |
| | | | | nucleotidyltransferase 1 | | | | |
| 202619_s_at | hg133a | | PLOD2 | procollagen-lysine, 2- | 0.787219 | 3.242858 | 4.186895 | 1.52E−04 |
| | | | | oxoglutarate 5- | | | | |
| | | | | dioxygenase 2 | | | | |
| 201202_at | hg133a | DNA | PCNA | proliferating cell nuclear | 0.959987 | 2.343228 | 7.565816 | 1.94E−09 |
| | | replication | | antigen | | | | |
| | | and | | | | | | |
| | | repair | | | | | | |
| 200830_at | hg133a | Proteosome | PSMD2 | proteasome (prosome, | 0.99878 | 2.53129 | 6.627885 | 7.05E−08 |
| | | pathway | | macropain) 26S subunit, | | | | |
| | | | | non-ATPase, 2 | | | | |
| 208694_at | hg133a | Kinase | PRKDC | protein kinase, DNA- | 0.976557 | 2.275786 | 6.675917 | 4.60E−08 |
| | | | | activated, catalytic | | | | |
| | | | | polypeptide | | | | |
| 201745_at | hg133a | Kinase | PTK9 | PTK9 protein tyrosine | 0.989531 | 2.205099 | 6.914447 | 2.15E−08 |
| | | | | kinase 9 | | | | |
| 226452_at | hg133b | Kinase | PDK1 | pyruvate dehydrogenase | 0.950745 | 3.103221 | 8.948826 | 1.13E−11 |
| | | | | kinase, isoenzyme 1 | | | | |
| 201251_at | hg133a | Kinase | PKM2 | pyruvate kinase, muscle | 0.961978 | 2.25298 | 9.25025 | 6.96E−12 |
| 222981_s_at | hg133b | RAS | RAB10 | RAB10, member RAS | 0.992082 | 2.383948 | 9.062732 | 1.51E−11 |
| | | oncogene | | oncogene family | | | | |
| | | family | | | | | | |
| 222077_s_at | hg133a | GTPase | RACGAP1 | Rac GTPase activating | 0.955106 | 3.100456 | 9.167409 | 1.18E−11 |
| | | | | protein 1 | | | | |
| 200750_s_at | hg133a | RAS | RAN | RAN, member RAS | 0.998715 | 2.033875 | 10.7408 | 4.47E−14 |
| | | oncogene | | oncogene family | | | | |
| | | family | | | | | | |
| 227897_at | hg133b | RAS | RAP2B | RAP2B, member of RAS | 0.849416 | 2.069148 | 5.508348 | 1.97E−06 |
| | | oncogene | | oncogene family | | | | |
| | | family | | | | | | |
| 204023_at | hg133a | DNA | RFC4 | replication factor C | 0.821644 | 4.045704 | 6.938005 | 2.66E−08 |
| | | repair | | (activator 1) 4, 37 kDa | | | | |
| 200903_s_at | hg133a | | AHCY | S-adenosylhomocysteine | 0.994348 | 2.073335 | 8.924151 | 5.63E−12 |
| | | | | hydrolase | | | | |
| 209875_s_at | hg133a | | SPP1 | secreted phosphoprotein 1 | 0.796275 | 8.675282 | 7.899683 | 6.63E−10 |
| | | | | (osteopontin, bone | | | | |
| | | | | sialoprotein I, early T- | | | | |
| | | | | lymphocyte activation 1) | | | | |
| 212190_at | hg133a | | SERPINE2 | serine (or cysteine) | 0.94817 | 3.007669 | 6.52343 | 6.91E−08 |
| | | | | proteinase inhibitor, clade | | | | |
| | | | | E (nexin, plasminogen | | | | |
| | | | | activator inhibitor type 1), | | | | |
| | | | | member 2 | | | | |
| 201563_at | hg133a | | SORD | sorbitol dehydrogenase | 0.975851 | 3.447372 | 7.045184 | 1.42E−08 |
| 202043_s_at | hg133a | | SMS | spermine synthase | 0.991843 | 2.322581 | 5.90577 | 6.78E−07 |
| 204675_at | hg133a | | SRD5A1 | steroid-5-alpha-reductase, | 0.813809 | 4.254906 | 6.187418 | 2.82E−07 |
| | | | | alpha polypeptide 1 (3- | | | | |
| | | | | oxo-5 alpha-steroid delta | | | | |
| | | | | 4-dehydrogenase alpha 1) | | | | |
| 224724_at | hg133b | | SULF2 | sulfatase 2 | 0.877198 | 2.65132 | 5.554637 | 1.95E−06 |
| 208864_s_at | hg133a | | TXN | thioredoxin | 0.999936 | 2.36316 | 6.250827 | 2.15E−07 |
| 201266_at | hg133a | | TXNRD1 | thioredoxin reductase 1 | 0.995633 | 2.211053 | 3.987273 | 2.82E−04 |
| 224511_s_at | hg133b | | TXNL5 | thioredoxin-like 5 | 0.923232 | 2.161844 | 8.089283 | 2.42E−10 |
| 202589_at | hg133a | | TYMS | thymidylate synthetase; | 0.919332 | 3.186399 | 8.197257 | 1.32E−11 |
| | | | | Inhibitor: 5-fluorouracil, | | | | |
| | | | | 5-fluoro-2-prime- | | | | |
| | | | | deoxyuridine, and some | | | | |
| | | | | folate analogs | | | | |
| 222633_at | hg133b | | TBL1XR1 | transducin (beta)-like 1X- | 0.763589 | 2.055267 | 4.642262 | 3.69E−05 |
| | | | | linked receptor 1 | | | | |
| 213011_s_at | hg133a | | TPI1 | triosephosphate isomerase 1 | 0.999294 | 2.451804 | 9.88241 | 1.05E−12 |
| 202779_s_at | hg133a | Proteosome/ | UBE2S | ubiquitin-conjugating | 0.743224 | 4.305175 | 7.200878 | 2.49E−09 |
| | | Ubiquitin | | enzyme E2S | | | | |
| | | pathway | | | | | | |

TABLE VIII
PARP1 Upregulated - Diff/X (Human); Name: Upregulated Ovary Adenocarcinoma Endometrioid
Type Primary (Minimum Fold Change: 2.0); Experiment: Ovary, Adenocarcinoma,
Endometrioid Type, Primary; control: normal ovary.

| Fragment | | | | | Pres. | Fold | | |
|---|---|---|---|---|---|---|---|---|
| Name | Array | Pathway | Symbol | Description | Freq. | Change | t-Score | p-Value |
| 207275_s_at | hg133a | | ACSL1 | acyl-CoA synthetase | 0.910212 | 2.036312 | 2.938972 | 7.69E−03 |
| | | | | long-chain family | | | | |
| | | | | member 1 | | | | |
| 201662_s_at | hg133a | | ACSL3 | acyl-CoA synthetase | 0.966346 | 2.09512 | 3.522611 | 1.95E−03 |
| | | | | long-chain family | | | | |
| | | | | member 3 | | | | |
| 225342_at | hg133b | Kinase | AK3L1 | adenylate kinase 3-like 1 | 0.998591 | 2.892264 | 4.645283 | 1.17E−04 |
| 216266_s_at | hg133a | ADP- | ARFGEF1 | ADP-ribosylation factor | 0.96307 | 2.139026 | 4.630324 | 1.26E−04 |
| | | ribosylation | | guanine nucleotide- | | | | |
| | | | | exchange factor | | | | |
| | | | | 1(brefeldin A-inhibited) | | | | |
| 202912_at | hg133a | | ADM | adrenomedullin | 0.835967 | 2.82046 | 3.007054 | 6.57E−03 |
| 227021_at | hg133b | | AOF1 | amine oxidase (flavin | 0.900953 | 2.030322 | 4.623665 | 1.25E−04 |
| | | | | containing) domain 1 | | | | |
| 204446_s_at | hg133a | | ALOX5 | arachidonate 5- | 0.752216 | 2.038583 | 4.413579 | 1.91E−04 |
| | | | | lipoxygenase | | | | |
| 207508_at | hg133a | ATP | ATP5G3 | ATP synthase, H+ | 0.99878 | 2.004593 | 3.671549 | 1.38E−03 |
| | | regulation | | transporting, | | | | |
| | | | | mitochondrial F0 | | | | |
| | | | | complex, subunit c | | | | |
| | | | | (subunit 9) isoform 3 | | | | |
| 202961_s_at | hg133a | ATP | ATP5J2 | ATP synthase, H+ | 0.993642 | 2.08818 | 6.363731 | 1.68E−06 |
| | | regulation | | transporting, | | | | |
| | | | | mitochondrial F0 | | | | |
| | | | | complex, subunit f, | | | | |
| | | | | isoform 2 | | | | |
| 209186_at | hg133a | ATP | ATP2A2 | ATPase, Ca++ | 0.999294 | 2.595367 | 9.047713 | 2.86E−09 |
| | | regulation | | transporting, cardiac | | | | |
| | | | | muscle, slow twitch 2 | | | | |
| 230875_s_at | hg133b | ATP | ATP11A | ATPase, Class VI, type | 0.944974 | 2.889093 | 2.991555 | 6.75E−03 |
| | | regulation | | 11A | | | | |
| 200078_s_at | hg133a | ATP | ATP6V0B | ATPase, H+ | 0.930893 | 2.201526 | 7.562541 | 7.69E−08 |
| | | regulation | | transporting, lysosomal | | | | |
| | | | | 21 kDa, V0 subunit c” | | | | |
| 225552_x_at | hg133b | aurora-A | AKIP | aurora-A kinase | 0.995571 | 2.072771 | 5.591353 | 1.24E−05 |
| | | kinase | | interacting protein | | | | |
| | | pathway | | | | | | |
| 212312_at | hg133a | BCL | BCL2L1 | BCL2-like 1 | 0.908863 | 2.659455 | 6.833241 | 7.49E−07 |
| | | oncogene | | | | | | |
| | | pathway | | | | | | |
| 222446_s_at | hg133b | | BACE2 | beta-site APP-cleaving | 0.878204 | 3.487594 | 6.030002 | 4.78E−06 |
| | | | | enzyme 2 | | | | |
| 225864_at | hg133b | DNA | NSE2 | breast cancer membrane | 0.914911 | 4.338772 | 8.188753 | 3.94E−08 |
| | | repair | | protein 101: Inhibitors | | | | |
| | | | | described in Mol Cell | | | | |
| | | | | Biol. 2005 | | | | |
| | | | | Aug; 25(16): 7021-32 | | | | |
| 36499_at | hg133a | | CELSR2 | cadherin, EGF LAG | 0.749583 | 3.1993 | 9.072154 | 1.79E−09 |
| | | | | seven-pass G-type | | | | |
| | | | | receptor 2 (flamingo | | | | |
| | | | | homolog, Drosophila) | | | | |
| 221059_s_at | hg133a | | CHST6 | carbohydrate (N- | 0.922415 | 2.234664 | 4.510118 | 1.61E−04 |
| | | | | acetylglucosamine 6-O) | | | | |
| | | | | sulfotransferase 6 | | | | |
| 201940_at | hg133a | | CPD | carboxypeptidase D | 0.862428 | 2.745212 | 3.587699 | 1.43E−03 |
| 210070_s_at | hg133a | | CPT1B | carnitine | 0.783622 | 2.138968 | 5.885634 | 4.22E−06 |
| | | | | palmitoyltransferase 1B | | | | |
| | | | | (muscle) | | | | |
| 200839_s_at | hg133a | | CTSB | cathepsin B | 0.992421 | 2.770155 | 5.545809 | 1.42E−05 |
| 209835_x_at | hg133a | | CD44 | CD44 antigen (homing | 0.976236 | 3.016266 | 4.832544 | 7.93E−05 |
| | | | | function and Indian | | | | |
| | | | | blood group system) | | | | |
| 211075_s_at | hg133a | | CD47 | CD47 antigen (Rh- | 0.997624 | 2.661029 | 4.454209 | 2.06E−04 |
| | | | | related antigen, integrin- | | | | |
| | | | | associated signal | | | | |
| | | | | transducer) | | | | |
| 205173_x_at | hg133a | | CD58 | CD58 antigen, | 0.745279 | 2.757295 | 2.880262 | 8.85E−03 |
| | | | | (lymphocyte function- | | | | |
| | | | | associated antigen 3) | | | | |
| 209619_at | hg133a | | CD74 | CD74 antigen (invariant | 0.939756 | 2.411928 | 5.148763 | 2.56E−05 |
| | | | | polypeptide of major | | | | |
| | | | | histocompatibility | | | | |
| | | | | complex, class II | | | | |
| | | | | antigen-associated) | | | | |
| 201005_at | hg133a | | CD9 | CD9 antigen (p24) | 0.922543 | 5.854125 | 6.458654 | 1.86E−06 |
| 226185_at | hg133b | | CDS1 | CDP-diacylglycerol | 0.877198 | 2.035304 | 5.337548 | 1.66E−05 |
| | | | | synthase (phosphatidate | | | | |
| | | | | cytidylyltransferase) 1 | | | | |
| 217028_at | hg133a | NFkB | CXCR4 | chemokine (C—X—C | 0.88876 | 3.799321 | 4.765092 | 9.00E−05 |
| | | anf | | motif) receptor 4; | | | | |
| | | hypoxia | | inhibitors described in | | | | |
| | | | | Nat Med. 2007 Apr 15 | | | | |
| 225009_at | hg133b | | CKLFSF4 | chemokine-like factor | 0.748088 | 2.657073 | 5.787083 | 7.69E−06 |
| | | | | super family 4 | | | | |
| 223047_at | hg133b | | CKLFSF6 | chemokine-like factor | 0.992618 | 2.938667 | 6.200167 | 2.29E−06 |
| | | | | super family 6 | | | | |
| 204620_s_at | hg133a | | CSPG2 | chondroitin sulfate | 0.893963 | 3.899678 | 3.917118 | 7.46E−04 |
| | | | | proteoglycan 2 | | | | |
| | | | | (versican) | | | | |
| 223020_at | hg133b | | CRR9 | cisplatin resistance | 0.937324 | 2.501359 | 4.135577 | 4.56E−04 |
| | | | | related protein CRR9p | | | | |
| 203359_s_at | hg133a | Myc | MYCBP | c-myc binding protein | 0.981888 | 2.122181 | 6.04642 | 3.75E−06 |
| | | oncogene | | | | | | |
| | | pathway | | | | | | |
| 217752_s_at | hg133a | | CNDP2 | CNDP dipeptidase 2 | 0.983558 | 3.771819 | 7.385651 | 2.34E−07 |
| | | | | (metallopeptidase M20 | | | | |
| | | | | family) | | | | |
| 203917_at | hg133a | | CXADR | coxsackie virus and | 0.83738 | 12.816638 | 7.376081 | 2.48E−07 |
| | | | | adenovirus receptor | | | | |
| 202613_at | hg133a | | CTPS | CTP synthase | 0.95228 | 2.189729 | 5.432328 | 1.31E−05 |
| 222996_s_at | hg133b | | CXXC5 | CXXC finger 5 | 0.872299 | 3.587127 | 5.592877 | 1.34E−05 |
| 201584_s_at | hg133a | | DDX39 | DEAD (Asp-Glu-Ala- | 0.999743 | 2.129179 | 5.644934 | 1.14E−05 |
| | | | | Asp) box polypeptide 39 | | | | |
| 209094_at | hg133a | | DDAH1 | dimethylarginine | 0.861207 | 2.566109 | 4.349263 | 2.32E−04 |
| | | | | dimethylaminohydrolase 1 | | | | |
| 210749_x_at | hg133a | | DDR1 | discoidin domain | 0.871098 | 2.028374 | 6.851084 | 3.25E−07 |
| | | | | receptor family, member 1 | | | | |
| 223054_at | hg133b | | DNAJB11 | DnaJ (Hsp40) homolog, | 0.987183 | 2.219687 | 8.881242 | 5.16E−09 |
| | | | | subfamily B, member 11 | | | | |
| 225174_at | hg133b | | DNAJC10 | DnaJ (Hsp40) homolog, | 0.980405 | 2.156119 | 4.968441 | 5.64E−05 |
| | | | | subfamily C, member 10 | | | | |
| 227808_at | hg133b | | DNAJD1 | DnaJ (Hsp40) homolog, | 0.858274 | 2.408959 | 3.036736 | 6.18E−03 |
| | | | | subfamily D, member 1 | | | | |
| 232353_s_at | hg133b | | DUSP24 | dual specificity | 0.744061 | 2.205015 | 6.367321 | 1.84E−06 |
| | | | | phosphatase 24 | | | | |
| | | | | (putative) | | | | |
| 208891_at | hg133a | | DUSP6 | dual specificity | 0.968401 | 4.24943 | 4.28216 | 3.18E−04 |
| | | | | phosphatase 6 | | | | |
| 204160_s_at | hg133a | | ENPP4 | ectonucleotide | 0.832627 | 2.649791 | 4.456779 | 1.94E−04 |
| | | | | pyrophosphatase/ | | | | |
| | | | | phosphodiesterase | | | | |
| | | | | 4 (putative | | | | |
| | | | | function) | | | | |
| 219017_at | hg133a | Kinase | ETNK1 | ethanolamine kinase 1 | 0.981888 | 2.139712 | 3.115929 | 4.99E−03 |
| 225764_at | hg133b | Tel | ETV6 | ets variant gene 6 (TEL | 0.745135 | 2.16324 | 5.94353 | 3.96E−06 |
| | | Ongogene | | oncogene) | | | | |
| 223000_s_at | hg133b | | F11R | F11 receptor | 0.89894 | 3.00523 | 6.920303 | 3.93E−07 |
| 202345_s_at | hg133a | Fatty | FABP5 | fatty acid binding | 0.938921 | 4.644953 | 3.841378 | 9.31E−04 |
| | | acids | | protein 5 (psoriasis- | | | | |
| | | pathway | | associated) | | | | |
| 212070_at | hg133a | GPCR | GPR56 | G protein-coupled | 0.797302 | 9.709793 | 6.500176 | 1.64E−06 |
| | | | | receptor 56 | | | | |
| 215438_x_at | hg133a | | GSPT1 | G1 to S phase transition 1 | 0.84271 | 2.069257 | 4.628571 | 1.08E−04 |
| 239761_at | hg133b | | GCNT1 | glucosaminyl (N-acetyl) | 0.849953 | 2.275141 | 6.843018 | 6.39E−07 |
| | | | | transferase 1, core 2 | | | | |
| | | | | (beta-1,6-N- | | | | |
| | | | | acetylglucosaminyltransferase) | | | | |
| 208308_s_at | hg133a | | GPI | glucose phosphate | 0.998715 | 2.46323 | 5.899777 | 6.63E−06 |
| | | | | isomerase | | | | |
| 203925_at | hg133a | | GCLM | glutamate-cysteine | 0.946757 | 2.075069 | 3.653241 | 1.18E−03 |
| | | | | ligase, modifier subunit | | | | |
| 202722_s_at | hg133a | | GFPT1 | glutamine-fructose-6- | 0.894541 | 2.677052 | 5.852784 | 7.14E−06 |
| | | | | phosphate transaminase 1 | | | | |
| 200736_s_at | hg133a | | GPX1 | glutathione peroxidase 1 | 0.989017 | 2.551562 | 6.173009 | 2.87E−06 |
| 211015_s_at | hg133a | Heat | HSPA4 | heat shock 70 kDa | 0.937893 | 2.903139 | 11.02266 | 7.94E−11 |
| | | shock | | protein 4 | | | | |
| 200896_x_at | hg133a | | HDGF | hepatoma-derived | 0.999422 | 2.204433 | 6.954696 | 5.21E−07 |
| | | | | growth factor (high- | | | | |
| | | | | mobility group protein | | | | |
| | | | | 1-like) | | | | |
| 217496_s_at | hg133a | | IDE | insulin-degrading | 0.770584 | 2.06495 | 7.632508 | 7.56E−08 |
| | | | | enzyme | | | | |
| 201587_s_at | hg133a | NFkB | IRAK1 | interleukin-1 receptor- | 0.978741 | 2.481482 | 4.986889 | 5.62E−05 |
| | | pathway | | associated kinase 1 | | | | |
| 210046_s_at | hg133a | | IDH2 | isocitrate dehydrogenase | 0.971034 | 7.321111 | 7.268362 | 3.44E−07 |
| | | | | 2 (NADP+), | | | | |
| | | | | mitochondrial | | | | |
| 201609_x_at | hg133a | | ICMT | isoprenylcysteine | 0.928452 | 2.128633 | 9.601435 | 1.10E−09 |
| | | | | carboxyl | | | | |
| | | | | methyltransferase | | | | |
| 200650_s_at | hg133a | | LDHA | lactate dehydrogenase A | 1 | 3.024234 | 6.242531 | 2.72E−06 |
| 217933_s_at | hg133a | | LAP3 | leucine aminopeptidase 3 | 0.995055 | 2.013408 | 2.888395 | 8.60E−03 |
| 228824_s_at | hg133b | | LTB4DH | leukotriene B4 12- | 0.836465 | 2.768501 | 3.167723 | 4.60E−03 |
| | | | | hydroxydehydrogenase | | | | |
| 217871_s_at | hg133a | NFkB | MIF | macrophage migration | 0.995633 | 2.874721 | 9.316017 | 2.68E−09 |
| | | anf | | inhibitory factor | | | | |
| | | hypoxia | | (glycosylation-inhibiting | | | | |
| | | | | factor); inhibitors | | | | |
| | | | | described in Nat Med. | | | | |
| | | | | 2007 Apr 15 | | | | |
| 203362_s_at | hg133a | | MAD2L1 | MAD2 mitotic arrest | 0.808863 | 3.694417 | 4.370425 | 2.62E−04 |
| | | | | deficient-like 1 (yeast) | | | | |
| 220189_s_at | hg133a | | MGAT4B | mannosyl (alpha-1,3-)- | 0.943353 | 2.324942 | 7.377243 | 1.30E−07 |
| | | | | glycoprotein beta-1,4-N- | | | | |
| | | | | acetylglucosaminyltransferase, | | | | |
| | | | | isoenzyme B | | | | |
| 203936_s_at | hg133a | Cell | MMP9 | matrix metalloproteinase | 0.995247 | 3.032626 | 3.037539 | 6.22E−03 |
| | | migration; | | 9 (gelatinase B, 92 kDa | | | | |
| | | angiogenesis; | | gelatinase, 92 kDa type | | | | |
| | | NFkB | | IV collagenase) | | | | |
| 222036_s_at | hg133a | DNA | MCM4 | MCM4 | 0.878035 | 3.05091 | 5.253903 | 3.09E−05 |
| | | replication | | minichromosome | | | | |
| | | | | maintenance deficient 4 | | | | |
| | | | | (S. cerevisiae) | | | | |
| 201761_at | hg133a | | MTHFD2 | methylenetetrahydrofolate | 0.752922 | 3.031198 | 5.838052 | 7.28E−06 |
| | | | | dehydrogenase | | | | |
| | | | | (NADP+ dependent) 2, | | | | |
| | | | | methenyltetrahydrofolate | | | | |
| | | | | cyclohydrolase | | | | |
| 225253_s_at | hg133b | | METTL2 | methyltransferase like 2 | 0.841296 | 2.064544 | 4.52665 | 1.65E−04 |
| 210058_at | hg133a | mitogen- | MAPK13 | mitogen-activated | 0.912267 | 3.084653 | 8.876498 | 8.04E−09 |
| | | activated | | protein kinase 13 | | | | |
| | | protein | | | | | | |
| | | kinase | | | | | | |
| 215498_s_at | hg133a | mitogen- | MAP2K3 | mitogen-activated | 0.966667 | 2.065987 | 6.255566 | 2.01E−06 |
| | | activated | | protein kinase 3 | | | | |
| | | protein | | | | | | |
| | | kinase | | | | | | |
| 205698_s_at | hg133a | mitogen- | MAP2K6 | mitogen-activated | 0.80957 | 3.709886 | 5.599329 | 1.43E−05 |
| | | activated | | protein kinase 6 | | | | |
| | | protein | | | | | | |
| | | kinase | | | | | | |
| 207847_s_at | hg133a | | MUC1 | mucin 1, transmembrane | 0.858703 | 14.795158 | 6.53168 | 1.57E−06 |
| 210519_s_at | hg133a | | NQO1 | NAD(P)H | 0.744894 | 5.350942 | 3.528042 | 1.93E−03 |
| | | | | dehydrogenase, quinone 1 | | | | |
| 224802_at | hg133b | Ubiquitin/ | NDFIP2 | Nedd4 family interacting | 0.938196 | 2.421513 | 5.486497 | 1.42E−05 |
| | | proteosome | | protein 2 | | | | |
| | | pathway | | | | | | |
| 201830_s_at | hg133a | | NET1 | neuroepithelial cell | 0.789017 | 2.022294 | 2.870849 | 8.87E−03 |
| | | | | transforming gene 1 | | | | |
| 223158_s_at | hg133b | Kinase | NEK6 | NIMA (never in mitosis | 0.817675 | 4.327528 | 8.392761 | 2.15E−08 |
| | | | | gene a)-related kinase 6 | | | | |
| 226649_at | hg133b | Kinase | PANK1 | pantothenate kinase 1 | 0.797611 | 2.462013 | 5.357904 | 2.32E−05 |
| 201876_at | hg133a | Kinase | PON2 | paraoxonase 2 | 0.915607 | 2.352264 | 4.786468 | 9.04E−05 |
| 208824_x_at | hg133a | Kinase | PCTK1 | PCTAIRE protein kinase 1 | 0.920424 | 2.126444 | 7.312529 | 1.87E−07 |
| 201954_at | hg133a | | PDAP1 | PDGFA associated | 0.901285 | 3.094466 | 6.243358 | 2.55E−06 |
| | | | | protein 1 | | | | |
| 201489_at | hg133a | | PPIF | peptidylprolyl isomerase | 0.90668 | 2.740565 | 4.447802 | 1.49E−04 |
| | | | | F (cyclophilin F) | | | | |
| 201037_at | hg133a | | PFKP | phosphofructokinase, | 0.953565 | 2.830169 | 5.168132 | 3.30E−05 |
| | | | | platelet | | | | |
| 238417_at | hg133b | | PGM2L1 | phosphoglucomutase 2- | 0.826802 | 2.168661 | 3.542651 | 1.83E−03 |
| | | | | like 1 | | | | |
| 201118_at | hg133a | | PGD | phosphogluconate | 0.835902 | 2.385404 | 4.214639 | 3.65E−04 |
| | | | | dehydrogenase | | | | |
| 227068_at | hg133b | Kinase | PGK1 | phosphoglycerate kinase 1 | 0.812911 | 3.801436 | 6.627594 | 1.15E−06 |
| 210145_at | hg133a | | PLA2G4A | phospholipase A2, group | 0.773796 | 5.267446 | 2.832646 | 9.93E−03 |
| | | | | IVA (cytosolic, calcium- | | | | |
| | | | | dependent) | | | | |
| 213222_at | hg133a | | PLCB1 | phospholipase C, beta 1 | 0.822993 | 3.108566 | 4.136349 | 4.50E−04 |
| | | | | (phosphoinositide- | | | | |
| | | | | specific) | | | | |
| 223062_s_at | hg133b | | PSAT1 | phosphoserine | 0.818749 | 12.223022 | 5.286096 | 3.03E−05 |
| | | | | aminotransferase 1 | | | | |
| 201928_at | hg133a | | PKP4 | plakophilin 4 | 0.984522 | 2.456331 | 5.493022 | 1.79E−05 |
| 200654_at | hg133a | | P4HB | procollagen-proline, 2- | 0.886705 | 2.156597 | 7.792758 | 4.79E−09 |
| | | | | oxoglutarate 4- | | | | |
| | | | | dioxygenase (proline 4- | | | | |
| | | | | hydroxylase), beta | | | | |
| | | | | polypeptide (protein | | | | |
| | | | | disulfide isomerase- | | | | |
| | | | | associated 1) | | | | |
| 205128_x_at | hg133a | | PTGS1 | prostaglandin- | 0.857868 | 2.673359 | 2.78959 | 1.09E−02 |
| | | | | endoperoxide synthase 1 | | | | |
| | | | | (prostaglandin G/H | | | | |
| | | | | synthase and | | | | |
| | | | | cyclooxygenase) | | | | |
| 212296_at | hg133a | Proteosome | PSMD14 | proteasome (prosome, | 0.997238 | 2.479988 | 7.569201 | 1.23E−07 |
| | | | | macropain) 26S subunit, | | | | |
| | | | | non-ATPase, 14 | | | | |
| 201400_at | hg133a | Proteosome | PSMB3 | proteasome (prosome, | 0.99878 | 2.156049 | 10.51732 | 2.34E−11 |
| | | | | macropain) subunit, beta | | | | |
| | | | | type, 3 | | | | |
| 200846_s_at | hg133a | | PPP1CA | protein phosphatase 1, | 0.929929 | 3.807386 | 8.692774 | 1.16E−08 |
| | | | | catalytic subunit, alpha | | | | |
| | | | | isoform | | | | |
| 202671_s_at | hg133a | | PDXK | pyridoxal (pyridoxine, | 0.95228 | 3.45706 | 5.396223 | 2.10E−05 |
| | | | | vitamin B6) kinase | | | | |
| 217848_s_at | hg133a | | PP | pyrophosphatase | 0.987797 | 3.379374 | 8.345857 | 3.12E−08 |
| | | | | (inorganic) | | | | |
| 201251_at | hg133a | Kinase | PKM2 | pyruvate kinase, muscle | 0.961978 | 3.415407 | 9.536555 | 3.16E−09 |
| 222981_s_at | hg133b | RAS | RAB10 | RAB10, member RAS | 0.992082 | 2.108531 | 6.293194 | 2.19E−06 |
| | | oncogene | | oncogene family | | | | |
| | | pathway/ | | | | | | |
| | | family | | | | | | |
| 225177_at | hg133b | RAS | RAB11 | RAB11 family | 0.984029 | 2.080782 | 4.933512 | 5.31E−05 |
| | | oncogene | FIP1 | interacting protein 1 | | | | |
| | | pathway/ | | (class I) | | | | |
| | | family | | | | | | |
| 223471_at | hg133b | RAS | RAB3IP | RAB3A interacting | 0.75567 | 4.756142 | 6.653864 | 1.21E−06 |
| | | oncogene | | protein (rabin3) | | | | |
| | | pathway/ | | | | | | |
| | | family | | | | | | |
| 222077_s_at | hg133a | RAS | RACGAP1 | Rac GTPase activating | 0.955106 | 3.111767 | 6.286547 | 2.68E−06 |
| | | oncogene | | protein 1 | | | | |
| | | pathway/ | | | | | | |
| | | family | | | | | | |
| 202483_s_at | hg133a | RAS | RANBP1 | RAN binding protein 1 | 0.838471 | 2.295085 | 3.065815 | 5.81E−03 |
| | | oncogene | | | | | | |
| | | pathway/ | | | | | | |
| | | family | | | | | | |
| 200750_s_at | hg133a | RAS | RAN | RAN, member RAS | 0.998715 | 2.209431 | 6.126863 | 3.28E−06 |
| | | oncogene | | oncogene family | | | | |
| | | pathway/ | | | | | | |
| | | family | | | | | | |
| 207525_s_at | hg133a | | RGS19IP1 | regulator of G-protein | 0.890687 | 2.40733 | 10.37019 | 2.19E−10 |
| | | | | signaling 19 interacting | | | | |
| | | | | protein 1 | | | | |
| 226021_at | hg133b | | RDH10 | retinol dehydrogenase | 0.852235 | 6.354083 | 7.072733 | 4.89E−07 |
| | | | | 10 (all-trans) | | | | |
| 202200_s_at | hg133a | Kinase | SRPK1 | SFRS protein kinase 1 | 0.996275 | 2.013796 | 8.84441 | 7.26E−09 |
| 201563_at | hg133a | | SORD | sorbitol dehydrogenase | 0.975851 | 5.210444 | 7.590281 | 1.52E−07 |
| 230333_at | hg133b | | SAT | spermidine/spermine | 0.9796 | 2.309113 | 3.516629 | 1.91E−03 |
| | | | | N1-acetyltransferase | | | | |
| 212321_at | hg133a | | SGPL1 | sphingosine-1-phosphate | 0.943995 | 2.20098 | 5.55781 | 1.48E−05 |
| | | | | lyase 1 | | | | |
| 226560_at | hg133b | | SGPP2 | sphingosine-1-phosphate | 0.812844 | 6.741899 | 6.040691 | 5.02E−06 |
| | | | | phosphatase 2 | | | | |
| 201998_at | hg133a | | ST6GAL1 | ST6 beta-galactosamide | 0.905909 | 2.323038 | 3.11495 | 5.18E−03 |
| | | | | alpha-2,6- | | | | |
| | | | | sialyltranferase 1 | | | | |
| 222750_s_at | hg133b | | SRD5A2L | steroid 5 alpha-reductase | 0.928332 | 5.349418 | 6.239489 | 3.27E−06 |
| | | | | 2-like | | | | |
| 202071_at | hg133a | | SDC4 | syndecan 4 | 0.88343 | 2.618198 | 3.972644 | 6.34E−04 |
| | | | | (amphiglycan, ryudocan) | | | | |
| 218763_at | hg133a | | STX18 | syntaxin 18 | 0.829672 | 2.55142 | 3.218284 | 4.03E−03 |
| 217979_at | hg133a | | TSPAN13 | tetraspanin 13 | 0.973732 | 5.938491 | 7.886676 | 4.40E−08 |
| 202589_at | hg133a | | TYMS | thymidylate synthetase; | 0.919332 | 4.386055 | 5.531397 | 1.57E−05 |
| | | | | inhibitor: 5-fluorouracil, | | | | |
| | | | | 5-fluoro-2-prime- | | | | |
| | | | | deoxyuridine, and some | | | | |
| | | | | folate analogs | | | | |
| 213011_s_at | hg133a | | TPI1 | triosephosphate | 0.999294 | 2.676732 | 7.608777 | 1.41E−07 |
| | | | | isomerase 1 | | | | |
| 202510_s_at | hg133a | | TNFAIP2 | tumor necrosis factor, | 0.798523 | 3.804343 | 5.14267 | 4.07E−05 |
| | | | | alpha-induced protein 2 | | | | |
| 208743_s_at | hg133a | | YWHAB | tyrosine 3- | 0.995504 | 2.110942 | 7.965989 | 4.54E−09 |
| | | | | monooxygenase/tryptophan | | | | |
| | | | | 5-monooxygenase | | | | |
| | | | | activation protein, beta | | | | |
| | | | | polypeptide | | | | |
| 200641_s_at | hg133a | | YWHAZ | tyrosine 3- | 0.993192 | 2.151934 | 4.459566 | 1.93E−04 |
| | | | | monooxygenase/tryptophan | | | | |
| | | | | 5-monooxygenase | | | | |
| | | | | activation protein, zeta | | | | |
| | | | | polypeptide | | | | |
| 202779_s_at | hg133a | Ubiquitin/ | UBE2S | ubiquitin-conjugating | 0.743224 | 2.098212 | 4.033033 | 5.20E−04 |
| | | proteosome | | enzyme E2S | | | | |
| 222870_s_at | hg133b | | B3GNT1 | UDP-GlcNAc:betaGal | 0.908938 | 2.677728 | 6.638119 | 9.19E−07 |
| | | | | beta-1,3-N- | | | | |
| | | | | acetylglucosaminyltransferase 1 | | | | |
| 226283_at | hg133b | | GALNT4 | UDP-N-acetyl-alpha-D- | 0.917461 | 2.189005 | 3.432649 | 2.41E−03 |
| | | | | galactosamine:polypeptide | | | | |
| | | | | N- | | | | |
| | | | | acetylgalactosaminyltransferase | | | | |
| | | | | 4 (GalNAc-T4) | | | | |
| 218313_s_at | hg133a | | GALNT7 | UDP-N-acetyl-alpha-D- | 0.90578 | 2.72546 | 4.712261 | 1.08E−04 |
| | | | | galactosamine:polypeptide | | | | |
| | | | | N- | | | | |
| | | | | acetylgalactosaminyltransferase | | | | |
| | | | | 7 (GalNAc-T7) | | | | |
| 210513_s_at | hg133a | VEGF | VEGF | vascular endothelial | 0.941105 | 2.382562 | 3.936258 | 7.29E−04 |
| | | | | growth factor; inhibitor: | | | | |
| | | | | Avastin | | | | |
| 218807_at | hg133a | VAV3 | VAV3 | vav 3 oncogene | 0.927489 | 4.987168 | 3.599801 | 1.68E−03 |
| | | oncogene; | | | | | | |
| | | NFkB | | | | | | |
| | | activator | | | | | | |
| 202454_s_at | hg133a | HER3 | ERBB3 | v-erb-b2 erythroblastic | 0.861207 | 4.424339 | 8.903786 | 6.26E−09 |
| | | | | leukemia viral oncogene | | | | |
| | | | | homolog 3 (avian); | | | | |
| | | | | inhibitor: Herceptin | | | | |
| 212038_s_at | hg133a | | VDAC1 | voltage-dependent anion | 0.999422 | 2.15422 | 8.071488 | 1.52E−08 |
| | | | | channel 1 | | | | |
| 202625_at | hg133a | Lyn | LYN | v-yes-1 Yamaguchi | 0.82903 | 2.470315 | 6.431013 | 1.09E−06 |
| | | oncogene | | sarcoma viral related | | | | |
| | | | | oncogene homolog | | | | |

TABLE IX
PARP1 Upregulated - Diff/X (Human); Name: Upregulated Ovary Serous Cystadenocarcinoma
Primary (Minimum Fold Change: 2.0); Experiment: Ovary, Serous Cystadenocarcinoma,
Primary; Control: normal ovary.

| Fragment Name | Array | Pathway | Symbol | Description | Pres. Freq. | Fold Change | t-Score | p-Value |
|---|---|---|---|---|---|---|---|---|
| 204998_s_at | hg133a | | ATF5 | activating transcription | 0.971227 | 2.062269 | 5.647318 | 6.37E−04 |
| | | | | factor 5 | | | | |
| 218987_at | hg133a | | ATF7IP | activating transcription | 0.994926 | 2.247265 | 8.332725 | 5.18E−05 |
| | | | | factor 7 interacting | | | | |
| | | | | protein | | | | |
| 208750_s_at | hg133a | ADP- | ARF1 | ADP-ribosylation factor 1 | 0.979062 | 2.004949 | 5.833401 | 3.54E−04 |
| | | ribosylation | | | | | | |
| 202207_at | hg133a | ADP- | ARL7 | ADP-ribosylation factor- | 0.808671 | 8.217095 | 4.67423 | 2.27E−03 |
| | | ribosylation | | like 7 | | | | |
| 227021_at | hg133b | | AOF1 | amine oxidase (flavin | 0.900953 | 2.557067 | 4.244951 | 3.60E−03 |
| | | | | containing) domain 1 | | | | |
| 222608_s_at | hg133b | | ANLN | anillin, actin binding | 0.752516 | 4.902239 | 5.667107 | 7.20E−04 |
| | | | | protein (scraps homolog, | | | | |
| | | | | Drosophila) | | | | |
| 213503_x_at | hg133a | | ANXA2 | annexin A2 | 0.911882 | 2.286595 | 3.874438 | 5.65E−03 |
| 207076_s_at | hg133a | | ASS | argininosuccinate | 0.844894 | 5.346931 | 4.605665 | 2.44E−03 |
| | | | | synthetase | | | | |
| 207507_s_at | hg133a | ATP | ATP5G3 | ATP synthase, H+ | 0.997174 | 2.330518 | 4.148467 | 4.13E−03 |
| | | synthase | | transporting, | | | | |
| | | | | mitochondrial F0 | | | | |
| | | | | complex, subunit c | | | | |
| | | | | (subunit 9) isoform 3 | | | | |
| 202961_s_at | hg133a | ATP | ATP5J2 | ATP synthase, H+ | 0.993642 | 2.198314 | 4.864669 | 1.60E−03 |
| | | synthase | | transporting, | | | | |
| | | | | mitochondrial F0 | | | | |
| | | | | complex, subunit f, | | | | |
| | | | | isoform 2 | | | | |
| 200078_s_at | hg133a | ATP | ATP6V0B | ATPase, H+ transporting, | 0.930893 | 2.00476 | 8.755436 | 8.09E−06 |
| | | synthase | | lysosomal 21 kDa, V0 | | | | |
| | | | | subunit c” | | | | |
| 218580_x_at | hg133a | aurora-A | AKIP | aurora-A kinase | 0.9842 | 2.028049 | 6.612785 | 2.23E−04 |
| | | kinase | | interacting protein | | | | |
| | | pathway | | | | | | |
| 212312_at | hg133a | BCL | BCL2L1 | BCL2-like 1 | 0.908863 | 2.716036 | 6.165821 | 4.07E−04 |
| | | oncogene | | | | | | |
| | | pathway | | | | | | |
| 222446_s_at | hg133b | | BACE2 | beta-site APP-cleaving | 0.878204 | 2.973236 | 3.340146 | 1.21E−02 |
| | | | | enzyme 2 | | | | |
| 225864_at | hg133b | DNA | NSE2 | breast cancer membrane | 0.914911 | 3.660408 | 4.020251 | 4.87E−03 |
| | | repair | | protein 101 | | | | |
| 204029_at | hg133a | | CELSR2 | cadherin, EGF LAG | 0.983943 | 3.375812 | 6.319244 | 3.02E−04 |
| | | | | seven-pass G-type | | | | |
| | | | | receptor 2 (flamingo | | | | |
| | | | | homolog, Drosophila) | | | | |
| 36499_at | hg133a | | CELSR2 | cadherin, EGF LAG | 0.749583 | 3.863029 | 4.149305 | 4.08E−03 |
| | | | | seven-pass G-type | | | | |
| | | | | receptor 2 (flamingo | | | | |
| | | | | homolog, Drosophila) | | | | |
| 212072_s_at | hg133a | Casein | CSNK2A1 | casein kinase 2, alpha 1 | 0.938793 | 2.401085 | 4.256318 | 3.65E−03 |
| | | kinase | | polypeptide | | | | |
| 226545_at | hg133b | | CD109 | CD109 antigen (Gov | 0.790565 | 3.095872 | 3.244071 | 1.40E−02 |
| | | | | platelet alloantigens) | | | | |
| 216379_x_at | hg133a | | CD24 | CD24 antigen (small cell | 0.830379 | 24.211609 | 5.110381 | 1.32E−03 |
| | | | | lung carcinoma cluster 4 | | | | |
| | | | | antigen) | | | | |
| 211075_s_at | hg133a | | CD47 | CD47 antigen (Rh-related | 0.997624 | 5.106641 | 4.470891 | 2.86E−03 |
| | | | | antigen, integrin- | | | | |
| | | | | associated signal | | | | |
| | | | | transducer) | | | | |
| 201005_at | hg133a | | CD9 | CD9 antigen (p24) | 0.922543 | 8.3639 | 5.945453 | 5.52E−04 |
| 201897_s_at | hg133a | Protein | CKS1B | CDC28 protein kinase | 0.761593 | 4.062966 | 6.916206 | 2.21E−04 |
| | | Kinase | | regulatory subunit 1B | | | | |
| 201938_at | hg133a | | CDK2AP1 | CDK2-associated protein 1 | 0.991972 | 2.897472 | 6.049333 | 4.34E−04 |
| 224240_s_at | hg133b | Chemokine | CCL28 | chemokine (C-C motif) | 0.868474 | 2.228058 | 4.083612 | 4.30E−03 |
| | | pathway | | ligand 28 | | | | |
| 217947_at | hg133a | Chemokine | CKLFSF6 | chemokine-like factor | 0.959345 | 3.865855 | 3.92362 | 5.63E−03 |
| | | pathway | | super family 6 | | | | |
| 212539_at | hg133a | | CHD1L | chromodomain helicase | 0.993577 | 2.175382 | 6.146211 | 3.89E−04 |
| | | | | DNA binding protein 1- | | | | |
| | | | | like | | | | |
| 223020_at | hg133b | | CRR9 | cisplatin resistance related | 0.937324 | 2.424563 | 4.547569 | 2.49E−03 |
| | | | | protein CRR9p | | | | |
| 203917_at | hg133a | | CXADR | coxsackie virus and | 0.83738 | 9.893099 | 5.092631 | 1.31E−03 |
| | | | | adenovirus receptor | | | | |
| 224516_s_at | hg133b | | CXXC5 | CXXC finger 5 | 0.932761 | 8.478176 | 6.067524 | 4.89E−04 |
| 224391_s_at | hg133b | | CSE-C | cytosolic sialic acid 9-O- | 0.947591 | 2.363881 | 3.529538 | 9.08E−03 |
| | | | | acetylesterase homolog | | | | |
| 201584_s_at | hg133a | | DDX39 | DEAD (Asp-Glu-Ala- | 0.999743 | 2.793412 | 4.813862 | 1.87E−03 |
| | | | | Asp) box polypeptide 39 | | | | |
| 223054_at | hg133b | | DNAJB11 | DnaJ (Hsp40) homolog, | 0.987183 | 2.581061 | 7.428122 | 1.13E−04 |
| | | | | subfamily B, member 11 | | | | |
| 221782_at | hg133a | | DNAJC10 | DnaJ (Hsp40) homolog, | 0.903468 | 2.127172 | 4.240343 | 3.48E−03 |
| | | | | subfamily C, member 10 | | | | |
| 218435_at | hg133a | | DNAJD1 | DnaJ (Hsp40) homolog, | 0.88587 | 3.229291 | 3.258658 | 1.31E−02 |
| | | | | subfamily D, member 1 | | | | |
| 232353_s_at | hg133b | | DUSP24 | dual specificity | 0.744061 | 2.452739 | 5.155434 | 1.19E−03 |
| | | | | phosphatase 24 (putative) | | | | |
| 204160_s_at | hg133a | | ENPP4 | ectonucleotide | 0.832627 | 2.140256 | 4.308833 | 2.74E−03 |
| | | | | pyrophosphatase/phosphodiesterase | | | | |
| | | | | 4 (putative | | | | |
| | | | | function) | | | | |
| 57163_at | hg133a | | ELOVL1 | elongation of very long | 0.915992 | 2.058741 | 5.839797 | 4.58E−04 |
| | | | | chain fatty acids | | | | |
| | | | | (FEN1/Elo2, SUR4/Elo3, | | | | |
| | | | | yeast)-like 1 | | | | |
| 217294_s_at | hg133a | | ENO1 | enolase 1, (alpha) | 0.932884 | 6.086585 | 3.682818 | 7.77E−03 |
| 227609_at | hg133b | | EPSTI1 | epithelial stromal | 0.988995 | 3.470329 | 4.827146 | 1.79E−03 |
| | | | | interaction 1 (breast) | | | | |
| 230518_at | hg133b | | EVA1 | epithelial V-like antigen 1 | 0.961549 | 2.397871 | 3.357759 | 1.18E−02 |
| 225764_at | hg133b | TEL | ETV6 | ets variant gene 6 (TEL | 0.745135 | 2.309754 | 10.44025 | 1.28E−06 |
| | | oncogene | | oncogene) | | | | |
| 223000_s_at | hg133b | | F11R | F11 receptor | 0.89894 | 2.660417 | 4.576852 | 2.09E−03 |
| 205661_s_at | hg133a | | PP591 | FAD-synthetase | 0.988568 | 2.455995 | 6.552392 | 2.60E−04 |
| 217916_s_at | hg133a | | FAM49B | family with sequence | 0.941426 | 2.368904 | 3.601202 | 8.10E−03 |
| | | | | similarity 49, member B | | | | |
| 220147_s_at | hg133a | | FAM60A | family with sequence | 0.98176 | 2.313353 | 4.049541 | 4.74E−03 |
| | | | | similarity 60, member A | | | | |
| 202345_s_at | hg133a | Fatty acid | FABP5 | fatty acid binding protein | 0.938921 | 2.247141 | 3.575225 | 7.69E−03 |
| | | pathway | | 5 (psoriasis-associated) | | | | |
| 212070_at | hg133a | GPCR | GPR56 | G protein-coupled | 0.797302 | 5.707014 | 5.344521 | 8.23E−04 |
| | | | | receptor 56 | | | | |
| 203560_at | hg133a | | GGH | gamma-glutamyl | 0.901028 | 2.243412 | 4.194093 | 3.66E−03 |
| | | | | hydrolase (conjugase, | | | | |
| | | | | folylpolygammaglutamyl | | | | |
| | | | | hydrolase) | | | | |
| 211015_s_at | hg133a | heat | HSPA4 | heat shock 70 kDa protein 4 | 0.937893 | 2.563586 | 5.41026 | 8.63E−04 |
| | | shock | | | | | | |
| 216484_x_at | hg133a | | HDGF | hepatoma-derived growth | 0.958638 | 3.113746 | 7.698256 | 1.06E−04 |
| | | | | factor (high-mobility | | | | |
| | | | | group protein 1-like) | | | | |
| 201587_s_at | hg133a | NFkB | IRAK1 | interleukin-1 receptor- | 0.978741 | 3.277708 | 4.328176 | 3.36E−03 |
| | | pathway | | associated kinase 1 | | | | |
| 210046_s_at | hg133a | | IDH2 | isocitrate dehydrogenase 2 | 0.971034 | 5.460502 | 6.910983 | 2.07E−04 |
| | | | | (NADP+), mitochondrial | | | | |
| 201609_x_at | hg133a | | ICMT | isoprenylcysteine | 0.928452 | 2.010629 | 7.961931 | 5.07E−05 |
| | | | | carboxyl | | | | |
| | | | | methyltransferase | | | | |
| 209212_s_at | hg133a | | KLF5 | Kruppel-like factor 5 | 0.841618 | 2.796807 | 3.365636 | 1.15E−02 |
| | | | | (intestinal) | | | | |
| 200650_s_at | hg133a | | LDHA | lactate dehydrogenase A | 1 | 2.457959 | 5.127703 | 1.10E−03 |
| 212449_s_at | hg133a | | LYPLA1 | lysophospholipase I | 0.997752 | 2.786006 | 4.549123 | 2.41E−03 |
| 215566_x_at | hg133a | | LYPLA2 | lysophospholipase II | 0.785935 | 2.016094 | 5.35557 | 8.94E−04 |
| 217871_s_at | hg133a | | MIF | macrophage migration | 0.995633 | 2.1591 | 5.062746 | 1.16E−03 |
| | | | | inhibitory factor | | | | |
| | | | | (glycosylation-inhibiting | | | | |
| | | | | factor) | | | | |
| 226039_at | hg133b | | MGAT4A | mannosyl (alpha-1,3-)- | 0.781841 | 2.611577 | 3.571194 | 8.73E−03 |
| | | | | glycoprotein beta-1,4-N- | | | | |
| | | | | acetylglucosaminyltransferase, | | | | |
| | | | | isoenzyme A | | | | |
| 224598_at | hg133b | | MGAT4B | mannosyl (alpha-1,3-)- | 0.99027 | 2.074382 | 4.758944 | 1.76E−03 |
| | | | | glycoprotein beta-1,4-N- | | | | |
| | | | | acetylglucosaminyltransferase, | | | | |
| | | | | isoenzyme B | | | | |
| 220189_s_at | hg133a | | MGAT4B | mannosyl (alpha-1,3-)- | 0.943353 | 2.863971 | 6.236433 | 3.56E−04 |
| | | | | glycoprotein beta-1,4-N- | | | | |
| | | | | acetylglucosaminyltransferase, | | | | |
| | | | | isoenzyme B | | | | |
| 203936_s_at | hg133a | NFkB; | MMP9 | matrix metalloproteinase | 0.995247 | 2.692718 | 4.154678 | 4.04E−03 |
| | | cell | | 9 (gelatinase B, 92 kDa | | | | |
| | | migration; | | gelatinase, 92 kDa type IV | | | | |
| | | angiogenesis | | collagenase) | | | | |
| 222036_s_at | hg133a | DNA | MCM4 | MCM4 minichromosome | 0.878035 | 3.023563 | 5.228523 | 1.13E−03 |
| | | replication | | maintenance deficient 4 | | | | |
| | | and | | (S. cerevisiae) | | | | |
| | | repair | | | | | | |
| 201761_at | hg133a | | MTHFD2 | methylenetetrahydrofolate | 0.752922 | 3.419406 | 7.516072 | 1.06E−04 |
| | | | | dehydrogenase (NADP+ | | | | |
| | | | | dependent) 2, | | | | |
| | | | | methenyltetrahydrofolate | | | | |
| | | | | cyclohydrolase | | | | |
| 210058_at | hg133a | MAP | MAPK13 | mitogen-activated protein | 0.912267 | 2.181056 | 3.321173 | 1.22E−02 |
| | | kinase | | kinase 13 | | | | |
| 215498_s_at | hg133a | MAP | MAP2K3 | mitogen-activated protein | 0.966667 | 2.012703 | 4.127083 | 3.94E−03 |
| | | kinase | | kinase 3 | | | | |
| 207847_s_at | hg133a | | MUC1 | mucin 1, transmembrane | 0.858703 | 10.919683 | 4.599795 | 2.31E−03 |
| 209421_at | hg133a | DNA | MSH2 | mutS homolog 2, colon | 0.807964 | 2.017856 | 3.906162 | 5.47E−03 |
| | | repair | | cancer, nonpolyposis type | | | | |
| | | | | 1 (E. coli) | | | | |
| 222992_s_at | hg133b | | NDUFB9 | NADH dehydrogenase | 0.998993 | 2.409564 | 4.050455 | 4.74E−03 |
| | | | | (ubiquinone) 1 beta | | | | |
| | | | | subcomplex, 9, 22 kDa | | | | |
| 224799_at | hg133b | Ubiquitin/ | NDFIP2 | Nedd4 family interacting | 0.750637 | 2.736941 | 4.774743 | 1.63E−03 |
| | | proteosome | | protein 2 | | | | |
| | | pathway | | | | | | |
| 225787_at | hg133b | | NCE2 | NEDD8-conjugating | 0.96383 | 2.00633 | 4.924297 | 1.44E−03 |
| | | | | enzyme | | | | |
| 202647_s_at | hg133a | (v-ras) | NRAS | neuroblastoma RAS viral | 0.803854 | 2.861847 | 7.041569 | 1.61E−04 |
| | | oncogene | | (v-ras) oncogene homolog | | | | |
| | | pathway | | | | | | |
| 203964_at | hg133a | Myc | NMI | N-myc (and STAT) | 0.861785 | 2.520095 | 7.118422 | 1.62E−04 |
| | | oncogene | | interactor | | | | |
| | | pathway | | | | | | |
| 210830_s_at | hg133a | | PON2 | paraoxonase 2 | 0.827617 | 2.538529 | 3.433416 | 1.07E−02 |
| 208824_x_at | hg133a | Kinase | PCTK1 | PCTAIRE protein kinase 1 | 0.920424 | 2.434793 | 3.691038 | 7.54E−03 |
| 201489_at | hg133a | | PPIF | peptidylprolyl isomerase | 0.90668 | 2.897949 | 6.494173 | 6.56E−05 |
| | | | | F (cyclophilin F) | | | | |
| 214129_at | hg133a | | PDE4DIP | phosphodiesterase 4D | 0.760758 | 3.749596 | 4.73573 | 2.07E−03 |
| | | | | interacting protein | | | | |
| | | | | (myomegalin) | | | | |
| 201037_at | hg133a | Kinase | PFKP | phosphofructokinase, | 0.953565 | 2.323225 | 3.654586 | 7.27E−03 |
| | | | | platelet | | | | |
| 227068_at | hg133b | | PGK1 | phosphoglycerate kinase 1 | 0.812911 | 4.206684 | 6.057108 | 4.56E−04 |
| 226245_at | hg133b | | KCTD1 | potassium channel | 0.800899 | 2.610148 | 3.269091 | 1.35E−02 |
| | | | | tetramerisation domain | | | | |
| | | | | containing 1 | | | | |
| 218302_at | hg133a | | PSENEN | presenilin enhancer 2 | 0.870135 | 3.607088 | 4.207406 | 3.87E−03 |
| | | | | homolog (C. elegans) | | | | |
| 204839_at | hg133a | | POP5 | processing of precursor 5, | 0.999037 | 2.040627 | 5.014888 | 1.29E−03 |
| | | | | ribonuclease P/MRP | | | | |
| | | | | subunit (S. cerevisiae) | | | | |
| 200656_s_at | hg133a | | P4HB | procollagen-proline, 2- | 0.981888 | 2.204421 | 11.24001 | 1.96E−11 |
| | | | | oxoglutarate 4- | | | | |
| | | | | dioxygenase (proline 4- | | | | |
| | | | | hydroxylase), beta | | | | |
| | | | | polypeptide (protein | | | | |
| | | | | disulfide isomerase- | | | | |
| | | | | associated 1) | | | | |
| 212694_s_at | hg133a | | PCCB | propionyl Coenzyme A | 0.846179 | 2.202604 | 4.256623 | 2.66E−03 |
| | | | | carboxylase, beta | | | | |
| | | | | polypeptide | | | | |
| 205128_x_at | hg133a | | PTGS1 | prostaglandin- | 0.857868 | 6.921237 | 4.05741 | 4.81E−03 |
| | | | | endoperoxide synthase 1 | | | | |
| | | | | (prostaglandin G/H | | | | |
| | | | | synthase and | | | | |
| | | | | cyclooxygenase) | | | | |
| 201267_s_at | hg133a | proteasome | PSMC3 | proteasome (prosome, | 0.793642 | 2.146145 | 5.029416 | 1.14E−03 |
| | | | | macropain) 26S subunit, | | | | |
| | | | | ATPase, 3 | | | | |
| 212296_at | hg133a | proteasome | PSMD14 | proteasome (prosome, | 0.997238 | 2.672869 | 4.06281 | 4.63E−03 |
| | | | | macropain) 26S subunit, | | | | |
| | | | | non-ATPase, 14 | | | | |
| 210460_s_at | hg133a | proteasome | PSMD4 | proteasome (prosome, | 0.978227 | 2.093454 | 3.60247 | 8.35E−03 |
| | | | | macropain) 26S subunit, | | | | |
| | | | | non-ATPase, 4 | | | | |
| 201762_s_at | hg133a | proteasome | PSME2 | proteasome (prosome, | 0.997238 | 2.468623 | 3.480573 | 9.98E−03 |
| | | | | macropain) activator | | | | |
| | | | | subunit 2 (PA28 beta) | | | | |
| 201400_at | hg133a | proteasome | PSMB3 | proteasome (prosome, | 0.99878 | 2.376467 | 4.758101 | 1.83E−03 |
| | | | | macropain) subunit, beta | | | | |
| | | | | type, 3 | | | | |
| 213518_at | hg133a | Kinase | PRKCI | protein kinase C, iota | 0.772704 | 4.41575 | 3.587967 | 8.82E−03 |
| 200846_s_at | hg133a | | PPP1CA | protein phosphatase 1, | 0.929929 | 4.45379 | 8.217329 | 6.04E−05 |
| | | | | catalytic subunit, alpha | | | | |
| | | | | isoform | | | | |
| 206687_s_at | hg133a | | PTPN6 | protein tyrosine | 0.850032 | 2.543142 | 4.426893 | 2.90E−03 |
| | | | | phosphatase, non-receptor | | | | |
| | | | | type 6 | | | | |
| 212640_at | hg133a | | PTPLB | protein tyrosine | 0.996789 | 2.055999 | 4.891242 | 1.24E−03 |
| | | | | phosphatase-like (proline | | | | |
| | | | | instead of catalytic | | | | |
| | | | | arginine), member b | | | | |
| 202671_s_at | hg133a | | PDXK | pyridoxal (pyridoxine, | 0.95228 | 2.74402 | 5.316618 | 8.82E−04 |
| | | | | vitamin B6) kinase | | | | |
| 217848_s_at | hg133a | | PP | pyrophosphatase | 0.987797 | 3.721445 | 4.691774 | 2.18E−03 |
| | | | | (inorganic) | | | | |
| 201251_at | hg133a | Kinase | PKM2 | pyruvate kinase, muscle | 0.961978 | 3.522671 | 7.490227 | 1.25E−04 |
| 223471_at | hg133b | | RAB3IP | RAB3A interacting | 0.75567 | 3.132951 | 3.852934 | 5.96E−03 |
| | | | | protein (rabin3) | | | | |
| 208819_at | hg133a | RAS | RAB8A | RAB8A, member RAS | 0.99544 | 2.016693 | 4.516892 | 2.54E−03 |
| | | oncogene | | oncogene family | | | | |
| | | family | | | | | | |
| | | pathway | | | | | | |
| 222077_s_at | hg133a | RAS | RACGAP1 | Rac GTPase activating | 0.955106 | 4.172509 | 4.227326 | 3.85E−03 |
| | | oncogene | | protein 1 | | | | |
| | | family | | | | | | |
| | | pathway | | | | | | |
| 202483_s_at | hg133a | RAS | RANBP1 | RAN binding protein 1 | 0.838471 | 2.071267 | 4.171917 | 3.89E−03 |
| | | oncogene | | | | | | |
| | | family | | | | | | |
| | | pathway | | | | | | |
| 200750_s_at | hg133a | RAS | RAN | RAN, member RAS | 0.998715 | 2.293692 | 7.382216 | 9.70E−05 |
| | | oncogene | | oncogene family | | | | |
| | | family | | | | | | |
| | | pathway | | | | | | |
| 35666_at | hg133a | | SEMA3F | sema domain, | 0.922672 | 2.040823 | 3.557648 | 8.69E−03 |
| | | | | immunoglobulin domain | | | | |
| | | | | (Ig), short basic domain, | | | | |
| | | | | secreted, (semaphorin) 3F | | | | |
| 212572_at | hg133a | serine/threonine | STK38L | serine/threonine kinase 38 | 0.995633 | 2.085237 | 3.515383 | 9.37E−03 |
| | | kinase | | like | | | | |
| 201563_at | hg133a | | SORD | sorbitol dehydrogenase | 0.975851 | 2.674268 | 3.924998 | 5.11E−03 |
| 212322_at | hg133a | | SGPL1 | sphingosine-1-phosphate | 0.813231 | 3.919159 | 5.633239 | 7.70E−04 |
| | | | | lyase 1 | | | | |
| 226560_at | hg133b | | SGPP2 | sphingosine-1-phosphate | 0.812844 | 3.929339 | 4.655677 | 2.10E−03 |
| | | | | phosphotase 2 | | | | |
| 201998_at | hg133a | | ST6GAL1 | ST6 beta-galactosamide | 0.905909 | 3.55091 | 3.545618 | 9.33E−03 |
| | | | | alpha-2,6-sialyltranferase 1 | | | | |
| 222750_s_at | hg133b | | SRD5A2L | steroid 5 alpha-reductase | 0.928332 | 2.432341 | 4.238749 | 3.44E−03 |
| | | | | 2-like | | | | |
| 202589_at | hg133a | | TYMS | thymidylate synthetase; | 0.919332 | 4.968409 | 6.711329 | 2.40E−04 |
| | | | | Inhibitor: 5-fluorouracil, | | | | |
| | | | | 5-fluoro-2-prime- | | | | |
| | | | | deoxyuridine, and some | | | | |
| | | | | folate analogs | | | | |
| 213011_s_at | hg133a | | TPI1 | triosephosphate isomerase 1 | 0.999294 | 3.205511 | 8.305632 | 6.16E−05 |
| 202510_s_at | hg133a | NFkB | TNFAIP2 | tumor necrosis factor, | 0.798523 | 4.43632 | 4.176119 | 4.09E−03 |
| | | patwhay | | alpha-induced protein 2 | | | | |
| 201688_s_at | hg133a | | TPD52 | tumor protein D52 | 0.812139 | 5.502568 | 4.799016 | 1.62E−03 |
| 208743_s_at | hg133a | | YWHAB | tyrosine 3- | 0.995504 | 2.515535 | 4.713777 | 1.78E−03 |
| | | | | monooxygenase/tryptophan | | | | |
| | | | | 5-monooxygenase | | | | |
| | | | | activation protein, beta | | | | |
| | | | | polypeptide | | | | |
| 200638_s_at | hg133a | | YWHAZ | tyrosine 3- | 0.998587 | 2.014093 | 3.890667 | 5.57E−03 |
| | | | | monooxygenase/tryptophan | | | | |
| | | | | 5-monooxygenase | | | | |
| | | | | activation protein, zeta | | | | |
| | | | | polypeptide | | | | |
| 214695_at | hg133a | Ubiquitin/ | UBAP2L | ubiquitin associated | 0.842903 | 2.251556 | 5.86933 | 5.50E−04 |
| | | proteosome | | protein 2-like | | | | |
| | | patwhay | | | | | | |
| 202779_s_at | hg133a | Ubiquitin/ | UBE2S | ubiquitin-conjugating | 0.743224 | 2.638422 | 4.503814 | 2.46E−03 |
| | | proteosome | | enzyme E2S | | | | |
| | | patwhay | | | | | | |
| 222870_s_at | hg133b | | B3GNT1 | UDP-GlcNAc:betaGal | 0.908938 | 3.325586 | 6.177822 | 3.90E−04 |
| | | | | beta-1,3-N- | | | | |
| | | | | acetylglucosaminyltransferase 1 | | | | |
| 210512_s_at | hg133a | VEGF | VEGF | vascular endothelial | 0.949133 | 3.871465 | 3.610859 | 8.34E−03 |
| | | | | growth factor | | | | |
| 226063_at | hg133b | vav 2 | VAV2 | vav 2 oncogene | 0.906187 | 2.037705 | 8.51942 | 1.63E−05 |
| | | oncogene | | | | | | |
| | | pathway | | | | | | |
| 202454_s_at | hg133a | HER3 | ERBB3 | v-erb-b2 erythroblastic | 0.861207 | 3.990508 | 4.35777 | 3.10E−03 |
| | | | | leukemia viral oncogene | | | | |
| | | | | homolog 3 (avian) | | | | |
| 214435_x_at | hg133a | Ral | RALA | v-ral simian leukemia | 0.97386 | 2.278349 | 3.552995 | 8.91E−03 |
| | | oncogene | | viral oncogene homolog | | | | |
| | | | | A (ras related) | | | | |

TABLE X
PARP1 Upregulated - Diff/X (Human); Name: Upregulated Breast Infiltrating Lobular Carcinoma vs.
normal Primary No Smoking History (Minimum Fold Change: 2.0); Experiment: Breast, Infiltrating
Lobular Carcinoma, Primary; No Smoking History; Control: normal breast, no smoking history.

| Fragment | | | | | Pres. | Fold | | |
|---|---|---|---|---|---|---|---|---|
| Name | Array | Pathway | Symbol | Description | Freq. | Change | t-Score | p-Value |
| 201261_x_at | hg133a | | BGN | biglycan | 0.825819 | 4.750057 | 4.30732 | 1.84E−03 |
| 202391_at | hg133a | | BASP1 | brain abundant, membrane | 0.968208 | 2.028573 | 3.74687 | 3.87E−03 |
| | | | | attached signal protein 1 | | | | |
| 212551_at | hg133a | | CAP2 | CAP, adenylate cyclase- | 0.753565 | 2.17528 | 3.45136 | 6.03E−03 |
| | | | | associated protein, 2 | | | | |
| | | | | (yeast) | | | | |
| 201584_s_at | hg133a | | DDX39 | DEAD (Asp-Glu-Ala- | 0.999743 | 2.016788 | 3.84071 | 3.54E−03 |
| | | | | Asp) box polypeptide 39 | | | | |
| 212303_x_at | hg133a | | KHSRP | KH-type splicing | 0.748491 | 2.297845 | 3.95656 | 2.36E−03 |
| | | | | regulatory protein (FUSE | | | | |
| | | | | binding protein 2) | | | | |
| 222212_s_at | hg133a | | LASS2 | LAG1 longevity | 0.928516 | 2.302124 | 4.30408 | 1.47E−03 |
| | | | | assurance homolog 2 (S. cerevisiae) | | | | |
| 218211_s_at | hg133a | | MLPH | melanophilin | 0.982852 | 2.830613 | 2.94365 | 1.57E−02 |
| 218039_at | hg133a | | NUSAP1 | nucleolar and spindle | 0.920938 | 3.719661 | 3.5004 | 6.42E−03 |
| | | | | associated protein 1 | | | | |
| 210004_at | hg133a | | OLR1 | oxidized low density | 0.890751 | 2.80369 | 4.31047 | 1.58E−03 |
| | | | | lipoprotein (lectin-like) | | | | |
| | | | | receptor 1 | | | | |
| 230097_at | hg133b | | GART | phosphoribosylglycinamide | 0.899074 | 2.141418 | 4.23441 | 1.78E−03 |
| | | | | formyltransferase, | | | | |
| | | | | phosphoribosylglycinamide | | | | |
| | | | | synthetase, | | | | |
| | | | | phosphoribosylaminoimidazole | | | | |
| | | | | synthetase | | | | |
| 224742_at | hg133b | | PYGB | phosphorylase, glycogen; | 0.756207 | 2.319074 | 4.9306 | 5.18E−04 |
| | | | | brain | | | | |
| 208874_x_at | hg133a | | PPP2R4 | protein phosphatase 2A, | 0.950996 | 2.140618 | 3.29413 | 8.72E−03 |
| | | | | regulatory subunit B′ (PR | | | | |
| | | | | 53) | | | | |
| 217763_s_at | hg133a | RAS | RAB31 | RAB31, member RAS | 0.802505 | 4.597113 | 4.43946 | 1.54E−03 |
| | | | | oncogene family | | | | |
| 35666_at | hg133a | | SEMA3F | sema domain, | 0.922672 | 2.656249 | 2.92337 | 1.66E−02 |
| | | | | immunoglobulin domain | | | | |
| | | | | (Ig), short basic domain, | | | | |
| | | | | secreted, (semaphorin) 3F | | | | |
| 36545_s_at | hg133a | | SFI1 | Sfi1 homolog, spindle | 0.762492 | 2.052907 | 3.38266 | 7.23E−03 |
| | | | | assembly associated | | | | |
| | | | | (yeast) | | | | |
| 218813_s_at | hg133a | | SH3GLB2 | SH3-domain GRB2-like | 0.757161 | 2.091861 | 2.88131 | 1.54E−02 |
| | | | | endophilin B2 | | | | |
| 201563_at | hg133a | | SORD | sorbitol dehydrogenase | 0.975851 | 2.70965 | 2.79542 | 1.97E−02 |
| 222651_s_at | hg133b | | TRPS1 | trichorhinophalangeal | 0.942357 | 2.426509 | 3.14684 | 1.13E−02 |
| | | | | syndrome I | | | | |
| 209413_at | hg133a | | B4GALT2 | UDP-Gal:betaGlcNAc | 0.903854 | 2.037055 | 5.13462 | 4.04E−04 |
| | | | | beta 1,4- | | | | |
| | | | | galactosyltransferase, | | | | |
| | | | | polypeptide 2 | | | | |
| 218807_at | hg133a | VAV | VAV3 | 0.927489 | | 2.161 | 2.99273 | 1.44E−02 |
| | | oncogene; | oncogene | | | | | |
| | | can | | | | | | |
| | | enhance | | | | | | |
| | | NFkB | | | | | | |

TABLE XI
PARP1 Upregulated - Diff/X (Human); Name: Upregulated Endometrium Mullerian Mixed Tumor
Primary (Minimum Fold Change: 2.0); Experiment: Endometrium, Mullerian Mixed Tumor,
Primary; control: normal endometrium.

| Fragment | | | | | Pres. | Fold | | |
|---|---|---|---|---|---|---|---|---|
| Name | Array | Pathway | Symbol | Description | Freq. | Change | t-Score | p-Value |
| 204998_s_at | hg133a | | ATF5 | activating transcription | 0.971227 | 2.199928 | 3.165348 | 1.75E−02 |
| | | | | factor 5 | | | | |
| 201281_at | hg133a | | ADRM1 | adhesion regulating | 0.994477 | 2.484654 | 3.547021 | 1.07E−02 |
| | | | | molecule 1 | | | | |
| 217791_s_at | hg133a | | ALDH18A1 | aldehyde dehydrogenase | 0.953565 | 2.155068 | 3.493927 | 1.00E−02 |
| | | | | 18 family, member A1 | | | | |
| 201272_at | hg133a | | AKR1B1 | aldo-keto reductase family | 0.981246 | 2.787189 | 4.036007 | 6.39E−03 |
| | | | | 1, member B1 (aldose | | | | |
| | | | | reductase) | | | | |
| 208002_s_at | hg133a | | BACH | brain acyl-CoA hydrolase | 0.840784 | 2.869609 | 4.005982 | 6.58E−03 |
| 201897_s_at | hg133a | Kinase | CKS1B | CDC28 protein kinase | 0.761593 | 3.453322 | 3.311789 | 1.53E−02 |
| | | | | regulatory subunit 1B | | | | |
| 212737_at | hg133a | | CSH2 | chorionic | 0.798651 | 2.059874 | 4.906849 | 1.78E−03 |
| | | | | somatomammotropin | | | | |
| | | | | hormone 2 | | | | |
| 223020_at | hg133b | | CRR9 | cisplatin resistance related | 0.937324 | 2.313138 | 4.443777 | 2.85E−03 |
| | | | | protein CRR9p | | | | |
| 233955_x_at | hg133b | | CXXC5 | CXXC finger 5 | 0.916991 | 2.256594 | 3.043573 | 1.84E−02 |
| 200881_s_at | hg133a | | DNAJA1 | DnaJ (Hsp40) homolog, | 0.998266 | 2.010298 | 4.815776 | 1.85E−03 |
| | | | | subfamily A, member 1 | | | | |
| 217294_s_at | hg133a | | ENO1 | enolase 1, (alpha) | 0.932884 | 2.580105 | 3.411278 | 9.80E−03 |
| 234464_s_at | hg133b | | EME1 | essential meiotic | 0.916119 | 2.208916 | 5.190527 | 7.77E−04 |
| | | | | endonuclease 1 homolog 1 | | | | |
| | | | | (S. pombe) | | | | |
| 225099_at | hg133b | | FBXO45 | F-box protein 45 | 0.87136 | 2.303275 | 3.098714 | 1.94E−02 |
| 213187_x_at | hg133a | | FTL | ferritin, light polypeptide | 0.99955 | 2.25979 | 3.343599 | 1.34E−02 |
| 213187_x_at | hg133a | | FTLL1 | ferritin, light polypeptide- | 0.99955 | 2.25979 | 3.343599 | 1.34E−02 |
| | | | | like 1 | | | | |
| 203560_at | hg133a | | GGH | gamma-glutamyl | 0.901028 | 4.671918 | 5.171402 | 1.84E−03 |
| | | | | hydrolase (conjugase, | | | | |
| | | | | folylpolygammaglutamyl | | | | |
| | | | | hydrolase) | | | | |
| 208308_s_at | hg133a | | GPI | glucose phosphate | 0.998715 | 2.184172 | 3.847515 | 5.58E−03 |
| | | | | isomerase | | | | |
| 214431_at | hg133a | | GMPS | guanine monophosphate | 0.921002 | 2.00813 | 3.029078 | 1.74E−02 |
| | | | | synthetase | | | | |
| 200052_s_at | hg133a | | ILF2 | interleukin enhancer | 0.997624 | 2.084774 | 5.364201 | 7.16E−04 |
| | | | | binding factor 2, 45 kDa | | | | |
| 203362_s_at | hg133a | | MAD2L1 | MAD2 mitotic arrest | 0.808863 | 5.128772 | 3.938453 | 6.99E−03 |
| | | | | deficient-like 1 (yeast) | | | | |
| 222036_s_at | hg133a | DNA | MCM4 | MCM4 minichromosome | 0.878035 | 2.73404 | 3.884012 | 7.07E−03 |
| | | replication | | maintenance deficient 4 | | | | |
| | | | | (S. cerevisiae) | | | | |
| 209014_at | hg133a | melanoma | MAGED1 | melanoma antigen family | 0.908028 | 2.589493 | 5.149489 | 9.65E−04 |
| | | antigen | | D, 1 | | | | |
| 222547_at | hg133b | | MAP4K4 | mitogen-activated protein | 0.932291 | 2.608968 | 5.636816 | 9.69E−04 |
| | | | | kinase 4 | | | | |
| 209421_at | hg133a | DNA | MSH2 | mutS homolog 2, colon | 0.807964 | 2.233455 | 3.754382 | 7.44E−03 |
| | | repair | | cancer, nonpolyposis type | | | | |
| | | | | 1 (E. coli) | | | | |
| 201669_s_at | hg133a | | MARCKS | myristoylated alanine-rich | 0.972704 | 2.77493 | 6.063445 | 2.86E−04 |
| | | | | protein kinase C substrate | | | | |
| 202647_s_at | hg133a | Ras | NRAS | neuroblastoma RAS viral | 0.803854 | 2.246652 | 3.597351 | 8.17E−03 |
| | | oncogene | | (v-ras) oncogene homolog | | | | |
| 202784_s_at | hg133a | | NNT | nicotinamide nucleotide | 0.745151 | 2.184823 | 3.623844 | 8.39E−03 |
| | | | | transhydrogenase | | | | |
| 226287_at | hg133b | | NY-REN- | NY-REN-41 antigen | 0.967119 | 2.972643 | 3.634791 | 1.04E−02 |
| | | | 41 | | | | | |
| 226649_at | hg133b | Kinase | PANK1 | pantothenate kinase 1 | 0.797611 | 2.993086 | 3.332211 | 1.47E−02 |
| 208938_at | hg133a | | PRCC | papillary renal cell | 0.854143 | 2.155869 | 4.299584 | 3.50E−03 |
| | | | | carcinoma (translocation- | | | | |
| | | | | associated) | | | | |
| 207239_s_at | hg133a | kinase | PCTK1 | PCTAIRE protein kinase 1 | 0.972511 | 2.31441 | 6.137455 | 8.80E−05 |
| 201118_at | hg133a | | PGD | phosphogluconate | 0.835902 | 2.397979 | 3.940177 | 5.15E−03 |
| | | | | dehydrogenase | | | | |
| 217356_s_at | hg133a | kinase | PGK1 | phosphoglycerate kinase 1 | 0.951252 | 2.569093 | 4.552546 | 2.01E−03 |
| 201050_at | hg133a | | PLD3 | phospholipase D3 | 0.871933 | 3.774503 | 3.303931 | 1.58E−02 |
| 200827_at | hg133a | | PLOD1 | procollagen-lysine 1,2- | 0.856005 | 2.186071 | 3.386693 | 1.29E−02 |
| | | | | oxoglutarate 5- | | | | |
| | | | | dioxygenase 1 | | | | |
| 201388_at | hg133a | proteasome | PSMD3 | proteasome (prosome, | 0.953243 | 2.129344 | 3.200389 | 1.53E−02 |
| | | | | macropain) 26S subunit, | | | | |
| | | | | non-ATPase, 3 | | | | |
| 210460_s_at | hg133a | proteasome | PSMD4 | proteasome (prosome, | 0.978227 | 2.159115 | 3.322456 | 1.49E−02 |
| | | | | macropain) 26S subunit, | | | | |
| | | | | non-ATPase, 4 | | | | |
| 200820_at | hg133a | proteasome | PSMD8 | proteasome (prosome, | 0.944444 | 2.352294 | 3.388362 | 1.36E−02 |
| | | | | macropain) 26S subunit, | | | | |
| | | | | non-ATPase, 8 | | | | |
| 216088_s_at | hg133a | proteasome | PSMA7 | proteasome (prosome, | 0.74894 | 2.138835 | 3.712278 | 8.60E−03 |
| | | | | macropain) subunit, alpha | | | | |
| | | | | type, 7 | | | | |
| 229606_at | hg133b | | PPP3CA | protein phosphatase 3 | 0.985572 | 2.383108 | 3.138387 | 1.81E−02 |
| | | | | (formerly 2B), catalytic | | | | |
| | | | | subunit, alpha isoform | | | | |
| | | | | (calcineurin A alpha) | | | | |
| 202671_s_at | hg133a | | PDXK | pyridoxal (pyridoxine, | 0.95228 | 2.524332 | 4.092174 | 4.54E−03 |
| | | | | vitamin B6) kinase | | | | |
| 222077_s_at | hg133a | Rho | RACGAP1 | Rac GTPase activating | 0.955106 | 3.974284 | 3.970845 | 6.97E−03 |
| | | GTPase | | protein 1 | | | | |
| | | pathway | | | | | | |
| 200750_s_at | hg133a | RAS | RAN | RAN, member RAS | 0.998715 | 2.177619 | 4.181275 | 4.59E−03 |
| | | oncogene | | oncogene family | | | | |
| | | pathway | | | | | | |
| 204023_at | hg133a | DNA | RFC4 | replication factor C | 0.821644 | 2.51157 | 4.484135 | 3.39E−03 |
| | | repair | | (activator 1) 4, 37 kDa | | | | |
| 225202_at | hg133b | Rho | RHOBTB3 | Rho-related BTB domain | 0.966246 | 2.076071 | 4.080449 | 6.98E−04 |
| | | GTPase | | containing 3 | | | | |
| | | pathway | | | | | | |
| 203022_at | hg133a | | RNASEH2A | ribonuclease H2, large | 0.991779 | 3.307084 | 3.531659 | 1.19E−02 |
| | | | | subunit | | | | |
| 213194_at | hg133a | beta- | ROBO1 | roundabout, axon guidance | 0.769685 | 2.213264 | 3.212126 | 1.60E−02 |
| | | catenin | | receptor, homolog 1 | | | | |
| | | pathway | | (Drosophila) | | | | |
| 201516_at | hg133a | | SRM | spermidine synthase | 0.900771 | 2.483222 | 3.196424 | 1.72E−02 |
| 218854_at | hg133a | | SART2 | squamous cell carcinoma | 0.887091 | 2.045827 | 3.387623 | 1.16E−02 |
| | | | | antigen recognized by T | | | | |
| | | | | cells 2 | | | | |
| 225639_at | hg133b | Src | SCAP2 | src family associated | 0.845256 | 2.392457 | 3.432378 | 8.94E−03 |
| | | oncogene | | phosphoprotein 2 | | | | |
| | | pathway | | | | | | |
| 202589_at | hg133a | | TYMS | thymidylate synthetase; | 0.919332 | 6.265697 | 3.799391 | 8.73E−03 |
| | | | | inhibitor: 5-fluorouracil, 5- | | | | |
| | | | | fluoro-2-prime- | | | | |
| | | | | deoxyuridine, and some | | | | |
| | | | | folate analogs | | | | |
| 204033_at | hg133a | | TRIP13 | thyroid hormone receptor | 0.792036 | 4.456018 | 3.205925 | 1.81E−02 |
| | | | | interactor 13 | | | | |
| 214695_at | hg133a | proteasome/ | UBAP2L | ubiquitin associated | 0.842903 | 2.011192 | 4.600983 | 2.96E−03 |
| | | ubiquitin | | protein 2-like | | | | |
| 201001_s_at | hg133a | proteasome/ | UBE2V1 | ubiquitin-conjugating | 0.954335 | 2.057304 | 4.585788 | 2.09E−03 |
| | | ubiquitin | | enzyme E2 variant 1 | | | | |
| 202779_s_at | hg133a | proteasome/ | UBE2S | ubiquitin-conjugating | 0.743224 | 5.046636 | 4.466871 | 3.94E−03 |
| | | ubiquitin | | enzyme E2S | | | | |
| 217788_s_at | hg133a | | GALNT2 | UDP-N-acetyl-alpha-D- | 0.979127 | 2.144073 | 3.58495 | 9.19E−03 |
| | | | | galactosamine:polypeptide | | | | |
| | | | | N- | | | | |
| | | | | acetylgalactosaminyltransferase | | | | |
| | | | | 2 (GalNAc-T2) | | | | |
| 212038_s_at | hg133a | | VDAC1 | voltage-dependent anion | 0.999422 | 2.213029 | 6.949417 | 6.35E−05 |
| | | | | channel 1 | | | | |

TABLE XII
PARP1 Upregulated - Diff/X (Human); Name: Upregulated Liver Hepatocellular Carcinoma (Minimum
Fold Change: 2.0); Experiment: Liver, Hepatocellular Carcinoma; control: Liver, Focal Nodular Hyperplasia.

| Fragment | | | | | Pres. | Fold | | |
|---|---|---|---|---|---|---|---|---|
| Name | Array | Pathway | Symbol | Description | Freq. | Change | t-Score | p-Value |
| 232007_at | hg133b | | AGPAT5 | 1-acylglycerol-3- | 0.768286 | 2.121421 | 2.677231 | 1.58E−02 |
| | | | | phosphate O- | | | | |
| | | | | acyltransferase 5 | | | | |
| | | | | (lysophosphatidic acid | | | | |
| | | | | acyltransferase, epsilon) | | | | |
| 201662_s_at | hg133a | Fatty | ACSL3 | acyl-CoA synthetase long- | 0.966346 | 2.203047 | 2.87949 | 1.00E−02 |
| | | acids | | chain family member 3 | | | | |
| | | pathway | | | | | | |
| 200966_x_at | hg133a | | ALDOA | aldolase A, fructose- | 0.991715 | 3.38799 | 3.488556 | 3.18E−03 |
| | | | | bisphosphate | | | | |
| 210896_s_at | hg133a | | ASPH | aspartate beta-hydroxylase | 0.795697 | 2.094409 | 2.781446 | 1.34E−02 |
| 220948_s_at | hg133a | ATPase | ATP1A1 | ATPase, Na+/K+ | 0.999615 | 2.035844 | 4.212664 | 3.59E−04 |
| | | | | transporting, alpha 1 | | | | |
| | | | | polypeptide | | | | |
| 201940_at | hg133a | | CPD | carboxypeptidase D | 0.862428 | 2.057728 | 3.355704 | 2.93E−03 |
| 203987_at | hg133a | Wnt-beta | FZD6 | frizzled homolog 6 | 0.958317 | 2.293808 | 2.923516 | 9.48E−03 |
| | | catenin | | (Drosophila) | | | | |
| | | pathway | | | | | | |
| 201816_s_at | hg133a | | GBAS | glioblastoma amplified | 0.994926 | 2.030767 | 2.823487 | 1.09E−02 |
| | | | | sequence | | | | |
| 209448_at | hg133a | | HTATIP2 | HIV-1 Tat interactive | 0.855427 | 2.128596 | 3.47696 | 2.15E−03 |
| | | | | protein 2, 30 kDa | | | | |
| 201587_s_at | hg133a | NFkB | IRAK1 | interleukin-1 receptor- | 0.978741 | 2.27196 | 5.230533 | 4.47E−05 |
| | | activation | | associated kinase 1 | | | | |
| 226350_at | hg133b | | KMO | kynurenine 3- | 0.850758 | 2.184306 | 2.984445 | 7.71E−03 |
| | | | | monooxygenase | | | | |
| | | | | (kynurenine 3- | | | | |
| | | | | hydroxylase) | | | | |
| 202651_at | hg133a | | LPGAT1 | lysophosphatidylglycerol | 0.993834 | 2.029841 | 4.138422 | 5.69E−04 |
| | | | | acyltransferase 1 | | | | |
| 203936_s_at | hg133a | NFkB; | matrix | 0.995247 | Liver, | 2.222868 | 3.10948 | 5.91E−03 |
| | | cell | metalloproteinase 9 | | Hepatocellular | | | |
| | | migration; | (MMP9; | | Carcinoma | | | |
| | | angiogenesis | gelatinase | | | | | |
| | | | B, 92 kDa | | | | | |
| | | | gelatinase, | | | | | |
| | | | 92 kDa | | | | | |
| | | | type IV | | | | | |
| | | | collagenase) | | | | | |
| 222036_s_at | hg133a | DNA | MCM4 | MCM4 minichromosome | 0.878035 | 3.097419 | 3.398078 | 3.51E−03 |
| | | replication | | maintenance deficient 4 | | | | |
| | | and | | (S. cerevisiae) | | | | |
| | | repair | | | | | | |
| 200790_at | hg133a | | ODC1 | ornithine decarboxylase 1 | 0.934682 | 2.555112 | 3.892729 | 1.23E−03 |
| 224937_at | hg133b | | PTGFRN | prostaglandin F2 receptor | 0.758891 | 2.184184 | 2.566146 | 1.86E−02 |
| | | | | negative regulator | | | | |
| 222077_s_at | hg133a | GTPase | RACGAP1 | Rac GTPase activating | 0.955106 | 3.23545 | 3.373596 | 4.00E−03 |
| | | | | protein 1 | | | | |
| 213194_at | hg133a | | ROBO1 | roundabout, axon | 0.769685 | 3.671868 | 3.494206 | 2.99E−03 |
| | | | | guidance receptor, | | | | |
| | | | | homolog 1 (Drosophila) | | | | |
| 209875_s_at | hg133a | | SPP1 | secreted phosphoprotein 1 | 0.796275 | 14.27776 | 4.37561 | 5.27E−04 |
| | | | | (osteopontin, bone | | | | |
| | | | | sialoprotein I, early T- | | | | |
| | | | | lymphocyte activation 1) | | | | |
| 214853_s_at | hg133a | | SHC1 | SHC (Src homology 2 | 0.992871 | 2.034756 | 4.756677 | 1.52E−04 |
| | | | | domain containing) | | | | |
| | | | | transforming protein 1 | | | | |
| 217979_at | hg133a | | TSPAN13 | tetraspanin 13 | 0.973732 | 3.346962 | 3.708996 | 1.81E−03 |
| 201266_at | hg133a | | TXNRD1 | thioredoxin reductase 1 | 0.995633 | 2.501586 | 3.009625 | 8.12E−03 |
| 208699_x_at | hg133a | | TKT | transketolase (Wernicke- | 0.933398 | 2.53584 | 2.779767 | 1.31E−02 |
| | | | | Korsakoff syndrome) | | | | |
| 202779_s_at | hg133a | Ubiquitin/ | UBE2S | ubiquitin-conjugating | 0.743224 | 2.300054 | 3.696056 | 1.45E−03 |
| | | proteosome | | enzyme E2S | | | | |

TABLE XIII
PARP1 Upregulated - Diff/X (Human); Name: Upregulated Endometrium Adenocarcinoma
Endometrioid Type Primary (Minimum Fold Change: 2.0); Experiment: Endometrium, Adenocarcinoma, Endometrioid
Type, Primary; control: normal endometrium.

| Fragment | | | | | Pres. | Fold | | |
|---|---|---|---|---|---|---|---|---|
| Name | Array | Pathway | Symbol | Description | Freq. | Change | t-Score | p-Value |
| 202912_at | hg133a | | ADM | adrenomedullin | 0.835967 | 2.736399 | 5.702427 | 3.94E−07 |
| 222416_at | hg133b | | ALDH18A1 | aldehyde dehydrogenase | 0.73923 | 2.256697 | 8.371288 | 5.53E−12 |
| | | | | 18 family, member A1 | | | | |
| 204976_s_at | hg133a | | AMMECR1 | Alport syndrome, mental | 0.818561 | 2.050739 | 7.130386 | 8.98E−10 |
| | | | | retardation, midface | | | | |
| | | | | hypoplasia and | | | | |
| | | | | elliptocytosis | | | | |
| | | | | chromosomal region, gene 1 | | | | |
| 201012_at | hg133a | | ANXA1 | annexin A1 | 0.980989 | 2.032341 | 4.241855 | 6.70E−05 |
| 222746_s_at | hg133b | | BSPRY | B-box and SPRY domain | 0.791974 | 2.126976 | 5.791611 | 2.29E−07 |
| | | | | containing | | | | |
| 201953_at | hg133a | | CIB1 | calcium and integrin | 0.997367 | 2.006992 | 6.899383 | 2.02E−09 |
| | | | | binding 1 (calmyrin) | | | | |
| 211657_at | hg133a | | CEACAM6 | carcinoembryonic antigen- | 0.740013 | 3.56842 | 3.04274 | 3.67E−03 |
| | | | | related cell adhesion | | | | |
| | | | | molecule 6 (non-specific | | | | |
| | | | | cross reacting antigen) | | | | |
| 203917_at | hg133a | | CXADR | coxsackie virus and | 0.83738 | 5.008366 | 9.373951 | 2.20E−13 |
| | | | | adenovirus receptor | | | | |
| 200606_at | hg133a | | DSP | desmoplakin | 0.914194 | 2.51112 | 5.671774 | 3.12E−07 |
| 221782_at | hg133a | | DNAJC10 | DnaJ (Hsp40) homolog, | 0.903468 | 2.532038 | 4.980326 | 6.36E−06 |
| | | | | subfamily C, member 10 | | | | |
| 204160_s_at | hg133a | | ENPP4 | ectonucleotide | 0.832627 | 2.562337 | 5.904651 | 1.73E−07 |
| | | | | pyrophosphatase/ | | | | |
| | | | | phosphodiesterase | | | | |
| | | | | 4 (putative | | | | |
| | | | | function) | | | | |
| 201231_s_at | hg133a | | ENO1 | enolase 1, (alpha) | 0.999743 | 2.337473 | 6.655107 | 6.34E−09 |
| 223000_s_at | hg133b | | F11R | F11 receptor | 0.89894 | 2.517537 | 8.344118 | 4.00E−12 |
| 239246_at | hg133b | | FARP1 | FERM, RhoGEF | 0.93464 | 2.108591 | 6.262126 | 2.67E−08 |
| | | | | (ARHGEF) and pleckstrin | | | | |
| | | | | domain protein 1 | | | | |
| | | | | (chondrocyte-derived) | | | | |
| 226145_s_at | hg133b | | FRAS1 | Fraser syndrome 1 | 0.780768 | 2.33359 | 5.114114 | 2.96E−06 |
| 212070_at | hg133a | GPCR | GPR56 | G protein-coupled receptor | 0.797302 | 2.196786 | 5.969663 | 1.24E−07 |
| | | | | 56 | | | | |
| 203560_at | hg133a | | GGH | gamma-glutamyl | 0.901028 | 3.400252 | 2.505948 | 1.55E−02 |
| | | | | hydrolase (conjugase, | | | | |
| | | | | folylpolygammaglutamyl | | | | |
| | | | | hydrolase) | | | | |
| 239761_at | hg133b | | GCNT1 | glucosaminyl (N-acetyl) | 0.849953 | 2.097008 | 4.560683 | 2.94E−05 |
| | | | | transferase 1, core 2 (beta- | | | | |
| | | | | 1,6-N- | | | | |
| | | | | acetylglucosaminyltransferase) | | | | |
| 225609_at | hg133b | | GSR | glutathione reductase | 0.942088 | 2.864759 | 7.929171 | 1.09E−10 |
| 204224_s_at | hg133a | | GCH1 | GTP cyclohydrolase 1 | 0.914258 | 2.156904 | 5.787298 | 2.34E−07 |
| | | | | (dopa-responsive | | | | |
| | | | | dystonia) | | | | |
| 204867_at | hg133a | | GCHFR | GTP cyclohydrolase I | 0.886063 | 2.620385 | 6.724541 | 1.03E−08 |
| | | | | feedback regulator | | | | |
| 44783_s_at | hg133a | | HEY1 | hairy/enhancer-of-split | 0.978613 | 2.502197 | 4.047892 | 1.74E−04 |
| | | | | related with YRPW motif 1 | | | | |
| 227262_at | hg133b | | HAPLN3 | hyaluronan and | 0.9745 | 2.006297 | 4.840556 | 8.24E−06 |
| | | | | proteoglycan link protein 3 | | | | |
| 205483_s_at | hg133a | | G1P2 | interferon, alpha-inducible | 0.934168 | 2.666766 | 3.127333 | 2.81E−03 |
| | | | | protein (clone IFI-15K) | | | | |
| 201193_at | hg133a | | IDH1 | isocitrate dehydrogenase 1 | 0.887283 | 2.074161 | 4.08532 | 1.20E−04 |
| | | | | (NADP+), soluble | | | | |
| 210046_s_at | hg133a | | IDH2 | isocitrate dehydrogenase 2 | 0.971034 | 3.789636 | 8.699199 | 2.51E−12 |
| | | | | (NADP+), mitochondrial | | | | |
| 209212_s_at | hg133a | | KLF5 | Kruppel-like factor 5 | 0.841618 | 2.146182 | 4.381283 | 4.58E−05 |
| | | | | (intestinal) | | | | |
| 208767_s_at | hg133a | | LAPTM4B | lysosomal associated | 0.98754 | 2.179677 | 3.987436 | 1.61E−04 |
| | | | | protein transmembrane 4 | | | | |
| | | | | beta | | | | |
| 221874_at | hg133a | | KIAA1324 | maba1 | 0.824149 | 3.100197 | 5.435425 | 1.16E−06 |
| 203362_s_at | hg133a | | MAD2L1 | MAD2 mitotic arrest | 0.808863 | 3.347217 | 5.915964 | 1.43E−07 |
| | | | | deficient-like 1 (yeast) | | | | |
| 218205_s_at | hg133a | MAP | MKNK2 | MAP kinase interacting | 1 | 2.170141 | 7.582505 | 1.30E−10 |
| | | kinase | | serine/threonine kinase 2 | | | | |
| 203936_s_at | hg133a | Angiogenesis; | MMP9 | matrix metalloproteinase 9 | 0.995247 | 3.777065 | 5.690025 | 2.89E−07 |
| | | NF- | | (gelatinase B, 92 kDa | | | | |
| | | kB target | | gelatinase, 92 kDa type IV | | | | |
| | | | | collagenase) | | | | |
| 222036_s_at | hg133a | DNA | MCM4 | MCM4 minichromosome | 0.878035 | 2.161679 | 5.655306 | 3.77E−07 |
| | | replication | | maintenance deficient 4 | | | | |
| | | | | (S. cerevisiae) | | | | |
| 202016_at | hg133a | | MEST | mesoderm specific | 0.955299 | 2.149282 | 4.364035 | 5.04E−05 |
| | | | | transcript homolog | | | | |
| | | | | (mouse) | | | | |
| 215498_s_at | hg133a | MAP | MAP2K3 | mitogen-activated protein | 0.966667 | 2.146212 | 7.523118 | 1.32E−10 |
| | | kinase | | kinase 3 | | | | |
| 205698_s_at | hg133a | MAP | MAP2K6 | mitogen-activated protein | 0.80957 | 2.647762 | 5.043075 | 3.42E−06 |
| | | kinase | | kinase 6 | | | | |
| 218883_s_at | hg133a | | MLF1IP | MLF1 interacting protein | 0.944637 | 2.435569 | 6.643637 | 6.16E−09 |
| 207847_s_at | hg133a | | MUC1 | mucin 1, transmembrane | 0.858703 | 3.80203 | 5.630422 | 4.74E−07 |
| 218189_s_at | hg133a | | NANS | N-acetylneuraminic acid | 0.985742 | 2.014633 | 7.394441 | 3.54E−10 |
| | | | | synthase (sialic acid | | | | |
| | | | | synthase) | | | | |
| 201468_s_at | hg133a | | NQO1 | NAD(P)H dehydrogenase, | 0.933654 | 2.844686 | 3.467474 | 9.24E−04 |
| | | | | quinone 1 | | | | |
| 218625_at | hg133a | | NRN1 | neuritin 1 | 0.912588 | 2.00039 | 3.917331 | 2.30E−04 |
| 218039_at | hg133a | | NUSAP1 | nucleolar and spindle | 0.920938 | 2.600532 | 6.227382 | 3.57E−08 |
| | | | | associated protein 1 | | | | |
| 226649_at | hg133b | kinase | PANK1 | pantothenate kinase 1 | 0.797611 | 2.298543 | 7.590671 | 9.72E−11 |
| 201489_at | hg133a | | PPIF | peptidylprolyl isomerase F | 0.90668 | 2.946223 | 5.911256 | 1.14E−07 |
| | | | | (cyclophilin F) | | | | |
| 201118_at | hg133a | | PGD | phosphogluconate | 0.835902 | 2.584108 | 5.299583 | 1.63E−06 |
| | | | | dehydrogenase | | | | |
| 200737_at | hg133a | kinase | PGK1 | phosphoglycerate kinase 1 | 0.976943 | 2.424245 | 7.092929 | 1.61E−09 |
| 210145_at | hg133a | | PLA2G4A | phospholipase A2, group | 0.773796 | 2.183904 | 3.371119 | 1.24E−03 |
| | | | | IVA (cytosolic, calcium- | | | | |
| | | | | dependent) | | | | |
| 212694_s_at | hg133a | | PCCB | propionyl Coenzyme A | 0.846179 | 2.00435 | 6.439523 | 1.42E−08 |
| | | | | carboxylase, beta | | | | |
| | | | | polypeptide | | | | |
| 202671_s_at | hg133a | | PDXK | pyridoxal (pyridoxine, | 0.95228 | 3.068155 | 7.419085 | 3.83E−10 |
| | | | | vitamin B6) kinase | | | | |
| 201251_at | hg133a | kinase | PKM2 | pyruvate kinase, muscle | 0.961978 | 2.862988 | 8.458819 | 3.24E−12 |
| 223471_at | hg133b | | RAB3IP | RAB3A interacting | 0.75567 | 2.311442 | 5.502764 | 5.60E−07 |
| | | | | protein (rabin3) | | | | |
| 226021_at | hg133b | | RDH10 | retinol dehydrogenase 10 | 0.852235 | 2.788311 | 5.967721 | 8.96E−08 |
| | | | | (all-trans) | | | | |
| 226576_at | hg133b | FAK | ARHGAP26 | Rho GTPase activating | 0.961079 | 2.342123 | 7.457975 | 4.72E−10 |
| | | tyrosine | | protein 26 | | | | |
| | | kinases | | | | | | |
| 217983_s_at | hg133a | | RNASET2 | ribonuclease T2 | 0.992486 | 2.380323 | 4.429634 | 3.38E−05 |
| 210715_s_at | hg133a | | SPINT2 | serine protease inhibitor, | 0.771612 | 2.357232 | 7.326805 | 3.77E−10 |
| | | | | Kunitz type, 2 | | | | |
| 201563_at | hg133a | | SORD | sorbitol dehydrogenase | 0.975851 | 3.272163 | 5.66846 | 4.08E−07 |
| 203509_at | hg133a | | SORL1 | sortilin-related receptor, | 0.944573 | 2.18248 | 7.46056 | 1.69E−10 |
| | | | | L(DLR class) A repeats- | | | | |
| | | | | containing | | | | |
| 226560_at | hg133b | | SGPP2 | sphingosine-1-phosphate | 0.812844 | 2.748831 | 4.56543 | 2.63E−05 |
| | | | | phosphotase 2 | | | | |
| 200832_s_at | hg133a | | SCD | stearoyl-CoA desaturase | 0.833076 | 3.597583 | 6.722352 | 3.79E−09 |
| | | | | (delta-9-desaturase) | | | | |
| 33323_r_at | hg133a | | SFN | stratifin | 0.955491 | 3.174759 | 4.017511 | 1.61E−04 |
| 218763_at | hg133a | | STX18 | syntaxin 18 | 0.829672 | 2.363415 | 4.886101 | 6.37E−06 |
| 226438_at | hg133b | | SNTB1 | syntrophin, beta 1 | 0.793249 | 2.061945 | 6.459 | 1.18E−08 |
| | | | | (dystrophin-associated | | | | |
| | | | | protein A1, 59 kDa, basic | | | | |
| | | | | component 1) | | | | |
| 202589_at | hg133a | | TYMS | thymidylate synthetase; | 0.919332 | 2.835631 | 5.833003 | 2.06E−07 |
| | | | | inhibitors: 5-fluorouracil, | | | | |
| | | | | 5-fluoro-2-prime- | | | | |
| | | | | deoxyuridine, and some | | | | |
| | | | | folate analogs | | | | |
| 208699_x_at | hg133a | | TKT | transketolase (Wernicke- | 0.933398 | 2.883285 | 5.472849 | 9.20E−07 |
| | | | | Korsakoff syndrome) | | | | |
| 209500_x_at | hg133a | endothelial | TNFSF12 | tumor necrosis factor | 0.871869 | 2.132305 | 8.88938 | 8.93E−13 |
| | | cell | | (ligand) superfamily, | | | | |
| | | growth | | member 12 | | | | |
| | | and | | | | | | |
| | | migration | | | | | | |
| 209500_x_at | hg133a | endothelial | TNFSF12- | tumor necrosis factor | 0.871869 | 2.132305 | 8.88938 | 8.93E−13 |
| | | cell | TNFSF13 | (ligand) superfamily, | | | | |
| | | growth | | member 12-member 13 | | | | |
| | | and | | | | | | |
| | | migration | | | | | | |
| 209500_x_at | hg133a | endothelial | TNFSF13 | tumor necrosis factor | 0.871869 | 2.132305 | 8.88938 | 8.93E−13 |
| | | cell | | (ligand) superfamily, | | | | |
| | | growth | | member 13 | | | | |
| | | and | | | | | | |
| | | migration | | | | | | |
| 223502_s_at | hg133b | endothelial | TNFSF13B | tumor necrosis factor | 0.852436 | 2.091912 | 6.490047 | 1.17E−08 |
| | | cell | | (ligand) superfamily, | | | | |
| | | growth | | member 13b | | | | |
| | | and | | | | | | |
| | | migration | | | | | | |
| 201688_s_at | hg133a | | TPD52 | tumor protein D52 | 0.812139 | 2.212542 | 4.49645 | 2.71E−05 |
| 202779_s_at | hg133a | Ubiquitin/ | UBE2S | ubiquitin-conjugating | 0.743224 | 2.257339 | 4.705354 | 1.35E−05 |
| | | proteosom | | enzyme E2S | | | | |
| 228498_at | hg133b | | B4GALT1 | UDP-Gal:betaGlcNAc | 0.804254 | 2.361267 | 4.023845 | 1.46E−04 |
| | | | | beta 1,4- | | | | |
| | | | | galactosyltransferase, | | | | |
| | | | | polypeptide 1 | | | | |
| 218313_s_at | hg133a | | GALNT7 | UDP-N-acetyl-alpha-D- | 0.90578 | 2.414458 | 6.440172 | 1.95E−08 |
| | | | | galactosamine:polypeptide | | | | |
| | | | | N- | | | | |
| | | | | acetylgalactosaminyltransferase | | | | |
| | | | | 7 (GalNAc-T7) | | | | |
| 218807_at | hg133a | VAV | VAV3 | vav 3 oncogene | 0.927489 | 2.591072 | 6.280577 | 2.39E−08 |
| | | oncogene; | | | | | | |
| | | can | | | | | | |
| | | enhance | | | | | | |
| | | NFkB | | | | | | |

TABLE XIV
PARP1 Upregulated - Diff/X (Human); Name: Upregulated Lung Large Cell Carcinoma Primary
(Minimum Fold Change: 2.0); Experiment: Lung, Large Cell Carcinoma, Primary; control: normal lung.

| Fragment | | | | | Pres. | Fold | | |
|---|---|---|---|---|---|---|---|---|
| Name | Array | Pathway | Symbol | Description | Freq. | Change | t-Score | p-Value |
| 209694_at | hg133a | | PTS | 6-pyruvoyltetrahydropterin | 0.951766 | 2.015958 | 3.56565 | 1.14E−02 |
| | | | | synthase | | | | |
| 218987_at | hg133a | | ATF7IP | activating transcription | 0.994926 | 2.026021 | 4.393671 | 4.37E−03 |
| | | | | factor 7 interacting protein | | | | |
| 204348_s_at | hg133a | Kinase | AK3L1 | adenylate kinase 3-like 1 | 0.742261 | 3.402295 | 3.605396 | 1.09E−02 |
| 204348_s_at | hg133a | Kinase | AK3L2 | adenylate kinase 3-like 2 | 0.742261 | 3.402295 | 3.605396 | 1.09E−02 |
| 222416_at | hg133b | | ALDH18A1 | aldehyde dehydrogenase 18 | 0.73923 | 2.204495 | 6.013427 | 5.58E−04 |
| | | | | family, member A1 | | | | |
| 209186_at | hg133a | ATPase | ATP2A2 | ATPase, Ca++ | 0.999294 | 2.400625 | 5.766126 | 9.37E−04 |
| | | | | transporting, cardiac | | | | |
| | | | | muscle, slow twitch 2 | | | | |
| 213088_s_at | hg133a | | DNAJC9 | DnaJ (Hsp40) homolog, | 0.996339 | 2.038625 | 4.293252 | 4.75E−03 |
| | | | | subfamily C, member 9 | | | | |
| 223531_x_at | hg133b | GPCR | GPR89 | G protein-coupled receptor | 0.777681 | 2.625728 | 3.348123 | 1.52E−02 |
| | | | | 89 | | | | |
| 200807_s_at | hg133a | Heat | HSPD1 | heat shock 60 kDa protein 1 | 1 | 2.048299 | 3.611712 | 1.04E−02 |
| | | shock | | (chaperonin) | | | | |
| | | proteins | | | | | | |
| 200825_s_at | hg133a | Hypoxia | HYOU1 | hypoxia up-regulated 1 | 0.9842 | 2.046587 | 3.711848 | 9.64E−03 |
| 200650_s_at | hg133a | | LDHA | lactate dehydrogenase A | 1 | 2.053377 | 4.717665 | 2.95E−03 |
| 217871_s_at | hg133a | NFkB; | MIF | macrophage migration | 0.995633 | 2.290487 | 6.489913 | 3.24E−04 |
| | | cell | | inhibitory factor | | | | |
| | | migration | | (glycosylation-inhibiting | | | | |
| | | | | factor) | | | | |
| 203936_s_at | hg133a | NFkB; | MMP9 | matrix metalloproteinase 9 | 0.995247 | 2.57064 | 3.022434 | 1.33E−02 |
| | | cell | | (gelatinase B, 92 kDa | | | | |
| | | migration | | gelatinase, 92 kDa type IV | | | | |
| | | | | collagenase) | | | | |
| 226760_at | hg133b | | MBTPS2 | Membrane-bound | 0.972151 | 2.023458 | 6.6453 | 4.36E−04 |
| | | | | transcription factor | | | | |
| | | | | protease, site 2 | | | | |
| 223577_x_at | hg133b | | MALAT1 | metastasis associated lung | 0.970071 | 2.290622 | 4.557104 | 3.52E−03 |
| | | | | adenocarcinoma transcript | | | | |
| | | | | 1 (non-coding RNA) | | | | |
| 201761_at | hg133a | | MTHFD2 | methylenetetrahydrofolate | 0.752922 | 2.473403 | 3.262411 | 1.66E−02 |
| | | | | dehydrogenase (NADP+ | | | | |
| | | | | dependent) 2, | | | | |
| | | | | methenyltetrahydrofolate | | | | |
| | | | | cyclohydrolase | | | | |
| 202647_s_at | hg133a | Ras | NRAS | neuroblastoma RAS viral | 0.803854 | 3.718247 | 3.996147 | 7.08E−03 |
| | | oncogene | | (v-ras) oncogene homolog 4 | | | | |
| 207239_s_at | hg133a | Kinase | PCTK1 | PCTAIRE protein kinase 1 | 0.972511 | 2.049254 | 3.177104 | 1.75E−02 |
| 201489_at | hg133a | | PPIF | peptidylprolyl isomerase F | 0.90668 | 2.00506 | 3.070338 | 1.92E−02 |
| | | | | (cyclophilin F) | | | | |
| 201037_at | hg133a | | PFKP | phosphofructokinase, | 0.953565 | 2.952199 | 6.011932 | 8.25E−04 |
| | | | | platelet | | | | |
| 201013_s_at | hg133a | | PAICS | phosphoribosylaminoimidazole | 0.993706 | 3.007346 | 5.616205 | 1.08E−03 |
| | | | | carboxylase, | | | | |
| | | | | phosphoribosylaminoimidazole | | | | |
| | | | | succinocarboxamide | | | | |
| | | | | synthetase | | | | |
| 202620_s_at | hg133a | | PLOD2 | procollagen-lysine, 2- | 0.864033 | 5.703536 | 4.198014 | 5.56E−03 |
| | | | | oxoglutarate 5-dioxygenase 2 | | | | |
| 202243_s_at | hg133a | proteosome | PSMB4 | proteasome (prosome, | 0.998908 | 2.123566 | 4.326836 | 4.77E−03 |
| | | | | macropain) subunit, beta | | | | |
| | | | | type, 4 | | | | |
| 226452_at | hg133b | kinase | PDK1 | pyruvate dehydrogenase | 0.950745 | 3.235952 | 3.743069 | 9.07E−03 |
| | | | | kinase, isoenzyme 1 | | | | |
| 201251_at | hg133a | | PKM2 | pyruvate kinase, muscle | 0.961978 | 2.027997 | 3.29022 | 1.60E−02 |
| 222077_s_at | hg133a | GTPase | RACGAP1 | Rac GTPase activating | 0.955106 | 3.445884 | 3.875033 | 7.97E−03 |
| | | | | protein 1 | | | | |
| 202483_s_at | hg133a | Ras | RANBP1 | RAN binding protein 1 | 0.838471 | 2.226249 | 4.425497 | 3.79E−03 |
| | | family | | | | | | |
| 200750_s_at | hg133a | Ras | RAN | RAN, member RAS | 0.998715 | 2.202252 | 3.46107 | 1.32E−02 |
| | | family | | oncogene family | | | | |
| 203209_at | hg133a | DNA | RFC5 | replication factor C | 0.865639 | 2.219817 | 3.461237 | 1.29E−02 |
| | | replication | | (activator 1) 5, 36.5 kDa | | | | |
| | | and | | | | | | |
| | | repair | | | | | | |
| 202200_s_at | hg133a | Kinase | SRPK1 | SFRS protein kinase 1 | 0.996275 | 2.195398 | 4.2174 | 5.28E−03 |
| 204675_at | hg133a | | SRD5A1 | steroid-5-alpha-reductase, | 0.813809 | 3.182621 | 3.733992 | 9.30E−03 |
| | | | | alpha polypeptide 1 (3-oxo- | | | | |
| | | | | 5 alpha-steroid delta 4- | | | | |
| | | | | dehydrogenase alpha 1) | | | | |
| 200822_x_at | hg133a | | TPI1 | triosephosphate isomerase 1 | 0.99955 | 2.393102 | 4.436549 | 4.13E−03 |
| 202779_s_at | hg133a | Ubiquitin/ | UBE2S | ubiquitin-conjugating | 0.743224 | 2.755398 | 3.32349 | 1.14E−02 |
| | | proteosome | | enzyme E2S | | | | |

TABLE XV
PARP1 Upregulated - Diff/X (Human); Name: Upregulated Lymph Node Non-Hodgkin's Lymphoma
All Types (Minimum Fold Change: 2.0); Experiment: Lymph Node, Non-Hodgkin's Lymphoma, All Types;
control: normal lymph node.

| | | | | | Pres. | | | p- |
|---|---|---|---|---|---|---|---|---|
| Fragment Name | Array | Pathway | Symbol | Description | Freq. | Fold Change | t-Score | Value |
| 229128_s_at | hg133b | | ANP32E | acidic (leucine-rich) | 0.802107 | 2.101182 | 7.541534 | 2.47E−08 |
| | | | | nuclear phosphoprotein 32 | | | | |
| | | | | family, member E | | | | |
| 226517_at | hg133b | | BCAT1 | branched chain | 0.742115 | 3.653676 | 7.113797 | 1.82E−10 |
| | | | | aminotransferase 1, | | | | |
| | | | | cytosolic | | | | |
| 204440_at | hg133a | | CD83 | CD83 antigen (activated B | 0.735067 | 3.27867 | 7.534241 | 3.08E−10 |
| | | | | lymphocytes, | | | | |
| | | | | immunoglobulin | | | | |
| | | | | superfamily) | | | | |
| 218549_s_at | hg133a | | CGI-90 | CGI-90 protein | 0.921644 | 2.082082 | 8.812863 | 1.01E−12 |
| 202329_at | hg133a | | CSK | c-src tyrosine kinase | 0.881374 | 2.118707 | 7.436565 | 2.07E−06 |
| 221482_s_at | hg133a | tyrosine | ARPP-19 | cyclic AMP | 0.983365 | 2.042533 | 10.375835 | 2.15E−09 |
| | | kinase/ | | phosphoprotein, 19 kD | | | | |
| | | Src | | | | | | |
| | | oncogene | | | | | | |
| 208152_s_at | hg133a | | DDX21 | DEAD (Asp-Glu-Ala-Asp) | 0.989981 | 2.413377 | 6.933367 | 5.76E−10 |
| | | | | box polypeptide 21 | | | | |
| 203302_at | hg133a | Kinase | DCK | deoxycytidine kinase | 0.970392 | 2.022343 | 6.294393 | 4.90E−06 |
| 202534_x_at | hg133a | | DHFR | dihydrofolate reductase; | 0.983687 | 2.092213 | 8.775685 | 4.05E−10 |
| | | | | Inhibitors: A variety of | | | | |
| | | | | drugs act on dihydrofolate | | | | |
| | | | | reductase: | | | | |
| | | | | the antibiotic trimethoprim. | | | | |
| | | | | the antimalarial drug | | | | |
| | | | | pyrimethamine. the | | | | |
| | | | | chemotherapeutic agents | | | | |
| | | | | methotrexate and | | | | |
| | | | | pemetrexed. Methotrexate, | | | | |
| | | | | the first anticancer drug | | | | |
| 216060_s_at | hg133a | | DAAM1 | dishevelled associated | 0.874952 | 2.197711 | 5.073686 | 2.53E−06 |
| | | | | activator of morphogenesis 1 | | | | |
| 221563_at | hg133a | | DUSP10 | dual specificity | 0.927425 | 2.267792 | 6.275085 | 9.39E−09 |
| | | | | phosphatase 10 | | | | |
| 201347_x_at | hg133a | | GRHPR | glyoxylate | 0.998394 | 3.075051 | 7.035188 | 2.99E−10 |
| | | | | reductase/hydroxypyruvate | | | | |
| | | | | reductase | | | | |
| 210658_s_at | hg133a | | GGA2 | golgi associated, gamma | 0.893899 | 2.079844 | 7.290699 | 2.25E−08 |
| | | | | adaptin ear containing, | | | | |
| | | | | ARF binding protein 2 | | | | |
| 204867_at | hg133a | | GCHFR | GTP cyclohydrolase I | 0.886063 | 2.361411 | 8.427763 | 8.16E−13 |
| | | | | feedback regulator | | | | |
| 211015_s_at | hg133a | Heat | HSPA4 | heat shock 70 kDa protein 4 | 0.937893 | 2.112118 | 10.878284 | 3.95E−17 |
| | | Shock | | | | | | |
| 203284_s_at | hg133a | | HS2ST1 | heparan sulfate 2-O- | 0.889403 | 2.448976 | 8.098059 | 1.71E−12 |
| | | | | sulfotransferase 1 | | | | |
| 201209_at | hg133a | | HDAC1 | histone deacetylase 1 | 0.907836 | 2.032943 | 8.680717 | 6.30E−10 |
| 202854_at | hg133a | purine | HPRT1 | hypoxanthine | 0.998587 | 2.149667 | 8.608762 | 2.08E−13 |
| | | metabolism. | | phosphoribosyltransferase 1 | | | | |
| | | | | (Lesch-Nyhan syndrome) | | | | |
| 201088_at | hg133a | | KPNA2 | karyopherin alpha 2 (RAG | 0.985934 | 2.058364 | 6.377564 | 4.71E−10 |
| | | | | cohort 1, importin alpha 1) | | | | |
| 203362_s_at | hg133a | | MAD2L1 | MAD2 mitotic arrest | 0.808863 | 2.903429 | 7.113883 | 4.12E−09 |
| | | | | deficient-like 1 (yeast) | | | | |
| 222036_s_at | hg133a | DNA | MCM4 | MCM4 minichromosome | 0.878035 | 2.586192 | 8.023728 | 1.09E−10 |
| | | replication | | maintenance deficient 4 (S. cerevisiae) | | | | |
| | | and | | | | | | |
| | | repair | | | | | | |
| 201298_s_at | hg133a | | MOBK1B | MOB1, Mps One Binder | 0.796468 | 2.01745 | 6.892692 | 6.63E−08 |
| | | | | kinase activator-like 1B | | | | |
| | | | | (yeast) | | | | |
| 209421_at | hg133a | DNA | MSH2 | mutS homolog 2, colon | 0.807964 | 2.422483 | 12.388963 | 4.82E−20 |
| | | repair | | cancer, nonpolyposis type 1 | | | | |
| | | | | (E. coli) | | | | |
| 218039_at | hg133a | | NUSAP1 | nucleolar and spindle | 0.920938 | 3.006645 | 9.984665 | 1.08E−13 |
| | | | | associated protein 1 | | | | |
| 200790_at | hg133a | | ODC1 | ornithine decarboxylase 1 | 0.934682 | 2.329386 | 7.843458 | 1.01E−08 |
| 204604_at | hg133a | Kinase | PFTK1 | PFTAIRE protein kinase 1 | 0.909377 | 2.035057 | 6.700944 | 2.63E−09 |
| 204613_at | hg133a | | PLCG2 | phospholipase C, gamma 2 | 0.901477 | 2.244898 | 7.816932 | 4.90E−09 |
| | | | | (phosphatidylinositol- | | | | |
| | | | | specific) | | | | |
| 203537_at | hg133a | | PRPSAP2 | phosphoribosyl | 0.967759 | 2.079838 | 6.717364 | 9.39E−09 |
| | | | | pyrophosphate synthetase- | | | | |
| | | | | associated protein 2 | | | | |
| 216525_x_at | hg133a | | PMS2L3 | postmeiotic segregation | 0.972961 | 2.029075 | 7.733492 | 1.50E−08 |
| | | | | increased 2-like 3 | | | | |
| 201202_at | hg133a | DNA | PCNA | proliferating cell nuclear | 0.959987 | 2.601181 | 10.287968 | 4.27E−16 |
| | | repair | | antigen | | | | |
| 213521_at | hg133a | | PTPN18 | protein tyrosine | 0.961207 | 2.283607 | 7.414702 | 4.04E−10 |
| | | | | phosphatase, non-receptor | | | | |
| | | | | type 18 (brain-derived) | | | | |
| 222077_s_at | hg133a | GTPase | RACGAP1 | Rac GTPase activating | 0.955106 | 2.69391 | 9.118326 | 1.07E−12 |
| | | | | protein 1 | | | | |
| 204207_s_at | hg133a | | RNGTT | RNA guanylyltransferase | 0.763006 | 3.403594 | 4.65097 | 1.05E−05 |
| | | | | and 5′-phosphatase | | | | |
| 202690_s_at | hg133a | | SNRPD1 | small nuclear | 0.992742 | 2.087974 | 11.650476 | 1.81E−19 |
| | | | | ribonucleoprotein D1 | | | | |
| | | | | polypeptide 16 kDa | | | | |
| 202043_s_at | hg133a | | SMS | spermine synthase | 0.991843 | 2.19221 | 9.043032 | 7.16E−14 |
| 223391_at | hg133b | | SGPP1 | sphingosine-1-phosphate | 0.894846 | 2.618172 | 8.021602 | 7.40E−12 |
| | | | | phosphatase 1 | | | | |
| 220232_at | hg133a | | SCD4 | stearoyl-CoA desaturase 4 | 0.903147 | 3.159006 | 5.788884 | 8.46E−08 |
| 209306_s_at | hg133a | | SWAP70 | SWAP-70 protein | 0.933269 | 3.014505 | 9.573432 | 1.25E−14 |
| 202816_s_at | hg133a | | SS18 | synovial sarcoma | 0.883622 | 2.172324 | 6.412457 | 9.87E−07 |
| | | | | translocation, chromosome | | | | |
| | | | | 18 | | | | |
| 239835_at | hg133b | | TA-KRP | T-cell activation kelch | 0.940813 | 2.332956 | 6.904923 | 1.09E−06 |
| | | | | repeat protein | | | | |
| 202589_at | hg133a | | TYMS | thymidylate synthetase; | 0.919332 | 2.429659 | 5.376319 | 1.64E−05 |
| | | | | inhibitors: 5-fluorouracil, 5- | | | | |
| | | | | fluoro-2-prime- | | | | |
| | | | | deoxyuridine, and some | | | | |
| | | | | folate analogs | | | | |
| 203432_at | hg133a | | TMPO | thymopoietin | 0.814965 | 2.174064 | 7.511561 | 6.57E−08 |
| 207332_s_at | hg133a | | TFRC | transferrin receptor (p90, | 0.98799 | 2.741458 | 4.326887 | 3.80E−05 |
| | | | | CD71) | | | | |
| 206907_at | hg133a | BCL | tumor | 0.749775 | Lymph | 2.508162 | 6.238358 | 1.45E−08 |
| | | oncogene | necrosis | | Node, | | | |
| | | signaling; | factor | | Non- | | | |
| | | activation | (ligand) | | Hodgkin's | | | |
| | | NFkB; | superfamily, | | Lymphoma, | | | |
| | | endothelial | member 9 | | All | | | |
| | | cell | (TNFSF9) | | Types | | | |
| | | migration; | | | | | | |
| | | angiogenesis | | | | | | |
| 202779_s_at | hg133a | proteosome/ | UBE2S | ubiquitin-conjugating | 0.743224 | 2.896169 | 6.265012 | 3.96E−08 |
| | | ubiquitin | | enzyme E2S | | | | |
| 202625_at | hg133a | yes | LYN | v-yes-1 Yamaguchi | 0.82903 | 2.186957 | 6.277163 | 9.49E−06 |
| | | oncogene | | sarcoma viral related | | | | |
| | | family | | oncogene homolog | | | | |

TABLE XVI
PARP1 Upregulated - Diff/X (Human); Name: Upregulated Lymph Node Non-Hodgkin's
Lymphoma Diffuse Large B-Cell Type (Minimum Fold Change: 2.0); Experiment: Lymph Node,
Non-Hodgkin's Lymphoma, Diffuse Large B-Cell Type; control: normal lymph node.

| Fragment | | | | | Pres. | Fold | | p- |
|---|---|---|---|---|---|---|---|---|
| Name | Array | Pathway | Symbol | Description | Freq. | Change | t-Score | Value |
| 232103_at | hg133b | | BPNT1 | 3′(2′), 5′-bisphosphate | 0.900282 | 2.27366 | 5.575893 | 2.35E−06 |
| | | | | nucleotidase 1 | | | | |
| 208758_at | hg133a | | ATIC | 5-aminoimidazole-4- | 0.986448 | 2.115381 | 9.40825 | 1.25E−11 |
| | | | | carboxamide | | | | |
| | | | | ribonucleotide | | | | |
| | | | | formyltransferase/IMP | | | | |
| | | | | cyclohydrolase | | | | |
| 204998_s_at | hg133a | | ATF5 | activating transcription | 0.971227 | 2.611824 | 4.157188 | 1.89E−04 |
| | | | | factor 5 | | | | |
| 202502_at | hg133a | | ACADM | acyl-Coenzyme A | 0.99422 | 2.318166 | 5.870059 | 9.65E−07 |
| | | | | dehydrogenase, C-4 to C- | | | | |
| | | | | 12 straight chain | | | | |
| 225421_at | hg133b | | ACY1L2 | aminoacylase 1-like 2 | 0.890954 | 2.008709 | 3.205156 | 2.95E−03 |
| 203140_at | hg133a | BCL | BCL6 | B-cell CLL/lymphoma 6 | 0.961143 | 2.043624 | 4.175383 | 1.57E−04 |
| | | oncogene | | (zinc finger protein 51) | | | | |
| 209406_at | hg133a | BCL | BAG2 | BCL2-associated | 0.760629 | 2.096459 | 6.547377 | 5.36E−07 |
| | | oncogene | | athanogene 2 | | | | |
| 226517_at | hg133b | | BCAT1 | branched chain | 0.742115 | 5.315009 | 5.901769 | 1.41E−06 |
| | | | | aminotransferase 1, | | | | |
| | | | | cytosolic | | | | |
| 210563_x_at | hg133a | | CFLAR | CASP8 and FADD-like | 0.859345 | 2.045713 | 3.223637 | 2.63E−03 |
| | | | | apoptosis regulator | | | | |
| 204440_at | hg133a | | CD83 | CD83 antigen (activated | 0.735067 | 3.388786 | 6.321404 | 1.68E−07 |
| | | | | B lymphocytes, | | | | |
| | | | | immunoglobulin | | | | |
| | | | | superfamily) | | | | |
| 201897_s_at | hg133a | kinase | CKS1B | CDC28 protein kinase | 0.761593 | 2.633651 | 8.101701 | 1.54E−09 |
| | | | | regulatory subunit 1B | | | | |
| 209057_x_at | hg133a | Polo-like | CDC5L | CDC5 cell division cycle | 0.963327 | 2.010159 | 2.966092 | 5.26E−03 |
| | | kinase | | 5-like (S. pombe) | | | | |
| 225082_at | hg133b | | CPSF3 | cleavage and | 0.935915 | 2.070844 | 6.850072 | 3.64E−08 |
| | | | | polyadenylation specific | | | | |
| | | | | factor 3, 73 kDa | | | | |
| 202697_at | hg133a | | CPSF5 | cleavage and | 0.919846 | 2.273114 | 6.953357 | 2.19E−08 |
| | | | | polyadenylation specific | | | | |
| | | | | factor 5, 25 kDa | | | | |
| 202469_s_at | hg133a | | CPSF6 | cleavage and | 0.993256 | 2.275203 | 11.824529 | 1.27E−14 |
| | | | | polyadenylation specific | | | | |
| | | | | factor 6, 68 kDa | | | | |
| 208910_s_at | hg133a | | C1QBP | complement component 1, | 0.971612 | 2.726219 | 7.762402 | 2.13E−09 |
| | | | | q subcomponent binding | | | | |
| | | | | protein | | | | |
| 218260_at | hg133a | | PCIA1 | cross-immune reaction | 0.764676 | 2.076875 | 5.537493 | 2.11E−06 |
| | | | | antigen PCIA1 | | | | |
| 202329_at | hg133a | c-src | CSK | c-src tyrosine kinase | 0.881374 | 2.252833 | 6.718699 | 2.80E−07 |
| | | tyrosine | | | | | | |
| | | kinase | | | | | | |
| 221482_s_at | hg133a | | ARPP- | cyclic AMP | 0.983365 | 2.100869 | 8.19224 | 1.08E−09 |
| | | | 19 | phosphoprotein, 19 kD | | | | |
| 202246_s_at | hg133a | cyclin- | CDK4 | cyclin-dependent kinase 4 | 0.924534 | 2.054399 | 7.848799 | 2.60E−09 |
| | | dependent | | | | | | |
| | | kinase 4 | | | | | | |
| 202534_x_at | hg133a | | DHFR | dihydrofolate reductase; | 0.983687 | 2.571491 | 8.370235 | 2.51E−10 |
| | | | | inhibitors: A variety of | | | | |
| | | | | drugs act on dihydrofolate | | | | |
| | | | | reductase | | | | |
| 213149_at | hg133a | | DLAT | dihydrolipoamide S- | 0.851766 | 2.156783 | 5.806236 | 1.08E−06 |
| | | | | acetyltransferase (E2 | | | | |
| | | | | component of pyruvate | | | | |
| | | | | dehydrogenase complex); | | | | |
| | | | | inhibitor: the antibiotic | | | | |
| | | | | trimethoprim | | | | |
| 218435_at | hg133a | | DNAJD1 | DnaJ (Hsp40) homolog, | 0.88587 | 2.048611 | 6.031258 | 7.43E−07 |
| | | | | subfamily D, member 1; | | | | |
| | | | | inhibitor: the antimalarial | | | | |
| | | | | drug pyrimethamine | | | | |
| 221563_at | hg133a | | DUSP10 | dual specificity | 0.927425 | 2.643395 | 4.783932 | 3.40E−05 |
| | | | | phosphatase 10; | | | | |
| | | | | inhibitors: the | | | | |
| | | | | chemotherapeutic agents | | | | |
| | | | | methotrexate and | | | | |
| | | | | pemetrexed. | | | | |
| | | | | Methotrexate, the first | | | | |
| | | | | anticancer drug | | | | |
| 217294_s_at | hg133a | | ENO1 | enolase 1, (alpha) | 0.932884 | 2.499587 | 6.0174 | 5.40E−07 |
| 215438_x_at | hg133a | | GSPT1 | G1 to S phase transition 1 | 0.84271 | 2.138906 | 5.033073 | 1.45E−05 |
| 218350_s_at | hg133a | | GMNN | geminin, DNA replication | 0.962364 | 2.617912 | 7.116693 | 1.33E−08 |
| | | | | inhibitor | | | | |
| 208308_s_at | hg133a | | GPI | glucose phosphate | 0.998715 | 2.020914 | 6.415779 | 1.25E−07 |
| | | | | isomerase | | | | |
| 214864_s_at | hg133a | | GRHPR | glyoxylate | 0.987347 | 3.429695 | 5.190155 | 1.08E−05 |
| | | | | reductase/hydroxypyruvate | | | | |
| | | | | reductase | | | | |
| 218239_s_at | hg133a | | GTPBP4 | GTP binding protein 4 | 0.9921 | 2.042254 | 6.813646 | 4.54E−08 |
| 204867_at | hg133a | | GCHFR | GTP cyclohydrolase I | 0.886063 | 2.553084 | 4.812134 | 2.97E−05 |
| | | | | feedback regulator | | | | |
| 206976_s_at | hg133a | Heat | HSPH1 | heat shock | 0.998009 | 2.326608 | 5.286037 | 4.79E−06 |
| | | shock | | 105 kDa/110 kDa protein 1 | | | | |
| 205133_s_at | hg133a | Heat | HSPE1 | heat shock 10 kDa protein | 0.998137 | 2.330077 | 8.224263 | 5.08E−10 |
| | | shock | | 1 (chaperonin 10) | | | | |
| 200806_s_at | hg133a | Heat | HSPD1 | heat shock 60 kDa protein | 0.970328 | 2.639501 | 8.787148 | 1.01E−10 |
| | | shock | | 1 (chaperonin) | | | | |
| 211015_s_at | hg133a | Heat | HSPA4 | heat shock 70 kDa protein 4 | 0.937893 | 2.640265 | 9.059092 | 8.80E−11 |
| | | shock | | | | | | |
| 211968_s_at | hg133a | Heat | HSPCA | heat shock 90 kDa protein | 0.999037 | 2.201077 | 7.324727 | 7.04E−09 |
| | | shock | | 1, alpha | | | | |
| 214359_s_at | hg133a | Heat | HSPCB | heat shock 90 kDa protein | 0.976814 | 2.297993 | 7.633586 | 2.72E−09 |
| | | shock | | 1, beta | | | | |
| 203284_s_at | hg133a | | HS2ST1 | heparan sulfate 2-O- | 0.889403 | 2.57044 | 5.348845 | 7.19E−06 |
| | | | | sulfotransferase 1 | | | | |
| 201209_at | hg133a | | HDAC1 | histone deacetylase 1; | 0.907836 | 2.141784 | 6.055195 | 4.00E−07 |
| | | | | inhibitor: Vorinostat; | | | | |
| | | | | trichostatin A | | | | |
| 206445_s_at | hg133a | | HRMT1L2 | HMT1 hnRNP | 0.75228 | 2.012121 | 6.295004 | 1.98E−07 |
| | | | | methyltransferase-like 2 | | | | |
| | | | | (S. cerevisiae) | | | | |
| 202854_at | hg133a | | HPRT1 | hypoxanthine | 0.998587 | 3.010065 | 7.539891 | 9.93E−09 |
| | | | | phosphoribosyltransferase | | | | |
| | | | | 1 (Lesch-Nyhan | | | | |
| | | | | syndrome) | | | | |
| 218507_at | hg133a | Hypoxia | HIG2 | hypoxia-inducible protein 2 | 0.854335 | 2.371388 | 2.698295 | 1.10E−02 |
| 201625_s_at | hg133a | | INSIG1 | insulin induced gene 1 | 0.740398 | 2.234379 | 3.320509 | 1.97E−03 |
| 200650_s_at | hg133a | | LDHA | lactate dehydrogenase A | 1 | 2.07996 | 7.0558 | 2.30E−08 |
| 203362_s_at | hg133a | | MAD2L1 | MAD2 mitotic arrest | 0.808863 | 4.236409 | 6.760406 | 5.52E−08 |
| | | | | deficient-like 1 (yeast) | | | | |
| 227416_s_at | hg133b | | MADP-1 | MADP-1 protein | 0.911623 | 2.041079 | 5.036759 | 2.43E−05 |
| 222393_s_at | hg133b | | MAK3 | Mak3 homolog (S. cerevisiae) | 0.780365 | 2.022479 | 4.87021 | 1.81E−05 |
| 200978_at | hg133a | | MDH1 | malate dehydrogenase 1, | 0.992486 | 2.001869 | 4.090009 | 2.56E−04 |
| | | | | NAD (soluble) | | | | |
| 209036_s_at | hg133a | | MDH2 | malate dehydrogenase 2, | 0.998844 | 2.089056 | 6.927699 | 7.14E−08 |
| | | | | NAD (mitochondrial) | | | | |
| 210153_s_at | hg133a | | ME2 | malic enzyme 2, NAD(+)- | 0.768208 | 2.147248 | 5.015695 | 1.20E−05 |
| | | | | dependent, mitochondrial | | | | |
| 218163_at | hg133a | | MCTS1 | malignant T cell amplified | 0.937765 | 2.560092 | 8.047262 | 1.27E−09 |
| | | | | sequence 1 | | | | |
| 218205_s_at | hg133a | MAP | MKNK2 | MAP kinase interacting | 1 | 2.085416 | 5.098583 | 8.96E−06 |
| | | kinase | | serine/threonine kinase 2 | | | | |
| 222036_s_at | hg133a | DNA | MCM4 | MCM4 minichromosome | 0.878035 | 3.793559 | 8.602283 | 1.81E−10 |
| | | replication | | maintenance deficient 4 | | | | |
| | | and | | (S. cerevisiae) | | | | |
| | | repair | | | | | | |
| 209861_s_at | hg133a | | METAP2 | methionyl aminopeptidase 2 | 0.967823 | 2.258253 | 4.764057 | 3.71E−05 |
| 201761_at | hg133a | | MTHFD2 | methylenetetrahydrofolate | 0.752922 | 2.894205 | 8.648312 | 1.39E−10 |
| | | | | dehydrogenase (NADP+ | | | | |
| | | | | dependent) 2, | | | | |
| | | | | methenyltetrahydrofolate | | | | |
| | | | | cyclohydrolase | | | | |
| 201298_s_at | hg133a | | MOBK1B | MOB1, Mps One Binder | 0.796468 | 2.540345 | 6.332088 | 1.69E−07 |
| | | | | kinase activator-like 1B | | | | |
| | | | | (yeast) | | | | |
| 201299_s_at | hg133a | | MOBK1B | MOB1, Mps One Binder | 0.7842 | 2.136819 | 6.506549 | 2.07E−07 |
| | | | | kinase activator-like 1B | | | | |
| | | | | (yeast) | | | | |
| 209421_at | hg133a | DNA | MSH2 | mutS homolog 2, colon | 0.807964 | 2.952645 | 8.832279 | 2.01E−10 |
| | | repair | | cancer, nonpolyposis type | | | | |
| | | | | 1 (E. coli) | | | | |
| 223158_s_at | hg133b | Kinase | NEK6 | NIMA (never in mitosis | 0.817675 | 2.154088 | 3.415394 | 1.58E−03 |
| | | | | gene a)-related kinase 6 | | | | |
| 201577_at | hg133a | | NME1 | non-metastatic cells 1, | 0.997559 | 2.492503 | 7.561599 | 3.57E−09 |
| | | | | protein (NM23A) | | | | |
| | | | | expressed in | | | | |
| 218039_at | hg133a | | NUSAP1 | nucleolar and spindle | 0.920938 | 3.94954 | 9.02774 | 5.01E−11 |
| | | | | associated protein 1 | | | | |
| 226287_at | hg133b | | NY- | NY-REN-41 antigen | 0.967119 | 2.280992 | 6.410836 | 1.37E−07 |
| | | | REN-41 | | | | | |
| 200790_at | hg133a | | ODC1 | ornithine decarboxylase 1 | 0.934682 | 2.809657 | 7.232717 | 9.01E−09 |
| 201037_at | hg133a | | PFKP | phosphofructokinase, | 0.953565 | 2.108153 | 6.031325 | 4.28E−07 |
| | | | | platelet | | | | |
| 217356_s_at | hg133a | Kinase | PGK1 | phosphoglycerate kinase 1 | 0.951252 | 2.694533 | 7.540861 | 8.29E−09 |
| 204613_at | hg133a | | PLCG2 | phospholipase C, gamma | 0.901477 | 2.608765 | 5.642482 | 1.64E−06 |
| | | | | 2 (phosphatidylinositol- | | | | |
| | | | | specific) | | | | |
| 203537_at | hg133a | | PRPSAP2 | phosphoribosyl | 0.967759 | 2.374371 | 5.198686 | 7.21E−06 |
| | | | | pyrophosphate synthetase- | | | | |
| | | | | associated protein 2 | | | | |
| 201013_s_at | hg133a | | PAICS | phosphoribosylaminoimidazole | 0.993706 | 2.764595 | 6.316633 | 3.39E−07 |
| | | | | carboxylase, | | | | |
| | | | | phosphoribosylaminoimidazole | | | | |
| | | | | succinocarboxamide | | | | |
| | | | | synthetase | | | | |
| 210317_s_at | hg133a | | PAFAH1B1 | platelet-activating factor | 0.950161 | 2.052006 | 5.393679 | 3.35E−06 |
| | | | | acetylhydrolase, isoform | | | | |
| | | | | Ib, alpha subunit 45 kDa | | | | |
| 201202_at | hg133a | DNA | PCNA | proliferating cell nuclear | 0.959987 | 3.498783 | 8.773777 | 2.13E−10 |
| | | repair | | antigen | | | | |
| 201317_s_at | hg133a | Proteosome | PSMA2 | proteasome (prosome, | 0.999229 | 2.037005 | 7.692206 | 2.08E−09 |
| | | | | macropain) subunit, alpha | | | | |
| | | | | type, 2 | | | | |
| 202732_at | hg133a | Protein | PKIG | protein kinase (cAMP- | 0.905395 | 2.077428 | 7.565193 | 3.16E−09 |
| | | Kinase | | dependent, catalytic) | | | | |
| | | | | inhibitor gamma | | | | |
| 218236_s_at | hg133a | Protein | PRKD3 | protein kinase D3 | 0.97842 | 2.208092 | 4.134563 | 2.12E−04 |
| | | Kinase | | | | | | |
| 208694_at | hg133a | Protein | PRKDC | protein kinase, DNA- | 0.976557 | 2.125992 | 6.409797 | 2.08E−07 |
| | | Kinase | | activated, catalytic | | | | |
| | | | | polypeptide | | | | |
| 213521_at | hg133a | | PTPN18 | protein tyrosine | 0.961207 | 2.219037 | 4.83044 | 2.18E−05 |
| | | | | phosphatase, non-receptor | | | | |
| | | | | type 18 (brain-derived) | | | | |
| 201251_at | hg133a | | PKM2 | pyruvate kinase, muscle | 0.961978 | 2.650295 | 7.798557 | 2.56E−09 |
| 222077_s_at | hg133a | Rac | RACGAP1 | Rac GTPase activating | 0.955106 | 3.580662 | 8.006371 | 1.27E−09 |
| | | GTPase | | protein 1 | | | | |
| | | pathway | | | | | | |
| 200750_s_at | hg133a | Ras | RAN | RAN, member RAS | 0.998715 | 2.577989 | 9.662825 | 5.48E−12 |
| | | pathway | | oncogene family | | | | |
| 212590_at | hg133a | Ras | RRAS2 | related RAS viral (r-ras) | 0.784843 | 2.540055 | 5.076458 | 1.12E−05 |
| | | pathway | | oncogene homolog 2 | | | | |
| 204127_at | hg133a | DNA | RFC3 | replication factor C | 0.90578 | 3.037702 | 6.623896 | 1.14E−07 |
| | | repair | | (activator 1) 3, 38 kDa | | | | |
| 204023_at | hg133a | DNA | RFC4 | replication factor C | 0.821644 | 2.379399 | 7.129607 | 1.34E−08 |
| | | repair | | (activator 1) 4, 37 kDa | | | | |
| 201092_at | hg133a | | RBBP7 | retinoblastoma binding | 0.99955 | 2.048146 | 6.219347 | 5.31E−07 |
| | | | | protein 7 | | | | |
| 203344_s_at | hg133a | | RBBP8 | retinoblastoma binding | 0.931278 | 2.241458 | 5.534298 | 2.13E−06 |
| | | | | protein 8 | | | | |
| 200903_s_at | hg133a | | AHCY | S-adenosylhomocysteine | 0.994348 | 2.047523 | 6.659386 | 5.72E−08 |
| | | | | hydrolase | | | | |
| 202591_s_at | hg133a | DNA | SSBP1 | single-stranded DNA | 0.998202 | 2.124924 | 9.268389 | 1.92E−11 |
| | | replication | | binding protein 1 | | | | |
| | | and | | | | | | |
| | | repair | | | | | | |
| 201664_at | hg133a | | SMC4L1 | SMC4 structural | 0.975915 | 2.312916 | 7.005807 | 1.98E−08 |
| | | | | maintenance of | | | | |
| | | | | chromosomes 4-like 1 | | | | |
| | | | | (yeast) | | | | |
| 202043_s_at | hg133a | | SMS | spermine synthase | 0.991843 | 2.917971 | 7.789894 | 3.81E−09 |
| 223391_at | hg133b | | SGPP1 | sphingosine-1-phosphate | 0.894846 | 2.270207 | 3.9326 | 3.64E−04 |
| | | | | phosphatase 1 | | | | |
| 225639_at | hg133b | SRC | SCAP2 | src family associated | 0.845256 | 2.446779 | 5.890024 | 6.78E−07 |
| | | oncogene | | phosphoprotein 2 | | | | |
| | | pathway | | | | | | |
| 209306_s_at | hg133a | | SWAP70 | SWAP-70 protein | 0.933269 | 3.145768 | 6.365967 | 2.09E−07 |
| 201075_s_at | hg133a | | SMARCC1 | SWI/SNF related, matrix | 0.967116 | 2.299167 | 6.431725 | 1.82E−07 |
| | | | | associated, actin | | | | |
| | | | | dependent regulator of | | | | |
| | | | | chromatin, subfamily c, | | | | |
| | | | | member 1 | | | | |
| 202816_s_at | hg133a | | SS18 | synovial sarcoma | 0.883622 | 2.659397 | 6.420228 | 1.35E−07 |
| | | | | translocation, | | | | |
| | | | | chromosome 18 | | | | |
| 214205_x_at | hg133a | | TXNL2 | thioredoxin-like 2 | 0.77553 | 3.36799 | 6.697244 | 5.84E−08 |
| 202589_at | hg133a | | TYMS | thymidylate synthetase; | 0.919332 | 3.436945 | 6.272954 | 2.10E−07 |
| | | | | inhibitors: 5-fluorouracil, | | | | |
| | | | | 5-fluoro-2-prime- | | | | |
| | | | | deoxyuridine, and some | | | | |
| | | | | folate analogs | | | | |
| 204529_s_at | hg133a | | TOX | thymus high mobility | 0.841683 | 4.414239 | 6.151787 | 6.54E−07 |
| | | | | group box protein TOX | | | | |
| 204033_at | hg133a | | TRIP13 | thyroid hormone receptor | 0.792036 | 2.180159 | 6.682497 | 1.49E−07 |
| | | | | interactor 13 | | | | |
| 221428_s_at | hg133a | | TBL1XR1 | transducin (beta)-like 1X- | 0.896724 | 2.023362 | 4.434993 | 8.30E−05 |
| | | | | linked receptor 1 | | | | |
| 208691_at | hg133a | | TFRC | transferrin receptor (p90, | 0.999229 | 4.258348 | 4.857726 | 2.50E−05 |
| | | | | CD71) | | | | |
| 207332_s_at | hg133a | | TFRC | transferrin receptor (p90, | 0.98799 | 5.038524 | 4.381443 | 1.13E−04 |
| | | | | CD71) | | | | |
| 208699_x_at | hg133a | | TKT | transketolase (Wernicke- | 0.933398 | 2.290241 | 5.493482 | 2.79E−06 |
| | | | | Korsakoff syndrome) | | | | |
| 213011_s_at | hg133a | | TPI1 | triosephosphate isomerase 1 | 0.999294 | 2.382566 | 8.021939 | 7.91E−10 |
| 206907_at | hg133a | NFkB | TNFSF9 | tumor necrosis factor | 0.749775 | 2.465129 | 6.720029 | 4.61E−08 |
| | | pathway | | (ligand) superfamily, | | | | |
| | | | | member 9 | | | | |
| 210317_s_at | hg133a | | YWHAE | tyrosine 3- | 0.950161 | 2.052006 | 5.393679 | 3.35E−06 |
| | | | | monooxygenase/tryptophan | | | | |
| | | | | 5-monooxygenase | | | | |
| | | | | activation protein, epsilon | | | | |
| | | | | polypeptide | | | | |
| 219960_s_at | hg133a | ubiquitin/ | UCHL5 | ubiquitin carboxyl- | 0.923828 | 2.078804 | 6.135742 | 3.51E−07 |
| | | proteosome | | terminal hydrolase L5 | | | | |
| | | pathway | | | | | | |
| 230623_x_at | hg133b | ubiquitin/ | USP28 | ubiquitin specific protease | 0.941753 | 2.323104 | 6.865617 | 3.44E−08 |
| | | proteosome | | 28 | | | | |
| | | pathway | | | | | | |
| 201898_s_at | hg133a | ubiquitin/ | UBE2A | ubiquitin-conjugating | 0.872511 | 2.287552 | 6.822285 | 3.37E−08 |
| | | proteosome | | enzyme E2A (RAD6 | | | | |
| | | pathway | | homolog) | | | | |
| 201343_at | hg133a | ubiquitin/ | UBE2D2 | ubiquitin-conjugating | 0.998073 | 2.024345 | 10.007018 | 1.91E−12 |
| | | proteosome | | enzyme E2D 2 (UBC4/5 | | | | |
| | | pathway | | homolog, yeast) | | | | |
| 209142_s_at | hg133a | ubiquitin/ | UBE2G1 | ubiquitin-conjugating | 0.974695 | 2.404522 | 6.683438 | 6.55E−08 |
| | | proteosome | | enzyme E2G 1 (UBC7 | | | | |
| | | pathway | | homolog, C. elegans) | | | | |
| 202779_s_at | hg133a | ubiquitin/ | UBE2S | ubiquitin-conjugating | 0.743224 | 4.527669 | 6.200439 | 3.69E−07 |
| | | proteosome | | enzyme E2S | | | | |
| | | pathway | | | | | | |
| 221514_at | hg133a | | UTP14A | UTP14, U3 small | 0.774374 | 2.003824 | 7.884918 | 1.45E−09 |
| | | | | nucleolar | | | | |
| | | | | ribonucleoprotein, | | | | |
| | | | | homolog A (yeast) | | | | |
| 214435_x_at | hg133a | Ral | RALA | v-ral simian leukemia | 0.97386 | 2.645153 | 7.292861 | 9.48E−09 |
| | | oncogene | | viral oncogene homolog | | | | |
| | | pathway | | A (ras related) | | | | |
| 210754_s_at | hg133a | Lyn | LYN | v-yes-1 Yamaguchi | 0.777714 | 2.24531 | 4.683368 | 3.25E−05 |
| | | oncogene | | sarcoma viral related | | | | |
| | | pathway | | oncogene homolog | | | | |

TABLE XVII
PARP1 Upregulated - Diff/X (Human); Name: Upregulated Ovary Mullerian Mixed Tumor Primary
(Minimum Fold Change: 2.0); experiment: Ovary, Mullerian Mixed Tumor, Primary; control: normal ovary.

| Fragment | | | | | | | | |
|---|---|---|---|---|---|---|---|---|
| Name | Array | Pathway | Symbol | Description | Pres. Freq. | Fold Change | t-Score | p-Value |
| 218102_at | hg133a | | DERA | 2-deoxyribose-5-phosphate | 0.982659 | 2.261239 | 3.73588 | 1.86E−02 |
| | | | | aldolase homolog (C. elegans) | | | | |
| 212312_at | hg133a | | BCL2L1 | BCL2-like 1 | 0.908863 | 2.56306 | 6.457771 | 2.56E−03 |
| 201897_s_at | hg133a | | CKS1B | CDC28 protein kinase | 0.761593 | 3.507105 | 3.842684 | 1.83E−02 |
| | | | | regulatory subunit 1B | | | | |
| 224516_s_at | hg133b | | CXXC5 | CXXC finger 5 | 0.932761 | 4.135411 | 4.953067 | 7.06E−03 |
| 202532_s_at | hg133a | | DHFR | dihydrofolate reductase; | 0.87341 | 2.076988 | 3.95417 | 1.51E−02 |
| | | | | inhibitors: A variety of drugs | | | | |
| | | | | act on dihydrofolate | | | | |
| | | | | reductase: the antibiotic | | | | |
| | | | | trimethoprim, the | | | | |
| | | | | antimalarial drug | | | | |
| | | | | pyrimethamine; the | | | | |
| | | | | chemotherapeutic agents | | | | |
| | | | | methotrexate and pemetrexed | | | | |
| 223054_at | hg133b | | DNAJB11 | DnaJ (Hsp40) homolog, | 0.987183 | 2.420047 | 8.133515 | 8.94E−04 |
| | | | | subfamily B, member 11 | | | | |
| 221782_at | hg133a | | DNAJC10 | DnaJ (Hsp40) homolog, | 0.903468 | 2.008272 | 3.811518 | 1.74E−02 |
| | | | | subfamily C, member 10 | | | | |
| 201231_s_at | hg133a | | ENO1 | enolase 1, (alpha) | 0.999743 | 3.41987 | 6.20317 | 3.20E−03 |
| 225764_at | hg133b | TEL | ETV6 | ets variant gene 6 (TEL | 0.745135 | 2.313131 | 3.678316 | 1.98E−02 |
| | | oncogene | | oncogene) | | | | |
| 205661_s_at | hg133a | | PP591 | FAD-synthetase | 0.988568 | 2.31234 | 3.999122 | 1.55E−02 |
| 200697_at | hg133a | Kinase | HK1 | hexokinase 1 | 0.859281 | 2.435525 | 4.505877 | 1.02E−02 |
| 210046_s_at | hg133a | | IDH2 | isocitrate dehydrogenase 2 | 0.971034 | 5.455361 | 4.961455 | 7.47E−03 |
| | | | | (NADP+), mitochondrial | | | | |
| 226039_at | hg133b | | MGAT4A | mannosyl (alpha-1,3-)- | 0.781841 | 2.153839 | 4.223534 | 1.17E−02 |
| | | | | glycoprotein beta-1,4-N- | | | | |
| | | | | acetylglucosaminyltransferase, | | | | |
| | | | | isoenzyme A | | | | |
| 222036_s_at | hg133a | DNA | MCM4 | MCM4 minichromosome | 0.878035 | 3.838156 | 4.460604 | 1.10E−02 |
| | | replication | | maintenance deficient 4 | | | | |
| | | | | (S. cerevisiae) | | | | |
| 209421_at | hg133a | DNA | MSH2 | mutS homolog 2, colon | 0.807964 | 2.105316 | 3.830791 | 1.77E−02 |
| | | repair | | cancer, nonpolyposis type 1 | | | | |
| | | | | (E. coli) | | | | |
| 235113_at | hg133b | | PPIL5 | peptidylprolyl isomerase | 0.887331 | 2.304155 | 5.641428 | 4.40E−03 |
| | | | | (cyclophilin)-like 5 | | | | |
| 212296_at | hg133a | proteosome | PSMD14 | proteasome (prosome, | 0.997238 | 2.435582 | 5.009506 | 6.80E−03 |
| | | | | macropain) 26S subunit, non- | | | | |
| | | | | ATPase, 14 | | | | |
| 200846_s_at | hg133a | RAS | PPP1CA | protein phosphatase 1, | 0.929929 | 2.933644 | 3.803453 | 1.80E−02 |
| | | oncogene | | catalytic subunit, alpha | | | | |
| | | family | | isoform | | | | |
| 200750_s_at | hg133a | | RAN | RAN, member RAS | 0.998715 | 2.688702 | 5.619585 | 4.50E−03 |
| | | | | oncogene family | | | | |
| 212724_at | hg133a | GTPase | RND3 | Rho family GTPase 3 | 0.851574 | 3.55359 | 5.226176 | 5.96E−03 |
| 35666_at | hg133a | | SEMA3F | sema domain, | 0.922672 | 2.070836 | 8.120797 | 5.31E−04 |
| | | | | immunoglobulin domain (Ig), | | | | |
| | | | | short basic domain, secreted, | | | | |
| | | | | (semaphorin) 3F | | | | |
| 203761_at | hg133a | | SLA | Src-like-adaptor | 0.735196 | 2.384187 | 3.875835 | 1.67E−02 |
| 213011_s_at | hg133a | | TPI1 | triosephosphate isomerase 1 | 0.999294 | 3.048039 | 6.175474 | 3.30E−03 |

TABLE XVIII
PARP1 Upregulated - Diff/X (Human); Name: Upregulated Breast Infiltrating Duct Carcinoma
(Minimum Fold Change: 2.0); Experiment: Breast, Infiltrating Ductal Carcinoma, Primary; Control: normal breast.

| Fragment | | Pathway/ | | | Pres. | Fold | | |
|---|---|---|---|---|---|---|---|---|
| Name | Array | phenotype | Symbol | Description | Freq. | Change | t-Score | p-Value |
| 211657_at | hg133a | | CEACAM6 | carcinoembryonic | 0.74 | 6.1995 | 6.5377 | 6.01E−10 |
| | | | | antigen-related cell | | | | |
| | | | | adhesion molecule 6 | | | | |
| | | | | (non-specific cross | | | | |
| | | | | reacting antigen) | | | | |
| 200766_at | hg133a | proteases | CTSD | cathepsin D (lysosomal | 0.9312 | 2.2859 | 5.2277 | 4.12E−07 |
| | | | | aspartyl protease) | | | | |
| 227094_at | hg133b | | DHTKD1 | dehydrogenase E1 and | 0.939 | 2.0672 | 10.846 | 1.76E−22 |
| | | | | transketolase domain | | | | |
| | | | | containing 1 | | | | |
| 222621_at | hg133b | | DNAJC1 | DnaJ (Hsp40) homolog, | 0.9558 | 2.0212 | 8.9955 | 1.49E−16 |
| | | | | subfamily C, member 1 | | | | |
| 202218_s_at | hg133a | fatty acid | FADS2 | fatty acid desaturase 2 | 0.8261 | 2.4369 | 6.8339 | 7.37E−11 |
| 200648_s_at | hg133a | | GLUL | glutamate-ammonia | 0.7897 | 2.0113 | 5.2625 | 3.34E−07 |
| | | | | ligase (glutamine | | | | |
| | | | | synthase) | | | | |
| 201841_s_at | hg133a | Heat shock | HSPB1 | heat shock 27 kDa | 0.9239 | 2.3522 | 7.7652 | 2.66E−13 |
| | | | | protein 1 | | | | |
| 203744_at | hg133a | | HMGB3 | high-mobility group box 3 | 0.9588 | 2.4051 | 8.5133 | 2.85E−15 |
| 205483_s_at | hg133a | | G1P2 | interferon, alpha- | 0.9342 | 4.0425 | 8.4916 | 2.35E−15 |
| | | | | inducible protein (clone | | | | |
| | | | | IFI-15K) | | | | |
| 202411_at | hg133a | | IFI27 | interferon, alpha- | 0.8202 | 2.455 | 6.7691 | 1.17E−10 |
| | | | | inducible protein 27 | | | | |
| 201088_at | hg133a | | KPNA2 | karyopherin alpha 2 | 0.9859 | 2.0066 | 7.1665 | 1.78E−11 |
| | | | | (RAG cohort 1, importin | | | | |
| | | | | alpha 1) | | | | |
| 203936_s_at | hg133a | Angiogenesis | MMP9 | matrix | 0.9952 | 2.1574 | 2.9646 | 3.51E−03 |
| | | and | | metalloproteinase 9 | | | | |
| | | NFkB | | (gelatinase B, 92 kDa | | | | |
| | | target | | gelatinase, 92 kDa type | | | | |
| | | | | IV collagenase) | | | | |
| 222036_s_at | hg133a | DNA | MCM4 | MCM4 | 0.878 | 2.0262 | 8.3579 | 7.90E−15 |
| | | Replication | | minichromosome | | | | |
| | | | | maintenance deficient 4 | | | | |
| | | | | (S. cerevisiae) | | | | |
| 224567_x_at | hg133b | | MALAT1 | metastasis associated | 0.9426 | 2.1521 | 5.1355 | 6.45E−07 |
| | | | | lung adenocarcinoma | | | | |
| | | | | transcript 1 (non-coding | | | | |
| | | | | RNA) | | | | |
| 207847_s_at | hg133a | | MUC1 | mucin 1, transmembrane | 0.8587 | 2.1904 | 6.2332 | 2.13E−09 |
| 202086_at | hg133a | | MX1 | myxovirus (influenza | 0.868 | 2.0896 | 5.7521 | 3.01E−08 |
| | | | | virus) resistance 1, | | | | |
| | | | | interferon-inducible | | | | |
| | | | | protein p78 (mouse) | | | | |
| 214440_at | hg133a | | NAT1 | N-acetyltransferase 1 | 0.9748 | 4.1277 | 6.9037 | 4.95E−11 |
| | | | | (arylamine N- | | | | |
| | | | | acetyltransferase) | | | | |
| 229353_s_at | hg133b | Casein | NUCKS | nuclear ubiquitous | 0.9585 | 2.1043 | 7.816 | 1.83E−13 |
| | | Kinase | | casein kinase and | | | | |
| | | | | cyclin-dependent kinase | | | | |
| | | | | substrate | | | | |
| 218039_at | hg133a | | NUSAP1 | nucleolar and spindle | 0.9209 | 2.693 | 9.815 | 1.81E−18 |
| | | | | associated protein 1 | | | | |
| 210004_at | hg133a | | OLR1 | oxidized low density | 0.8908 | 2.0584 | 8.6672 | 3.60E−15 |
| | | | | lipoprotein (lectin-like) | | | | |
| | | | | receptor 1 | | | | |
| 218302_at | hg133a | | PSENEN | presenilin enhancer 2 | 0.8701 | 2.0147 | 11.65 | 1.90E−24 |
| | | | | homolog (C. elegans) | | | | |
| 217763_s_at | hg133a | Ras | RAB31 | RAB31, member RAS | 0.8025 | 3.1113 | 7.8645 | 2.02E−13 |
| | | | | oncogene family | | | | |
| 209875_s_at | hg133a | | SPP1 | secreted phosphoprotein | 0.7963 | 2.8649 | 7.5204 | 1.72E−12 |
| | | | | 1 (osteopontin, bone | | | | |
| | | | | sialoprotein I, early T- | | | | |
| | | | | lymphocyte activation | | | | |
| | | | | 1) | | | | |
| 201563_at | hg133a | | SORD | sorbitol dehydrogenase | 0.9759 | 2.4665 | 8.8312 | 2.49E−16 |
| 209218_at | hg133a | | SQLE | squalene epoxidase | 0.9956 | 2.2077 | 5.8463 | 5.50E−08 |
| 217979_at | hg133a | | TSPAN13 | tetraspanin 13 | 0.9737 | 2.0879 | 9.6982 | 7.05E−19 |
| 36936_at | hg133a | | TSTA3 | tissue specific | 0.9741 | 2.2268 | 13.076 | 2.09E−29 |
| | | | | transplantation antigen | | | | |
| | | | | P35B | | | | |
| 201688_s_at | hg133a | | TPD52 | tumor protein D52 | 0.8121 | 2.3836 | 8.7569 | 5.04E−16 |
| 202779_s_at | hg133a | ubiquitin- | UBE2S | ubiquitin-conjugating | 0.7432 | 2.7685 | 6.5763 | 3.86E−10 |
| | | proteasome | | enzyme E2S | | | | |

### Techniques for Analysis of Differentially Expressed Genes

Analysis of co-regulated expressed genes includes analysis of PARP gene expression, and all genes differentially expressed in human tumor tissues, including IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, CDK1, CDK2, CDK9, farnesyl transferase, UBE2A, UBE2D2, UBE2G1, USP28 or UBE2S, which may include an analysis of DNA, RNA, analysis of the level of the co-regulated genes and/or analysis of the activity of protein product of the co-regulated genes, for example, measuring the level of mono- and poly-ADP-ribosylation for PARP gene expression, or enzymatic activity of other co-regulated genes coding for enzymes. Other co-differentially expressed genes may also include without limitation IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, CDK1, CDK2, CDK9, farnesyl transferase, UBE2A, UBE2D2, UBE2G1, USP28, UBE2S, ABCC1, ABCC5, ABCD4, ACADM, ACLSL1, ACSL3, ACY1L2, ADM, ADRM1, AGPAT5, AHCY, AK3L1, AK3L2, AKIIP, AKR1B1, AKR1C1, AKR1C2, AKR1C3, ALDH18A1, ALDOA, ALOX5, ALPL, ANP32E, AOF1, APG5L, ARFGEF1, ARL5, ARPP-19, ASPH, ATF5, ATF7IP, ATIC, ATP11A, ATP11C, ATP1A1, ATP1B1, ATP2A2, ATP5G3, ATP5J2, ATP6V0B, B3GNT1, B4GALT2, BACE2, BACH, BAG2, BASP1, BCAT1, BCL2L1, BCL6, BGN, BPNT1, C1QBP, CACNB3, CAMK2D, CAP2, CCAR1, CD109, CD24, CD44, CD47, CD58, CD74, CD83, CD9, CDC14B, CDC42EP4, CDC5L, CDK4, CDK6, CDS1, CDW92, CEACAM6, CELSR2, CFLAR, CGI-90, CHST6, CHSY1, CKLFSF4, CKLFSF6, CKS1B, CMKOR1, CNDP2, CPD, CPE, CPSF3, CPSF5, CPSF6, CPT1B, CRR9, CSH2, CSK, CSNK2A1, CSPG2, CTPSCTSB, CTSD, CXADR, CXCR4, CXXC5, CXXC6, DAAM1, DCK, DDAH1, DDIT4, DDR1, DDX21, DDX39, DHTKD1, DLAT, DNAJA1, DNAJB11, DNAJC1, DNAJC10, DNAJC9, DNAJD1, DUSP10, DUSP24, DUSP6, DVL3, ELOVL6, EME1, ENO1, ENPP4, EPS8, ETNK1, ETV6, F11R, FA2H, FABP5, FADS2, FAS, FBXO45, FBXO7, FLJ23091, FTL, FTLL1, FZD6, G1P2, GALNT2, GALNT4, GALNT7, GANAB, GART, GBAS, GCHFR, GCLC, GCLM, GCNT1, GFPT1, GGA2, GGH, GLUL, GMNN, GMPS, GPI, GPR56, GPR89, GPX1, GRB10, GRHPR, GSPT1, GSR, GTPBP4, HDAC1, HDGF, HIG2, HMGB3, HPRT1, HPS5, HRMT1L2, HS2ST1, HSPA4, HSPA8, HSPB1, HSPCA, HSPCAL3, HSPCB, HSPD1, HSPE1, HSPH1, HTATIP2, HYOU1, ICMT, IDE, IDH2, IFI27, IGFBP3, IGSF4, ILF2, INPP5F, INSIG1, KHSRP, KLF4, KMO, KPNA2, KTN1, LAP3, LASS2, LDHA, LDHB, LGR4, LPGAT1, LTB4DH, LYN, MAD2L1, MADP-1, MAGED1, MAK3, MALAT1, MAP2K3, MAP2K6, MAP3K13, MAP4K4, MAPK13, MARCKS, MBTPS2, MCM4, MCTS1, MDH1, MDH2, ME1, ME2, METAP2, METTL2, MGAT4B, MKNK2. MLPH, MOBK1B, MOBKL1A, MSH2, MTHFD2, MUC1, MX1, MYCBP, NAJD1, NAT1, NBS1, NDFIP2, NEK6, NET1, NME1, NNT, NQO1, NRAS, NSE2, NUCKS, NUSAP1, NY-REN-41, ODC1, OLR1, P4HB, PAFAH1B1, PAICS, PANK1, PCIA1, PCNA, PCTK1, PDAP1, PDIA4, PDIA6, PDXK, PERP, PFKP, PFTK1, PGD, PGK1, PGM2L1, PHCA, PKIG, PKM2, PKP4, PLA2G4A, PLCB1, PLCG2, PLD3, PLOD1, PLOD2, PMS2L3, PNK1, PNPT1, PON2, PP, PPIF, PPP1CA, PPP2R4, PPP3CA, PRCC, PRKD3, PRKDC, PRPSAP2, PSAT1, PSENEN, PSMA2, PSMA5, PSMA7, PSMB3, PSMB4, PSMD14, PSMD2, PSMD3, PSMD4, PSMD8, PTGFRN, PTGS1, PTK9, PTPN12, PTPN18, PTS, PYGB, RAB10, RAB11FIP1, RAB14, RAB31, RAB3IP, RACGAP1, RAN, RANBP1, RAP2B, RBBP4, RBBP7, RBBP8, RDH10, RFC3, RFC4, RFC5, RGS19IP1, RHOBTB3, RNASEH2A, RNGTT, RNPEP, ROBO1, RRAS2, SART2, SAT, SCAP2, SCD4, SDC2, SDC4, SEMA3F, SERPINE2, SFI1, SGPL1, SGPP1, SGPP2, SH3GLB2, SHC1, SMARCC1, SMC4L1, SMC4L1, SMS, SNRPD1, SORD, SORL1, SPP1, SQLE, SRD5A1, SRD5A2L, SRM, SRPK1, SS18, SSBP1, SSR3, ST3GAL5, ST6GAL1, ST6GALNAC2, STX18, SULF2, SWAP70, TA-KRP, TALA, TBL1XR1, TFRC, TIAM1, TKT, TMPO, TNFAIP2, TNFSF9, TOX, TPD52, TPI1, TPP1, TRA1, TRIP13, TRPS1, TSPAN13, TSTA3, TXN, TXNL2, TXNL5, TXNRD1, UBAP2L, UBE2A, UBE2D2, UBE2G1, UBE2V1, UCHL5, UGDH, UNC5CL, USP28, USP47, UTP14A, VDAC1, WIG1, YWHAB, YWHAE and YWHAZ. Without limiting the scope of the present embodiments, any number of techniques known in the art can be employed for the analysis of the co-regulated genes, and they are all within the scope of the present embodiments. Some of the examples of such detection techniques are given below but these examples are in no way limiting to the various detection techniques that can be used in the present embodiments.

Gene Expression Profiling: Methods of gene expression profiling include methods based on hybridization analysis of polynucleotides, polyribonucleotides methods based on sequencing of polynucleotides, polyribonucleotides and proteomics-based methods. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS), Comparative Genome Hybridization (CGH), Chromatin Immunoprecipitation (ChIP), Single nucleotide polymorphism (SNP) and SNP arrays, Fluorescent in situ Hybridization (FISH), Protein binding arrays and DNA microarray (also commonly known as gene or genome chip, DNA chip, or gene array), RNA microarrays.

Reverse Transcriptase PCR(RT-PCR): One of the most sensitive and most flexible quantitative PCR-based gene expression profiling methods is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. For example, the starting material can be typically total RNA isolated from human tumors or tumor cell lines, and corresponding normal tissues or cell lines, respectively. Thus RNA can be isolated from a variety of normal and diseased cells and tissues, for example tumors, including breast, lung, colorectal, prostate, brain, liver, kidney, pancreas, spleen, thymus, testis, ovary, uterus, etc., or tumor cell lines. If the source of mRNA is a primary tumor, mRNA can be extracted, for example, from frozen or archived fixed tissues, for example paraffin-embedded and fixed (e.g. formalin-fixed) tissue samples. General methods for mRNA extraction are well known in the art and are disclosed in standard textbooks of molecular biology, including Ausubel et al., Current Protocols of Molecular Biology, John Wiley and Sons (1997).

In particular, RNA isolation can be performed using purification kit, buffer set and protease from commercial manufacturers, according to the manufacturer's instructions. RNA prepared from tumor can be isolated, for example, by cesium chloride density gradient centrifugation. As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. The derived cDNA can then be used as a template in the subsequent PCR reaction.

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe. Real time PCR is compatible both with quantitative competitive PCR, where internal competitor for each target sequence is used for normalization, and with quantitative comparative PCR using a normalization gene contained within the sample, or a housekeeping gene for RT-PCR.

Microscopy: Some embodiments include microscopy for analysis of differentially expressed genes, including at least PARP. For example, fluorescence microscopy enables the molecular composition of the structures being observed to be identified through the use of fluorescently-labeled probes of high chemical specificity such as antibodies. It can be done by directly conjugating a fluorophore to a protein and introducing this back into a cell. Fluorescent analogue may behave like the native protein and can therefore serve to reveal the distribution and behavior of this protein in the cell. Along with NMR, infrared spectroscopy, circular dichroism and other techniques, protein intrinsic fluorescence decay and its associated observation of fluorescence anisotropy, collisional quenching and resonance energy transfer are techniques for protein detection. The naturally fluorescent proteins can be used as fluorescent probes. The jellyfish aequorea victoria produces a naturally fluorescent protein known as green fluorescent protein (GFP). The fusion of these fluorescent probes to a target protein enables visualization by fluorescence microscopy and quantification by flow cytometry.

By way of example only, some of the probes are labels such as, fluorescein and its derivatives, carboxyfluoresceins, rhodamines and their derivatives, atto labels, fluorescent red and fluorescent orange: cy3/cy5 alternatives, lanthanide complexes with long lifetimes, long wavelength labels—up to 800 nm, DY cyanine labels, and phycobili proteins. By way of example only, some of the probes are conjugates such as, isothiocyanate conjugates, streptavidin conjugates, and biotin conjugates. By way of example only, some of the probes are enzyme substrates such as, fluorogenic and chromogenic substrates. By way of example only, some of the probes are fluorochromes such as, FITC (green fluorescence, excitation/emission=506/529 nm), rhodamine B (orange fluorescence, excitation/emission=560/584 nm), and nile blue A (red fluorescence, excitation/emission=636/686 nm). Fluorescent nanoparticles can be used for various types of immunoassays. Fluorescent nanoparticles are based on different materials, such as, polyacrylonitrile, and polystyrene etc. Fluorescent molecular rotors are sensors of micro environmental restriction that become fluorescent when their rotation is constrained. Few examples of molecular constraint include increased dye (aggregation), binding to antibodies, or being trapped in the polymerization of actin. IEF (isoelectric focusing) is an analytical tool for the separation of ampholytes, mainly proteins. An advantage for IEF-gel electrophoresis with fluorescent IEF-marker is the possibility to directly observe the formation of gradient. Fluorescent IEF-marker can also be detected by UV-absorption at 280 nm (20° C.).

A peptide library can be synthesized on solid supports and, by using coloring receptors, subsequent dyed solid supports can be selected one by one. If receptors cannot indicate any color, their binding antibodies can be dyed. The method can not only be used on protein receptors, but also on screening binding ligands of synthesized artificial receptors and screening new metal binding ligands as well. Automated methods for HTS and FACS (fluorescence activated cell sorter) can also be used. A FACS machine originally runs cells through a capillary tube and separate cells by detecting their fluorescent intensities.

Immunoassays: Some embodiments include immunoassay for the analysis of the differentially regulated genes. In immunoblotting like the western blot of electrophoretically separated proteins a single protein can be identified by its antibody. Immunoassay can be competitive binding immunoassay where analyte competes with a labeled antigen for a limited pool of antibody molecules (e.g. radioimmunoassay, EMIT). Immunoassay can be non-competitive where antibody is present in excess and is labeled. As analyte antigen complex is increased, the amount of labeled antibody-antigen complex may also increase (e.g. ELISA). Antibodies can be polyclonal if produced by antigen injection into an experimental animal, or monoclonal if produced by cell fusion and cell culture techniques. In immunoassay, the antibody may serve as a specific reagent for the analyte antigen.

Without limiting the scope and content of the present embodiments, some of the types of immunoassays are, by way of example only, RIAs (radioimmunoassay), enzyme immunoassays like ELISA (enzyme-linked immunosorbent assay), EMIT (enzyme multiplied immunoassay technique), microparticle enzyme immunoassay (MEIA), LIA (luminescent immunoassay), and FIA (fluorescent immunoassay). These techniques can be used to detect biological substances in the nasal specimen. The antibodies—either used as primary or secondary ones—can be labeled with radioisotopes (e.g. 125I), fluorescent dyes (e.g. FITC) or enzymes (e.g. HRP or AP) which may catalyze fluorogenic or luminogenic reactions.

Biotin, or vitamin H is a co-enzyme which inherits a specific affinity towards avidin and streptavidin. This interaction makes biotinylated peptides a useful tool in various biotechnology assays for quality and quantity testing. To improve biotin/streptavidin recognition by minimizing steric hindrances, it can be necessary to enlarge the distance between biotin and the peptide itself. This can be achieved by coupling a spacer molecule (e.g., 6-aminohexanoic acid) between biotin and the peptide.

The biotin quantitation assay for biotinylated proteins provides a sensitive fluorometric assay for accurately determining the number of biotin labels on a protein. Biotinylated peptides are widely used in a variety of biomedical screening systems requiring immobilization of at least one of the interaction partners onto streptavidin coated beads, membranes, glass slides or microtiter plates. The assay is based on the displacement of a ligand tagged with a quencher dye from the biotin binding sites of a reagent. To expose any biotin groups in a multiply labeled protein that are sterically restricted and inaccessible to the reagent, the protein can be treated with protease for digesting the protein.

EMIT is a competitive binding immunoassay that avoids the usual separation step. A type of immunoassay in which the protein is labeled with an enzyme, and the enzyme-protein-antibody complex is enzymatically inactive, allowing quantitation of unlabelled protein. Some embodiments include an ELISA assay to analyze the differentially expressed genes, including at least PARP. ELISA is based on selective antibodies attached to solid supports combined with enzyme reactions to produce systems capable of detecting low levels of proteins. It is also known as enzyme immunoassay or EIA. The protein is detected by antibodies that have been made against it, that is, for which it is the antigen. Monoclonal antibodies are often used.

The test may require the antibodies to be fixed to a solid surface, such as the inner surface of a test tube, and a preparation of the same antibodies coupled to an enzyme. The enzyme may be one (e.g., β-galactosidase) that produces a colored product from a colorless substrate. The test, for example, may be performed by filling the tube with the antigen solution (e.g., protein) to be assayed. Any antigen molecule present may bind to the immobilized antibody molecules. The antibody-enzyme conjugate may be added to the reaction mixture. The antibody part of the conjugate binds to any antigen molecules that were bound previously, creating an antibody-antigen-antibody “sandwich”. After washing away any unbound conjugate, the substrate solution may be added. After a set interval, the reaction is stopped (e.g., by adding 1 N NaOH) and the concentration of colored product formed is measured in a spectrophotometer. The intensity of color is proportional to the concentration of bound antigen.

ELISA can also be adapted to measure the concentration of antibodies, in which case, the wells are coated with the appropriate antigen. The solution (e.g., serum) containing antibody may be added. After it has had time to bind to the immobilized antigen, an enzyme-conjugated anti-immunoglobulin may be added, consisting of an antibody against the antibodies being tested for. After washing away unreacted reagent, the substrate may be added. The intensity of the color produced is proportional to the amount of enzyme-labeled antibodies bound (and thus to the concentration of the antibodies being assayed).

Some embodiments include radioimmunoassays to analyze the levels of the differentially expressed genes, including at least PARP. Isotopes can be used to study in vivo metabolism, distribution, as well as binding of ligands to target proteins. Isotopes of ¹H, ¹²C, ¹³C, ³¹P, ³²S, and ¹²⁷I in body are used such as ³H, ¹⁴C, ¹³C, ³²P, ³⁵S, and ¹²⁵I. In receptor fixation method in 96 well plates, receptors may be fixed in each well by using antibody or chemical methods and radioactive labeled ligands may be added to each well to induce binding. Unbound ligands may be washed out and then the standard can be determined by quantitative analysis of radioactivity of bound ligands or that of washed-out ligands. Then, addition of screening target compounds may induce competitive binding reaction with receptors. If the compounds show higher affinity to receptors than standard radioactive ligands, most of radioactive ligands would not bind to receptors and may be left in solution. Therefore, by analyzing quantity of bound radioactive ligands (or washed-out ligands), testing compounds' affinity to receptors can be indicated.

The filter membrane method may be needed when receptors cannot be fixed to 96 well plates or when ligand binding needs to be done in solution phase. In other words, after ligand-receptor binding reaction in solution, if the reaction solution is filtered through nitrocellulose filter paper, small molecules including ligands may go through it and only protein receptors may be left on the paper. Only ligands that strongly bound to receptors may stay on the filter paper and the relative affinity of added compounds can be identified by quantitative analysis of the standard radioactive ligands.

Some embodiments include fluorescence immunoassays for the analysis of differentially expressed genes, including at least PARP. Fluorescence based immunological methods are based upon the competitive binding of labeled ligands versus unlabeled ones on highly specific receptor sites. The fluorescence technique can be used for immunoassays based on changes in fluorescence lifetime with changing analyte concentration. This technique may work with short lifetime dyes like fluorescein isothiocyanate (FITC) (the donor) whose fluorescence may be quenched by energy transfer to eosin (the acceptor). A number of photoluminescent compounds may be used, such as cyanines, oxazines, thiazines, porphyrins, phthalocyanines, fluorescent infrared-emitting polynuclear aromatic hydrocarbons, phycobiliproteins, squaraines and organo-metallic complexes, hydrocarbons and azo dyes.

Fluorescence based immunological methods can be, for example, heterogeneous or homogenous. Heterogeneous immunoassays comprise physical separation of bound from free labeled analyte. The analyte or antibody may be attached to a solid surface. The technique can be competitive (for a higher selectivity) or noncompetitive (for a higher sensitivity). Detection can be direct (only one type of antibody used) or indirect (a second type of antibody is used). Homogenous immunoassays comprise no physical separation. Double-antibody fluorophore-labeled antigen participates in an equilibrium reaction with antibodies directed against both the antigen and the fluorophore. Labeled and unlabeled antigen may compete for a limited number of anti-antigen antibodies.

Some of the fluorescence immunoassay methods include simple fluorescence labeling method, fluorescence resonance energy transfer (FRET), time resolved fluorescence (TRF), and scanning probe microscopy (SPM). The simple fluorescence labeling method can be used for receptor-ligand binding, enzymatic activity by using pertinent fluorescence, and as a fluorescent indicator of various in vivo physiological changes such as pH, ion concentration, and electric pressure. TRF is a method that selectively measures fluorescence of the lanthanide series after the emission of other fluorescent molecules is finished. TRF can be used with FRET and the lanthanide series can become donors or acceptors. In scanning probe microscopy, in the capture phase, for example, at least one monoclonal antibody is adhered to a solid phase and a scanning probe microscope is utilized to detect antigen/antibody complexes which may be present on the surface of the solid phase. The use of scanning tunneling microscopy eliminates the need for labels which normally is utilized in many immunoassay systems to detect antigen/antibody complexes.

Protein identification methods: By way of example only, protein identification methods include low-throughput sequencing through Edman degradation, mass spectrometry techniques, peptide mass fingerprinting, de novo sequencing, and antibody-based assays. The protein quantification assays include fluorescent dye gel staining, tagging or chemical modification methods (i.e. isotope-coded affinity tags (ICATS), combined fractional diagonal chromatography (COFRADIC)). The purified protein may also be used for determination of three-dimensional crystal structure, which can be used for modeling intermolecular interactions. Common methods for determining three-dimensional crystal structure include x-ray crystallography and NMR spectroscopy. Characteristics indicative of the three-dimensional structure of proteins can be probed with mass spectrometry. By using chemical cross-linking to couple parts of the protein that are close in space, but far apart in sequence, information about the overall structure can be inferred. By following the exchange of amide protons with deuterium from the solvent, it is possible to probe the solvent accessibility of various parts of the protein.

In one embodiment, fluorescence-activated cell-sorting (FACS) is used to identify cells that differentially express the identified genes, including at least PARP. FACS is a specialized type of flow cytometry. It provides a method for sorting a heterogeneous mixture of biological cells into two or more containers, one cell at a time, based upon the specific light scattering and fluorescent characteristics of each cell. It provides quantitative recording of fluorescent signals from individual cells as well as physical separation of cells of particular interest. In yet another embodiment, microfluidic based devices are used to evaluate expression of the identified differentially regulated genes.

Mass spectrometry can also be used to characterize expression of the differentially regulated genes, including at least PARP, from patient samples. The two methods for ionization of whole proteins are electrospray ionization (ESI) and matrix-assisted laser desorption/ionization (MALDI). In the first, intact proteins are ionized by either of the two techniques described above, and then introduced to a mass analyzer. In the second, proteins are enzymatically digested into smaller peptides using an agent such as trypsin or pepsin. Other proteolytic digest agents are also used. The collection of peptide products are then introduced to the mass analyzer. This is often referred to as the “bottom-up” approach of protein analysis.

Whole protein mass analysis is conducted using either time-of-flight (TOF) MS, or Fourier transform ion cyclotron resonance (FT-ICR). The instrument used for peptide mass analysis is the quadrupole ion trap. Multiple stage quadrupole-time-of-flight and MALDI time-of-flight instruments also find use in this application.

Two methods used to fractionate proteins, or their peptide products from an enzymatic digestion. The first method fractionates whole proteins and is called two-dimensional gel electrophoresis. The second method, high performance liquid chromatography is used to fractionate peptides after enzymatic digestion. In some situations, it may be necessary to combine both of these techniques.

There are two ways mass spectroscopy can be used to identify proteins. Peptide mass uses the masses of proteolytic peptides as input to a search of a database of predicted masses that would arise from digestion of a list of known proteins. If a protein sequence in the reference list gives rise to a significant number of predicted masses that match the experimental values, there is some evidence that this protein was present in the original sample.

Tandem MS is also a method for identifying proteins. Collision-induced dissociation is used in mainstream applications to generate a set of fragments from a specific peptide ion. The fragmentation process primarily gives rise to cleavage products that break along peptide bonds.

A number of different algorithmic approaches have been described to identify peptides and proteins from tandem mass spectrometry (MS/MS), peptide de novo sequencing and sequence tag based searching. One option that combines a comprehensive range of data analysis features is PEAKS. Other existing mass spec analysis software include: Peptide fragment fingerprinting SEQUEST, Mascot, OMSSA and X!Tandem).

Proteins can also be quantified by mass spectrometry. Typically, stable (e.g. non-radioactive) heavier isotopes of carbon (C¹³) or nitrogen (N¹⁵) are incorporated into one sample while the other one is labeled with corresponding light isotopes (e.g. C¹² and N¹⁴). The two samples are mixed before the analysis. Peptides derived from the different samples can be distinguished due to their mass difference. The ratio of their peak intensities corresponds to the relative abundance ratio of the peptides (and proteins). The methods for isotope labeling are SILAC (stable isotope labeling with amino acids in cell culture), trypsin-catalyzed O¹⁸ labeling, ICAT (isotope coded affinity tagging), ITRAQ (isotope tags for relative and absolute quantitation). “Semi-quantitative” mass spectrometry can be performed without labeling of samples. Typically, this is done with MALDI analysis (in linear mode). The peak intensity, or the peak area, from individual molecules (typically proteins) is here correlated to the amount of protein in the sample. However, the individual signal depends on the primary structure of the protein, on the complexity of the sample, and on the settings of the instrument.

N-terminal sequencing aids in the identification of unknown proteins, confirm recombinant protein identity and fidelity (reading frame, translation start point, etc.), aid the interpretation of NMR and crystallographic data, demonstrate degrees of identity between proteins, or provide data for the design of synthetic peptides for antibody generation, etc. N-terminal sequencing utilizes the Edman degradative chemistry, sequentially removing amino acid residues from the N-terminus of the protein and identifying them by reverse-phase HPLC. Sensitivity can be at the level of 100s femtomoles and long sequence reads (20-40 residues) can often be obtained from a few 10s picomoles of starting material. Pure proteins (>90%) can generate easily interpreted data, but insufficiently purified protein mixtures may also provide useful data, subject to rigorous data interpretation. N-terminally modified (especially acetylated) proteins cannot be sequenced directly, as the absence of a free primary amino-group prevents the Edman chemistry. However, limited proteolysis of the blocked protein (e.g. using cyanogen bromide) may allow a mixture of amino acids to be generated in each cycle of the instrument, which can be subjected to database analysis in order to interpret meaningful sequence information. C-terminal sequencing is a post-translational modification, affecting the structure and activity of a protein. Various disease situations can be associated with impaired protein processing and C-terminal sequencing provides an additional tool for the investigation of protein structure and processing mechanisms.

### Identifying Diseases Treatable by Modulators of the Differentially Regulated Genes

Some embodiments relate to identifying a disease treatable by modulators of co-regulated genes comprising identifying a level of expression of the co-regulated genes, including at least PARP, in a sample of a subject, making a decision regarding identifying the disease treatable by modulators of the co-regulated genes, wherein the decision is made based on the level of expression of the co-regulated genes, including at least PARP. The identification of the level of the co-regulated genes may include analysis of RNA, analysis of level of proteins expressed by the regulated genes and/or analysis of activity said proteins. When the levels of the regulated genes are up-regulated in a disease, the disease may be treated with inhibitors of the co-regulated genes.

In other embodiments, the level of the regulated expressed genes is determined in samples from a patient population and compared with samples from a normal population in order to correlate any changes in expression levels of these regulated genes, including at least PARP, with the existence of a disease. The identification and analysis of the level of these regulated genes may also include analysis of RNA, analysis of the level of proteins expressed by the regulated genes as well as analysis of activity these proteins. When the levels of expression of the regulated genes are increased in a number of samples from a patient population in comparison to samples from a normal population, the disease may be treated with inhibitors to the regulated genes. In some embodiments, an increase of at least 25%, at least 30%, at least 35%, at least 40%, at least 45%, at least 50%, at least 55%, at least 60%, at least 65%, at least 70% or more may indicate sufficient correlation of upregulation of the co-regulated genes for a specific disease or group of diseases.

In one embodiment, upregulation of the regulated genes identified is used as an embodiment of BRCA deficient cancer, especially PARP upregulation. Accordingly, the methods can be used to identify for example a BRCA mediated cancer treatable by modulators of the upregulated identified genes, including PARP inhibitors and modulators of co-regulated expressed genes, including IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, CDK1, CDK2, CDK9, farnesyl transferase, UBE2A, UBE2D2, UBE2G1, USP28 or UBE2S. The identification of a level of expression of the co-regulated genes may involve one or more comparisons with reference samples. The reference samples may be obtained from the same subject or from a different subject who is either not affected with the disease (such as, normal subject) or is a patient. The reference sample could be obtained from one subject, multiple subjects or is synthetically generated. The identification may also involve comparison of the identification data with the databases. One embodiment relates to identifying the level of regulated expressed genes, including at least PARP, in a subject afflicted with disease and correlating it with the expression level of the same set of co-regulated expressed genes in normal subjects. In some embodiments, the step of correlating the level of co-regulated expressed genes is performed by a software algorithm. The data generated can be transformed into computer readable form; and an algorithm is executed that classifies the data according to user input parameters, for detecting signals that represent level of expression of regulated expressed genes in diseased patients or patient populations, and correspondingly levels of expression in normal subjects or populations.

The identification and analysis of the expression level of the regulated expressed genes, including at least PARP, identified through the methods described herein have numerous therapeutic and diagnostic applications. Clinical applications include, for example, detection of disease, distinguishing disease states to inform prognosis, selection of therapy such as, treatment with PARP inhibitors and modulators of co-regulated expressed genes, and/or prediction of therapeutic response, disease staging, identification of disease processes, prediction of efficacy of therapy, monitoring of patients trajectories (e.g., prior to onset of disease), prediction of adverse response, monitoring of therapy associated efficacy and toxicity, and detection of recurrence.

The identification of the level of expression of regulated expressed genes, including at least PARP, and the subsequent identification of a disease in a subject or subject population treatable by PARP inhibitors and modulators of regulated expressed genes, as disclosed herein can be used to enable or assist in the pharmaceutical drug development process for therapeutic agents. The identification of the expression level of the regulated expressed genes, for example, can be used to diagnose disease for patients enrolling in a clinical trial, for example in a patient population. The identification of the expression level of regulated expressed genes, including at least PARP, can indicate the state of the disease of patients undergoing treatment in clinical trials, and show changes in the state during the treatment. The identification of the expression level of regulated expressed genes can demonstrate the efficacy of treatment with modulators of the regulated expressed genes, and can be used to stratify patients according to their responses to various therapies.

The methods described herein can be used to identify the state of a disease in a patient or a patient population. In one embodiment, the methods are used to detect the earliest stages of disease. In other embodiments, the methods are used to grade the identified disease. In certain embodiments, patients, health care providers, such as doctors and nurses, or health care managers, use the expression level of the identified regulated expressed genes, including at least PARP, in a subject to make a diagnosis, prognosis, and/or select treatment options, such as treatment with PARP inhibitors. In other embodiments, health care providers and patients may use the expression levels of each identified target regulated expressed gene obtained in a patient population to also make a diagnosis, prognosis, and/or select treatment options, such as treatment with a combination of PARP inhibitors and modulators of co-regulated expressed genes.

In other embodiments, the methods described herein can be used to predict the likelihood of response for any individual or patient population to a particular treatment, select a treatment, or to preempt the possible adverse effects of treatments on a particular individual. Also, the methods can be used to evaluate the efficacy of treatments over time. For example, biological samples can be obtained from a patient over a period of time as the patient is undergoing treatment. The expression level of each identified gene in a panel of gene targets in the different samples can be compared to each other to determine the efficacy of the treatment. Also, the methods described herein can be used to compare the efficacies of different disease therapies and/or responses to one or more treatments in different populations (e.g., ethnicities, family histories, etc.).

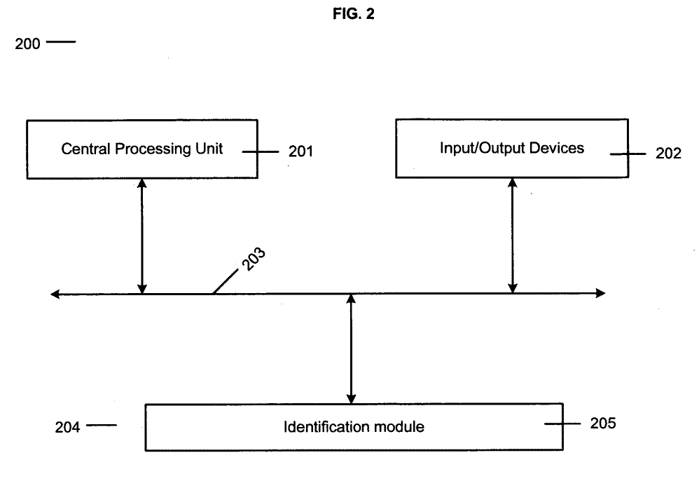

In some embodiments, at least one step of the methods described herein is performed using a computer as depicted in FIG. 2. FIG. 2 illustrates a computer for implementing selected operations associated with the methods described herein. The computer 200 includes a central processing unit 201 connected to a set of input/output devices 202 via a system bus 203. The input/output devices 202 may include a keyboard, mouse, scanner, data port, video monitor, liquid crystal display, printer, and the like. A memory 204 in the form of primary and/or secondary memory is also connected to the system bus 203. These components of FIG. 2 characterize a standard computer. This standard computer is programmed in accordance with the methods described herein. In particular, the computer 200 can be programmed to perform various operations of the methods described herein.

The memory 204 of the computer 200 may store an identification module 205. In other words, the identification module 205 can perform the operations associated with step 102, 103, and 104 of FIG. 1. The term “identification module” used herein includes, but is not limited to, analyzing expression levels of regulated expressed genes, including at least PARP, in a sample of a subject; optionally comparing the expression level data of the test sample with the reference sample; identifying the expression level of each identified co-regulated expressed gene in the sample; identifying the disease; and further identifying the disease treatable by a combination of PARP inhibitors and modulators of co-regulated expressed genes. The identification module may also include a decision module where the decision module includes executable instructions to make a decision regarding identifying the disease treatable by modulators of co-regulated expressed genes and/or provide a conclusion regarding the disease to a patient, a health care provider or a health care manager. The executable code of the identification module 205 may utilize any number of numerical techniques to perform the comparisons and diagnosis.

Some embodiments include a computer readable medium with information regarding a disease in a subject treatable by modulators of identified co-regulated expressed genes, including at least PARP, the information being derived by identifying expression levels of each identified co-regulated expressed gene, including at least PARP, in the sample of the subject, and making a decision based on the expression levels of each identified co-regulated expressed gene, regarding treating the disease by modulators of the identified co-regulated expressed genes. The medium may contain a reference pattern of one or more of expression levels of each identified co-regulated expressed gene in a sample. This reference pattern can be used to compare the pattern obtained from a test subject and an analysis of the disease can be made based on this comparison. This reference pattern can be from normal subjects, i.e., subjects with no disease, subjects with different levels of disease, subjects with disease of varying severity. These reference patterns can be used for diagnosis, prognosis, evaluating efficacy of treatment, and/or determining the severity of the disease state of a subject. The methods described herein also include sending information regarding expression levels of each identified co-regulated expressed gene in a sample in a subject and/or decision regarding identifying the disease treatable by modulators or inhibitors described herein, between one or more computers, for example with the use of the internet.

### Diseases

Various diseases include, but are not limited to, cancer types including adrenal cortical cancer, anal cancer, aplastic anemia, bile duct cancer, bladder cancer, bone cancer, bone metastasis, adult CNS brain tumors, children CNS brain tumors, breast cancer, castleman disease, cervical cancer, childhood Non-Hodgkin's lymphoma, colon and rectum cancer, endometrial cancer, esophagus cancer, Ewing's family of tumors, eye cancer, gallbladder cancer, gastrointestinal carcinoid tumors, gastrointestinal stromal tumors, gestational trophoblastic disease, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, nonmelanoma skin cancer, stomach cancer, testicular cancer, thymus cancer, thyroid cancer, uterine sarcoma, vaginal cancer, vulvar cancer, Waldenstrom's macroglobulinemia, chronic lymphocyte leukemia, and reactive lymphoid hyperplasia.

Diseases include angiogenesis in cancers, inflammation, cardiovascular diseases, degenerative diseases, CNS diseases, autoimmune diseases, and viral diseases, including HIV. The compounds described herein are also useful in the modulation of cellular response to pathogens. Also provided herein are methods to treat other diseases, such as, viral diseases. Some of the viral diseases are, but not limited to, human immunodeficiency virus (HIV), herpes simplex virus type-1 and 2 and cytomegalovirus (CMV), a dangerous co-infection of HIV.

Some examples of the diseases are set forth herein, but without limiting the scope of the present embodiments, there may be other diseases known in the art and are within the scope of the present embodiments.

Examples of Cancers

Examples of cancers include, but are not limited to, lymphomas, carcinomas and hormone-dependent tumors (e.g., breast, prostate or ovarian cancer). Abnormal cellular proliferation conditions or cancers that may be treated in either adults or children include solid phase tumors/malignancies, locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female reproductive tract including ovarian carcinoma, uterine (including endometrial) cancers, and solid tumor in the ovarian follicle, kidney cancers including renal cell carcinoma, brain cancers including intrinsic brain tumors, neuroblastoma, astrocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Karposi's sarcoma.

In some embodiments, cancer includes colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, intraductal carcinoma, mucinous carcinoma, phyllodes tumor, ovarian adenocarcinoma, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung adenocarcinoma, kidney carcinoma, urinary bladder carcinoma, and Wilm's tumor.

In still further embodiments, cancer includes mullerian mixed tumor of the endometrium, infiltrating carcinoma of mixed ductal and lobular type, Wilm's tumor, mullerian mixed tumor of the ovary, serous cystadenocarcinoma, ovary adenocarcinoma (papillary serous type), ovary adenocarcinoma (endometrioid type), metastatic infiltrating lobular carcinoma of breast, testis seminoma, prostate benign nodular hyperplasia, lung squamous cell carcinoma, lung large cell carcinoma, lung adenocarcinoma, endometrium adenocarcinoma (endometrioid type), infiltrating ductal carcinoma, skin basal cell carcinoma, breast infiltrating lobular carcinoma, fibrocystic disease, fibroadenoma, glioma, chronic myeloid leukemia, liver hepatocellular carcinoma, mucinous carcinoma, Schwannoma, kidney transitional cell carcinoma, Hashimoto's thyroiditis, metastatic infiltrating ductal carcinoma of breast, esophagus adenocarcinoma, thymoma, phyllodes tumor, rectum adenocarcinoma, osteosarcoma, colon adenocarcinoma, thyroid gland papillary carcinoma, leiomyoma, and stomach adenocarcinoma.

Breast Infiltrating Ductal Carcinoma:

It has been previously shown that the expression of PARP1 in infiltrating ductal carcinoma (IDC) of the breast is elevated compared to normals. See Example 2 and FIG. 5 herein and U.S. application Ser. No. 11/818,210. For example, in more than two-thirds of IDC cases, PARP1 expression was above the 95% upper confidence limit of the control non-diseased matched normal population of specimens (“over expression). Estrogen receptor (ER)-negative and Her2-neu-negative subgroups of IDC had an incidence of PARP1 over-expression in approximately 90% of tumors.

In addition, breast cancer subjects also depict elevated levels of co-regulated genes, including IGF1-receptor, IGF-1 and EGFR. Other co-regulated expressed genes that are upregulated at least two-fold as compared to controls include CEACAM6, CTSD, DHTKD1, DNAJC1, FADS2, GLUL, HSPB1, HMGB3, G1P2, IFI27, KPNA2, MMP9, MCM4, MALAT1, MUC1, MX1, NAT1, NUCKS, NUSAP1, OLR1, PSENEN, RAB31, SPP1, SORD, SQLE, TSPAN13, TSTA3, TPD52 and UBE2S.

Thus, in one aspect, IDC breast cancer patients are treated with a combination of PARP modulators and modulators of other co-regulated genes, including IFG1-receptor, IGF-1, EGFR, CEACAM6, CTSD, DHTKD1, DNAJC1, FADS2, GLUL, HSPB1, HMGB3, G1P2, IFI27, KPNA2, MMP9, MCM4, MALAT1, MUC1, MX1, NAT1, NUCKS, NUSAP1, OLR1, PSENEN, RAB31, SPP1, SORD, SQLE, TSPAN13, TSTA3, TPD52 and UBE2S. The combination therapy includes at least one PARP inhibitor. In addition, the combination therapy includes at least one modulator of a co-regulated gene. In one embodiment, PARP expression and ER and/or progesterone receptor (PR) and/or Her2-neu status is evaluated, prior to administration of a combination therapy of PARP inhibitor and modulators of co-regulated genes. In one embodiment, the combination therapy is used to treat estrogen receptor-negative and Her2-neu-negative subgroups of IDC. In another embodiment, the combination therapy is used to treat cancers that do not qualify for anti-hormone (e.g. anti-estrogen or anti-progesterone) or anti-Her2-neu therapies. In yet another embodiment, the combination therapy is used to treat triple negative breast cancers, such as triple negative infiltrating ductal carcinomas.

Infiltrating Breast Lobular Carcinoma

Infiltrating lobular breast carcinoma subjects depict elevated levels of PARP expression, and co-regulated expressed genes including genes of the IGF1-receptor pathway, including IGF1, IGF2 and EGFR. Other co-regulated expressed genes that are upregulated at least two-fold as compared to controls include BGN, BASP1, CAP2, DDX39, KHSRP, LASS2, MLPH, NUSAP1, OLR1, GART, PYGB, PPP2R4, RAB31, SEMA3F, SFI1, SH3GLB2, SORD, TRPS1, B4GALT2 and vav3 oncogene.

Thus, in one aspect, infiltrating lobular breast cancer patients are treated with a combination of PARP modulators and modulators of other co-regulated genes, including IFG1-receptor, IGF1, IGF2, EGFR, BGN, BASP1, CAP2, DDX39, KHSRP, LASS2, MLPH, NUSAP1, OLR1, GART, PYGB, PPP2R4, RAB31, SEMA3F, SFI1, SH3GLB2, SORD, TRPS1, B4GALT2 and vav3 oncogene. The combination therapy includes at least one PARP inhibitor. In addition, the combination therapy includes at least one modulator of a co-regulated gene.

Triple Negative Cancers

In one embodiment, triple negative cancers are treated with combination therapy of PARP modulators and modulators of co-regulated genes. The level of PARP and other identified co-regulated genes are evaluated in the triple negative cancer and if an over expression of the identified co-regulated genes is observed, the cancer is treated with a combination of PARP inhibitor and at least one modulator of co-regulated expressed genes. “Triple negative” breast cancer, means the tumors lack receptors for the hormones estrogen (ER-negative) and progesterone (PR-negative), and for the protein HER2. This makes them resistant to several powerful cancer-fighting drugs like tamoxifen, aromatase inhibitors, and Herceptin. Surgery and chemotherapy are standard treatment options for most forms of triple-negative cancer. In one embodiment, the standard of care for triple negative cancers is combined with the combination therapy of PARP modulators and modulators of co-regulated genes to treat these cancers.

Ovarian Adenocarcinoma

Ovarian adenocarcinoma subjects depict elevated levels of PARP expression, and co-regulated genes of the IGF1-receptor pathway, such as IGF1, IGF2 and EGFR. Other co-regulated genes that are upregulated at least two-fold as compared to controls include ACLSL1, ACSL3, AK3L1, ARFGEF1, ADM, AOF1, ALOX5, ATP5G3, ATP5J2, ATP2A2, ATP11A, ATP6V0B, AKIIP, BCL2L1, BACE2, NSE2, CELSR2, CHST6, CPD, CPT1B, CTSB, CD44, CD47, CD58, CD74, CD9, CDS1, CXCR4, CKLFSF4, CKLFSF6, CSPG2, CRR9, MYCBP, CNDP2, CXADR, CTPS, CXXC5, DDX39, DDAH1, DDR1, DNAJB11, DNAJC10, DNAJD1, DUSP24, DUSP6, ENPP4, ETNK1, ETV6, F11R, FABP5, GPR56, GSPT1, GCNT1, GPI, GCLM, GFPT1, GPX1, HSPA4, HDGF, IDE, IRAK1, IDH2, ICMT, LDHA, LAP3, LTB4DH, MIF, MAD2L1, MGAT4B, MMP9, MCM4, MTHFD2, METTL2, MAPK13, MAP2K3, MAP2K6, MUC1, NQO1, NDFIP2, NET1, NEK6, PANK1, PON2, PCTK1, PDAP1, PPIF, PFKP, PGM2L1, PGD, PGK1, PLA2G4A, PLCB1, PSAT1, PKP4, P4HB, PTGS1, PSMD14, PSMB3, PPP1CA, PDXK, PP, PKM2, RAB10, RAB11FIP1, RAB3IP, RACGAP1, RANBP1, RAN, RGS19IP1, RDH10, SRPK1, SORD, SAT, SGPL1, SGPP2, ST6GAL1, SRD5A2L, SDC4, STX18, TSPAN13, TYMS, TPI1, TNFAIP2, YWHAB, YWHAZ, UBE2S, B3GNT1, GALNT4, GALNT7, VEGF, VAV3, ERBB3, VDAC1 or LYN.

Thus, in one aspect, ovarian adenocarcinoma cancer patients are treated with a combination of PARP modulators and modulators of other co-regulated genes, including IFG1-receptor, IGF1, IGF2, EGFR, ACLSL1, ACSL3, AK3L1, ARFGEF1, ADM, AOF1, ALOX5, ATP5G3, ATP5J2, ATP2A2, ATP11A, ATP6V0B, AKIIP, BCL2L1, BACE2, NSE2, CELSR2, CHST6, CPD, CPT1B, CTSB, CD44, CD47, CD58, CD74, CD9, CDS1, CXCR4, CKLFSF4, CKLFSF6, CSPG2, CRR9, MYCBP, CNDP2, CXADR, CTPS, CXXC5, DDX39, DDAH1, DDR1, DNAJB11, DNAJC10, DNAJD1, DUSP24, DUSP6, ENPP4, ETNK1, ETV6, F11R, FABP5, GPR56, GSPT1, GCNT1, GPI, GCLM, GFPT1, GPX1, HSPA4, HDGF, IDE, IRAK1, IDH2, ICMT, LDHA, LAP3, LTB4DH, MIF, MAD2L1, MGAT4B, MMP9, MCM4, MTHFD2, METTL2, MAPK13, MAP2K3, MAP2K6, MUC1, NQO1, NDFIP2, NET1, NEK6, PANK1, PON2, PCTK1, PDAP1, PPIF, PFKP, PGM2L1, PGD, PGK1, PLA2G4A, PLCB1, PSAT1, PKP4, P4HB, PTGS1, PSMD14, PSMB3, PPP1CA, PDXK, PP, PKM2, RAB10, RAB11FIP1, RAB3IP, RACGAP1, RANBP1, RAN, RGS19IP1, RDH10, SRPK1, SORD, SAT, SGPL1, SGPP2, ST6GAL1, SRD5A2L, SDC4, STX18, TSPAN13, TYMS, TPI1, TNFAIP2, YWHAB, YWHAZ, UBE2S, B3GNT1, GALNT4, GALNT7, VEGF, VAV3, ERBB3, VDAC1 or LYN. The combination therapy includes at least one PARP inhibitor. In addition, the combination therapy includes at least one modulator of a co-regulated gene.

Endometrium Mullerian Mixed Tumor

Endometrium mullerian mixed tumor subjects depict elevated levels of PARP expression, and co-regulated genes that are upregulated at least two-fold as compared to controls, including ATF5, ADRM1, ALDH18A1 AKR1B1, BACH, CKS1B, CSH2, CRR9CXXC5, DNAJA1, ENO1, EME1, FBXO45, FTL, FTLL1, GGH, GPI, GMPS, ILF2, MAD2L1, MCM4, MAGED1, MAP4K4, MSH2, MARCKS, NRAS, NNT, NY-REN-41, PNK1, PRCC, PCTK1, PGD, PGK1, PLD3, PLOD1, PSMD3, PSMD4, PSMD8, PSMA7, PPP3CA, PDXK, RACGAP1, RAN, RFC4, RHOBTB3, RNASEH2A, ROBO1, SRM, SART2, SCAP2, TYMS, TRIP13, UBAP2L, UBE2V1, UBE2S, GALNT2 OR VDAC1.

Thus, in yet another aspect, endometrium mullerian mixed tumor patients are treated with a combination of PARP modulators and modulators of other co-regulated genes, including ATF5, ADRM1, ALDH18A1 AKR1B1, BACH, CKS1B, CSH2, CRR9CXXC5, DNAJA1, ENO1, EME1, FBXO45, FTL, FTLL1, GGH, GPI, GMPS, ILF2, MAD2L1, MCM4, MAGED1, MAP4K4, MSH2, MARCKS, NRAS, NNT, NY-REN-41, PNK1, PRCC, PCTK1, PGD, PGK1, PLD3, PLOD1, PSMD3, PSMD4, PSMD8, PSMA7, PPP3CA, PDXK, RACGAP1, RAN, RFC4, RHOBTB3, RNASEH2A, ROBO1, SRM, SART2, SCAP2, TYMS, TRIP13, UBAP2L, UBE2V1, UBE2S, GALNT2 OR VDAC1.

Testis Seminoma

Testis seminoma subjects depict elevated levels of PARP expression, and co-regulated genes that are upregulated at least two-fold as compared to controls, including ARL5, ALPL, APG5L, RNPEP, ATP11C, ABCD4, CACNB3, CD109, CDC14B, CXXC6, ELOVL6, GRB10, HSPCB, INPP5F, KLF4, MOBKL1A, MSH2, PLOD1, PTPN12, ST6GALNAC2, SDC2, TIAM1, TSPAN13 or ERBB3.

Thus, in yet another aspect, testis seminoma patients are treated with a combination of PARP modulators and modulators of other co-regulated genes, including ARL5, ALPL, APG5L, RNPEP, ATP11C, ABCD4, CACNB3, CD109, CDC14B, CXXC6, ELOVL6, GRB10, HSPCB, INPP5F, KLF4, MOBKL1A, MSH2, PLOD1, PTPN12, ST6GALNAC2, SDC2, TIAM1, TSPAN13 or ERBB3.

Lung Squamous Cell Carcinoma

Lung squamous cell carcinoma subjects depict elevated levels of PARP expression, and co-regulated genes that are upregulated at least two-fold as compared to controls, including PTS, AK3L2, AKR1C1, AKR1C2, AKR1C3, ATP2A2, ABCC1, ABCC5 CSNK2A1, CKS1B, CDW92, CMKOR1, CSPG2, CDK4, DVL3, DUSP24, ELOVL6, GGH, GPI, GCLC, GSR, GMPS, HSPB1, HSPD1, HPRT1, HIG2, IGFBP3, IDH2, MIF, ME1, MMP9, MCM4, MAP3K13, NQO1, ODC1, PPIF, PFKP, PGD, PAICS, PSAT1, PNPT1, PLOD2, PCNA, PSMD2, PRKDC, PTK9, PDK1, PKM2, RAB10, RACGAP1, RAN, RAP2B, RFC4, AHCY, SPP1, SERPINE2, SORD, SMS, SRD5A1, SULF2, TXN, TXNRD1, TXNL5, TYMS, TBL1XR1, TPI1, UBE2S.

Thus, in yet another aspect, lung squamous cell carcinoma patients are treated with a combination of PARP modulators and modulators of other co-regulated genes, including PTS, AK3L2, AKR1C1, AKR1C2, AKR1C3, ATP2A2, ABCC1, ABCC5 CSNK2A1, CKS1B, CDW92, CMKOR1, CSPG2, CDK4, DVL3, DUSP24, ELOVL6, GGH, GPI, GCLC, GSR, GMPS, HSPB1, HSPD1, HPRT1, HIG2, IGFBP3, IDH2, MIF, ME1, MMP9, MCM4, MAP3K13, NQO1, ODC1, PPIF, PFKP, PGD, PAICS, PSAT1, PNPT1, PLOD2, PCNA, PSMD2, PRKDC, PTK9, PDK1, PKM2, RAB10, RACGAP1, RAN, RAP2B, RFC4, AHCY, SPP1, SERPINE2, SORD, SMS, SRD5A1, SULF2, TXN, TXNRD1, TXNL5, TYMS, TBL1XR1, TPI1, UBE2S.

Lung Adenocarcinoma

Lung adenocarcinoma subjects depict elevated levels of PARP expression, and co-regulated genes that are upregulated at least two-fold as compared to controls, including ALDH18A1, AKR1C1, AKR1C2, AKR1C3, ATP2A2, ATP1B1, CPE, CD24, CKS1B, FA2H, GCLC, GFPT1, IGFBP3, IDH2, KMO, LGR4, MIF, MCM4, MTHFD2, NQO1, ODC1, PFKP, PLA2G4A, PAICS, PSAT1, PLOD2, PDIA4, PDIA6, PDK1, SRD5A2L, SRD5A1, TYMS, UBE2S, UGDH, GALNT7 or UNC5CL.

Thus, in yet another aspect, lung adenocarcinoma patients are treated with a combination of PARP modulators and modulators of other co-regulated genes, including ALDH18A1, AKR1C1, AKR1C2, AKR1C3, ATP2A2, ATP1B1, CPE, CD24, CKS1B, FA2H, GCLC, GFPT1, IGFBP3, IDH2, KMO, LGR4, MIF, MCM4, MTHFD2, NQO1, ODC1, PFKP, PLA2G4A, PAICS, PSAT1, PLOD2, PDIA4, PDIA6, PDK1, SRD5A2L, SRD5A1, TYMS, UBE2S, UGDH, GALNT7 or UNC5CL.

Lung Large Cell Carcinoma

Lung large cell carcinoma subjects depict elevated levels of PARP expression, and co-regulated genes that are upregulated at least two-fold as compared to controls, including PTS, ATF7IP, AK3L1, AK3L2, ALDH18A1, ATP2A2, DNAJC9, GPR89, HSPD1, HYOU1, LDHA, MIF, MMP9, MBTPS2, MALAT1, MTHFD2, NRAS, PCTK1, PPIF, PFKP, PAICS, PLOD2, PSMB4, PDK1, PKM2, RACGAP1, RANBP1, RAN, RFC5, SRPK1, SRD5A1, TPI1, or UBE2S.

Thus, in yet another aspect, lung large cell carcinoma patients are treated with a combination of PARP modulators and modulators of other co-regulated genes, including PTS, ATF7IP, AK3L1, AK3L2, ALDH18A1, ATP2A2, DNAJC9, GPR89, HSPD1, HYOU1, LDHA, MIF, MMP9, MBTPS2, MALAT1, MTHFD2, NRAS, PCTK1, PPIF, PFKP, PAICS, PLOD2, PSMB4, PDK1, PKM2, RACGAP1, RANBP1, RAN, RFC5, SRPK1, SRD5A1, TPI1, or UBE2S.

Lymph Node Non-Hodgkin's Lymphoma

Lymph node Non-Hodgkin's Lymphoma subjects depict elevated levels of PARP expression, and co-regulated genes that are upregulated at least two-fold as compared to controls, including ANP32E, BCAT1, CD83, CGI-90, CSK, ARPP-19, DDX21, DCK, DHFR, DAAM1, DUSP10, GRHPR, GGA2, GCHFR, HSPA4, HS2ST1, HDAC1, HPRT1, KPNA2, MAD2L1, MCM4, MOBK1B, MSH2, NUSAP1, ODC1, PFTK1, PLCG2, PRPSAP2, PMS2L3, PCNA, PTPN18, RACGAP1, RNGTT, SNRPD1, SMS, SGPP1, SCD4, SWAP70, SS18, TA-KRP, TYMS, TMPO, TFRC, TNFSF9, UBE2S or LYN.

Thus, in yet another aspect, lymph node Non-Hodgkin's Lymphoma patients are treated with a combination of PARP modulators and modulators of other co-regulated genes, including ANP32E, BCAT1, CD83, CGI-90, CSK, ARPP-19, DDX21, DCK, DHFR, DAAM1, DUSP10, GRHPR, GGA2, GCHFR, HSPA4, HS2ST1, HDAC1, HPRT1, KPNA2, MAD2L1, MCM4, MOBK1B, MSH2, NUSAP1, ODC1, PFTK1, PLCG2, PRPSAP2, PMS2L3, PCNA, PTPN18, RACGAP1, RNGTT, SNRPD1, SMS, SGPP1, SCD4, SWAP70, SS18, TA-KRP, TYMS, TMPO, TFRC, TNFSF9, UBE2S or LYN.

Lymph Node Non-Hodgkin's Lymphoma Diffuse Large B-Cell Type

Lymph node Non-Hodgkin's Lymphoma diffuse large B-cell type subjects depict elevated levels of PARP expression, and co-regulated expressed genes that are upregulated at least two-fold as compared to controls, including BPNT1, ATIC, ATF5, ACADM, ACY1L2, BCL6, BAG2, BCAT1, CFLAR, CD83, CKS1B, CDC5L, CPSF3, CPSF5, CPSF6, C1QBP, PCIA1, CSK, ARPP-19, CDK4, DHFR, DLAT, DNAJD1, DUSP10, ENO1, GSPT1, GMNN, GPI, GRHPR, GTPBP4, GCHFR, HSPH1, HSPE1, HSPD1, HSPA4, HSPCA, HSPCB, HS2ST1, HDAC1, HRMT1L2, HPRT1, HIG2, INSIG1, LDHA, MAD2L1, MADP-1, MAK3, MDH1, MDH2, ME2, MCTS1, MKNK2, MCM4, METAP2, MTHFD2, MOBK1B, MSH2, NEK6, NME1, NUSAP1, NY-REN-41, ODC1, PFKP, PGK1, PLCG2, PRPSAP2, PAICS, PAFAH1B1, PCNA, PSMA2, PKIG, PRKD3, PRKDC, PTPN18, PKM2, RACGAP1, RAN, RRAS2, RFC3, RFC4, RBBP7, RBBP8, AHCY, SSBP1, SMC4L1, SMS, SGPP1, SCAP2, SWAP70, SMARCC1, SS18, TXNL2, TYMS, TOX, TRIP13, TBL1XR1, TFRC, TKT, TPI1, TNFSF9, YWHAE, UCHL5, USP28, UBE2A, UBE2D2, UBE2G1, UBE2S, UTP14A, TALA, LYN.

Thus, another aspect, lymph node Non-Hodgkin's Lymphoma diffuse large B-cell type patients are treated with a combination of PARP modulators and modulators of other co-regulated genes, including BPNT1, ATIC, ATF5, ACADM, ACY1L2, BCL6, BAG2, BCAT1, CFLAR, CD83, CKS1B, CDC5L, CPSF3, CPSF5, CPSF6, C1QBP, PCIA1, CSK, ARPP-19, CDK4, DHFR, DLAT, DNAJD1, DUSP10, ENO1, GSPT1, GMNN, GPI, GRHPR, GTPBP4, GCHFR, HSPH1, HSPE1, HSPD1, HSPA4, HSPCA, HSPCB, HS2ST1, HDAC1, HRMT1L2, HPRT1, HIG2, INSIG1, LDHA, MAD2L1, MADP-1, MAK3, MDH1, MDH2, ME2, MCTS1, MKNK2, MCM4, METAP2, MTHFD2, MOBK1B, MSH2, NEK6, NME1, NUSAP1, NY-REN-41, ODC1, PFKP, PGK1, PLCG2, PRPSAP2, PAICS, PAFAH1B1, PCNA, PSMA2, PKIG, PRKD3, PRKDC, PTPN18, PKM2, RACGAP1, RAN, RRAS2, RFC3, RFC4, RBBP7, RBBP8, AHCY, SSBP1, SMC4L1, SMS, SGPP1, SCAP2, SWAP70, SMARCC1, SS18, TXNL2, TYMS, TOX, TRIP13, TBL1XR1, TFRC, TKT, TPI1, TNFSF9, YWHAE, UCHL5, USP28, UBE2A, UBE2D2, UBE2G1, UBE2S, UTP14A, TALA, LYN.

Liver Hepatocellular Carcinoma

Liver hepatocellular carcinoma subjects depict elevated levels of PARP expression, and co-regulated genes that are upregulated at least two-fold as compared to controls, including AGPAT5, ACSL3, ALDOA, ASPH, ATP1A1, CPD, FZD6, GBAS, HTATIP2, IRAK1, KMO, LPGAT1, MMP9, MCM4, ODC1, PTGFRN, RACGAP1, ROBO1, SPP1, SHC1, TSPAN13, TXNRD1, TKT or UBE2S.

Thus, in one aspect, liver hepatocellular carcinoma patients are treated with a combination of PARP modulators and modulators of other co-regulated genes, including AGPAT5, ACSL3, ALDOA, ASPH, ATP1A1, CPD, FZD6, GBAS, HTATIP2, IRAK1, KMO, LPGAT1, MMP9, MCM4, ODC1, PTGFRN, RACGAP1, ROBO1, SPP1, SHC1, TSPAN13, TXNRD1, TKT or UBE2S.

Thyroid Gland Papillary Carcinoma Follicular Variant

Thyroid gland papillary carcinoma follicular variant subjects also depict elevated levels of PARP expression, and co-regulated genes that are upregulated at least two-fold as compared to controls, including CAMK2D, CTSB, DUSP6, EPS8, FAS, MGAT4B, WIG1, PERP, PLD3, RAB14, SSR3, ST3GAL5 or TPP1.

Thus, in yet another aspect, thyroid gland papillary carcinoma follicular variant patients are treated with a combination of PARP modulators and modulators of other co-regulated genes, including CAMK2D, CTSB, DUSP6, EPS8, FAS, MGAT4B, WIG1, PERP, PLD3, RAB14, SSR3, ST3GAL5 or TPP1. Skin Malignant Melanoma

Skin malignant melanoma subjects depict elevated levels of PARP expression, and co-regulated genes that are upregulated at least two-fold as compared to controls, including EME1, FBXO7, GPR89, GANAB, HSPD1, HSPA8, HPS5, LDHB, MAD2L1, MLPH, NBS1, NEK6, NME1, NUSAP1, PAICS, PSMA5, RFC3, AHCY, SMC4L1, SAT, TYMS, TKT or TRA1 .

Thus, in yet another aspect, skin malignant melanoma patients are treated with a combination of PARP modulators and modulators of other co-regulated genes, including EME1, FBXO7, GPR89, GANAB, HSPD1, HSPA8, HPS5, LDHB, MAD2L1, MLPH, NBS1, NEK6, NME1, NUSAP1, PAICS, PSMA5, RFC3, AHCY, SMC4L1, SAT, TYMS, TKT or TRA1.

Skin Basal Cell Carcinoma

Skin basal cell carcinoma subjects depict elevated levels of PARP expression, and co-regulated genes that are upregulated at least two-fold as compared to controls, including ACY1 L2, CHSY1, CDC42EP4, CCAR1, CSPG2, CXADR, CXXC6, CDK6, DDIT4, GPR56, HSPCA, HSPCAL3, HS2ST1, IGSF4, KTN1, KMO, MARCKS, NNT, PHCA, PAFAH1B1, FLJ23091, RFC3, RBBP4, SORL1, YWHAE, USP47 or UBE2S.

Thus, in yet another aspect, skin basal cell carcinoma patients are treated with a combination of PARP modulators and modulators of other co-regulated genes, including ACY1L2, CHSY1, CDC42EP4, CCAR1, CSPG2, CXADR, CXXC6, CDK6, DDIT4, GPR56, HSPCA, HSPCAL3, HS2ST1, IGSF4, KTN1, KMO, MARCKS, NNT, PHCA, PAFAH1B1, FLJ23091, RFC3, RBBP4, SORL1, YWHAE, USP47 or UBE2S.

Examples of Inflammation

Examples of inflammation include, but are not limited to, systemic inflammatory conditions and conditions associated locally with migration and attraction of monocytes, leukocytes and/or neutrophils. Inflammation may result from infection with pathogenic organisms (including gram-positive bacteria, gram-negative bacteria, viruses, fungi, and parasites such as protozoa and helminths), transplant rejection (including rejection of solid organs such as kidney, liver, heart, lung or cornea, as well as rejection of bone marrow transplants including graft-versus-host disease (GVHD)), or from localized chronic or acute autoimmune or allergic reactions. Autoimmune diseases include acute glomerulonephritis; rheumatoid or reactive arthritis; chronic glomerulonephritis; inflammatory bowel diseases such as Crohn's disease, ulcerative colitis and necrotizing enterocolitis; granulocyte transfusion associated syndromes; inflammatory dermatoses such as contact dermatitis, atopic dermatitis, psoriasis; systemic lupus erythematosus (SLE), autoimmune thyroiditis, multiple sclerosis, and some forms of diabetes, or any other autoimmune state where attack by the subject's own immune system results in pathologic tissue destruction. Allergic reactions include allergic asthma, chronic bronchitis, acute and delayed hypersensitivity. Systemic inflammatory disease states include inflammation associated with trauma, burns, reperfusion following ischemic events (e.g. thrombotic events in heart, brain, intestines or peripheral vasculature, including myocardial infarction and stroke), sepsis, ARDS or multiple organ dysfunction syndrome. Inflammatory cell recruitment also occurs in atherosclerotic plaques.

In one embodiment, provided herein is a method of treating inflammation with modulators of PARP and modulators of other co-regulated genes of inflammation. Inflammation includes, but is not limited to, Non-Hodgkin's lymphoma, Wegener's granulomatosis, Hashimoto's thyroiditis, hepatocellular carcinoma, thymus atrophy, chronic pancreatitis, rheumatoid arthritis, reactive lymphoid hyperplasia, osteoarthritis, ulcerative colitis, papillary carcinoma, Crohn's disease, ulcerative colitis, acute cholecystitis, chronic cholecystitis, cirrhosis, chronic sialadenitis, peritonitis, acute pancreatitis, chronic pancreatitis, chronic Gastritis, adenomyosis, endometriosis, acute cervicitis, chronic cervicitis, lymphoid hyperplasia, multiple sclerosis, hypertrophy secondary to idiopathic thrombocytopenic purpura, primary IgA nephropathy, systemic lupus erythematosus, psoriasis, pulmonary emphysema, chronic pyelonephritis, and chronic cystitis.

Examples of Endocrine and Neuroendocrine Disorders

Examples of endocrine disorders include disorders of adrenal, breast, gonads, pancreas, parathyroid, pituitary, thyroid, dwarfism etc. The adrenal disorders include, but are not limited to, Addison's disease, hirutism, cancer, multiple endocrine neoplasia, congenital adrenal hyperplasia, and pheochromocytoma. The breast disorders include, but are not limited to, breast cancer, fibrocystic breast disease, and gynecomastia. The gonad disorders include, but are not limited to, congenital adrenal hyperplasia, polycystic ovarian syndrome, and turner syndrome. The pancreas disorders include, but are not limited to, diabetes (type I and type II), hypoglycemia, and insulin resistance. The parathyroid disorders include, but are not limited to, hyperparathyroidism, and hypoparathyroidism. The pituitary disorders include, but are not limited to, acromegaly, Cushing's syndrome, diabetes insipidus, empty sella syndrome, hypopituitarism, and prolactinoma. The thyroid disorders include, but are not limited to, cancer, goiter, hyperthyroid, hypothyroid, nodules, thyroiditis, and Wilson's syndrome. The examples of neuroendocrine disorders include, but are not limited to, depression and anxiety disorders related to a hormonal imbalance, catamenial epilepsy, menopause, menstrual migraine, reproductive endocrine disorders, gastrointestinal disorders such as, gut endocrine tumors including carcinoid, gastrinoma, and somatostatinoma, achalasia, and Hirschsprung's disease. In some embodiments, the endocrine and neuroendocrine disorders include nodular hyperplasia, Hashimoto's thyroiditis, islet cell tumor, and papillary carcinoma.

The endocrine and neuroendocrine disorders in children include endocrinologic conditions of growth disorder and diabetes insipidus. Growth delay may be observed with congenital ectopic location or aplasia/hypoplasia of the pituitary gland, as in holoprosencephaly, septo-optic dysplasia and basal encephalocele. Acquired conditions, such as craniopharyngioma, optic/hypothalamic glioma may be present with clinical short stature and diencephalic syndrome. Precocious puberty and growth excess may be seen in the following conditions: arachnoid cyst, hydrocephalus, hypothalamic hamartoma and germinoma. Hypersecretion of growth hormone and adrenocorticotropic hormone by a pituitary adenoma may result in pathologically tall stature and truncal obesity in children. Diabetes insipidus may occur secondary to infiltrative processes such as Langerhans cell of histiocytosis, tuberculosis, germinoma, post traumatic/surgical injury of the pituitary stalk and hypoxic ischemic encephalopathy.

In one embodiment, provided herein is a method of treating endocrine and neuroendocrine disorders with modulators of PARP and modulators of other co-regulated genes of endocrine and neuroendocrine disorders.

Examples of Nutritional and Metabolic Disorders

The examples of nutritional and metabolic disorders include, but are not limited to, aspartylglusomarinuria, biotinidase deficiency, carbohydrate deficient glycoprotein syndrome (CDGS), Crigler-Najjar syndrome, cystinosis, diabetes insipidus, fabry, fatty acid metabolism disorders, galactosemia, gaucher, glucose-6-phosphate dehydrogenase (G6PD), glutaric aciduria, hurler, hurler-scheie, hunter, hypophosphatemia, I-cell, krabbe, lactic acidosis, long chain 3 hydroxyacyl CoA dehydrogenase deficiency (LCHAD), lysosomal storage diseases, mannosidosis, maple syrup urine, maroteaux-lamy, metachromatic leukodystrophy, mitochondrial, morquio, mucopolysaccharidosis, neuro-metabolic, niemann-pick, organic acidemias, purine, phenylketonuria (PKU), pompe, pseudo-hurler, pyruvate dehydrogenase deficiency, sandhoff, sanfilippo, scheie, sly, tay-sachs, trimethylaminuria (fish-malodor syndrome), urea cycle conditions, vitamin D deficiency rickets, metabolic disease of muscle, inherited metabolic disorders, acid-base imbalance, acidosis, alkalosis, alkaptonuria, alpha-mannosidosis, amyloidosis, anemia, iron-deficiency, ascorbic acid deficiency, avitaminosis, beriberi, biotinidase deficiency, deficient glycoprotein syndrome, carnitine disorders, cystinosis, cystinuria, fabry disease, fatty acid oxidation disorders, fucosidosis, galactosemias, gaucher disease, Gilbert disease, glucosephosphate dehydrogenase deficiency, glutaric academia, glycogen storage disease, hartnup disease, hemochromatosis, hemosiderosis, hepatolenticular degeneration, histidinemia, homocystinuria, hyperbilirubinemia, hypercalcemia, hyperinsulinism, hyperkalemia, hyperlipidemia, hyperoxaluria, hypervitaminosis A, hypocalcemia, hypoglycemia, hypokalemia, hyponatremia, hypophosphotasia, insulin resistance, iodine deficiency, iron overload, jaundice, chronic idiopathic, leigh disease, Lesch-Nyhan syndrome, leucine metabolism disorders, lysosomal storage diseases, magnesium deficiency, maple syrup urine disease, MELAS syndrome, menkes kinky hair syndrome, metabolic syndrome X, mucolipidosis, mucopolysacchabridosis, Niemann-Pick disease, obesity, ornithine carbamoyltransferase deficiency disease, osteomalacia, pellagra, peroxisomal disorders, porphyria, erythropoietic, porphyries, progeria, pseudo-gaucher disease, refsum disease, reye syndrome, rickets, sandhoff disease, tangier disease, Tay-sachs disease, tetrahydrobiopterin deficiency, trimethylaminuria (fish odor syndrome), tyrosinemias, urea cycle disorders, water-electrolyte imbalance, wernicke encephalopathy, vitamin A deficiency, vitamin B12 deficiency, vitamin B deficiency, wolman disease, and zellweger syndrome.

In one embodiment, provided herein is a method of treating nutritional or metabolic disorders with modulators of PARP and modulators of other co-regulated genes of nutritional or metabolic disorders. In some embodiments, the metabolic diseases include diabetes and obesity.

Examples of Hematolymphoid System

A hematolymphoid system includes hemic and lymphatic diseases. A “hematological disorder” includes a disease, disorder, or condition which affects a hematopoietic cell or tissue. Hematological disorders include diseases, disorders, or conditions associated with aberrant hematological content or function. Examples of hematological disorders include disorders resulting from bone marrow irradiation or chemotherapy treatments for cancer, disorders such as pernicious anemia, hemorrhagic anemia, hemolytic anemia, aplastic anemia, sickle cell anemia, sideroblastic anemia, anemia associated with chronic infections such as malaria, trypanosomiasis, HIV, hepatitis virus or other viruses, myelophthisic anemias caused by marrow deficiencies, renal failure resulting from anemia, anemia, polycethemia, infectious mononucleosis (IM), acute non-lymphocytic leukemia (ANLL), acute Myeloid Leukemia (AML), acute promyelocytic leukemia (APL), acute myelomonocytic leukemia (AMMoL), polycethemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalessemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as plasmodium, chemical injuries from, e.g., lead poisoning, and hypersplenism.

Lymphatic diseases include, but are not limited to, lymphadenitis, lymphagiectasis, lymphangitis, lymphedema, lymphocele, lymphoproliferative disorders, mucocutaneous lymph node syndrome, reticuloendotheliosis, splenic diseases, thymus hyperplasia, thymus neoplasms, tuberculosis, lymph node, pseudolymphoma, and lymphatic abnormalities.

In one embodiment, provided herein is a method of treating a hematological disorder with modulators of PARP and modulators of other co-regulated genes of hematological disorders. Disorders of hematolymphoid system include, but are not limited to, non-Hodgkin's lymphoma, chronic lymphocytic leukemia, and reactive lymphoid hyperplasia.

Examples of CNS Diseases

The examples of CNS diseases include, but are not limited to, neurodegenerative diseases, drug abuse such as, cocaine abuse, multiple sclerosis, schizophrenia, acute disseminated encephalomyelitis, transverse myelitis, demyelinating genetic diseases, spinal cord injury, virus-induced demyelination, progressive multifocal leucoencephalopathy, human lymphotrophic T-cell virus I (HTLVI)-associated myelopathy, and nutritional metabolic disorders.

In one embodiment, provided herein is a method of treating CNS diseases with modulators of PARP and modulators of other co-regulated genes of CNS diseases. In some embodiments, the CNS diseases include Parkinson disease, Alzheimer's disease, cocaine abuse, and schizophrenia.

Examples of Neurodegenerative Diseases

Neurodegenerative diseases include, but are not limited to, Alzheimer's disease, Pick's disease, diffuse lewy body disease, progressive supranuclear palsy (Steel-Richardson syndrome), multisystem degeneration (Shy-Drager syndrome), motor neuron diseases including amyotrophic lateral sclerosis, degenerative ataxias, cortical basal degeneration, ALS-Parkinson's-dementia complex of guam, subacute sclerosing panencephalitis, Huntington's disease, Parkinson's disease, synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, and prion diseases (including Creutzfeldt-Jakob, Gerstmann-Straussler-Scheinker disease, kuru and fatal familial insomnia), Alexander disease, alper's disease, amyotrophic lateral sclerosis, ataxia telangiectasia, batten disease, canavan disease, cockayne syndrome, corticobasal degeneration, Creutzfeldt-Jakob disease, Huntington disease, Kennedy's disease, Krabbe disease, lewy body dementia, Machado-Joseph disease, spinocerebellar ataxia type 3, multiple sclerosis, multiple system atrophy, Parkinson disease, Pelizaeus-Merzbacher Disease, Refsum's disease, Schilder's disease, Spielmeyer-Vogt-Sjogren-Batten disease, Steele-Richardson-Olszewski disease, and tabes dorsalis.

In one embodiment, provided herein is a method of treating a veurodegenerative diseases with modulators of PARP and modulators of other co-regulated genes of veurodegenerative diseases.

Examples of Disorders of Urinary Tract

Disorders of urinary tract include, but are not limited to, disorders of kidney, ureters, bladder, and urethra. For example, urethritis, cystitis, pyelonephritis, renal agenesis, hydronephrosis, polycystic kidney disease, multicystic kidneys, low urinary tract obstruction, bladder exstrophy and epispadias, hypospadias, bacteriuria, prostatitis, intrarenal and peripheral abscess, benign prostate hypertrophy, renal cell carcinoma, transitional cell carcinoma, Wilm's tumor, uremia, and glomerolonephritis.

In one embodiment, provided herein is a method of treating disorders of urinary tract with modulators of PARP and modulators of other co-regulated genes of disorders of urinary tract.

Examples of Respiratory Diseases

The respiratory diseases and conditions include, but are not limited to, asthma, chronic obstructive pulmonary disease (COPD), adenocarcinoma, adenosquamous carcinoma, squamous cell carcinoma, large cell carcinoma, cystic fibrosis (CF), dispnea, emphysema, wheezing, pulmonary hypertension, pulmonary fibrosis, hyper-responsive airways, increased adenosine or adenosine receptor levels, pulmonary bronchoconstriction, lung inflammation and allergies, and surfactant depletion, chronic bronchitis, bronchoconstriction, difficult breathing, impeded and obstructed lung airways, adenosine test for cardiac function, pulmonary vasoconstriction, impeded respiration, acute respiratory distress syndrome (ARDS), administration of certain drugs, such as adenosine and adenosine level increasing drugs, and other drugs for, e.g. treating supraventricular tachycardia (SVT), and the administration of adenosine stress tests, infantile respiratory distress syndrome (infantile RDS), pain, allergic rhinitis, decreased lung surfactant, decreased ubiquinone levels, or chronic bronchitis, among others.

In one embodiment, provided herein is a method of treating respiratory diseases and conditions with modulators of PARP and modulators of other co-regulated genes of disorders of respiratory diseases and conditions.

Examples of Disorders of Female Reproductive System

The disorders of the female reproductive system include diseases of the vulva, vagina, cervix uteri, corpus uteri, fallopian tube, and ovary. Some of the examples include, adnexal diseases such as, fallopian tube disease, ovarian disease, leiomyoma, mucinous cystadenocarcinoma, serous cystadenocarcinoma, parovarian cyst, and pelvic inflammatory disease; endometriosis; reproductive neoplasms such as, fallopian tube neoplasms, uterine neoplasms, vaginal neoplasms, vulvar neoplasms, and ovarian neoplasms; gynatresia; reproductive herpes; infertility; sexual dysfunction such as, dyspareunia, and impotence; tuberculosis; uterine diseases such as, cervix disease, endometrial hyperplasia, endometritis, hematometra, uterine hemorrhage, uterine neoplasms, uterine prolapse, uterine rupture, and uterine inversion; vaginal diseases such as, dyspareunia, hematocolpos, vaginal fistula, vaginal neoplasms, vaginitis, vaginal discharge, and candidiasis or vulvovaginal; vulvar diseases such as, kraurosis vulvae, pruritus, vulvar neoplasm, vulvitis, and candidiasis; and urogenital diseases such as urogenital abnormalities and urogenital neoplasms.

In one embodiment, provided herein is a method of treating disorders of the female reproductive system with modulators of PARP and modulators of other co-regulated genes of disorders of the female reproductive system.

Examples of Disorders of Male Reproductive System

The disorders of the male reproductive system include, but are not limited to, epididymitis; reproductive neoplasms such as, penile neoplasms, prostatic neoplasms, and testicular neoplasms; hematocele; reproductive herpes; hydrocele; infertility; penile diseases such as, balanitis, hypospadias, peyronie disease, penile neoplasms, phimosis, and priapism; prostatic diseases such as, prostatic hyperplasia, prostatic neoplasms, and prostatitis; organic sexual dysfunction such as, dyspareunia, and impotence; spermatic cord torsion; spermatocele; testicular diseases such as, cryptorchidism, orchitis, and testicular neoplasms; tuberculosis; varicocele; urogenital diseases such as, urogenital abnormalities, and urogenital neoplasms; and fournier gangrene.

In one embodiment, provided herein is a method of treating disorders of the male reproductive system with modulators of PARP and modulators of other co-regulated genes of disorders of the male reproductive system.

Examples of Cardiovascular Disorders (CVS)

The cardiovascular disorders include those disorders that can either cause ischemia or are caused by reperfusion of the heart. Examples include, but are not limited to, atherosclerosis, coronary artery disease, granulomatous myocarditis, chronic myocarditis (non-granulomatous), primary hypertrophic cardiomyopathy, peripheral artery disease (PAD), stroke, angina pectoris, myocardial infarction, cardiovascular tissue damage caused by cardiac arrest, cardiovascular tissue damage caused by cardiac bypass, cardiogenic shock, and related conditions that would be known by those of ordinary skill in the art or which involve dysfunction of or tissue damage to the heart or vasculature, especially, but not limited to, tissue damage related to PARP activation.

In one embodiment, provided herein is a method of treating cardiovascular disorders with modulators of PARP and modulators of other co-regulated genes of cardiovascular disorders. In some embodiments, CVS diseases include, but are not limited to, atherosclerosis, granulomatous myocarditis, myocardial infarction, myocardial fibrosis secondary to valvular heart disease, myocardial fibrosis without infarction, primary hypertrophic cardiomyopathy, and chronic myocarditis (non-granulomatous).

Examples of Viral Disorders

Viral disorders include, but are not limited to, disorders that are caused by viral infection and subsequent replication. Examples of viral disorders include, but are not limited to, infections caused by the following viral agents: human immunodeficiency virus, hepatitis C virus, hepatitis B virus, herpes virus, varicella-zoster, adenovirus, cytomegalovirus, enteroviruses, rhinoviruses, rubella virus, influenza virus and encephalitis viruses. In some embodiments, HIV infection and replication is targeted by the combination therapies described herein. In one embodiment, provided herein is a method of treating viral disorders with modulators of PARP and modulators of other co-regulated genes of viral disorders.

### PARP and Disease Pathways

The poly(ADP-ribose)polymerase (PARP) is also known as poly(ADP-ribose) synthase and poly ADP-ribosyltransferase. PARP catalyzes the formation of poly(ADP-ribose) polymers which can attach to nuclear proteins (as well as to itself) and thereby modify the activities of those proteins. The enzyme plays a role in enhancing DNA repair, but it also plays a role in regulating chromatin in the nuclei (for review see: D. D'amours et al. “Poly(ADP-ribosylation reactions in the regulation of nuclear functions,” Biochem. J. 342: 249-268 (1999)).

PARP-1 comprises an N-terminal DNA binding domain, an automodification domain and a C-terminal catalytic domain; various cellular proteins interact with PARP-1. The N-terminal DNA binding domain contains two zinc finger motifs. Transcription enhancer factor-1 (TEF-1), retinoid X receptor α, DNA polymerase α, X-ray repair cross-complementing factor-1 (XRCC1) and PARP-1 itself interact with PARP-1 in this domain. The automodification domain contains a BRCT motif, one of the protein-protein interaction modules. This motif is originally found in the C-terminus of BRCA1 (breast cancer susceptibility protein 1) and is present in various proteins related to DNA repair, recombination and cell-cycle checkpoint control. POU-homeodomain-containing octamer transcription factor-1 (Oct-1), Yin Yang (YY)1 and ubiquitin-conjugating enzyme 9 (ubc9) could interact with this BRCT motif in PARP-1.

More than 15 members of the PARP family of genes are present in the mammalian genome. PARP family proteins and poly(ADP-ribose) glycohydrolase (PARG), which degrades poly(ADP-ribose) to ADP-ribose, could be involved in a variety of cell regulatory functions including DNA damage response and transcriptional regulation and may be related to carcinogenesis and the biology of cancer in many respects.

Several PARP family proteins have been identified. Tankyrase has been found as an interacting protein of telomere regulatory factor 1 (TRF-1) and is involved in telomere regulation. Vault PARP (VPARP) is a component in the vault complex, which acts as a nuclear-cytoplasmic transporter. PARP-2, PARP-3 and 2,3,7,8-tetrachlorodibenzo-p-dioxin inducible PARP (TiPARP) have also been identified. Therefore, poly(ADP-ribose) metabolism could be related to a variety of cell regulatory functions.

A member of this gene family is PARP-1. The PARP-1 gene product is expressed at high levels in the nuclei of cells and is dependent upon DNA damage for activation. Without being bound by any theory, it is believed that PARP-1 binds to DNA single or double stranded breaks through an amino terminal DNA binding domain. The binding activates the carboxy terminal catalytic domain and results in the formation of polymers of ADP-ribose on target molecules. PARP-1 is itself a target of poly ADP-ribosylation by virtue of a centrally located automodification domain. The ribosylation of PARP-1 causes dissociation of the PARP-1 molecules from the DNA. The entire process of binding, ribosylation, and dissociation occurs very rapidly. It has been suggested that this transient binding of PARP-1 to sites of DNA damage results in the recruitment of DNA repair machinery or may act to suppress the recombination long enough for the recruitment of repair machinery.

The source of ADP-ribose for the PARP reaction is nicotinamide adenosine dinucleotide (NAD). NAD is synthesized in cells from cellular ATP stores and thus high levels of activation of PARP activity can rapidly lead to depletion of cellular energy stores. It has been demonstrated that induction of PARP activity can lead to cell death that is correlated with depletion of cellular NAD and ATP pools. PARP activity is induced in many instances of oxidative stress or during inflammation. For example, during reperfusion of ischemic tissues reactive nitric oxide is generated and nitric oxide results in the generation of additional reactive oxygen species including hydrogen peroxide, peroxynitrate and hydroxyl radical. These latter species can directly damage DNA and the resulting damage induces activation of PARP activity. Frequently, it appears that sufficient activation of PARP activity occurs such that the cellular energy stores are depleted and the cell dies. A similar mechanism is believed to operate during inflammation when endothelial cells and pro-inflammatory cells synthesize nitric oxide which results in oxidative DNA damage in surrounding cells and the subsequent activation of PARP activity. The cell death that results from PARP activation is believed to be a major contributing factor in the extent of tissue damage that results from ischemia-reperfusion injury or from inflammation.

Inhibition of PARP activity can be potentially useful in the treatment of cancer. De-inhibition of the DNAase (by PARP-1 inhibition) may initiate DNA breakdown that is specific for cancer cells and induce apoptosis in cancer cells only. PARP small molecule inhibitors may sensitize treated tumor cell lines to killing by ionizing radiation and by some DNA damaging chemotherapeutic drugs. A monotherapy by PARP inhibitors or a combination therapy with a chemotherapeutic or radiation may be an effective treatment. Combination therapy with a chemotherapeutic can induce tumor regression at concentrations of the chemotherapeutic that are ineffective by themselves. Further, PARP-1 mutant mice and PARP-1 mutant cell lines may be sensitive to radiation and similar types of chemotherapeutic drugs.

The level of PARP and co-regulated gene expression may be indicative of the disease state, stage or prognosis of an individual patient. For example, a relative level of PARP-1 expression in subjects with prostrate cancer and breast cancer is up-regulated as compared to normal subjects. Similarly, a relative level of PARP-1 expression in subjects with ovarian cancer and endometrium cancer is up-regulated as compared to normal subjects. Within different cancers, each cancer type shows up-regulation to a different extent from each other. For example, different breast cancers show up-regulation to different extent. Similarly, different ovarian cancers show up-regulation to a different extent. It indicates that PARP-1 up-regulation is not only helpful in identifying PARP-1 mediated diseases treatable by PARP-1 inhibitors, but it may also be helpful in predicting/determining the efficacy of the treatment with PARP-1 inhibitors depending on the extent of up-regulation of PARP-1 in a subject. Assessment of PARP and co-regulated gene expression, therefore, can be an indicator of tumor sensitivity to PARP-1 inhibitors and co-regulated genes. It may also be helpful in personalizing the dose regimen for a subject.

PARP Related Pathways

As discussed, other genes that are co-regulated along with PARP expression may also be useful in identifying and treating diseases that may be treatable by a combination of PARP and co-regulated gene modulators. For example, a relative level of PARP-1 expression, along with an indicated upregulation of IGF1R and EGFR expression in a tumor tissue sample, as compared to normal subjects, may indicate a cancer that is treatable with a combination of PARP inhibitor and IGF1R and EGFR inhibitors. In addition, a relative level of PARP-1, IGF1R and EGFR expression in subjects with an inflammatory disease, as compared to normal subjects, may indicate an inflammatory disease that is treatable with a combination of PARP inhibitor and IGF1R and EGFR inhibitors.

Co-regulation of other identified genes may be detected independently of the analysis of PARP level expression. For example, a practitioner from the teachings presented herein, would combine a PARP inhibitor with an IGF1R inhibitor in breast cancer tissue because of the demonstrated correlation of co-upregulation with PARP-1 and IGF1R expression. Accordingly, one treatment embodiment includes the administration of co-regulation gene modulators, such as inhibitors to IGF1R and EGFR, independent of the measurement of PARP level expression for the treatment of diseases, including cancer. Such administration of co-regulated gene modulators could occur in tandem with, or separate from, the administration of PARP modulators.

Thus, one embodiment disclosed herein is to demonstrate the interrelationship of various pathways with PARP regulation, to identify potential targets of co-modulation combinatory therapy. The following genetic targets are exemplary, but are not exhaustive, of genes that are co-regulated with PARP expression in disease states.

Insulin-Like Growth Factor Receptor 1

The insulin-like growth factor receptor (IGF1R) is a transmembrane receptor tyrosine kinase that mediates IGF biological activity and signaling through several critical cellular molecular networks including RAS0RAF-ERK and P13-AKT-mTOR pathways. A functional IGF1R is required for transformation, and has been shown to promote tumor cell growth and survival. Several genes that have been shown to promote cell proliferation in response to IGF-1/IGF-2 binding in the IGF1R pathway include Shc, IRS, Grb2, SOS, Ras, Raf, MEK and ERK. Genes that have been implicated in the cell proliferation, motility and survival functions of IGF1 R signaling include IRS, PI3-K, PIP2, PTEN, PTP-2, PDK and Akt.

IGF1R is frequently overexpressed in human tumors, including melanomas, cancers of the colon, pancreas, prostate and kidney. Overexpression of IGF1R may function as an oncogene, where such overexpression of IGF1R can be the result of loss of tumor suppressors, including wild-type p53, BRCA1 and VHL. IGF1R activation protects cells from a variety of apotosis-inducing agents, including osmotic stress, hypoxia and anti-cancer drugs. The level of expression of functional IGF1R appears to be a critical determinant of resistance to apoptosis in vitro and in vivo. IGFs are known to protect tumor cells against killing by cytotoxic drugs. This effect can be attributed to the well-recognized ability of the IGF axis to suppress apoptosis, and also to an apparent ability to influence aspects of the DNA damage response. Consistent with this, sensitivity to chemotherapy may be enhanced by various approaches to block the IGF axis. The IGF axis could potentially be blocked at several different levels, including interference with the expression and function of ligands, binding proteins and receptors. Small molecule inhibitors, antibodies, dominant negative to IGF1 R, antisense and siRNA representative examples of inhibitors that may enhance sensitivity to chemotherapy through the IGF axis.

Experiments were conducted to verify the correlative relationship exists between PARP and IGF-1R expression in a variety of tissue samples. Table XIX depicts the level of expression in a variety of tissues, including adrenal gland, bone, breast tumor tissue, including IDC and infiltrating lobular carcinoma, among others. As seen, upregulation of IGF1-R can be seen in the same tissues as that for PARP1 upregulation, for example in breast, ovarian and skin cancers. Accordingly, an embodiment is the treatment of susceptible cancers with a combination of PARP and IGF1R modulators. Moreover, IGF1R related genes, including genes that are co-regulated along the IGF1R pathway, are also contemplated herein.

TABLE XIX
Expression of IGF1R (Insulin-like growth factor 1 receptor) in human primary tumors
in comparison with normal tissues tested on Array hg133a.

| | Sample | | | |
|---|---|---|---|---|
| Sample Set | Count | Mean | Std. Dev. | Median |
| Adrenal Gland, Adrenal Cortical Carcinoma, Primary | 3 | 65.828 | 35.85 | 75.958 |
| Adrenal Gland, Normal | 13 | 85.341 | 37.713 | 92.31 |
| Bone, Giant Cell Tumor of Bone, Primary | 10 | 57.201 | 25.847 | 45.959 |
| Bone, Normal | 8 | 46.953 | 14.046 | 43.164 |
| Bone, Osteosarcoma, Primary | 4 | 64.269 | 20.188 | 60.848 |
| Breast, Infiltrating Carcinoma of Mixed Ductal and Lobular Type, | 8 | 112.111 | 69.247 | 99 |
| Primary | | | | |
| Breast, Infiltrating Ductal Carcinoma, Primary | 169 | 124.036 | 95.462 | 97.339 |
| Breast, Infiltrating Lobular Carcinoma, Primary | 17 | 114.33 | 66.461 | 99.947 |
| Breast, Intraductal Carcinoma | 3 | 214.121 | 100.275 | 208.348 |
| Breast, Mucinous Carcinoma, Primary | 4 | 163.719 | 127.018 | 146.328 |
| Breast, Normal | 68 | 87.822 | 58.73 | 70.932 |
| Breast, Phyllodes Tumor (Cystosarcoma Phyllodes), Primary | 5 | 99.977 | 33.553 | 117.663 |
| Colon, Adenocarcinoma (Excluding Mucinous Type), Primary | 77 | 47.25 | 24.702 | 41.896 |
| Colon, Adenocarcinoma, Mucinous Type, Primary | 7 | 54.155 | 32.766 | 48.534 |
| Colon, Normal | 180 | 41.474 | 19.577 | 38.744 |
| Endometrium, Adenocarcinoma, Endometrioid Type, Primary | 50 | 77.703 | 34.7 | 70.791 |
| Endometrium, Mullerian Mixed Tumor, Primary | 7 | 103.11 | 112.968 | 58.225 |
| Endometrium, Normal | 23 | 109.476 | 61.449 | 86.356 |
| Esophagus, Adenocarcinoma, Primary | 3 | 76.404 | 89.219 | 33.085 |
| Esophagus, Normal | 22 | 54.934 | 22.855 | 46.997 |
| Kidney, Carcinoma, Chromophobe Type, Primary | 3 | 79.838 | 38.577 | 98.029 |
| Kidney, Normal | 81 | 94.875 | 39.237 | 90.24 |
| Kidney, Renal Cell Carcinoma, Clear Cell Type, Primary | 45 | 69.441 | 44.919 | 57.36 |
| Kidney, Renal Cell Carcinoma, Non-Clear Cell Type, Primary | 15 | 86.186 | 50.4 | 70.631 |
| Kidney, Transitional Cell Carcinoma, Primary | 4 | 41 | 20.564 | 42.229 |
| Kidney, Wilm's Tumor, Primary | 8 | 104.733 | 47.828 | 89.439 |
| Larynx, Normal | 4 | 54.531 | 7.301 | 54.091 |
| Larynx, Squamous Cell Carcinoma, Primary | 4 | 111.113 | 89.014 | 97.039 |
| Liver, Hepatocellular Carcinoma | 16 | 22.266 | 7.512 | 21.544 |
| Liver, Normal | 42 | 27.576 | 25.82 | 22.895 |
| Lung, Adenocarcinoma, Primary | 46 | 65.452 | 47.363 | 55.441 |
| Lung, Adenosquamous Carcinoma, Primary | 3 | 56.079 | 34.038 | 47.214 |
| Lung, Large Cell Carcinoma, Primary | 7 | 61.764 | 46.439 | 31.328 |
| Lung, Neuroendocrine Carcinoma (Non-Small Cell Type), Primary | 3 | 37.427 | 24.31 | 27.517 |
| Lung, Normal | 126 | 57.277 | 29.69 | 52.18 |
| Lung, Small Cell Carcinoma, Primary | 3 | 57.647 | 23.035 | 62.91 |
| Lung, Squamous Cell Carcinoma, Primary | 39 | 81.713 | 50.819 | 66.414 |
| Oral Cavity, Squamous Cell Carcinoma, Primary | 3 | 136.372 | 93.9 | 93.936 |
| Ovary, Adenocarcinoma, Clear Cell Type, Primary | 6 | 93.691 | 43.793 | 75.009 |
| Ovary, Adenocarcinoma, Endometrioid Type, Primary | 22 | 73.115 | 32.45 | 75.949 |
| Ovary, Adenocarcinoma, Papillary Serous Type, Primary | 36 | 126.618 | 261.068 | 75.962 |
| Ovary, Granulosa Cell Tumor, Primary | 3 | 169.841 | 60.705 | 169.927 |
| Ovary, Mucinous Cystadenocarcinoma, Primary | 7 | 75.393 | 66.713 | 50.779 |
| Ovary, Mullerian Mixed Tumor, Primary | 5 | 126.91 | 121.824 | 79.955 |
| Ovary, Normal | 89 | 115.666 | 53.302 | 108.304 |
| Pancreas, Adenocarcinoma, Primary | 23 | 63.885 | 16.923 | 60.04 |
| Pancreas, Islet Cell Tumor, Malignant, Primary | 7 | 56.924 | 63.772 | 30.551 |
| Pancreas, Normal | 46 | 93.076 | 37.674 | 89.188 |
| Prostate, Adenocarcinoma, Primary | 86 | 119.495 | 53.987 | 114.899 |
| Prostate, Normal | 57 | 108.233 | 58.456 | 93.388 |
| Rectum, Adenocarcinoma (Excluding Mucinous Type), Primary | 29 | 59.204 | 19.34 | 65.388 |
| Rectum, Adenocarcinoma, Mucinous Type, Primary | 3 | 62.573 | 31.476 | 57.951 |
| Rectum, Normal | 44 | 50.965 | 19.969 | 48.972 |
| Skin, Basal Cell Carcinoma, Primary | 4 | 179.37 | 85.237 | 202.634 |
| Skin, Malignant Melanoma, Primary | 7 | 87.475 | 42.005 | 86.499 |
| Skin, Normal | 61 | 55.948 | 23.541 | 49.106 |
| Skin, Squamous Cell Carcinoma, Primary | 4 | 66.185 | 17.746 | 69.936 |
| Small Intestine, Gastrointestinal Stromal Tumor (GIST), Primary | 4 | 10.347 | 3.768 | 10.282 |
| Small Intestine, Normal | 97 | 36.769 | 20.176 | 32.341 |
| Stomach, Adenocarcinoma (Excluding Signet Ring Cell Type), | 27 | 44.607 | 29.077 | 37.317 |
| Primary | | | | |
| Stomach, Adenocarcinoma, Signet Ring Cell Type, Primary | 9 | 50.232 | 16.902 | 52.252 |
| Stomach, Gastrointestinal Stromal Tumor (GIST), Primary | 9 | 36.869 | 61.155 | 15.828 |
| Stomach, Normal | 52 | 58.767 | 28.497 | 47.439 |
| Thyroid Gland, Follicular Carcinoma, Primary | 3 | 120.042 | 41.591 | 130.814 |
| Thyroid Gland, Normal | 24 | 81.333 | 49.295 | 71.732 |
| Thyroid Gland, Papillary Carcinoma, Primary; All Variants | 29 | 83.359 | 51.903 | 63.894 |
| Urinary Bladder, Normal | 9 | 62.521 | 20.653 | 55.34 |
| Urinary Bladder, Transitional Cell Carcinoma, Primary | 4 | 64.6 | 12.927 | 59.941 |
| Uterine Cervix, Adenocarcinoma, Primary | 3 | 103.944 | 95.785 | 55.348 |
| Uterine Cervix, Normal | 115 | 71.105 | 24.883 | 66.647 |
| Vulva, Normal | 4 | 63.062 | 21.067 | 69.51 |
| Vulva, Squamous Cell Carcinoma, Primary | 5 | 141.052 | 129.493 | 84.436 |

Insulin-like Growth Factor 2 (IGF2)

As discussed above, overexpression of IGF1R may function as an oncogene, where such overexpression of IGF1R can be the result of loss of tumor suppressors, including wild type p53, BRCA1 and VHL (Werner and Roberts, 2003, Genes, Chromo and Cancer, 36:112-120; Riedemann and Macaulay, 2006, Endocr. Relat. Cancer, 13:S3343). Consistent with the role of IGF1R in the development of cancer, it has been previously shown that blocking of the IGF axis may enhance the sensitivity to chemotherapy. The IGF axis could potentially be blocked at several different levels, including interference with the expression and function of ligands, including IGF2. Thus, the role of IGF ligand inhibitors, such as IGF2, may also play a role in cancer development.

Experiments were thus conducted to determine if a correlative relationship exists between PARP and IGF2 expression in a variety of tissue samples. Table XX depicts the level of expression in a variety of tissues, including adrenal gland, bone, breast tumor tissue, including IDC and infiltrating lobular carcinoma, among others. As seen, upregulation of IGF2 is demonstrated in the same tissues as that for PARP1 upregulation, for example in breast, liver, lung and ovarian cancers. Accordingly, one embodiment is the treatment of susceptible diseases with a combination of PARP and IGF2 modulators. Moreover, IGF2 related genes, including IGF1, IGF3, IGF4, IGF5, IGF6 and other insulin-like growth factor receptor ligands are also contemplated herein.

TABLE XX
Expression of IGF2 (insulin-like growth factor 2) in human primary tumors
in comparison with normal tissues

| | Sample | | |
|---|---|---|---|
| Sample Set | Count | Mean | Std. Dev. |
| Adrenal Gland, Adrenal Cortical Carcinoma, Primary | 3 | 1848.834 | 3090.534 |
| Adrenal Gland, Normal | 13 | 529.291 | 547.211 |
| Bone, Giant Cell Tumor of Bone, Primary | 10 | 92.575 | 46.504 |
| Bone, Normal | 8 | 541.963 | 363.888 |
| Bone, Osteosarcoma, Primary | 4 | 563.184 | 570.075 |
| Breast, Infiltrating Carcinoma of Mixed Ductal and Lobular Type, | 8 | 266.772 | 222.345 |
| Primary | | | |
| Breast, Infiltrating Ductal Carcinoma, Primary | 169 | 302.565 | 404.769 |
| Breast, Infiltrating Lobular Carcinoma, Primary | 17 | 427.307 | 267.766 |
| Breast, Intraductal Carcinoma | 3 | 309.277 | 169.406 |
| Breast, Mucinous Carcinoma, Primary | 4 | 323.68 | 104.134 |
| Breast, Normal | 68 | 625.371 | 391.936 |
| Breast, Phyllodes Tumor (Cystosarcoma Phyllodes), Primary | 5 | 4635.806 | 758.39 |
| Colon, Adenocarcinoma (Excluding Mucinous Type), Primary | 77 | 404.074 | 990.572 |
| Colon, Adenocarcinoma, Mucinous Type, Primary | 7 | 142.852 | 115.826 |
| Colon, Normal | 180 | 124.294 | 164.11 |
| Endometrium, Adenocarcinoma, Endometrioid Type, Primary | 50 | 262.408 | 261.542 |
| Endometrium, Mullerian Mixed Tumor, Primary | 7 | 4298.005 | 3973.436 |
| Endometrium, Normal | 23 | 962.379 | 568.949 |
| Esophagus, Adenocarcinoma, Primary | 3 | 88.334 | 23.213 |
| Esophagus, Normal | 22 | 147.307 | 93.47 |
| Kidney, Carcinoma, Chromophobe Type, Primary | 3 | 98.284 | 49.051 |
| Kidney, Normal | 81 | 180.318 | 173.522 |
| Kidney, Renal Cell Carcinoma, Clear Cell Type, Primary | 45 | 172.314 | 293.9 |
| Kidney, Renal Cell Carcinoma, Non-Clear Cell Type, Primary | 15 | 81.293 | 74.054 |
| Kidney, Transitional Cell Carcinoma, Primary | 4 | 5620.705 | 4310.083 |
| Kidney, Wilm's Tumor, Primary | 8 | 5461.075 | 2837.742 |
| Larynx, Normal | 4 | 501.856 | 381.37 |
| Larynx, Squamous Cell Carcinoma, Primary | 4 | 309.574 | 200.901 |
| Liver, Hepatocellular Carcinoma | 16 | 1912.226 | 3539.841 |
| Liver, Normal | 42 | 1505.288 | 632.644 |
| Lung, Adenocarcinoma, Primary | 46 | 81.16 | 86.841 |
| Lung, Adenosquamous Carcinoma, Primary | 3 | 202.216 | 248.096 |
| Lung, Large Cell Carcinoma, Primary | 7 | 1233.22 | 1890.947 |
| Lung, Neuroendocrine Carcinoma (Non-Small Cell Type), Primary | 3 | 22.408 | 8.574 |
| Lung, Normal | 126 | 116.73 | 221.406 |
| Lung, Small Cell Carcinoma, Primary | 3 | 307.962 | 315.514 |
| Lung, Squamous Cell Carcinoma, Primary | 39 | 81.715 | 74.222 |
| Oral Cavity, Squamous Cell Carcinoma, Primary | 3 | 341.49 | 278.662 |
| Ovary, Adenocarcinoma, Clear Cell Type, Primary | 6 | 211.816 | 243.491 |
| Ovary, Adenocarcinoma, Endometrioid Type, Primary | 22 | 229.471 | 416.059 |
| Ovary, Adenocarcinoma, Papillary Serous Type, Primary | 36 | 1154.231 | 1834.815 |
| Ovary, Granulosa Cell Tumor, Primary | 3 | 77.318 | 59.672 |
| Ovary, Mucinous Cystadenocarcinoma, Primary | 7 | 97.436 | 32.315 |
| Ovary, Mullerian Mixed Tumor, Primary | 5 | 2463.327 | 3493.894 |
| Ovary, Normal | 89 | 416.275 | 283.767 |
| Pancreas, Adenocarcinoma, Primary | 23 | 917.465 | 3230.5 |
| Pancreas, Islet Cell Tumor, Malignant, Primary | 7 | 1209.737 | 2927.581 |
| Pancreas, Normal | 46 | 199.883 | 170.572 |
| Prostate, Adenocarcinoma, Primary | 86 | 66.905 | 51.16 |
| Prostate, Normal | 57 | 172.881 | 141.803 |
| Rectum, Adenocarcinoma (Excluding Mucinous Type), Primary | 29 | 1360.42 | 1973.822 |
| Rectum, Adenocarcinoma, Mucinous Type, Primary | 3 | 140.862 | 95.539 |
| Rectum, Normal | 44 | 122.072 | 76.08 |
| Skin, Basal Cell Carcinoma, Primary | 4 | 519.235 | 445.788 |
| Skin, Malignant Melanoma, Primary | 7 | 78.738 | 30.463 |
| Skin, Normal | 61 | 238.046 | 254.135 |
| Skin, Squamous Cell Carcinoma, Primary | 4 | 414.236 | 175.126 |
| Small Intestine, Gastrointestinal Stromal Tumor (GIST), Primary | 4 | 5792.309 | 2849.492 |
| Small Intestine, Normal | 97 | 100.364 | 82.367 |
| Stomach, Adenocarcinoma (Excluding Signet Ring Cell Type), | 27 | 424.297 | 1312.845 |
| Primary | | | |
| Stomach, Adenocarcinoma, Signet Ring Cell Type, Primary | 9 | 189.732 | 95.09 |
| Stomach, Gastrointestinal Stromal Tumor (GIST), Primary | 9 | 6297.024 | 3314.963 |
| Stomach, Normal | 52 | 100.862 | 49.616 |
| Thyroid Gland, Follicular Carcinoma, Primary | 3 | 105.778 | 110.206 |
| Thyroid Gland, Normal | 24 | 123.019 | 67.385 |
| Thyroid Gland, Papillary Carcinoma, Primary; All Variants | 29 | 53.051 | 33.209 |
| Urinary Bladder, Normal | 9 | 589.553 | 501.207 |
| Urinary Bladder, Transitional Cell Carcinoma, Primary | 4 | 148.173 | 100.896 |
| Uterine Cervix, Adenocarcinoma, Primary | 3 | 1137.023 | 593.279 |
| Uterine Cervix, Normal | 115 | 608.103 | 352.223 |
| Vulva, Normal | 4 | 283.469 | 232.196 |
| Vulva, Squamous Cell Carcinoma, Primary | 5 | 398.101 | 277.493 |

Epidermal Growth Factor Receptor

The expression of Epidermal Growth Factor Receptor (EGFR), a tyrosine kinase receptor, has been implicated as necessary in the development of adenomas and carcinomas in intestinal tumors, and subsequent expansion of initiated tumors (Roberts et al., 2002, PNAS, 99:1521-1526). Overexpression of EGFR also plays a role in neoplasia, especially in tumors of epithelial origin (Kari et al., 2003, Cancer Res., 63:1-5). EGFR overexpression has also been implicated in colorectal cancer, pancreatic cancer, gliomal development, small-cell lung cancer, and other carcinomas (Karamouzis et al., 2007, JAMA 298:70-82; Toschi et al., 2007, Oncologist, 12:211-220; Sequist et al., 2007, Oncologist, 12:325-330; Hatake et al., 2007, Breast Cancer, 14:132-149). EGFR is a member of the ErbB family of receptors, which includes HER2c/neu, Her2 and Her3 receptor tyrosine kinases. The molecular signaling pathway of EGFR activation has been mapped through experimental and computer modeling, involving other 200 reactions and 300 chemical species interactions (see Oda et al., Epub 2005, Mol. Sys. Biol., 1:2005.0010). Moreover, EGFR, through its signaling cascade pathway, stimulates PARP activation to initiate downstream cellular events mediated through the PARP pathway (Hagan et al., 2007, J. Cell. Biochem., 101:1384-1393.

Experiments were conducted to verify the correlative relationship between PARP and EGFR expression in a variety of tissue samples. Table XXI depicts the level of expression in a variety of tissues, including adrenal gland, bone, breast tumor tissue, including IDC and infiltrating lobular carcinoma, among others. As seen, upregulation of EGFR can be seen in the same tissues as that for PARP1 upregulation, for example in breast, ovarian and lung cancers. Accordingly, one embodiment is the treatment of susceptible diseases with a combination of PARP and EGFR modulators. Moreover, EGFR related genes, including genes that are co-regulated along the EGFR pathway, are also contemplated herein.

TABLE XXI
Expression of EGFR (Epidermal Growth Factor Receptor; erythroblastic leukemia
viral (v-erb-b) oncogene homolog, avian) in human primary tumors in comparison
with normal tissues tested on Array hg133a.

| | Sample | | | |
|---|---|---|---|---|
| Sample Set | Count | Mean | Std. Dev. | Median |
| Adrenal Gland, Adrenal Cortical Carcinoma, Primary | 3 | 129.704 | 68.212 | 98.678 |
| Adrenal Gland, Normal | 13 | 206.012 | 141.491 | 218.327 |
| Bone, Giant Cell Tumor of Bone, Primary | 10 | 75.665 | 48.088 | 65.433 |
| Bone, Normal | 8 | 56.238 | 60.711 | 37.849 |
| Bone, Osteosarcoma, Primary | 4 | 120.054 | 48.685 | 105.045 |
| Breast, Infiltrating Carcinoma of Mixed Ductal and | 8 | 41.399 | 47.671 | 22.832 |
| Lobular Type, Primary | | | | |
| Breast, Infiltrating Ductal Carcinoma, Primary | 169 | 99.864 | 205.802 | 61.254 |
| Breast, Infiltrating Lobular Carcinoma, Primary | 17 | 95.073 | 86.523 | 74.745 |
| Breast, Intraductal Carcinoma | 3 | 76.167 | 20.435 | 78.839 |
| Breast, Mucinous Carcinoma, Primary | 4 | 53.4 | 53.594 | 40.467 |
| Breast, Normal | 68 | 245.198 | 215.156 | 205.936 |
| Breast, Phyllodes Tumor (Cystosarcoma Phyllodes), | 5 | 393.825 | 154.773 | 467.458 |
| Primary | | | | |
| Colon, Adenocarcinoma (Excluding Mucinous Type), | 77 | 120.497 | 94.693 | 103.941 |
| Primary | | | | |
| Colon, Adenocarcinoma, Mucinous Type, Primary | 7 | 93.805 | 74.634 | 83.1 |
| Colon, Normal | 180 | 171.561 | 111.035 | 183.725 |
| Endometrium, Adenocarcinoma, Endometrioid Type, | 50 | 159.77 | 123.307 | 141.211 |
| Primary | | | | |
| Endometrium, Mullerian Mixed Tumor, Primary | 7 | 279.821 | 425.216 | 71.541 |
| Endometrium, Normal | 23 | 247.392 | 190.703 | 207.384 |
| Esophagus, Adenocarcinoma, Primary | 3 | 65.199 | 53.315 | 70.837 |
| Esophagus, Normal | 22 | 284.301 | 195.112 | 296.05 |
| Kidney, Carcinoma, Chromophobe Type, Primary | 3 | 199.572 | 175.321 | 149.855 |
| Kidney, Normal | 81 | 167.833 | 111.603 | 166.218 |
| Kidney, Renal Cell Carcinoma, Clear Cell Type, Primary | 45 | 475.552 | 460.868 | 363.274 |
| Kidney, Renal Cell Carcinoma, Non-Clear Cell Type, | 15 | 438.275 | 312.272 | 363.517 |
| Primary | | | | |
| Kidney, Transitional Cell Carcinoma, Primary | 4 | 128.624 | 102.806 | 127.813 |
| Kidney, Wilm's Tumor, Primary | 8 | 71.286 | 82.021 | 28.815 |
| Larynx, Normal | 4 | 370.959 | 186.229 | 396.688 |
| Larynx, Squamous Cell Carcinoma, Primary | 4 | 1310.153 | 1353.765 | 967.125 |
| Liver, Hepatocellular Carcinoma | 16 | 220.168 | 276.906 | 183.839 |
| Liver, Normal | 42 | 283.048 | 211.77 | 213.125 |
| Lung, Adenocarcinoma, Primary | 46 | 297.437 | 489.456 | 155.995 |
| Lung, Adenosquamous Carcinoma, Primary | 3 | 128.766 | 91.833 | 100.892 |
| Lung, Large Cell Carcinoma, Primary | 7 | 145.19 | 174.142 | 58.306 |
| Lung, Neuroendocrine Carcinoma (Non-Small Cell | 3 | 24.308 | 17.541 | 24.732 |
| Type), Primary | | | | |
| Lung, Normal | 126 | 214.472 | 136.084 | 199.47 |
| Lung, Small Cell Carcinoma, Primary | 3 | 38.594 | 44.361 | 17.537 |
| Lung, Squamous Cell Carcinoma, Primary | 39 | 234.471 | 241.841 | 175.944 |
| Oral Cavity, Squamous Cell Carcinoma, Primary | 3 | 710.2 | 417.391 | 487.112 |
| Ovary, Adenocarcinoma, Clear Cell Type, Primary | 6 | 110.201 | 69.532 | 80.94 |
| Ovary, Adenocarcinoma, Endometrioid Type, Primary | 22 | 106.113 | 76.106 | 108.206 |
| Ovary, Adenocarcinoma, Papillary Serous Type, Primary | 36 | 125.456 | 131.366 | 91.677 |
| Ovary, Granulosa Cell Tumor, Primary | 3 | 330.038 | 171.65 | 304.702 |
| Ovary, Mucinous Cystadenocarcinoma, Primary | 7 | 256.915 | 196.875 | 201.768 |
| Ovary, Mullerian Mixed Tumor, Primary | 5 | 173.476 | 217.763 | 128.913 |
| Ovary, Normal | 89 | 226.521 | 106.329 | 232.277 |
| Pancreas, Adenocarcinoma, Primary | 23 | 159.08 | 123.238 | 94.418 |
| Pancreas, Islet Cell Tumor, Malignant, Primary | 7 | 55.68 | 51.943 | 48.9 |
| Pancreas, Normal | 46 | 137.569 | 117.347 | 117.425 |
| Prostate, Adenocarcinoma, Primary | 86 | 170.831 | 100.727 | 158.375 |
| Prostate, Normal | 57 | 194.519 | 129.737 | 179.636 |
| Rectum, Adenocarcinoma (Excluding Mucinous Type), | 29 | 170.452 | 87.615 | 174.248 |
| Primary | | | | |
| Rectum, Adenocarcinoma, Mucinous Type, Primary | 3 | 195.563 | 149.368 | 111.354 |
| Rectum, Normal | 44 | 202.086 | 106.159 | 233.46 |
| Skin, Basal Cell Carcinoma, Primary | 4 | 510.675 | 294.101 | 465.462 |
| Skin, Malignant Melanoma, Primary | 7 | 77.052 | 102.515 | 28.869 |
| Skin, Normal | 61 | 296.749 | 214.128 | 265.763 |
| Skin, Squamous Cell Carcinoma, Primary | 4 | 205.607 | 109.906 | 165.561 |
| Small Intestine, Gastrointestinal Stromal Tumor (GIST), | 4 | 87.92 | 60.244 | 91.574 |
| Primary | | | | |
| Small Intestine, Normal | 97 | 112.607 | 75.33 | 110.804 |
| Stomach, Adenocarcinoma (Excluding Signet Ring Cell | 27 | 159.547 | 90.62 | 141.751 |
| Type), Primary | | | | |
| Stomach, Adenocarcinoma, Signet Ring Cell Type, | 9 | 156.941 | 66.185 | 156.444 |
| Primary | | | | |
| Stomach, Gastrointestinal Stromal Tumor (GIST), | 9 | 79.845 | 49.667 | 73.449 |
| Primary | | | | |
| Stomach, Normal | 52 | 130.321 | 87.634 | 120.267 |
| Thyroid Gland, Follicular Carcinoma, Primary | 3 | 128.064 | 21.149 | 127.098 |
| Thyroid Gland, Normal | 24 | 181.933 | 105.446 | 166.104 |
| Thyroid Gland, Papillary Carcinoma, Primary; All | 29 | 242.517 | 160.473 | 192.848 |
| Variants | | | | |
| Urinary Bladder, Normal | 9 | 155.559 | 151.518 | 131.99 |
| Urinary Bladder, Transitional Cell Carcinoma, Primary | 4 | 223.719 | 200.354 | 167.709 |
| Uterine Cervix, Adenocarcinoma, Primary | 3 | 86.934 | 98.416 | 30.427 |
| Uterine Cervix, Normal | 115 | 205.156 | 149.735 | 173.903 |
| Vulva, Normal | 4 | 352.591 | 203.2 | 276.016 |
| Vulva, Squamous Cell Carcinoma, Primary | 5 | 863.035 | 591.738 | 558.964 |

Thymidylate Synthase

Thymidylate synthase (TYMS) uses the 5,10-methylenetetrahydrofolate (methylene-THF) as a cofactor to maintain the dTMP (thymidine-5-prime monophosphate) pool critical for DNA replication and repair. The enzyme has been of interest as a target for cancer chemotherapeutic agents. It is considered to be the primary site of action for 5-fluorouracil, 5-fluoro-2-prime-deoxyuridine, and some folate analogs. Resistance to chemotherapy is a major factor in the mortality in advanced cancer patients.

Wang et al. (2004) used digital karyotyping to search for genomic alterations in liver metastases that were clinically resistant to 5-fluorouracil (5-FU). In 2 of 4 patients, they identified the amplification of a region of approximately 100 kb on chromosome 18 p11.32 that was of particular interest because it contains the TYMS gene, a molecular target of 5-FU. Analysis of TYMS by FISH identified TYMS gene amplification in 7 of 31 (23%) 5-FU-treated cancers, whereas no amplification was observed in metastases of patients who had not been treated with 5-FU. Patients with metastases containing TYMS amplification had a substantially shorter median survival (329 days) than those without amplification (1,021 days, P less than 0.01). These data suggested that genetic amplification of TYMS is a major mechanism of 5-FU resistance in vivo, and may have important implications for the management of colorectal cancer patients with recurrent disease.

One of the mechanisms of 5-FU resistance is the activation of DNA repair, where 5-FU is efficiently removed from DNA by the base excision and mismatch repair systems (Fisher et al., 2007). Because PARP1 is a key enzyme of base excision DNA repair, the combination of PARP1 inhibitors with 5-FU can be beneficial in anticancer therapy, especially for tumors that are clinically resistant to 5-fluorouracil. However, treatment of cancer cells with PARP1 inhibitors in combination with 5-FU can also increase the intracellular concentration of 5-FU and thus exacerbate cytotoxicity. Reduction in 5-FU amounts or concomitant treatment with PARP1 inhibitors and a modulator of TYMS may be useful in the reduction of side effects that may occur with increased cytotoxicity, while maintaining the efficacy of 5-FU as a cancer chemotherapeutic agent.

Experiments were conducted to verify the correlative relationship between PARP and TYMS expression in a variety of tissue samples. Table XXII depicts the level of expression in a variety of tissues, including adrenal gland, bone, breast tumor tissue, including IDC and infiltrating lobular carcinoma, among others. As seen, TYMS is upregulated and coregulated with PARP1 in the same subset of primary human tumors such as tumors of skin, breast, lung, ovarian, esophagus, endometrium and lymphoid tumors and sarcomas. Accordingly, one embodiment is the treatment of susceptible diseases with a combination of PARP and TYMS modulators. Moreover, TYMS-related genes, including genes that are co-regulated along the TYMS pathway, are also contemplated herein.

TABLE XXII
Expression of TYMS (thymidylate synthetase) in human primary tumors in
comparison with normal tissues.

| | Sample | | | |
|---|---|---|---|---|
| Sample Set | Count | Mean | Std. Dev. | Median |
| Adrenal Gland, Adrenal Cortical Carcinoma, Primary | 3 | 132.055 | 80.029 | 94.132 |
| Adrenal Gland, Normal | 13 | 112.2 | 125.033 | 69.718 |
| Bone, Giant Cell Tumor of Bone, Primary | 10 | 442.203 | 142.143 | 426.813 |
| Bone, Normal | 8 | 694.953 | 431.602 | 790.188 |
| Bone, Osteosarcoma, Primary | 4 | 1437.891 | 682.273 | 1471.017 |
| Breast, Infiltrating Carcinoma of Mixed Ductal and Lobular Type, | 8 | 421.25 | 115.564 | 405.456 |
| Primary | | | | |
| Breast, Infiltrating Ductal Carcinoma, Primary | 169 | 378.192 | 296.349 | 289.609 |
| Breast, Infiltrating Lobular Carcinoma, Primary | 17 | 304.073 | 198.812 | 236.622 |
| Breast, Intraductal Carcinoma | 3 | 155.269 | 125.42 | 112.061 |
| Breast, Mucinous Carcinoma, Primary | 4 | 389.638 | 269.167 | 268.04 |
| Breast, Normal | 68 | 211.465 | 208.685 | 137.409 |
| Breast, Phyllodes Tumor (Cystosarcoma Phyllodes), Primary | 5 | 382.787 | 240.871 | 325.51 |
| Colon, Adenocarcinoma (Excluding Mucinous Type), Primary | 77 | 548.493 | 382.288 | 403.87 |
| Colon, Adenocarcinoma, Mucinous Type, Primary | 7 | 512.226 | 272.655 | 390.405 |
| Colon, Normal | 180 | 372.032 | 164.29 | 344.596 |
| Endometrium, Adenocarcinoma, Endometrioid Type, Primary | 50 | 436.551 | 317.309 | 345.238 |
| Endometrium, Mullerian Mixed Tumor, Primary | 7 | 964.617 | 562.444 | 791.133 |
| Endometrium, Normal | 23 | 153.952 | 87.587 | 125.089 |
| Esophagus, Adenocarcinoma, Primary | 3 | 381.495 | 152.442 | 385.147 |
| Esophagus, Normal | 22 | 276.286 | 81.626 | 251.979 |
| Kidney, Carcinoma, Chromophobe Type, Primary | 3 | 72.47 | 18.244 | 73.02 |
| Kidney, Normal | 81 | 141.763 | 57.283 | 136.178 |
| Kidney, Renal Cell Carcinoma, Clear Cell Type, Primary | 45 | 382.754 | 189.427 | 363.738 |
| Kidney, Renal Cell Carcinoma, Non-Clear Cell Type, Primary | 15 | 303.375 | 176.847 | 307.655 |
| Kidney, Transitional Cell Carcinoma, Primary | 4 | 412.684 | 93.512 | 427.31 |
| Kidney, Wilm's Tumor, Primary | 8 | 1476.481 | 439.652 | 1525.669 |
| Larynx, Normal | 4 | 223.235 | 153.725 | 225.307 |
| Larynx, Squamous Cell Carcinoma, Primary | 4 | 438.591 | 147.061 | 444.474 |
| Liver, Hepatocellular Carcinoma | 16 | 339.718 | 312.097 | 186.297 |
| Liver, Normal | 42 | 97.609 | 55.053 | 76.779 |
| Lung, Adenocarcinoma, Primary | 46 | 395.333 | 277.394 | 321.811 |
| Lung, Adenosquamous Carcinoma, Primary | 3 | 289.903 | 126.881 | 288.952 |
| Lung, Large Cell Carcinoma, Primary | 7 | 711.327 | 689.444 | 461.744 |
| Lung, Neuroendocrine Carcinoma (Non-Small Cell Type), Primary | 3 | 774.576 | 1219.221 | 84.446 |
| Lung, Normal | 126 | 148.916 | 221.609 | 87.398 |
| Lung, Small Cell Carcinoma, Primary | 3 | 2588.806 | 571.104 | 2303.79 |
| Lung, Squamous Cell Carcinoma, Primary | 39 | 474.506 | 215.236 | 411.88 |
| Oral Cavity, Squamous Cell Carcinoma, Primary | 3 | 487.365 | 162.008 | 451.582 |
| Ovary, Adenocarcinoma, Clear Cell Type, Primary | 6 | 311.964 | 130.948 | 347.086 |
| Ovary, Adenocarcinoma, Endometrioid Type, Primary | 22 | 416.111 | 270.493 | 350.067 |
| Ovary, Adenocarcinoma, Papillary Serous Type, Primary | 36 | 455.821 | 264.365 | 437.236 |
| Ovary, Granulosa Cell Tumor, Primary | 3 | 418.185 | 134.782 | 444.559 |
| Ovary, Mucinous Cystadenocarcinoma, Primary | 7 | 240.015 | 98.597 | 206.486 |
| Ovary, Mullerian Mixed Tumor, Primary | 5 | 893.972 | 723.698 | 759.005 |
| Ovary, Normal | 89 | 94.871 | 64.692 | 72.971 |
| Pancreas, Adenocarcinoma, Primary | 23 | 225.254 | 85.825 | 226.028 |
| Pancreas, Islet Cell Tumor, Malignant, Primary | 7 | 135.288 | 67.946 | 157.649 |
| Pancreas, Normal | 46 | 142.844 | 58.552 | 127.242 |
| Prostate, Adenocarcinoma, Primary | 86 | 86.485 | 31.51 | 80.935 |
| Prostate, Normal | 57 | 114.079 | 54.25 | 99.422 |
| Rectum, Adenocarcinoma (Excluding Mucinous Type), Primary | 29 | 494.755 | 246.677 | 458.696 |
| Rectum, Adenocarcinoma, Mucinous Type, Primary | 3 | 735.218 | 490.808 | 880.833 |
| Rectum, Normal | 44 | 370.889 | 136.132 | 367.675 |
| Skin, Basal Cell Carcinoma, Primary | 4 | 330.685 | 104.388 | 299.771 |
| Skin, Malignant Melanoma, Primary | 7 | 689.139 | 197.955 | 693.518 |
| Skin, Normal | 61 | 150.4 | 70.711 | 140.82 |
| Skin, Squamous Cell Carcinoma, Primary | 4 | 487.68 | 411.122 | 359.363 |
| Small Intestine, Gastrointestinal Stromal Tumor (GIST), Primary | 4 | 141.255 | 100.778 | 140.167 |
| Small Intestine, Normal | 97 | 303.491 | 125.797 | 290.568 |
| Stomach, Adenocarcinoma (Excluding Signet Ring Cell Type), | 27 | 510.892 | 294.791 | 463.295 |
| Primary | | | | |
| Stomach, Adenocarcinoma, Signet Ring Cell Type, Primary | 9 | 395.57 | 185.806 | 327.718 |
| Stomach, Gastrointestinal Stromal Tumor (GIST), Primary | 9 | 280.21 | 203.266 | 248.372 |
| Stomach, Normal | 52 | 233.257 | 147.033 | 184.606 |
| Thyroid Gland, Follicular Carcinoma, Primary | 3 | 165.154 | 166.032 | 71.214 |
| Thyroid Gland, Normal | 24 | 75.569 | 58.227 | 54.852 |
| Thyroid Gland, Papillary Carcinoma, Primary; All Variants | 29 | 199.353 | 100.226 | 208.498 |
| Urinary Bladder, Normal | 9 | 122.017 | 41.588 | 121.504 |
| Urinary Bladder, Transitional Cell Carcinoma, Primary | 4 | 929.875 | 676.766 | 763.497 |
| Uterine Cervix, Adenocarcinoma, Primary | 3 | 396.607 | 320.83 | 492.964 |
| Uterine Cervix, Normal | 115 | 139.799 | 168.179 | 96.579 |
| Vulva, Normal | 4 | 219.039 | 93.687 | 174.65 |
| Vulva, Squamous Cell Carcinoma, Primary | 5 | 514.322 | 465.291 | 319.74 |

Dihydrofolate Reductase

Folates play a key role in one-carbon metabolism essential for the biosynthesis of purines, thymidylate and hence DNA replication. The antifolate methotrexate was rationally-designed nearly 60 years ago to potently block the folate-dependent enzyme dihydrofolate reductase (DHFR), achieving temporary remissions in childhood acute leukemia. Dihydrofolate reductase converts dihydrofolate into tetrahydrofolate, a methyl group shuttle required for the de novo synthesis of purines, thymidylic acid, and certain amino acids. While the functional dihydrofolate reductase gene has been mapped to chromosome 5, multiple intronless processed pseudogenes or dihydrofolate reductase-like genes have been identified on separate chromosomes. DNA sequence amplification is one of the most frequent manifestations of genomic instability in human tumors. However resistance to folates is a major obstacle towards curative cancer chemotherapy. The mechanisms of antifolate resistance are frequently associated with alterations in influx/efflux transporters of antifolates as well as in regulation of folate-dependent enzymes such as DHFR.

Experiments were conducted to determine if a correlative relationship exists between PARP and DHFR expression in a variety of tissue samples. Table XXIII depicts the level of expression of DHFR in a variety of tissues. As seen, DHFR is co-regulated with PARP1 in ovarian, breast endometrium, skin, lung, kidney, lymph tumors sarcomas and Kidney, Wilm's Tumor and other primary human tumor tissues. Accordingly, one embodiment is the treatment of susceptible diseases with a combination of PARP and DHFR modulators. Moreover, DHFR related genes, including genes that are co-regulated along the DHFR pathway, are also contemplated herein.

TABLE XXIII
Expression of DHFR (dihydrofolate reductase) in human primary tumors in
comparison with normal tissues

| | Sample | | | |
|---|---|---|---|---|
| Sample Set | Count | Mean | Std. Dev. | Median |
| Adrenal Gland, Adrenal Cortical Carcinoma, Primary | 3 | 53.061 | 37.548 | 57.399 |
| Adrenal Gland, Normal | 13 | 22.945 | 16.408 | 19.555 |
| Bone, Giant Cell Tumor of Bone, Primary | 10 | 38.484 | 9.626 | 41.785 |
| Bone, Normal | 8 | 82.832 | 44.371 | 74.682 |
| Bone, Osteosarcoma, Primary | 4 | 87.758 | 29.643 | 78.453 |
| Breast, Infiltrating Carcinoma of Mixed Ductal and Lobular Type, | 8 | 58.62 | 32.781 | 49.355 |
| Primary | | | | |
| Breast, Infiltrating Ductal Carcinoma, Primary | 169 | 52.827 | 29.75 | 44.657 |
| Breast, Infiltrating Lobular Carcinoma, Primary | 17 | 58.29 | 53.061 | 38.56 |
| Breast, Intraductal Carcinoma | 3 | 44.978 | 22.862 | 57.325 |
| Breast, Mucinous Carcinoma, Primary | 4 | 40.964 | 16.635 | 47.057 |
| Breast, Normal | 68 | 38.129 | 15.455 | 35.202 |
| Breast, Phyllodes Tumor (Cystosarcoma Phyllodes), Primary | 5 | 51.482 | 17.856 | 44.299 |
| Colon, Adenocarcinoma (Excluding Mucinous Type), Primary | 77 | 70.123 | 41.505 | 59.975 |
| Colon, Adenocarcinoma, Mucinous Type, Primary | 7 | 81.11 | 57.656 | 58.015 |
| Colon, Normal | 180 | 56.486 | 21.806 | 54.762 |
| Endometrium, Adenocarcinoma, Endometrioid Type, Primary | 50 | 70.055 | 34.502 | 70.361 |
| Endometrium, Mullerian Mixed Tumor, Primary | 7 | 85.451 | 61.922 | 77.752 |
| Endometrium, Normal | 23 | 28.606 | 11.427 | 27.791 |
| Esophagus, Adenocarcinoma, Primary | 3 | 45.832 | 23.407 | 47.507 |
| Esophagus, Normal | 22 | 37.982 | 11.676 | 37.601 |
| Kidney, Carcinoma, Chromophobe Type, Primary | 3 | 17.625 | 11.558 | 23.875 |
| Kidney, Normal | 81 | 39.648 | 13.897 | 38.936 |
| Kidney, Renal Cell Carcinoma, Clear Cell Type, Primary | 45 | 37.43 | 22.148 | 32.293 |
| Kidney, Renal Cell Carcinoma, Non-Clear Cell Type, Primary | 15 | 33.744 | 17.337 | 32.808 |
| Kidney, Transitional Cell Carcinoma, Primary | 4 | 41.028 | 22.893 | 45.222 |
| Kidney, Wilm's Tumor, Primary | 8 | 174.762 | 79.335 | 176.578 |
| Larynx, Normal | 4 | 46.161 | 13.723 | 44.058 |
| Larynx, Squamous Cell Carcinoma, Primary | 4 | 46.204 | 34.758 | 32.263 |
| Liver, Hepatocellular Carcinoma | 16 | 78.036 | 43.038 | 74.708 |
| Liver, Normal | 42 | 86.709 | 31.903 | 89.705 |
| Lung, Adenocarcinoma, Primary | 46 | 45.462 | 19.855 | 41.378 |
| Lung, Adenosquamous Carcinoma, Primary | 3 | 32.97 | 6.387 | 30.038 |
| Lung, Large Cell Carcinoma, Primary | 7 | 50.102 | 13.56 | 51.152 |
| Lung, Neuroendocrine Carcinoma (Non-Small Cell Type), Primary | 3 | 39.58 | 22.283 | 32.609 |
| Lung, Normal | 126 | 30.627 | 18.138 | 27.496 |
| Lung, Small Cell Carcinoma, Primary | 3 | 207.21 | 116.1 | 172.329 |
| Lung, Squamous Cell Carcinoma, Primary | 39 | 44.442 | 20.418 | 38.266 |
| Oral Cavity, Squamous Cell Carcinoma, Primary | 3 | 50.591 | 48.384 | 22.788 |
| Ovary, Adenocarcinoma, Clear Cell Type, Primary | 6 | 52.468 | 11.372 | 50.238 |
| Ovary, Adenocarcinoma, Endometrioid Type, Primary | 22 | 63.741 | 28.237 | 56.181 |
| Ovary, Adenocarcinoma, Papillary Serous Type, Primary | 36 | 70.085 | 42.998 | 53.931 |
| Ovary, Granulosa Cell Tumor, Primary | 3 | 66.06 | 17.895 | 58.1 |
| Ovary, Mucinous Cystadenocarcinoma, Primary | 7 | 59.345 | 17.46 | 58.75 |
| Ovary, Mullerian Mixed Tumor, Primary | 5 | 51.93 | 11.264 | 55.106 |
| Ovary, Normal | 89 | 29.295 | 13.071 | 27.128 |
| Pancreas, Adenocarcinoma, Primary | 23 | 31.801 | 18.707 | 28.935 |
| Pancreas, Islet Cell Tumor, Malignant, Primary | 7 | 32.128 | 14.69 | 25.704 |
| Pancreas, Normal | 46 | 20.131 | 10.056 | 19.465 |
| Prostate, Adenocarcinoma, Primary | 86 | 44.128 | 22.422 | 39.503 |
| Prostate, Normal | 57 | 32.561 | 9.798 | 31.657 |
| Rectum, Adenocarcinoma (Excluding Mucinous Type), Primary | 29 | 79.861 | 39.471 | 72.342 |
| Rectum, Adenocarcinoma, Mucinous Type, Primary | 3 | 65.662 | 30.635 | 69.424 |
| Rectum, Normal | 44 | 48.55 | 17.727 | 45.586 |
| Skin, Basal Cell Carcinoma, Primary | 4 | 71.724 | 31.055 | 69.857 |
| Skin, Malignant Melanoma, Primary | 7 | 76.207 | 40.33 | 63.72 |
| Skin, Normal | 61 | 34.889 | 12.719 | 32.547 |
| Skin, Squamous Cell Carcinoma, Primary | 4 | 59.489 | 33.534 | 48.304 |
| Small Intestine, Gastrointestinal Stromal Tumor (GIST), Primary | 4 | 35.594 | 9.378 | 34.778 |
| Small Intestine, Normal | 97 | 73.068 | 29.842 | 71.135 |
| Stomach, Adenocarcinoma (Excluding Signet Ring Cell Type), | 27 | 61.852 | 33.329 | 51.711 |
| Primary | | | | |
| Stomach, Adenocarcinoma, Signet Ring Cell Type, Primary | 9 | 58.447 | 26.841 | 54.011 |
| Stomach, Gastrointestinal Stromal Tumor (GIST), Primary | 9 | 45.187 | 44.147 | 27.267 |
| Stomach, Normal | 52 | 35.652 | 22.821 | 31.295 |
| Thyroid Gland, Follicular Carcinoma, Primary | 3 | 35.569 | 12.886 | 29.585 |
| Thyroid Gland, Normal | 24 | 32.666 | 11.093 | 32.857 |
| Thyroid Gland, Papillary Carcinoma, Primary; All Variants | 29 | 37.14 | 14.107 | 34.082 |
| Urinary Bladder, Normal | 9 | 22.458 | 7.004 | 21.109 |
| Urinary Bladder, Transitional Cell Carcinoma, Primary | 4 | 89.141 | 107.591 | 38.967 |
| Uterine Cervix, Adenocarcinoma, Primary | 3 | 30.539 | 8.38 | 35.371 |
| Uterine Cervix, Normal | 115 | 31.69 | 19.096 | 28.354 |
| Vulva, Normal | 4 | 37.254 | 7.095 | 35.127 |
| Vulva, Squamous Cell Carcinoma, Primary | 5 | 65.844 | 39.414 | 55.885 |

NFkB

NFKB has been detected in numerous cell types that express cytokines, chemokines, growth factors, cell adhesion molecules, and some acute phase proteins in health, as well as in many disease states. NFKB is activated by a wide variety of stimuli such as cytokines, oxidant-free radicals, inhaled particles, ultraviolet irradiation, and bacterial or viral products. Nuclear factor-κB (NF-κB) is the generic name for a family of dimers formed by several proteins: NF-κB1 (also known as p50/p105), NF-κB2 (also known as p52/p100), REL, RELA (also known as p65/NF-κB3) and RELB. The different heterodimers bind to specific promoters to initiate transcription of a wide range of genes that influence the inflammatory response as well as cell death and survival and tissue repair. NF-κB is active in the nucleus and is inhibited through its sequestration in the cytoplasm by the inhibitor of κB (IκB). IκB binds to NF-κB and is important for the maintenance of NF-κB in the cytoplasm. NF-κB becomes active once it is released from IκB (FIG. 1). IκB is a target of several well-characterized kinase cascades that activate IκB kinase (IKK). The IKKα and IKKβ subunits preferentially form heterodimers, and both can directly phosphorylate IκB, which results in its ubiquitylation and degradation by the proteosome. The IKK subunit IKKγ has a structural and regulatory function and is thought to mediate interactions with upstream kinases in response to cellular activation signals. Growth factors, cytokines such as interleukin-1 (IL-1) and tumor-necrosis factor (TNF), hormones and other signals activate NF-κB by the phosphorylation of IκB.

Substantial evidence indicates that NF-κB regulates oncogenesis and tumor progression. Two mouse models of inflammation-associated cancer further support the link between NF-κB activity and cancer formation and progression. For example, studies in Mdr2-knockout mice, which spontaneously develop an inflammatory condition known as cholestatic hepatitis, show that these mice develop hepatocellular carcinoma. The survival of hepatocytes and their progression to malignancy is regulated by NF-κB7. Moreover, in a mouse model of colitis-associated cancer, the deletion of IKKβ in intestinal epithelial cells results in a marked decrease in tumor incidence. All these results indicate that NF-κB activation, which is often seen in inflammatory-based disease, is associated with an increased incidence of cancer.

Although chemotherapeutic agents have been successfully used in treating patients with many different types of cancer, acquisition of resistance to the cytotoxic effects of chemotherapy has emerged as a significant impediment to effective cancer treatment. Most chemotherapy agents trigger the cell-death process through activation of the tumor-suppressor protein p53. However, NF-κB is also activated in response to treatment with cytotoxic drugs, such as taxanes, Vinca alkaloids and topoisomerase inhibitors. The NF-κB pathway impinges on many aspects of cell growth and apoptosis. For example, in HeLa cells, the topoisomerase I inhibitor SN38 (7-ethyl-10-hydroxycamptothecin), which is an active metabolite of irinotecan, and the topoisomerase II inhibitor doxorubicin both induce NF-κB nuclear translocation and activation of NF-κB target genes directly through mobilization and stimulation of the IKK complex, but not through the secondary production of NF-κB activators such as cytokines, leading to cell survival.

In vivo models of ovarian cancer, colorectal cancer and pancreatic cancer have shown that NF-kB inhibition increases the efficacy of anticancer drugs (Mabuchi et al., 2004, J. Biol. Chem. 279:23477-23485; Cusack et al., 2001, Cancer Res. 61:3535-3540; Shah et al., 2001, J. Cell Biochem. 82:110-122; Bold et al., 2001, J. Surg. Res. 100:11-17). It is thought that NF-κB inhibition prevents tumors from becoming resistant to chemotherapeutic agents. Therefore, development of NF-κB inhibitors could increase the efficacy of many anticancer drugs.

Recent studies suggest that the synthesis of protein bound ADP-ribose polymers catalyzed by poly(ADP-ribose) polymerase-1 (PARP-1) regulates the NF-kB-dependent pathway. NF-kB-p50 DNA binding is protein-poly(ADP-ribosyl)-ation dependent. Co-immunoprecipitation and immunoblot analysis revealed that PARP-1 physically interacts with NF-kB-p50 with high specificity (Chang W J, Alvarez-Gonzalez R., J. Biol. Chem. 2001 Dec. 14; 276(50):47664-70. The sequence-specific DNA binding of NF-kappa B is reversibly regulated by the automodification reaction of poly(ADP-ribose) polymerase 1). Besides direct interaction with PARP1, NF-kB pathways are co-regulated in several tumor types where PARP1 upregulation was also observed (see Tables I-XVIII). Moreover, NFκB is a ubiquitous transcriptional factor and promotes the transcription of 150 genes (Mori et al., 2002, Blood 100:1828-1834; Mori et al., 1999, Blood 93:2360-2368). NF-kB molecular pathway covers several crucial cellular proteins involved in the regulation of inflammation, apoptosis, cell proliferation and differentiation such as IRAK1, Bcl-2 (Yang et al., 2006, Clin Cancer Res. 12:950-60), Bcl-6 (Li et al., 2005, J Immunol. 174(1):205-14), VEGF (Tong et al., 2006, Respir Res. 2:7:37), Aurora kinase and VAV3 oncogene.

Accordingly, one embodiment is the treatment of susceptible diseases with a combination of PARP and NFKB modulators. Moreover, NFKB related genes, IRAK1, Bcl-2, Bcl-6, Aurora kinase, VAV3 oncogene and other genes co-regulated in the NFKB pathway, are also contemplated herein.

Endothelial Cell Factors/VEGF

Endothelial cells provide nutrients and oxygen and removing catabolites, and produce multiple growth factors that can promote tumor growth, invasion, and survival. Angiogenesis, therefore, provides both a perfusion effect and a paracrine effect to a growing tumor and tumor cells, and endothelial cells can drive each other to amplify the malignant phenotype. Ovarian cancer is a major source of cancer morbidity and mortality despite modern advances in surgical and chemotherapeutic management. The molecular pathways that control angiogenesis are key to the pathogenesis of ovarian cancer and have been shown to have prognostic significance. Understanding of molecular pathways that are involved in the regulation of angiogenesis leads to the identification of a number of targets for antiangiogenic therapies. Antiangiogenic agents are currently in clinical trials and several have now been approved or are pending approval for clinical use in the treatment of cancer and other angiogenesis dependent diseases. One target of angiogenesis is VEGF and its receptors. VEGF, initially called VPF due to its ability to increase vascular permeability, stimulates proliferation and migration of endothelial cells and plays a pivotal role in vasculogenesis, angiogenesis, and endothelial integrity and survival. VEGF plays a significant role in other biological signaling functions, including tumor cell survival and motility, hematopoiesis, immune function, hepatic integrity, and neurological function. The multiple effects of VEGF are mediated through several different receptors, including tyrosine kinase receptors VEGFR1 (flt-1), VEGFR2 (KDR, flk-1), and VEGFR3 (flt4) with differing binding specificities for each form of VEGF.

Experiments were conducted to determine if a correlative relationship exists between PARP and VEGF expression in a variety of tumor tissue samples. Table XXIV depicts the level of expression in a variety of tissues. As seen, VEGF is upregulated and co-regulated in the same subtype of tumors as PARP1 is upregulated, such as tumors of breast, ovarian and skin tumors and sarcomas. Accordingly, one embodiment is the treatment of susceptible diseases with a combination of PARP and VEGF modulators. Moreover, VEGF related genes, including genes co-regulated in the VEGF pathway, are also contemplated herein.

TABLE XXIV
Expression of VEGF (Vascular Endothelial Growth Factor) in human primary
tumors in comparison with normal tissues.

| | Sample | | | |
|---|---|---|---|---|
| Sample Set | Count | Mean | Std. Dev. | Median |
| Adrenal Gland, Adrenal Cortical Carcinoma, Primary | 3 | 386.427 | 220.704 | 275.803 |
| Adrenal Gland, Normal | 13 | 534.83 | 424.117 | 485.418 |
| Bone, Giant Cell Tumor of Bone, Primary | 10 | 325.043 | 304.973 | 215.554 |
| Bone, Normal | 8 | 195.529 | 73.331 | 187.259 |
| Bone, Osteosarcoma, Primary | 4 | 602.198 | 578.869 | 452.353 |
| Breast, Infiltrating Carcinoma of Mixed Ductal and Lobular Type, | 8 | 191.214 | 66.208 | 171.42 |
| Primary | | | | |
| Breast, Infiltrating Ductal Carcinoma, Primary | 169 | 307.37 | 185.757 | 255.532 |
| Breast, Infiltrating Lobular Carcinoma, Primary | 17 | 305.927 | 201.926 | 241.604 |
| Breast, Intraductal Carcinoma | 3 | 252.557 | 113.835 | 305.515 |
| Breast, Mucinous Carcinoma, Primary | 4 | 207.89 | 79.708 | 202.417 |
| Breast, Normal | 68 | 225.756 | 177.612 | 190.945 |
| Breast, Phyllodes Tumor (Cystosarcoma Phyllodes), Primary | 5 | 379.044 | 247.428 | 340.865 |
| Colon, Adenocarcinoma (Excluding Mucinous Type), Primary | 77 | 403.428 | 291.03 | 331.978 |
| Colon, Adenocarcinoma, Mucinous Type, Primary | 7 | 343.139 | 227.791 | 363.118 |
| Colon, Normal | 180 | 193.049 | 123.726 | 162.853 |
| Endometrium, Adenocarcinoma, Endometrioid Type, Primary | 50 | 429.783 | 250.521 | 368.132 |
| Endometrium, Mullerian Mixed Tumor, Primary | 7 | 376.359 | 163.596 | 382.885 |
| Endometrium, Normal | 23 | 575.093 | 382.852 | 476.946 |
| Esophagus, Adenocarcinoma, Primary | 3 | 464.866 | 319.11 | 455.746 |
| Esophagus, Normal | 22 | 294.149 | 150.077 | 282.678 |
| Kidney, Carcinoma, Chromophobe Type, Primary | 3 | 455.21 | 63.48 | 467.21 |
| Kidney, Normal | 81 | 494.861 | 235.446 | 464.756 |
| Kidney, Renal Cell Carcinoma, Clear Cell Type, Primary | 45 | 2068.059 | 1272.634 | 2000.188 |
| Kidney, Renal Cell Carcinoma, Non-Clear Cell Type, Primary | 15 | 937.413 | 931.299 | 654.782 |
| Kidney, Transitional Cell Carcinoma, Primary | 4 | 975.47 | 808.737 | 754.803 |
| Kidney, Wilm's Tumor, Primary | 8 | 239.096 | 134.285 | 190.813 |
| Larynx, Normal | 4 | 256.177 | 200.315 | 177.084 |
| Larynx, Squamous Cell Carcinoma, Primary | 4 | 253.816 | 104.837 | 217.95 |
| Liver, Hepatocellular Carcinoma | 16 | 471.428 | 322.779 | 382.127 |
| Liver, Normal | 42 | 498.101 | 210.551 | 497.388 |
| Lung, Adenocarcinoma, Primary | 46 | 565.451 | 310.102 | 490.923 |
| Lung, Adenosquamous Carcinoma, Primary | 3 | 579.793 | 730.484 | 222.619 |
| Lung, Large Cell Carcinoma, Primary | 7 | 514.945 | 302.189 | 452.012 |
| Lung, Neuroendocrine Carcinoma (Non-Small Cell Type), Primary | 3 | 180.059 | 54.684 | 189.478 |
| Lung, Normal | 126 | 473.02 | 210.329 | 446.044 |
| Lung, Small Cell Carcinoma, Primary | 3 | 341.097 | 216.97 | 383.485 |
| Lung, Squamous Cell Carcinoma, Primary | 39 | 426.689 | 273.396 | 389.508 |
| Oral Cavity, Squamous Cell Carcinoma, Primary | 3 | 336.828 | 172.021 | 272.722 |
| Ovary, Adenocarcinoma, Clear Cell Type, Primary | 6 | 189.693 | 85.656 | 161.422 |
| Ovary, Adenocarcinoma, Endometrioid Type, Primary | 22 | 475.62 | 316.071 | 419.278 |
| Ovary, Adenocarcinoma, Papillary Serous Type, Primary | 36 | 529.555 | 283.552 | 476.174 |
| Ovary, Granulosa Cell Tumor, Primary | 3 | 235.513 | 64.065 | 228.599 |
| Ovary, Mucinous Cystadenocarcinoma, Primary | 7 | 282.313 | 120.574 | 298.024 |
| Ovary, Mullerian Mixed Tumor, Primary | 5 | 421.141 | 195.681 | 308.7 |
| Ovary, Normal | 89 | 100.699 | 72.854 | 86.687 |
| Pancreas, Adenocarcinoma, Primary | 23 | 524.075 | 227.812 | 478.653 |
| Pancreas, Islet Cell Tumor, Malignant, Primary | 7 | 639.243 | 499.434 | 530.466 |
| Pancreas, Normal | 46 | 407.617 | 115.931 | 425.551 |
| Prostate, Adenocarcinoma, Primary | 86 | 547.601 | 377.291 | 460.667 |
| Prostate, Normal | 57 | 805.882 | 540.435 | 715.723 |
| Rectum, Adenocarcinoma (Excluding Mucinous Type), Primary | 29 | 371.234 | 162.844 | 344.84 |
| Rectum, Adenocarcinoma, Mucinous Type, Primary | 3 | 262.932 | 88.046 | 215.869 |
| Rectum, Normal | 44 | 182.564 | 103.8 | 164.297 |
| Skin, Basal Cell Carcinoma, Primary | 4 | 300.302 | 270.286 | 240.215 |
| Skin, Malignant Melanoma, Primary | 7 | 127.179 | 84.561 | 97.95 |
| Skin, Normal | 61 | 123.011 | 59.089 | 119.897 |
| Skin, Squamous Cell Carcinoma, Primary | 4 | 212.813 | 94.938 | 192.998 |
| Small Intestine, Gastrointestinal Stromal Tumor (GIST), Primary | 4 | 265.372 | 271.901 | 203.655 |
| Small Intestine, Normal | 97 | 257.186 | 170.574 | 215.101 |
| Stomach, Adenocarcinoma (Excluding Signet Ring Cell Type), | 27 | 413.359 | 296.365 | 317.794 |
| Primary | | | | |
| Stomach, Adenocarcinoma, Signet Ring Cell Type, Primary | 9 | 288.769 | 80.831 | 288.931 |
| Stomach, Gastrointestinal Stromal Tumor (GIST), Primary | 9 | 242.777 | 381.025 | 102.627 |
| Stomach, Normal | 52 | 362.303 | 159.695 | 328.802 |
| Thyroid Gland, Follicular Carcinoma, Primary | 3 | 841.322 | 697.265 | 925.178 |
| Thyroid Gland, Normal | 24 | 1134.377 | 286.605 | 1134.341 |
| Thyroid Gland, Papillary Carcinoma, Primary; All Variants | 29 | 836.596 | 350.532 | 873.247 |
| Urinary Bladder, Normal | 9 | 262.966 | 166.1 | 173.303 |
| Urinary Bladder, Transitional Cell Carcinoma, Primary | 4 | 719.789 | 248.426 | 735.062 |
| Uterine Cervix, Adenocarcinoma, Primary | 3 | 428.006 | 164.593 | 467.605 |
| Uterine Cervix, Normal | 115 | 259.71 | 271.623 | 197.708 |
| Vulva, Normal | 4 | 203.085 | 146.444 | 154.186 |
| Vulva, Squamous Cell Carcinoma, Primary | 5 | 329.278 | 108.746 | 291.862 |

Matrix Metalloproteinase Family

Matrix metalloproteinase-9 (matrix metallopeptidease-9; MMP9), also known as 92-kD gelatinase or type V collagenase, is a 92-kD type IV collagenase that degrades collagen in the extracellular matrix. MMP9 expression plays a role in allowing angiogenesis and invasion by different pituitary tumor types, where MMP9 expression is present in some invasive and recurrent pituitary adenomas and in the majority of pituitary carcinoma. In addition, invasive macroprolactinomas are significantly more likely to express MMP9 than noninvasive macroprolactinomas. Invasive macroprolactinomas show higher-density MMP9 staining than noninvasive tumors and normal pituitary gland, or between different sized prolactinomas. MMP9 expression is also related to aggressive tumor behavior. MMP-9 also belongs to the molecular network of transcription factor nuclear-factor kappa B (NF-kappaB) that is a hallmark of many highly malignant tumors (St-Pierre et al., 2004, Expert Opin. Therp. Targets 8:473-489).

Concentrations of MMP9 are also increased in the bronchoalveolar lavage fluid (BAL), sputum, bronchi, and serum of asthmatic subjects compared with normal individuals. Using segmental bronchoprovocation (SBP) and ELISA analysis of BAL from allergic subjects (Kelly et al., 2000, Am. J. Resp. Crit. Care Med. 162:1157-1161), increased MMP9 was detected in antigen-challenged patients compared with saline-challenged patients. The same study also concluded that MMP9 may contribute not only to inflammation but also to eventual airway remodeling in asthma.

The link between MMP9 expression and tumor recurrence and tumor invasiveness, as well as its association with angiogenesis, suggests a potential therapeutic strategy for application of MMP9 inhibitors. MMP-9 overexpression in cancer and various inflammatory conditions points to the molecular mechanisms controlling its expression as a potential target for eventual rational therapeutic intervention.

Experiments were conducted to determine if a correlative relationship exists between PARP and MMP9 expression in a variety of tumor tissue samples. Table XXV depicts the level of expression in a variety of tissues. As seen, MMP9 is upregulated and co-regulated in the same subtype of tumors as PARP1 is upregulated, such as tumors of breast, endometrium, lung, ovarian and skin tumors and sarcomas. Accordingly, one embodiment is the treatment of susceptible diseases with a combination of PARP and MMP9 modulators. Moreover, MMP9 related genes, including genes co-regulated in the MMP9 pathway, are also contemplated herein.

TABLE XXV
Expression of MMP9 (matrix metalloproteinase 9; matrix metallopeptidase 9; gelatinase B, 92 kDa
gelatinase, 92 kDa type IV collagenase) in human primary tumors in comparison with normal tissues.

| | Sample | | Std. | |
|---|---|---|---|---|
| Sample Set | Count | Mean | Dev. | Median |
| Adrenal Gland, Adrenal Cortical Carcinoma, Primary | 3 | 309.003 | 363.776 | 111.922 |
| Adrenal Gland, Normal | 13 | 252.092 | 641.203 | 78.986 |
| Bone, Giant Cell Tumor of Bone, Primary | 10 | 8416.738 | 2667.464 | 7897.901 |
| Bone, Normal | 8 | 2879.804 | 1459.135 | 3104.17 |
| Bone, Osteosarcoma, Primary | 4 | 4257.056 | 4017.873 | 3840.443 |
| Breast, Infiltrating Carcinoma of Mixed Ductal and Lobular Type, | 8 | 365.875 | 238.051 | 297.772 |
| Primary | | | | |
| Breast, Infiltrating Ductal Carcinoma, Primary | 169 | 458.281 | 676.915 | 312.815 |
| Breast, Infiltrating Lobular Carcinoma, Primary | 17 | 242.394 | 186.712 | 184.418 |
| Breast, Intraductal Carcinoma | 3 | 174.671 | 131.922 | 118.519 |
| Breast, Mucinous Carcinoma, Primary | 4 | 554.482 | 474.424 | 531.033 |
| Breast, Normal | 68 | 212.419 | 532.284 | 109.432 |
| Breast, Phyllodes Tumor (Cystosarcoma Phyllodes), Primary | 5 | 152.665 | 73.258 | 173.198 |
| Colon, Adenocarcinoma (Excluding Mucinous Type), Primary | 77 | 281.312 | 182.492 | 243.195 |
| Colon, Adenocarcinoma, Mucinous Type, Primary | 7 | 506.083 | 504.14 | 208.984 |
| Colon, Normal | 180 | 146.424 | 76.77 | 125.097 |
| Endometrium, Adenocarcinoma, Endometrioid Type, Primary | 50 | 280.906 | 226.62 | 184.995 |
| Endometrium, Mullerian Mixed Tumor, Primary | 7 | 2130.553 | 4421.419 | 152.861 |
| Endometrium, Normal | 23 | 74.372 | 81.725 | 52.858 |
| Esophagus, Adenocarcinoma, Primary | 3 | 162.76 | 119.022 | 126.363 |
| Esophagus, Normal | 22 | 99.099 | 43.267 | 87.497 |
| Kidney, Carcinoma, Chromophobe Type, Primary | 3 | 74.455 | 12.548 | 74.468 |
| Kidney, Normal | 81 | 65.316 | 29.326 | 53.621 |
| Kidney, Renal Cell Carcinoma, Clear Cell Type, Primary | 45 | 207.592 | 264.124 | 118.489 |
| Kidney, Renal Cell Carcinoma, Non-Clear Cell Type, Primary | 15 | 132.558 | 168.005 | 83.409 |
| Kidney, Transitional Cell Carcinoma, Primary | 4 | 111.546 | 77.957 | 85.9 |
| Kidney, Wilm's Tumor, Primary | 8 | 100.97 | 58.478 | 88.166 |
| Larynx, Normal | 4 | 162.638 | 197.338 | 77.062 |
| Larynx, Squamous Cell Carcinoma, Primary | 4 | 675.211 | 526.673 | 461.672 |
| Liver, Hepatocellular Carcinoma | 16 | 182.726 | 121.648 | 140.502 |
| Liver, Normal | 42 | 91.165 | 56.079 | 78.537 |
| Lung, Adenocarcinoma, Primary | 46 | 382.767 | 295.098 | 269.92 |
| Lung, Adenosquamous Carcinoma, Primary | 3 | 157.601 | 24.124 | 169.713 |
| Lung, Large Cell Carcinoma, Primary | 7 | 513.391 | 243.603 | 389.392 |
| Lung, Neuroendocrine Carcinoma (Non-Small Cell Type), Primary | 3 | 169.638 | 135.354 | 144.106 |
| Lung, Normal | 126 | 199.713 | 537.561 | 113.429 |
| Lung, Small Cell Carcinoma, Primary | 3 | 116.438 | 20.137 | 123.616 |
| Lung, Squamous Cell Carcinoma, Primary | 39 | 458.118 | 327.988 | 389.82 |
| Oral Cavity, Squamous Cell Carcinoma, Primary | 3 | 888.299 | 613.909 | 784.061 |
| Ovary, Adenocarcinoma, Clear Cell Type, Primary | 6 | 84.894 | 28.076 | 97 |
| Ovary, Adenocarcinoma, Endometrioid Type, Primary | 22 | 240.36 | 248.189 | 132.824 |
| Ovary, Adenocarcinoma, Papillary Serous Type, Primary | 36 | 306.398 | 377.337 | 200.176 |
| Ovary, Granulosa Cell Tumor, Primary | 3 | 54.976 | 11.932 | 60.659 |
| Ovary, Mucinous Cystadenocarcinoma, Primary | 7 | 141.805 | 147.638 | 75.617 |
| Ovary, Mullerian Mixed Tumor, Primary | 5 | 173.381 | 132.143 | 87.017 |
| Ovary, Normal | 89 | 79.258 | 34.05 | 74.142 |
| Pancreas, Adenocarcinoma, Primary | 23 | 771.454 | 2575.291 | 170.842 |
| Pancreas, Islet Cell Tumor, Malignant, Primary | 7 | 94.33 | 64.615 | 78.529 |
| Pancreas, Normal | 46 | 114.647 | 45.476 | 107.669 |
| Prostate, Adenocarcinoma, Primary | 86 | 97.399 | 54.502 | 89.814 |
| Prostate, Normal | 57 | 88.492 | 62.469 | 76.093 |
| Rectum, Adenocarcinoma (Excluding Mucinous Type), Primary | 29 | 263.49 | 137.758 | 225.801 |
| Rectum, Adenocarcinoma, Mucinous Type, Primary | 3 | 243.039 | 77.917 | 261.742 |
| Rectum, Normal | 44 | 138.354 | 57.909 | 134.267 |
| Skin, Basal Cell Carcinoma, Primary | 4 | 310.963 | 41.044 | 316.027 |
| Skin, Malignant Melanoma, Primary | 7 | 438.656 | 524.74 | 226.982 |
| Skin, Normal | 61 | 178.343 | 140.519 | 131.711 |
| Skin, Squamous Cell Carcinoma, Primary | 4 | 623.436 | 372.054 | 519.425 |
| Small Intestine, Gastrointestinal Stromal Tumor (GIST), Primary | 4 | 123.403 | 136.145 | 71.538 |
| Small Intestine, Normal | 97 | 159.231 | 138.833 | 115.218 |
| Stomach, Adenocarcinoma (Excluding Signet Ring Cell Type), | 27 | 278.681 | 198.698 | 199.374 |
| Primary | | | | |
| Stomach, Adenocarcinoma, Signet Ring Cell Type, Primary | 9 | 248.745 | 135.248 | 190.314 |
| Stomach, Gastrointestinal Stromal Tumor (GIST), Primary | 9 | 92.783 | 24.101 | 86.242 |
| Stomach, Normal | 52 | 111.717 | 50.627 | 99.757 |
| Thyroid Gland, Follicular Carcinoma, Primary | 3 | 107.466 | 29.565 | 123.712 |
| Thyroid Gland, Normal | 24 | 109.347 | 67.108 | 93.531 |
| Thyroid Gland, Papillary Carcinoma, Primary; All Variants | 29 | 219.295 | 167.203 | 143.996 |
| Urinary Bladder, Normal | 9 | 96.898 | 51.823 | 93.024 |
| Urinary Bladder, Transitional Cell Carcinoma, Primary | 4 | 318.932 | 441.905 | 120.076 |
| Uterine Cervix, Adenocarcinoma, Primary | 3 | 98.137 | 20.265 | 93.975 |
| Uterine Cervix, Normal | 115 | 118.874 | 156.193 | 81.22 |
| Vulva, Normal | 4 | 174.167 | 131.037 | 134.115 |
| Vulva, Squamous Cell Carcinoma, Primary | 5 | 361.991 | 143.537 | 284.436 |

Vascular Endothelial Growth Factor Receptor (VEGFR)

As discussed above, the molecular pathways that control angiogenesis are key to the pathogenesis of cancers, including ovarian cancer, and have been shown to have prognostic significance. Understanding of molecular pathways that are involved in the regulation of angiogenesis has lead to the identification of a number of targets for antiangiogenic therapies. Antiangiogenic agents are currently in clinical trials and several have now been approved or are pending approval for clinical use in the treatment of cancer and other angiogenesis dependent diseases. One of the most abundant targets of angiogenesis is VEGF and its receptors. The multiple effects of VEGF are mediated through several different receptors including the tyrosine kinase receptors VEGFR1 (flt-1), VEGFR2 (KDR, flk-1), and VEGFR3 (flt4) with differing binding specificities for each form of VEGF.

Experiments were conducted to determine if a correlative relationship exists between PARP and VEGFR expression in a variety of tumor tissue samples. Table XXVI depicts the level of expression in a variety of tissues. As seen, VEGFR is upregulated and co-regulated in the same subtype of tumors as PARP1 is upregulated, such as tumors of breast, ovarian and skin tumors and sarcomas. Accordingly, one embodiment is the treatment of susceptible diseases with a combination of PARP and VEGFR modulators. Moreover, VEGFR related genes, including genes co-regulated in the VEGFR pathway, are also contemplated herein.

TABLE XXVI
Expression of VEGFR (vascular endothelial growth factor receptor; fms-related
tyrosine kinase 1; vascular permeability factor receptor) in human primary
tumors in comparison with normal tissues.

| | Sample | | | |
|---|---|---|---|---|
| Sample Set | Count | Mean | Std. Dev. | Median |
| Adrenal Gland, Adrenal Cortical Carcinoma, Primary | 3 | 164.936 | 4.48 | 166.572 |
| Adrenal Gland, Normal | 13 | 152.418 | 86.102 | 125.14 |
| Bone, Giant Cell Tumor of Bone, Primary | 10 | 208.978 | 82.892 | 212.244 |
| Bone, Normal | 8 | 124.117 | 48.471 | 120.579 |
| Bone, Osteosarcoma, Primary | 4 | 172.903 | 40.099 | 187.677 |
| Breast, Infiltrating Carcinoma of Mixed Ductal and Lobular | 8 | 108.947 | 17.335 | 108.756 |
| Type, Primary | | | | |
| Breast, Infiltrating Ductal Carcinoma, Primary | 169 | 139.716 | 54.83 | 131.223 |
| Breast, Infiltrating Lobular Carcinoma, Primary | 17 | 140.044 | 71.903 | 132.439 |
| Breast, Intraductal Carcinoma | 3 | 127.712 | 66.629 | 138.567 |
| Breast, Mucinous Carcinoma, Primary | 4 | 177.408 | 128.251 | 162.643 |
| Breast, Normal | 68 | 144.957 | 49.448 | 139.707 |
| Breast, Phyllodes Tumor (Cystosarcoma Phyllodes), Primary | 5 | 194.148 | 80.426 | 143.412 |
| Colon, Adenocarcinoma (Excluding Mucinous Type), | 77 | 147.279 | 80.655 | 130.934 |
| Primary | | | | |
| Colon, Adenocarcinoma, Mucinous Type, Primary | 7 | 129.576 | 76.123 | 117.097 |
| Colon, Normal | 180 | 109.609 | 50.48 | 107.287 |
| Endometrium, Adenocarcinoma, Endometrioid Type, | 50 | 162 | 71.111 | 142.101 |
| Primary | | | | |
| Endometrium, Mullerian Mixed Tumor, Primary | 7 | 155.66 | 62.996 | 134.38 |
| Endometrium, Normal | 23 | 154.482 | 60.008 | 158.068 |
| Esophagus, Adenocarcinoma, Primary | 3 | 158.602 | 117.853 | 104.145 |
| Esophagus, Normal | 22 | 140.646 | 63.48 | 119.305 |
| Kidney, Carcinoma, Chromophobe Type, Primary | 3 | 141.386 | 41.858 | 148.401 |
| Kidney, Normal | 81 | 179.173 | 82.344 | 166.604 |
| Kidney, Renal Cell Carcinoma, Clear Cell Type, Primary | 45 | 763.988 | 488.604 | 817.291 |
| Kidney, Renal Cell Carcinoma, Non-Clear Cell Type, | 15 | 315.641 | 258.129 | 239.351 |
| Primary | | | | |
| Kidney, Transitional Cell Carcinoma, Primary | 4 | 137.1 | 70.462 | 139.443 |
| Kidney, Wilm's Tumor, Primary | 8 | 133.696 | 41.772 | 119.966 |
| Larynx, Normal | 4 | 134.412 | 62.546 | 118.376 |
| Larynx, Squamous Cell Carcinoma, Primary | 4 | 161.819 | 39.718 | 177.312 |
| Liver, Hepatocellular Carcinoma | 16 | 211.309 | 113.676 | 202.537 |
| Liver, Normal | 42 | 163.819 | 194.899 | 118.909 |
| Lung, Adenocarcinoma, Primary | 46 | 190.999 | 63.168 | 186.342 |
| Lung, Adenosquamous Carcinoma, Primary | 3 | 118.837 | 36.286 | 125.858 |
| Lung, Large Cell Carcinoma, Primary | 7 | 225.434 | 125.006 | 208.652 |
| Lung, Neuroendocrine Carcinoma (Non-Small Cell Type), | 3 | 128.331 | 15.91 | 132.63 |
| Primary | | | | |
| Lung, Normal | 126 | 206.081 | 103.97 | 186.79 |
| Lung, Small Cell Carcinoma, Primary | 3 | 129.72 | 27.533 | 139.847 |
| Lung, Squamous Cell Carcinoma, Primary | 39 | 203.882 | 76.374 | 193.402 |
| Oral Cavity, Squamous Cell Carcinoma, Primary | 3 | 187.011 | 56.588 | 217.093 |
| Ovary, Adenocarcinoma, Clear Cell Type, Primary | 6 | 117.336 | 30.027 | 124.267 |
| Ovary, Adenocarcinoma, Endometrioid Type, Primary | 22 | 141.227 | 70.984 | 120.492 |
| Ovary, Adenocarcinoma, Papillary Serous Type, Primary | 36 | 127.796 | 60.599 | 120.385 |
| Ovary, Granulosa Cell Tumor, Primary | 3 | 100.205 | 32.533 | 81.852 |
| Ovary, Mucinous Cystadenocarcinoma, Primary | 7 | 130.879 | 33.579 | 146.784 |
| Ovary, Mullerian Mixed Tumor, Primary | 5 | 157.225 | 75.293 | 164.511 |
| Ovary, Normal | 89 | 92.269 | 45.755 | 84.056 |
| Pancreas, Adenocarcinoma, Primary | 23 | 231.983 | 77.716 | 221.626 |
| Pancreas, Islet Cell Tumor, Malignant, Primary | 7 | 250.136 | 96.966 | 195.835 |
| Pancreas, Normal | 46 | 143.642 | 55.219 | 132.551 |
| Prostate, Adenocarcinoma, Primary | 86 | 129.853 | 91.797 | 108.61 |
| Prostate, Normal | 57 | 167.226 | 71.922 | 169.295 |
| Rectum, Adenocarcinoma (Excluding Mucinous Type), | 29 | 139.189 | 56.884 | 124.772 |
| Primary | | | | |
| Rectum, Adenocarcinoma, Mucinous Type, Primary | 3 | 89.556 | 31.809 | 72.237 |
| Rectum, Normal | 44 | 117.38 | 49.095 | 109.924 |
| Skin, Basal Cell Carcinoma, Primary | 4 | 133.536 | 71.765 | 126.292 |
| Skin, Malignant Melanoma, Primary | 7 | 105.148 | 56.109 | 75.886 |
| Skin, Normal | 61 | 127.806 | 44.362 | 118.749 |
| Skin, Squamous Cell Carcinoma, Primary | 4 | 173.046 | 30.208 | 174.057 |
| Small Intestine, Gastrointestinal Stromal Tumor (GIST), | 4 | 212.338 | 88.898 | 177.183 |
| Primary | | | | |
| Small Intestine, Normal | 97 | 120.66 | 42.031 | 112.947 |
| Stomach, Adenocarcinoma (Excluding Signet Ring Cell | 27 | 151.819 | 53.342 | 138.801 |
| Type), Primary | | | | |
| Stomach, Adenocarcinoma, Signet Ring Cell Type, Primary | 9 | 181.654 | 47.637 | 181.526 |
| Stomach, Gastrointestinal Stromal Tumor (GIST), Primary | 9 | 155.728 | 107.806 | 113.455 |
| Stomach, Normal | 52 | 135.918 | 42.117 | 139.831 |
| Thyroid Gland, Follicular Carcinoma, Primary | 3 | 222.44 | 128.368 | 277.516 |
| Thyroid Gland, Normal | 24 | 372.974 | 102.414 | 337.823 |
| Thyroid Gland, Papillary Carcinoma, Primary; All Variants | 29 | 297.717 | 136.673 | 247.497 |
| Urinary Bladder, Normal | 9 | 190.26 | 93.234 | 152.274 |
| Urinary Bladder, Transitional Cell Carcinoma, Primary | 4 | 273.824 | 262.168 | 161.156 |
| Uterine Cervix, Adenocarcinoma, Primary | 3 | 160.544 | 59.888 | 128.978 |
| Uterine Cervix, Normal | 115 | 183.173 | 96.843 | 170.376 |
| Vulva, Normal | 4 | 190.585 | 45.15 | 188.274 |
| Vulva, Squamous Cell Carcinoma, Primary | 5 | 220.708 | 42.917 | 234.018 |

Vascular Endothelial Growth Factor Receptor 2 (VEGFR2)

As discussed above, the tyrosine kinase receptor family of VEGFR, which plays a role in angiogenesis, is a potential target for the development of anticancer therapeutic agents. Experiments were thus conducted to determine if a correlative relationship exists between PARP and VEGFR2 expression in a variety of tumor tissue samples. Table XXVII depicts the level of expression in a variety of tissues. As seen, VEGFR2 is upregulated and co-regulated in the same subtype of tumors as PARP1 is upregulated, such as tumors of breast, ovarian and skin tumors and sarcomas. Accordingly, one embodiment is the treatment of susceptible diseases with a combination of PARP and VEGFR modulators. Moreover, VEGFR2 related genes, including genes co-regulated in the VEGFR2 pathway, are also contemplated herein.

TABLE XXVII
Expression of VEGFR2 (vascular endothelial growth factor receptor 2,
kinase insert domain receptor (a type III receptor tyrosine kinase)) in
human primary tumors in comparison with normal tissues.

| | Sample | | | |
|---|---|---|---|---|
| Sample Set | Count | Mean | Std. Dev. | Median |
| Adrenal Gland, Adrenal Cortical Carcinoma, Primary | 3 | 54.418 | 16.608 | 54.696 |
| Adrenal Gland, Normal | 13 | 111.67 | 121.562 | 66.839 |
| Bone, Giant Cell Tumor of Bone, Primary | 10 | 54.808 | 21.963 | 52.183 |
| Bone, Normal | 8 | 72.551 | 29.122 | 64.245 |
| Bone, Osteosarcoma, Primary | 4 | 55.346 | 17.552 | 55.116 |
| Breast, Infiltrating Carcinoma of Mixed Ductal and | 8 | 38.151 | 9.897 | 40.119 |
| Lobular Type, Primary | | | | |
| Breast, Infiltrating Ductal Carcinoma, Primary | 169 | 45.243 | 17.55 | 44.149 |
| Breast, Infiltrating Lobular Carcinoma, Primary | 17 | 57.124 | 23.57 | 52.747 |
| Breast, Intraductal Carcinoma | 3 | 55.079 | 16.518 | 61.707 |
| Breast, Mucinous Carcinoma, Primary | 4 | 49.099 | 33.814 | 40.821 |
| Breast, Normal | 68 | 72.812 | 29.255 | 66.472 |
| Breast, Phyllodes Tumor (Cystosarcoma Phyllodes), | 5 | 88.855 | 36.644 | 73.775 |
| Primary | | | | |
| Colon, Adenocarcinoma (Excluding Mucinous Type), | 77 | 33.293 | 16.994 | 30.262 |
| Primary | | | | |
| Colon, Adenocarcinoma, Mucinous Type, Primary | 7 | 33.315 | 8.847 | 32.644 |
| Colon, Normal | 180 | 31.22 | 15.867 | 27.868 |
| Endometrium, Adenocarcinoma, Endometrioid Type, | 50 | 42.819 | 27.836 | 36.227 |
| Primary | | | | |
| Endometrium, Mullerian Mixed Tumor, Primary | 7 | 35.176 | 14.565 | 30.606 |
| Endometrium, Normal | 23 | 118.847 | 90.297 | 105.117 |
| Esophagus, Adenocarcinoma, Primary | 3 | 36.744 | 14.795 | 33.667 |
| Esophagus, Normal | 22 | 34.456 | 10.861 | 33.479 |
| Kidney, Carcinoma, Chromophobe Type, Primary | 3 | 45.755 | 28.875 | 32.784 |
| Kidney, Normal | 81 | 78.391 | 29.358 | 75.001 |
| Kidney, Renal Cell Carcinoma, Clear Cell Type, Primary | 45 | 178.44 | 145.319 | 142.553 |
| Kidney, Renal Cell Carcinoma, Non-Clear Cell Type, | 15 | 102.066 | 105.1 | 56.906 |
| Primary | | | | |
| Kidney, Transitional Cell Carcinoma, Primary | 4 | 28.451 | 12.694 | 24.175 |
| Kidney, Wilm's Tumor, Primary | 8 | 49.808 | 24.211 | 51.614 |
| Larynx, Normal | 4 | 49.429 | 6.255 | 51.377 |
| Larynx, Squamous Cell Carcinoma, Primary | 4 | 44.504 | 20.342 | 35.819 |
| Liver, Hepatocellular Carcinoma | 16 | 67.244 | 28.225 | 68.843 |
| Liver, Normal | 42 | 87.754 | 40.675 | 84.103 |
| Lung, Adenocarcinoma, Primary | 46 | 61.276 | 31.117 | 51.565 |
| Lung, Adenosquamous Carcinoma, Primary | 3 | 56.68 | 35.265 | 43.723 |
| Lung, Large Cell Carcinoma, Primary | 7 | 40.867 | 38.503 | 29.793 |
| Lung, Neuroendocrine Carcinoma (Non-Small Cell | 3 | 53.965 | 39.357 | 40.297 |
| Type), Primary | | | | |
| Lung, Normal | 126 | 111.651 | 47.136 | 107.643 |
| Lung, Small Cell Carcinoma, Primary | 3 | 22.696 | 9.35 | 24.654 |
| Lung, Squamous Cell Carcinoma, Primary | 39 | 37.921 | 16.918 | 35.459 |
| Oral Cavity, Squamous Cell Carcinoma, Primary | 3 | 27.326 | 5.753 | 24.035 |
| Ovary, Adenocarcinoma, Clear Cell Type, Primary | 6 | 35.485 | 19.253 | 30.079 |
| Ovary, Adenocarcinoma, Endometrioid Type, Primary | 22 | 32.288 | 14.611 | 29.366 |
| Ovary, Adenocarcinoma, Papillary Serous Type, Primary | 36 | 29.226 | 11.714 | 25.12 |
| Ovary, Granulosa Cell Tumor, Primary | 3 | 38.018 | 6.286 | 34.969 |
| Ovary, Mucinous Cystadenocarcinoma, Primary | 7 | 34.894 | 7.065 | 34.569 |
| Ovary, Mullerian Mixed Tumor, Primary | 5 | 19.053 | 7.903 | 16.049 |
| Ovary, Normal | 89 | 44.58 | 15.589 | 43.665 |
| Pancreas, Adenocarcinoma, Primary | 23 | 40.994 | 16.987 | 38.622 |
| Pancreas, Islet Cell Tumor, Malignant, Primary | 7 | 76.18 | 45.816 | 68.714 |
| Pancreas, Normal | 46 | 43.239 | 15.192 | 40.642 |
| Prostate, Adenocarcinoma, Primary | 86 | 37.848 | 16.065 | 32.759 |
| Prostate, Normal | 57 | 52.378 | 22.855 | 50.076 |
| Rectum, Adenocarcinoma (Excluding Mucinous Type), | 29 | 35.377 | 12.352 | 35.386 |
| Primary | | | | |
| Rectum, Adenocarcinoma, Mucinous Type, Primary | 3 | 28.283 | 11.811 | 21.766 |
| Rectum, Normal | 44 | 28.944 | 14.854 | 25.861 |
| Skin, Basal Cell Carcinoma, Primary | 4 | 42.488 | 20.683 | 43.236 |
| Skin, Malignant Melanoma, Primary | 7 | 39.168 | 10.039 | 40.545 |
| Skin, Normal | 61 | 59.014 | 24.546 | 54.485 |
| Skin, Squamous Cell Carcinoma, Primary | 4 | 50.418 | 15.958 | 54.986 |
| Small Intestine, Gastrointestinal Stromal Tumor (GIST), | 4 | 31.127 | 12.326 | 31.387 |
| Primary | | | | |
| Small Intestine, Normal | 97 | 31.744 | 15.843 | 28.931 |
| Stomach, Adenocarcinoma (Excluding Signet Ring Cell | 27 | 39.251 | 18.89 | 36.631 |
| Type), Primary | | | | |
| Stomach, Adenocarcinoma, Signet Ring Cell Type, | 9 | 33.975 | 12.855 | 29.06 |
| Primary | | | | |
| Stomach, Gastrointestinal Stromal Tumor (GIST), | 9 | 70.241 | 131.243 | 23.443 |
| Primary | | | | |
| Stomach, Normal | 52 | 38.534 | 13.998 | 35.883 |
| Thyroid Gland, Follicular Carcinoma, Primary | 3 | 56.578 | 7.441 | 54.753 |
| Thyroid Gland, Normal | 24 | 137.266 | 40.699 | 137.41 |
| Thyroid Gland, Papillary Carcinoma, Primary; All | 29 | 95.774 | 49.594 | 87 |
| Variants | | | | |
| Urinary Bladder, Normal | 9 | 51.661 | 30.22 | 36.98 |
| Urinary Bladder, Transitional Cell Carcinoma, Primary | 4 | 38.644 | 12.864 | 33.928 |
| Uterine Cervix, Adenocarcinoma, Primary | 3 | 59.629 | 5.755 | 59.743 |
| Uterine Cervix, Normal | 115 | 82.943 | 40.489 | 75.229 |
| Vulva, Normal | 4 | 55.41 | 9.211 | 53.173 |
| Vulva, Squamous Cell Carcinoma, Primary | 5 | 53.617 | 25.435 | 47.715 |

Interleukin 1 Receptor Associated Kinase 1 (IRAK1)

Interleukin-1 is a proinflammatory cytokine that functions in the generation of systemic and local response to infection, injury, and immunologic challenges. IL1, produced mainly by induced macrophages and monocytes, participates in lymphocyte activation, fever, leukocyte trafficking, the acute phase response, and cartilage remodeling. The biologic activities of IL1 are mediated by its type I receptor located on the plasma membrane of responsive cells. Binding of IL1 to its receptor triggers activation of nuclear factor kappa-B, a family of related transcription factors that regulates the expression of genes bearing cognate DNA binding sites. NF-kappa-B is retained in the cytoplasm of most cells by the inhibitory kappa-B proteins. The inhibitory protein is degraded in response to a variety of extracellular stimuli, including IL1, releasing NF-kappa-B to enter the nucleus where it activates an array of genes. Interleukin-1 receptor activated kinases (IRAKs) are key mediators in the signaling pathways of IL-1 receptors. IRAK1 is an essential mechanism of NF-kB activation as was found in the experiments with Irak-deficient mice that demonstrated diminished NFKB activation.

Experiments were conducted to determine if a correlative relationship exists between PARP and IRAK1 expression in a variety of tumor tissue samples. Table XXVIII depicts the level of expression in a variety of tissues. As seen, IRAK1 is upregulated and co-regulated in the same subtype of tumors as PARP1 is upregulated, such as tumors of breast, endometrium, ovarian and lung tumors and sarcomas. Accordingly, one embodiment is the treatment of susceptible diseases with a combination of PARP and IRAK1 modulators. Moreover, IRAK1 related genes, including genes co-regulated in the VEGFR pathway, are also contemplated herein.

TABLE XXVIII
Expression of IRAK1 (interleukin 1 receptor associated kinase 1) in human primary tumors in
comparison with normal tissues.

| | Sample | | | |
|---|---|---|---|---|
| Sample Set | Count | Mean | Std. Dev. | Median |
| Adrenal Gland, Adrenal Cortical Carcinoma, Primary | 3 | 673.561 | 474.546 | 500.804 |
| Adrenal Gland, Normal | 13 | 459.673 | 151.366 | 454.364 |
| Bone, Giant Cell Tumor of Bone, Primary | 10 | 391.207 | 133.291 | 371.409 |
| Bone, Normal | 8 | 397.607 | 117.151 | 372.114 |
| Bone, Osteosarcoma, Primary | 4 | 479.645 | 49.624 | 465.032 |
| Breast, Infiltrating Carcinoma of Mixed Ductal and | 8 | 636.321 | 642.372 | 413.28 |
| Lobular Type, Primary | | | | |
| Breast, Infiltrating Ductal Carcinoma, Primary | 169 | 456.616 | 211.377 | 401.965 |
| Breast, Infiltrating Lobular Carcinoma, Primary | 17 | 350.163 | 151.82 | 314.908 |
| Breast, Intraductal Carcinoma | 3 | 245.276 | 70.2 | 209.671 |
| Breast, Mucinous Carcinoma, Primary | 4 | 335.537 | 79.055 | 316.279 |
| Breast, Normal | 68 | 323.839 | 107.498 | 301.842 |
| Breast, Phyllodes Tumor (Cystosarcoma Phyllodes), | 5 | 292.625 | 53.779 | 286.932 |
| Primary | | | | |
| Colon, Adenocarcinoma (Excluding Mucinous Type), | 77 | 621.857 | 244.1 | 569.836 |
| Primary | | | | |
| Colon, Adenocarcinoma, Mucinous Type, Primary | 7 | 599.666 | 189.643 | 504.995 |
| Colon, Normal | 180 | 388.56 | 124.057 | 365.397 |
| Endometrium, Adenocarcinoma, Endometrioid Type, | 50 | 326.862 | 132.076 | 310.135 |
| Primary | | | | |
| Endometrium, Mullerian Mixed Tumor, Primary | 7 | 442.289 | 171.683 | 475.694 |
| Endometrium, Normal | 23 | 237.621 | 106.731 | 219.986 |
| Esophagus, Adenocarcinoma, Primary | 3 | 1091.677 | 116.454 | 1149.642 |
| Esophagus, Normal | 22 | 376.737 | 120.868 | 360.387 |
| Kidney, Carcinoma, Chromophobe Type, Primary | 3 | 281.963 | 27.212 | 280.497 |
| Kidney, Normal | 81 | 302.706 | 88.382 | 305.896 |
| Kidney, Renal Cell Carcinoma, Clear Cell Type, Primary | 45 | 365.557 | 116.429 | 348.144 |
| Kidney, Renal Cell Carcinoma, Non-Clear Cell Type, | 15 | 469.698 | 204.005 | 385.459 |
| Primary | | | | |
| Kidney, Transitional Cell Carcinoma, Primary | 4 | 451.774 | 131.753 | 493.001 |
| Kidney, Wilm's Tumor, Primary | 8 | 306.802 | 105.516 | 307.513 |
| Larynx, Normal | 4 | 437.626 | 182.359 | 452.501 |
| Larynx, Squamous Cell Carcinoma, Primary | 4 | 535.586 | 192.651 | 499.768 |
| Liver, Hepatocellular Carcinoma | 16 | 398.31 | 157.464 | 395.092 |
| Liver, Normal | 42 | 177.604 | 62.495 | 168.052 |
| Lung, Adenocarcinoma, Primary | 46 | 573.945 | 263.63 | 529.26 |
| Lung, Adenosquamous Carcinoma, Primary | 3 | 422.739 | 45.237 | 425.833 |
| Lung, Large Cell Carcinoma, Primary | 7 | 548.695 | 222.506 | 499.715 |
| Lung, Neuroendocrine Carcinoma (Non-Small Cell Type), | 3 | 362.296 | 228.291 | 283.294 |
| Primary | | | | |
| Lung, Normal | 126 | 299.378 | 105.865 | 281.969 |
| Lung, Small Cell Carcinoma, Primary | 3 | 302.829 | 71.079 | 274.84 |
| Lung, Squamous Cell Carcinoma, Primary | 39 | 586.278 | 231.736 | 546.641 |
| Oral Cavity, Squamous Cell Carcinoma, Primary | 3 | 652.55 | 484.533 | 377.583 |
| Ovary, Adenocarcinoma, Clear Cell Type, Primary | 6 | 403.469 | 165.346 | 345.298 |
| Ovary, Adenocarcinoma, Endometrioid Type, Primary | 22 | 480.493 | 267.492 | 420.408 |
| Ovary, Adenocarcinoma, Papillary Serous Type, Primary | 36 | 550.768 | 297.353 | 518.682 |
| Ovary, Granulosa Cell Tumor, Primary | 3 | 204.326 | 9.245 | 199.434 |
| Ovary, Mucinous Cystadenocarcinoma, Primary | 7 | 446.244 | 157.448 | 408.978 |
| Ovary, Mullerian Mixed Tumor, Primary | 5 | 459.58 | 261.132 | 387.474 |
| Ovary, Normal | 89 | 193.631 | 70.936 | 183.31 |
| Pancreas, Adenocarcinoma, Primary | 23 | 408.518 | 108.348 | 409.698 |
| Pancreas, Islet Cell Tumor, Malignant, Primary | 7 | 616.628 | 260.06 | 494.256 |
| Pancreas, Normal | 46 | 337.27 | 109.44 | 306.728 |
| Prostate, Adenocarcinoma, Primary | 86 | 437.337 | 128.249 | 424.415 |
| Prostate, Normal | 57 | 337.15 | 75.629 | 324.359 |
| Rectum, Adenocarcinoma (Excluding Mucinous Type), | 29 | 667.234 | 209.823 | 644.219 |
| Primary | | | | |
| Rectum, Adenocarcinoma, Mucinous Type, Primary | 3 | 641.685 | 183.696 | 707.031 |
| Rectum, Normal | 44 | 376.082 | 118.912 | 357.174 |
| Skin, Basal Cell Carcinoma, Primary | 4 | 240.874 | 35.248 | 238.726 |
| Skin, Malignant Melanoma, Primary | 7 | 358.732 | 136.687 | 357.463 |
| Skin, Normal | 61 | 405.686 | 109.659 | 389.601 |
| Skin, Squamous Cell Carcinoma, Primary | 4 | 417.131 | 49.109 | 410.967 |
| Small Intestine, Gastrointestinal Stromal Tumor (GIST), | 4 | 207.223 | 71.481 | 192.011 |
| Primary | | | | |
| Small Intestine, Normal | 97 | 496.133 | 169.772 | 480.523 |
| Stomach, Adenocarcinoma (Excluding Signet Ring Cell | 27 | 616.382 | 262.711 | 548.388 |
| Type), Primary | | | | |
| Stomach, Adenocarcinoma, Signet Ring Cell Type, | 9 | 783.841 | 628.775 | 572.466 |
| Primary | | | | |
| Stomach, Gastrointestinal Stromal Tumor (GIST), | 9 | 232.296 | 75.708 | 242.608 |
| Primary | | | | |
| Stomach, Normal | 52 | 380.597 | 157.268 | 340.104 |
| Thyroid Gland, Follicular Carcinoma, Primary | 3 | 257.712 | 97.865 | 292.424 |
| Thyroid Gland, Normal | 24 | 161.685 | 52.119 | 146.901 |
| Thyroid Gland, Papillary Carcinoma, Primary; All | 29 | 197.349 | 99.501 | 185.737 |
| Variants | | | | |
| Urinary Bladder, Normal | 9 | 235.241 | 107.541 | 204.569 |
| Urinary Bladder, Transitional Cell Carcinoma, Primary | 4 | 302.469 | 150.232 | 270.951 |
| Uterine Cervix, Adenocarcinoma, Primary | 3 | 309.646 | 106.687 | 289.85 |
| Uterine Cervix, Normal | 115 | 232.08 | 96.727 | 214.625 |
| Vulva, Normal | 4 | 328.463 | 119.872 | 280.431 |
| Vulva, Squamous Cell Carcinoma, Primary | 5 | 363.919 | 110.84 | 399.783 |

V-ErbB2 Erythroblastic Leukemia Viral Oncogene Homolog 3 (ERBB3)

The expression of Epidermal Growth Factor Receptor (EGFR), a tyrosine kinase receptor, has been implicated as necessary in the development of adenomas and carcinomas in intestinal tumors, and subsequent expansion of initiated tumors (Roberts et al., 2002, PNAS, 99:1521-1526). Overexpression of EGFR also plays a role in neoplasia, especially in tumors of epithelial origin (Kari et al., 2003, Cancer Res., 63:1-5). EGFR is a member of the ErbB family of receptors, which includes HER2c/neu, Her2 and Her3 receptor tyrosine kinases.

One critical EGFR pathway involves the oncogene ERBB3 (also known as HER23), which is a member of the HER-family of receptor tyrosine kinases, including HER1/EGFR/c-erbB2, HER4/c-erbB4. The HER-family shares a high degree of structural and functional homology. HER signaling promotes tumorigenesis, mostly through activation of the PI3K/Akt pathway, and is driven predominantly through phosphorylation in trans of the kinase inactive member HER3, highlighting the functional significance of HER3 in the regulation of tumor cell proliferation. Moreover, the HER-family constitutes a complex network, coupling various extracellular ligands to intracellular signal transduction pathways, resulting in receptor interaction and cross activation of the members of the HER-family. For example, the formation of HER2/HER3 heterodimers creates mitogenic and transforming receptor complexes within the HER (erbB) family.

Experiments were conducted to determine if a correlative relationship exists between PARP and ERBB3 expression exists in a variety of tissue samples. Table XXIX depicts the level of expression in a variety of tissues. As seen, ERBB3 is upregulated and co-regulated in the same subtype of tumors as PARP1 is upregulated, such as tumors of breast, ovary, and skin tumors and sarcomas. Accordingly, one embodiment is the treatment of susceptible diseases with a combination of PARP and ERBB3 modulators. Moreover, ERBB3 related genes, including genes co-regulated in the ERBB3 pathway, are also contemplated herein.

TABLE XXIX
Expression of ERBB3 (v-erb-b2 erythroblastic leukemia viral oncogene homolog 3) in human primary
tumors in comparison with normal tissues

| | Sample | | | |
|---|---|---|---|---|
| Sample Set | Count | Mean | Std. Dev. | Median |
| Adrenal Gland, Adrenal Cortical Carcinoma, Primary | 3 | 577.882 | 980.547 | 14.285 |
| Adrenal Gland, Normal | 13 | 125.524 | 343.556 | 18.187 |
| Bone, Giant Cell Tumor of Bone, Primary | 10 | 10.336 | 7.223 | 9.132 |
| Bone, Normal | 8 | 37.284 | 57.615 | 14.053 |
| Bone, Osteosarcoma, Primary | 4 | 20.579 | 17.253 | 18.759 |
| Breast, Infiltrating Carcinoma of Mixed Ductal and | 8 | 2280.914 | 1187.289 | 2134.499 |
| Lobular Type, Primary | | | | |
| Breast, Infiltrating Ductal Carcinoma, Primary | 169 | 1548.723 | 857.043 | 1416.273 |
| Breast, Infiltrating Lobular Carcinoma, Primary | 17 | 2063.404 | 1228.354 | 1905.583 |
| Breast, Intraductal Carcinoma | 3 | 2912.882 | 391.626 | 2915.354 |
| Breast, Mucinous Carcinoma, Primary | 4 | 1540.657 | 647.821 | 1335.309 |
| Breast, Normal | 68 | 1113.455 | 580.417 | 1092.339 |
| Breast, Phyllodes Tumor (Cystosarcoma Phyllodes), | 5 | 537.381 | 166.451 | 530.115 |
| Primary | | | | |
| Colon, Adenocarcinoma (Excluding Mucinous Type), | 77 | 1971.768 | 746.859 | 1840.703 |
| Primary | | | | |
| Colon, Adenocarcinoma, Mucinous Type, Primary | 7 | 1430.242 | 808.398 | 1351.427 |
| Colon, Normal | 180 | 1458.433 | 515.98 | 1383.82 |
| Endometrium, Adenocarcinoma, Endometrioid Type, | 50 | 758.705 | 441.307 | 671.915 |
| Primary | | | | |
| Endometrium, Mullerian Mixed Tumor, Primary | 7 | 391.366 | 552.712 | 92.314 |
| Endometrium, Normal | 23 | 499.473 | 409.346 | 332.495 |
| Esophagus, Adenocarcinoma, Primary | 3 | 1853.052 | 965.33 | 1968.129 |
| Esophagus, Normal | 22 | 1013.875 | 393.124 | 1017.246 |
| Kidney, Carcinoma, Chromophobe Type, Primary | 3 | 449.46 | 159.14 | 375.862 |
| Kidney, Normal | 81 | 980.48 | 349.951 | 991.148 |
| Kidney, Renal Cell Carcinoma, Clear Cell Type, | 45 | 942.527 | 714.444 | 765.094 |
| Primary | | | | |
| Kidney, Renal Cell Carcinoma, Non-Clear Cell Type, | 15 | 1184.511 | 985.788 | 1181.861 |
| Primary | | | | |
| Kidney, Transitional Cell Carcinoma, Primary | 4 | 1881.073 | 1688.566 | 1149.255 |
| Kidney, Wilm's Tumor, Primary | 8 | 174.465 | 102.523 | 156.7 |
| Larynx, Normal | 4 | 987.72 | 681.018 | 1184.756 |
| Larynx, Squamous Cell Carcinoma, Primary | 4 | 399.736 | 136.302 | 449.028 |
| Liver, Hepatocellular Carcinoma | 16 | 1623.121 | 904.592 | 1607.987 |
| Liver, Normal | 42 | 963.955 | 470.103 | 837.661 |
| Lung, Adenocarcinoma, Primary | 46 | 1121.085 | 690.427 | 852.101 |
| Lung, Adenosquamous Carcinoma, Primary | 3 | 1110.685 | 512.485 | 1073.488 |
| Lung, Large Cell Carcinoma, Primary | 7 | 772.418 | 399.168 | 558.1 |
| Lung, Neuroendocrine Carcinoma (Non-Small Cell | 3 | 593.582 | 515.062 | 802.766 |
| Type), Primary | | | | |
| Lung, Normal | 126 | 664.625 | 297.552 | 607.42 |
| Lung, Small Cell Carcinoma, Primary | 3 | 314.576 | 136.305 | 383.976 |
| Lung, Squamous Cell Carcinoma, Primary | 39 | 535.679 | 349.395 | 464.982 |
| Oral Cavity, Squamous Cell Carcinoma, Primary | 3 | 632.589 | 681.131 | 255.479 |
| Ovary, Adenocarcinoma, Clear Cell Type, Primary | 6 | 1334.761 | 700.043 | 1133.209 |
| Ovary, Adenocarcinoma, Endometrioid Type, Primary | 22 | 880.946 | 425.324 | 770.453 |
| Ovary, Adenocarcinoma, Papillary Serous Type, | 36 | 982.248 | 604.01 | 779.513 |
| Primary | | | | |
| Ovary, Granulosa Cell Tumor, Primary | 3 | 12.718 | 5.99 | 13.055 |
| Ovary, Mucinous Cystadenocarcinoma, Primary | 7 | 1448.166 | 459.784 | 1443.369 |
| Ovary, Mullerian Mixed Tumor, Primary | 5 | 537.117 | 543.134 | 496.456 |
| Ovary, Normal | 89 | 62.734 | 174.184 | 26.506 |
| Pancreas, Adenocarcinoma, Primary | 23 | 1127.646 | 680.621 | 889.292 |
| Pancreas, Islet Cell Tumor, Malignant, Primary | 7 | 1230.09 | 1379.954 | 844.986 |
| Pancreas, Normal | 46 | 466.353 | 163.486 | 426.184 |
| Prostate, Adenocarcinoma, Primary | 86 | 1655.44 | 477.053 | 1574.154 |
| Prostate, Normal | 57 | 992.882 | 394.393 | 1007.848 |
| Rectum, Adenocarcinoma (Excluding Mucinous Type), | 29 | 1844.5 | 734.105 | 1699.542 |
| Primary | | | | |
| Rectum, Adenocarcinoma, Mucinous Type, Primary | 3 | 1159.982 | 1067.734 | 838.012 |
| Rectum, Normal | 44 | 1328.401 | 449.394 | 1237.417 |
| Skin, Basal Cell Carcinoma, Primary | 4 | 635.797 | 278.09 | 622.684 |
| Skin, Malignant Melanoma, Primary | 7 | 2547.3 | 2402.871 | 1875.538 |
| Skin, Normal | 61 | 783.091 | 377.959 | 747.794 |
| Skin, Squamous Cell Carcinoma, Primary | 4 | 301.374 | 121.643 | 335.271 |
| Small Intestine, Gastrointestinal Stromal Tumor (GIST), | 4 | 11.31 | 10.04 | 8.432 |
| Primary | | | | |
| Small Intestine, Normal | 97 | 1790.03 | 773.198 | 1825.371 |
| Stomach, Adenocarcinoma (Excluding Signet Ring Cell | 27 | 1411.513 | 670.095 | 1388.222 |
| Type), Primary | | | | |
| Stomach, Adenocarcinoma, Signet Ring Cell Type, | 9 | 1138.628 | 228.311 | 1053.921 |
| Primary | | | | |
| Stomach, Gastrointestinal Stromal Tumor (GIST), | 9 | 13.944 | 11.315 | 7.565 |
| Primary | | | | |
| Stomach, Normal | 52 | 1148.508 | 506.496 | 1140.674 |
| Thyroid Gland, Follicular Carcinoma, Primary | 3 | 535.996 | 284.787 | 420.907 |
| Thyroid Gland, Normal | 24 | 160.13 | 77.384 | 139.421 |
| Thyroid Gland, Papillary Carcinoma, Primary; All | 29 | 368.881 | 394.066 | 205.043 |
| Variants | | | | |
| Urinary Bladder, Normal | 9 | 304.776 | 186.305 | 250.217 |
| Urinary Bladder, Transitional Cell Carcinoma, Primary | 4 | 1698.328 | 860.141 | 1647.78 |
| Uterine Cervix, Adenocarcinoma, Primary | 3 | 533.276 | 625.49 | 206.731 |
| Uterine Cervix, Normal | 115 | 353.483 | 199.167 | 290.434 |
| Vulva, Normal | 4 | 671.006 | 249.678 | 757.337 |
| Vulva, Squamous Cell Carcinoma, Primary | 5 | 345.409 | 144.583 | 390.85 |

Migration Inhibitory Factor

Tumor-associated macrophages may influence tumor progression, angiogenesis and invasion. Migration inhibitory factor (MIF) is a pleotropic cytokine which plays a pivotal role in inflammatory and immune-mediated diseases, such as rheumatoid arthritis (RA) and atherosclerosis. MIF is secreted by T lymphocytes and macrophages on lipopolysaccharide (LPS) exposure and induces secretion of tumor necrosis factor-α (TNF-α) by mouse macrophages. MIF is highly expressed in macrophages, endothelial cells, synovial tissue (ST) fibroblasts, serum, and synovial fluids. MIF stimulates macrophage release of proinflammatory cytokines such as TNF-α, interleukin 1 β (IL-1β), IL-6, and IL-8. MIF up-regulates IL-1β, matrix metalloproteinases (MMPs) MMP-1, MMP-3, MMP-9, and MMP-13 in RAST fibroblasts. In rodent arthritis models, administration of anti-MIF antibody ameliorates arthritis, with profound inhibition of clinical and histologic features of disease. Anti-MIF treatment also improves the outcome of acute encephalomyelitis and experimental autoimmune myocarditis in mice. These studies show a key role of MIF in the pathogenesis of immunologic and inflammatory diseases. It was also that that MIF is a potent angiogenic factor. MIF can up-regulate VCAM-1 and ICAM-1 via Src, PI3K, and NFκB activation.

Because of MIF's key role in disease progression, modulation of MIF expression is seen as a likely therapeutic target. Accordingly, one embodiment is the treatment of susceptible diseases with a combination of PARP and MIF modulators. Moreover, MIF related genes, including genes co-regulated in the MIF pathway, are also contemplated herein.

VAV3 Oncogene

VAV proteins are guanine nucleotide exchange factors (GEFs) for Rho family GTPases that activate pathways leading to actin cytoskeletal rearrangements and transcriptional alterations. VAV3 acts as a GEF preferentially on RhoG (ARHG), RhoA (ARHA, and, to a lesser extent, RAC1, and it associates maximally with these GTPases in the nucleotide-free state. Investigators have identified a splice variant of VAV3, which they termed VAV3.1, that contains only the C-terminal SH3-SH2-SH3 region. VAV3.1 appeared to be downregulated by EGF and transforming growth factor-beta (TGFB). VAV3 was also shown to enhance nuclear factor kappa-B (NFKB)-dependent transcription.

Because of VAV3's key role in disease progression, modulation of VAV3 expression is seen as a likely therapeutic target. Accordingly, one embodiment is the treatment of susceptible diseases with a combination of PARP and VAV3 modulators. Moreover, VAV3 related genes, including genes co-regulated in the VAV3 pathway, are also contemplated herein.

Aurora Kinase

Aurora kinase A (AURKA) is a mitotic centrosomal protein kinase (Kimura et al., 1997, J. Biol. Chem. 272:13766-13771). The main role of AURKA in tumor development is in controlling chromosome segregation during mitosis (Bischoff and Plowman, 1999, Trends Cell Biol. 9:454-459). AURKA is frequently amplified in cancer, and induces phosphorylation of IkappaBa, thereby mediating its degradation. Loss of IkappaBa leads to activation of NF-kappaB target gene transcription. In human primary breast cancers, 13.6% of samples showed AURKA gene amplification, all of which exhibited nuclear localization of NF-kappaB, suggesting that this particular subgroup of breast cancer patients might benefit from inhibiting AURKA.

Moreover, the analysis of different human tumor cell types for NF-kappaB activity has showed that there is an association between cell resistance to chemotherapeutic agents and NF-kappaB activation. For example, A549 human lung adenocarcinoma cells and SKOV3 human ovarian cancer cells have high levels of NF-kappaB and are resistant to cytotoxic agents such as adriamycin and VP-16 (etoposide). It was also shown that in A549 and SKOV3 cells treated with a small molecule inhibitor towards Aurora kinases, NF-kappaB, Bcl-XL and Bcl-2 activity was downregulated along with the concomitant increase in efficacy of cytotoxic drugs. These findings have important implications for cancer chemotherapy. AURKA-inhibition enhances the efficacy of chemotherapeutic agents and reverses acquired resistance resulting from the activation of NF-kappaB. Consequently, preventing NF-kappaB activation by inhibition of AURKA may provide a valuable enhancement to specific chemotherapeutic regimens (Linardopoulos, 2007, J BUON. 12(Suppl 1):S67-70).

Experiments were conducted to determine if a correlative relationship exists between PARP and AURKA expression exists in a variety of tissue samples. Table XXX depicts the level of expression in a variety of tissues. As seen, AURKA is upregulated and co-regulated in the same subtype of tumors as PARP1 is upregulated, such as tumors of breast, endometrium, lung and ovarian tumors and sarcomas. Accordingly, one embodiment is the treatment of susceptible diseases with a combination of PARP and AURKA modulators. Moreover, AURKA related genes, including genes co-regulated in the AURKA pathway, are also contemplated herein.

TABLE XXX
Expression of Aurora Kinase A in human primary tumors in comparison with normal tissues.

| | Sample | | | |
|---|---|---|---|---|
| Sample Set | Count | Mean | Std. Dev. | Median |
| Adrenal Gland, Adrenal Cortical Carcinoma, Primary | 3 | 44.754 | 8.862 | 43.392 |
| Adrenal Gland, Normal | 13 | 25.672 | 15.905 | 22.076 |
| Bone, Giant Cell Tumor of Bone, Primary | 10 | 51.061 | 18.222 | 48.306 |
| Bone, Normal | 8 | 143.441 | 110.647 | 130.871 |
| Bone, Osteosarcoma, Primary | 4 | 178.04 | 83.591 | 187.41 |
| Breast, Infiltrating Carcinoma of Mixed Ductal and | 8 | 95.51 | 47.454 | 86.491 |
| Lobular Type, Primary | | | | |
| Breast, Infiltrating Ductal Carcinoma, Primary | 169 | 89.343 | 82.104 | 73.288 |
| Breast, Infiltrating Lobular Carcinoma, Primary | 17 | 74.299 | 55.943 | 60.594 |
| Breast, Intraductal Carcinoma | 3 | 74.636 | 71.118 | 49.292 |
| Breast, Mucinous Carcinoma, Primary | 4 | 51.741 | 45.158 | 34.593 |
| Breast, Normal | 68 | 28.743 | 42.088 | 18.843 |
| Breast, Phyllodes Tumor (Cystosarcoma Phyllodes), | 5 | 34.084 | 14.567 | 29.148 |
| Primary | | | | |
| Colon, Adenocarcinoma (Excluding Mucinous Type), | 77 | 162.923 | 85.18 | 142.004 |
| Primary | | | | |
| Colon, Adenocarcinoma, Mucinous Type, Primary | 7 | 112.896 | 42.873 | 101.745 |
| Colon, Normal | 180 | 70.295 | 38.393 | 63.784 |
| Endometrium, Adenocarcinoma, Endometrioid Type, | 50 | 69.564 | 45.648 | 57.714 |
| Primary | | | | |
| Endometrium, Mullerian Mixed Tumor, Primary | 7 | 169.364 | 72.819 | 197.607 |
| Endometrium, Normal | 23 | 36.878 | 56.805 | 20.135 |
| Esophagus, Adenocarcinoma, Primary | 3 | 859.368 | 1198.639 | 203.561 |
| Esophagus, Normal | 22 | 36.408 | 16.133 | 41.23 |
| Kidney, Carcinoma, Chromophobe Type, Primary | 3 | 42.363 | 25.248 | 41.311 |
| Kidney, Normal | 81 | 16.64 | 9.488 | 15.193 |
| Kidney, Renal Cell Carcinoma, Clear Cell Type, | 45 | 34.884 | 24.019 | 27.772 |
| Primary | | | | |
| Kidney, Renal Cell Carcinoma, Non-Clear Cell Type, | 15 | 36.489 | 24.565 | 30.32 |
| Primary | | | | |
| Kidney, Transitional Cell Carcinoma, Primary | 4 | 62.951 | 43.077 | 53.03 |
| Kidney, Wilm's Tumor, Primary | 8 | 134.715 | 48.472 | 137.996 |
| Larynx, Normal | 4 | 38.267 | 7.859 | 40.105 |
| Larynx, Squamous Cell Carcinoma, Primary | 4 | 106.771 | 33.873 | 100.127 |
| Liver, Hepatocellular Carcinoma | 16 | 80.374 | 59.267 | 64.87 |
| Liver, Normal | 42 | 19.333 | 13.529 | 17.57 |
| Lung, Adenocarcinoma, Primary | 46 | 92.449 | 68.175 | 72.573 |
| Lung, Adenosquamous Carcinoma, Primary | 3 | 43.065 | 23.707 | 38.673 |
| Lung, Large Cell Carcinoma, Primary | 7 | 110.99 | 39.237 | 113.89 |
| Lung, Neuroendocrine Carcinoma (Non-Small Cell | 3 | 93.442 | 119.109 | 44.063 |
| Type), Primary | | | | |
| Lung, Normal | 126 | 27.345 | 35.968 | 19.32 |
| Lung, Small Cell Carcinoma, Primary | 3 | 147.378 | 13.136 | 154.126 |
| Lung, Squamous Cell Carcinoma, Primary | 39 | 111.537 | 50.622 | 106.782 |
| Oral Cavity, Squamous Cell Carcinoma, Primary | 3 | 122.089 | 70.313 | 159.159 |
| Ovary, Adenocarcinoma, Clear Cell Type, Primary | 6 | 70.834 | 31.287 | 76.297 |
| Ovary, Adenocarcinoma, Endometrioid Type, Primary | 22 | 64.496 | 36.983 | 57.426 |
| Ovary, Adenocarcinoma, Papillary Serous Type, | 36 | 107.434 | 98.927 | 88.224 |
| Primary | | | | |
| Ovary, Granulosa Cell Tumor, Primary | 3 | 24.753 | 19.999 | 27.065 |
| Ovary, Mucinous Cystadenocarcinoma, Primary | 7 | 33.119 | 14.621 | 31.509 |
| Ovary, Mullerian Mixed Tumor, Primary | 5 | 184.608 | 181.022 | 102.966 |
| Ovary, Normal | 89 | 70.168 | 68.424 | 46.725 |
| Pancreas, Adenocarcinoma, Primary | 23 | 48.758 | 30.381 | 43.699 |
| Pancreas, Islet Cell Tumor, Malignant, Primary | 7 | 39.542 | 25.776 | 28.543 |
| Pancreas, Normal | 46 | 29.429 | 28.901 | 22.729 |
| Prostate, Adenocarcinoma, Primary | 86 | 15.487 | 7.05 | 15.689 |
| Prostate, Normal | 57 | 11.147 | 5.557 | 10.483 |
| Rectum, Adenocarcinoma (Excluding Mucinous Type), | 29 | 158.666 | 66.032 | 153.322 |
| Primary | | | | |
| Rectum, Adenocarcinoma, Mucinous Type, Primary | 3 | 109.484 | 70.156 | 126.287 |
| Rectum, Normal | 44 | 55.244 | 21.11 | 51.151 |
| Skin, Basal Cell Carcinoma, Primary | 4 | 50.118 | 8.463 | 52.27 |
| Skin, Malignant Melanoma, Primary | 7 | 111.153 | 57.768 | 111.744 |
| Skin, Normal | 61 | 21.863 | 32.713 | 15.678 |
| Skin, Squamous Cell Carcinoma, Primary | 4 | 91.039 | 80.277 | 67.971 |
| Small Intestine, Gastrointestinal Stromal Tumor (GIST), | 4 | 27.262 | 20.437 | 23.665 |
| Primary | | | | |
| Small Intestine, Normal | 97 | 61.336 | 31.207 | 59.736 |
| Stomach, Adenocarcinoma (Excluding Signet Ring Cell | 27 | 164.992 | 102.295 | 158.801 |
| Type), Primary | | | | |
| Stomach, Adenocarcinoma, Signet Ring Cell Type, | 9 | 106.468 | 45.98 | 128.174 |
| Primary | | | | |
| Stomach, Gastrointestinal Stromal Tumor (GIST), | 9 | 21.34 | 13.545 | 15.836 |
| Primary | | | | |
| Stomach, Normal | 52 | 51.789 | 28.173 | 47.535 |
| Thyroid Gland, Follicular Carcinoma, Primary | 3 | 36.25 | 50.475 | 12.917 |
| Thyroid Gland, Normal | 24 | 15.556 | 7.707 | 14.658 |
| Thyroid Gland, Papillary Carcinoma, Primary; All | 29 | 23.949 | 13.406 | 21.053 |
| Variants | | | | |
| Urinary Bladder, Normal | 9 | 16.597 | 11.305 | 12.724 |
| Urinary Bladder, Transitional Cell Carcinoma, Primary | 4 | 108.368 | 60.835 | 92.147 |
| Uterine Cervix, Adenocarcinoma, Primary | 3 | 107.466 | 96.964 | 115.821 |
| Uterine Cervix, Normal | 115 | 18.21 | 32.776 | 11.183 |
| Vulva, Normal | 4 | 29.709 | 15.366 | 23.056 |
| Vulva, Squamous Cell Carcinoma, Primary | 5 | 94.718 | 13.914 | 104.197 |

Bcl-2

BCL-2 can promote lymphomagenesis and influence the sensitivity of tumor cells to chemotherapy and radiotherapy. The Bcl-2 family of proteins together are known to include more than 30 proteins with either pro-apoptotic or anti-apoptotic functions, suggesting that they might also play different roles in carcinogenesis (Cory et al., 2003, Oncogene 22:8590-8607). Pro-survival Bcl-2 family members act as oncogenes. Expression of Bcl-2 in transgenic mice confirmed that inhibition of apoptosis can lead to cancer, as these mice develop B cell lymphomas and leukemias. The lifespan of B-lymphoid tumors is significantly prolonged by bcl-2 transgene expression, suggesting that Bcl-2 overexpression provides a predisposition for the development of B-cell lymphomas.

Experiments were conducted to determine if a correlative relationship exists between PARP and Bcl-2 expression exists in a variety of tissue samples. Table XXXI depicts the level of expression in a variety of tissues. As seen, Bcl-2 is upregulated and co-regulated in the same subtype of tumors as PARP1 is upregulated, such as tumors of breast, ovary, and skin tumors and sarcomas. Accordingly, one embodiment is the treatment of susceptible diseases with a combination of PARP and Bcl-2 modulators. Moreover, Bcl-2 related genes, including genes co-regulated in the Bcl-2 pathway, are also contemplated herein.

TABLE XXXI
Expression of BCL2 (B-cell CLL/lymphoma 2) in human primary tumors in comparison
with normal tissues.

| | Sample | | | |
|---|---|---|---|---|
| Sample Set | Count | Mean | Std. Dev. | Median |
| Adrenal Gland, Adrenal Cortical Carcinoma, Primary | 3 | 41.369 | 13.086 | 39.567 |
| Adrenal Gland, Normal | 13 | 76.565 | 79.915 | 57.591 |
| Bone, Giant Cell Tumor of Bone, Primary | 10 | 67.268 | 25.075 | 60.992 |
| Bone Normal | 8 | 93.551 | 37.089 | 101.793 |
| Bone, Osteosarcoma, Primary | 4 | 86.148 | 46.86 | 87.134 |
| Breast, Infiltrating Carcinoma of Mixed Ductal and | 8 | 165.395 | 79.131 | 129.186 |
| Lobular Type, Primary | | | | |
| Breast, Infiltrating Ductal Carcinoma, Primary | 169 | 185.081 | 137.681 | 153.948 |
| Breast, Infiltrating Lobular Carcinoma, Primary | 17 | 253.721 | 170.271 | 188.582 |
| Breast, Intraductal Carcinoma | 3 | 304.094 | 82.093 | 320.92 |
| Breast, Mucinous Carcinoma, Primary | 4 | 231.889 | 174.353 | 202.309 |
| Breast, Normal | 68 | 180.278 | 62.194 | 184.029 |
| Breast, Phyllodes Tumor (Cystosarcoma Phyllodes), | 5 | 156.731 | 53.76 | 158.242 |
| Primary | | | | |
| Colon, Adenocarcinoma (Excluding Mucinous Type), | 77 | 58.51 | 25.967 | 52.622 |
| Primary | | | | |
| Colon, Adenocarcinoma, Mucinous Type, Primary | 7 | 78.225 | 59.629 | 58.656 |
| Colon, Normal | 180 | 99.747 | 38.155 | 94.906 |
| Endometrium, Adenocarcinoma, Endometrioid Type, | 50 | 118.084 | 82.562 | 91.368 |
| Primary | | | | |
| Endometrium, Mullerian Mixed Tumor, Primary | 7 | 76.471 | 24.044 | 80.782 |
| Endometrium, Normal | 23 | 243.099 | 126.075 | 215.948 |
| Esophagus, Adenocarcinoma, Primary | 3 | 37.097 | 14.877 | 32.719 |
| Esophagus, Normal | 22 | 76.845 | 21.677 | 71.56 |
| Kidney, Carcinoma, Chromophobe Type, Primary | 3 | 291.793 | 82.103 | 264.825 |
| Kidney, Normal | 81 | 160.415 | 44.839 | 158.151 |
| Kidney, Renal Cell Carcinoma, Clear Cell Type, | 45 | 213.18 | 109.86 | 185.721 |
| Primary | | | | |
| Kidney, Renal Cell Carcinoma, Non-Clear Cell Type, | 15 | 225.067 | 108.419 | 240.49 |
| Primary | | | | |
| Kidney, Transitional Cell Carcinoma, Primary | 4 | 23.076 | 9.024 | 20.267 |
| Kidney, Wilm's Tumor, Primary | 8 | 150.344 | 52.247 | 132.065 |
| Larynx, Normal | 4 | 108.966 | 91.936 | 68.871 |
| Larynx, Squamous Cell Carcinoma, Primary | 4 | 52.95 | 15.864 | 50.99 |
| Liver, Hepatocellular Carcinoma | 16 | 61.05 | 32.886 | 54.112 |
| Liver, Normal | 42 | 63.025 | 84.148 | 47.745 |
| Lung, Adenocarcinoma, Primary | 46 | 73.211 | 70.81 | 56.933 |
| Lung, Adenosquamous Carcinoma, Primary | 3 | 78.094 | 28.561 | 64.352 |
| Lung, Large Cell Carcinoma, Primary | 7 | 64.283 | 28.099 | 68.291 |
| Lung, Neuroendocrine Carcinoma (Non-Small Cell | 3 | 32.677 | 25.312 | 35.5 |
| Type), Primary | | | | |
| Lung, Normal | 126 | 70.777 | 32.745 | 66.795 |
| Lung, Small Cell Carcinoma, Primary | 3 | 256.362 | 121.664 | 188.266 |
| Lung, Squamous Cell Carcinoma, Primary | 39 | 86.702 | 94.356 | 68.855 |
| Oral Cavity, Squamous Cell Carcinoma, Primary | 3 | 41.448 | 23.986 | 43.03 |
| Ovary, Adenocarcinoma, Clear Cell Type, Primary | 6 | 143.916 | 160.188 | 76.602 |
| Ovary, Adenocarcinoma, Endometrioid Type, Primary | 22 | 116.538 | 91.275 | 85.27 |
| Ovary, Adenocarcinoma, Papillary Serous Type, | 36 | 64.043 | 39.388 | 52.971 |
| Primary | | | | |
| Ovary, Granulosa Cell Tumor, Primary | 3 | 291.661 | 18.052 | 295.117 |
| Ovary, Mucinous Cystadenocarcinoma, Primary | 7 | 96.739 | 102.705 | 67.26 |
| Ovary, Mullerian Mixed Tumor, Primary | 5 | 138.111 | 123.538 | 86.269 |
| Ovary, Normal | 89 | 189.339 | 72.787 | 174.35 |
| Pancreas, Adenocarcinoma, Primary | 23 | 70.77 | 33.311 | 61.929 |
| Pancreas, Islet Cell Tumor, Malignant, Primary | 7 | 44.424 | 16.346 | 42.696 |
| Pancreas, Normal | 46 | 61.713 | 18.442 | 58.003 |
| Prostate, Adenocarcinoma, Primary | 86 | 80.779 | 30.717 | 76.884 |
| Prostate, Normal | 57 | 126.448 | 44.583 | 115.617 |
| Rectum, Adenocarcinoma (Excluding Mucinous Type), | 29 | 49.829 | 13.682 | 47.972 |
| Primary | | | | |
| Rectum, Adenocarcinoma, Mucinous Type, Primary | 3 | 53.416 | 27.606 | 45.316 |
| Rectum, Normal | 44 | 99.686 | 25.97 | 101.939 |
| Skin, Basal Cell Carcinoma, Primary | 4 | 136.707 | 30.101 | 123.82 |
| Skin, Malignant Melanoma, Primary | 7 | 140.862 | 116.907 | 125.858 |
| Skin, Normal | 61 | 104.32 | 35.887 | 99.801 |
| Skin, Squamous Cell Carcinoma, Primary | 4 | 149.226 | 168.298 | 74.5 |
| Small Intestine, Gastrointestinal Stromal Tumor | 4 | 781.493 | 120.352 | 786.203 |
| (GIST), Primary | | | | |
| Small Intestine, Normal | 97 | 98.346 | 51.187 | 92.945 |
| Stomach, Adenocarcinoma (Excluding Signet Ring Cell | 27 | 61.502 | 22.173 | 57.512 |
| Type), Primary | | | | |
| Stomach, Adenocarcinoma, Signet Ring Cell Type, | 9 | 69.446 | 34.59 | 67.033 |
| Primary | | | | |
| Stomach, Gastrointestinal Stromal Tumor (GIST), | 9 | 260.615 | 127.994 | 241.293 |
| Primary | | | | |
| Stomach, Normal | 52 | 65.716 | 26.897 | 58.761 |
| Thyroid Gland, Follicular Carcinoma, Primary | 3 | 315.749 | 209.219 | 435.183 |
| Thyroid Gland, Normal | 24 | 470.013 | 98.75 | 503.828 |
| Thyroid Gland, Papillary Carcinoma, Primary; All | 29 | 209.72 | 107.891 | 214.138 |
| Variants | | | | |
| Urinary Bladder, Normal | 9 | 104.859 | 39.085 | 88.841 |
| Urinary Bladder, Transitional Cell Carcinoma, Primary | 4 | 42.722 | 14.206 | 46.577 |
| Uterine Cervix, Adenocarcinoma, Primary | 3 | 185.839 | 58.711 | 166.966 |
| Uterine Cervix, Normal | 115 | 169.441 | 50.511 | 167.885 |
| Vulva, Normal | 4 | 104.927 | 25.708 | 103.02 |
| Vulva, Squamous Cell Carcinoma, Primary | 5 | 51.488 | 4.185 | 52.544 |

Ubiquitin Proteasome Pathway

The UBIQUITIN-proteasome pathway is the principle mechanism by which cellular proteins are degraded. The proteasome enables a rapid clearance of proteins that are important for cell-cycle progression, including cyclins, cyclin-dependent kinase inhibitors and NF-κB. IkB is polyubiquitylated in response to its phosphorylation by IKK and cleaved by the 26S proteasome. Inhibition of the ubiquitin proteasome pathway results in dysregulation of the cellular proteins involved in cell-cycle control, promotion of tumor growth, and induction of apoptosis. Recently, proteasome inhibitors that have shown promising anticancer responses both in vitro and in vivo have been introduced into the treatment of malignancy. Proteasome inhibitors were originally considered as therapies because they have potential protein targets that are known to be deregulated in tumor cells. Proteasome inhibitors have been reported to alter the levels of the cyclin-dependent kinase inhibitors p21 and p27 (also known as WAF1 and KIP1, respectively) and several pro- and anti-apoptotic proteins leading to cell cycle arrest and apoptosis in several tumor types. Malignant cells are more susceptible to certain proteasome inhibitors and this might be explained, in part, by the destabilization of CDC25A, CDC25C, p27 and the cyclins that are often activated in cancer cells. The orderly and temporal degradation of these regulatory molecules is required for continued cell growth. Therefore, inhibition of proteasome-mediated degradation of these molecules might arrest or retard cell growth. p53 accumulates in response to cellular stress such as chemical- or radiation-induced DNA damage, oncogene activation and hypoxia. MDM2 inhibits the activity of p53, in part by enabling the export of p53 into the cytoplasm, where it can be degraded by the proteasome. p53 becomes stabilized following proteasome inhibition, which can simulate p53-mediated tumor-suppressor activity. Other explanations for the anticancer activity of proteasome inhibitors include the inhibition of IkB degradation, which leads to the maintenance of NFκB in the cytoplasm. NF-κB is considered to be one of the molecules with a central role in mediating many of the effects of proteasome inhibition. An interesting study has demonstrated the extent to which the efficacy of proteasome inhibitors is due to the inhibition of NF-κB. Using multiple myeloma cells, Hideshima et al. compared the effects of an IKK inhibitor, PS-1145, and bortezomib, a proteasome inhibitor that inhibits the chymotryptic activity of the proteasome in a potent, reversible and selective manner (Hideshima et al., 2002, J. Biol. Chem. 277:16639-16647). Although both PS-1145 and bortezomib blocked NFκB activation, bortezomib completely.

Experiments were conducted to determine if a correlative relationship exists between PARP expression and expression of ubiquitin proteasome pathway proteins exists in a variety of tissue samples. Table XXXII depicts the level of expression of UBE2S in a variety of tissues. As seen, UBE2S is upregulated and co-regulated in the same subtype of tumors as PARP1 is upregulated, such as tumors of breast, ovary, and skin tumors and sarcomas. Accordingly, one embodiment is the treatment of susceptible diseases with a combination of PARP and UBE2S modulators. Moreover, UBE2S related genes, including genes co-regulated in the ubiquitin proteasome pathway proteins, are also contemplated herein.

TABLE XXXII
Expression of UBE2S (ubiquitin conjugating enzyme E2S; similar to Ubiquitin-conjugating
enzyme E2S (Ubiquitin-conjugating enzyme E2-24 kDa) (Ubiquitin-protein ligase) (Ubiquitin
carrier protein) (E2-EPF5)) in human primary tumors in comparison with normal tissues.

| | Sample | | | |
|---|---|---|---|---|
| Sample Set | Count | Mean | Std. Dev. | Median |
| Adrenal Gland, Adrenal Cortical Carcinoma, Primary | 3 | 129.097 | 46.893 | 137.935 |
| Adrenal Gland, Normal | 13 | 82.156 | 34.849 | 82.309 |
| Bone, Giant Cell Tumor of Bone, Primary | 10 | 137.94 | 33.664 | 147.67 |
| Bone, Normal | 8 | 145.715 | 104.824 | 122.049 |
| Bone, Osteosarcoma, Primary | 4 | 623.943 | 421.543 | 591.478 |
| Breast, Infiltrating Carcinoma of Mixed Ductal and Lobular | 8 | 150.452 | 73.597 | 149.141 |
| Type, Primary | | | | |
| Breast, Infiltrating Ductal Carcinoma, Primary | 169 | 211.898 | 198.18 | 136.568 |
| Breast, Infiltrating Lobular Carcinoma, Primary | 17 | 121.074 | 102.75 | 98.11 |
| Breast, Intraductal Carcinoma | 3 | 88.188 | 37.496 | 107.824 |
| Breast, Mucinous Carcinoma, Primary | 4 | 228.67 | 158.594 | 184.996 |
| Breast, Normal | 68 | 76.54 | 114.038 | 54.967 |
| Breast, Phyllodes Tumor (Cystosarcoma Phyllodes), Primary | 5 | 151.531 | 44.68 | 144.279 |
| Colon, Adenocarcinoma (Excluding Mucinous Type), Primary | 77 | 292.319 | 191.312 | 239.821 |
| Colon, Adenocarcinoma, Mucinous Type, Primary | 7 | 233.435 | 124.977 | 212.778 |
| Colon, Normal | 180 | 94.723 | 43.203 | 87.05 |
| Endometrium, Adenocarcinoma, Endometrioid Type, Primary | 50 | 189.219 | 143.485 | 151.341 |
| Endometrium, Mullerian Mixed Tumor, Primary | 7 | 423.028 | 199.339 | 377.047 |
| Endometrium, Normal | 23 | 83.824 | 45.485 | 79.293 |
| Esophagus, Adenocarcinoma, Primary | 3 | 176.663 | 36.089 | 193.352 |
| Esophagus, Normal | 22 | 106.996 | 30.476 | 108.666 |
| Kidney, Carcinoma, Chromophobe Type, Primary | 3 | 108.286 | 24.187 | 97.844 |
| Kidney, Normal | 81 | 36.839 | 18.515 | 37.16 |
| Kidney, Renal Cell Carcinoma, Clear Cell Type, Primary | 45 | 66.31 | 43.833 | 55.188 |
| Kidney, Renal Cell Carcinoma, Non-Clear Cell Type, Primary | 15 | 64.572 | 27.295 | 64.618 |
| Kidney, Transitional Cell Carcinoma, Primary | 4 | 270.505 | 281.828 | 149.683 |
| Kidney, Wilm's Tumor, Primary | 8 | 412.566 | 188.967 | 427.328 |
| Larynx, Normal | 4 | 123.45 | 59.992 | 136.237 |
| Larynx, Squamous Cell Carcinoma, Primary | 4 | 330.967 | 173.065 | 276.574 |
| Liver, Hepatocellular Carcinoma | 16 | 93.342 | 52.304 | 81.455 |
| Liver, Normal | 42 | 44.982 | 30.912 | 44.236 |
| Lung, Adenocarcinoma, Primary | 46 | 168.798 | 162.569 | 107.818 |
| Lung, Adenosquamous Carcinoma, Primary | 3 | 79.825 | 12.277 | 78.251 |
| Lung, Large Cell Carcinoma, Primary | 7 | 218.032 | 104.354 | 255.401 |
| Lung, Neuroendocrine Carcinoma (Non-Small Cell Type), | 3 | 543.348 | 731.846 | 141.593 |
| Primary | | | | |
| Lung, Normal | 126 | 79.129 | 155.169 | 57.522 |
| Lung, Small Cell Carcinoma, Primary | 3 | 1071.102 | 211.415 | 1060.096 |
| Lung, Squamous Cell Carcinoma, Primary | 39 | 340.664 | 209.747 | 257.964 |
| Oral Cavity, Squamous Cell Carcinoma, Primary | 3 | 280.816 | 167.057 | 318.621 |
| Ovary, Adenocarcinoma, Clear Cell Type, Primary | 6 | 103.755 | 36.619 | 99.987 |
| Ovary, Adenocarcinoma, Endometrioid Type, Primary | 22 | 183.702 | 109.354 | 146.8 |
| Ovary, Adenocarcinoma, Papillary Serous Type, Primary | 36 | 174.4 | 102.164 | 154.5 |
| Ovary, Granulosa Cell Tumor, Primary | 3 | 156.848 | 16.187 | 159.53 |
| Ovary, Mucinous Cystadenocarcinoma, Primary | 7 | 84.611 | 15.699 | 84.895 |
| Ovary, Mullerian Mixed Tumor, Primary | 5 | 363.898 | 221.096 | 403.494 |
| Ovary, Normal | 89 | 87.552 | 46.998 | 79.653 |
| Pancreas, Adenocarcinoma, Primary | 23 | 113.283 | 54.941 | 97.892 |
| Pancreas, Islet Cell Tumor, Malignant, Primary | 7 | 146.32 | 69.165 | 139.025 |
| Pancreas, Normal | 46 | 41.189 | 32.682 | 39.683 |
| Prostate, Adenocarcinoma, Primary | 86 | 84.105 | 31.659 | 78.611 |
| Prostate, Normal | 57 | 62.336 | 21.869 | 62.386 |
| Rectum, Adenocarcinoma (Excluding Mucinous Type), | 29 | 243.362 | 136.269 | 203.98 |
| Primary | | | | |
| Rectum, Adenocarcinoma, Mucinous Type, Primary | 3 | 162.35 | 72.122 | 153.531 |
| Rectum, Normal | 44 | 87.534 | 33.51 | 88.558 |
| Skin, Basal Cell Carcinoma, Primary | 4 | 144.053 | 35.538 | 145.552 |
| Skin, Malignant Melanoma, Primary | 7 | 413.489 | 334.748 | 233.006 |
| Skin, Normal | 61 | 54.469 | 80.562 | 44.588 |
| Skin, Squamous Cell Carcinoma, Primary | 4 | 318.382 | 401.815 | 147.191 |
| Small Intestine, Gastrointestinal Stromal Tumor (GIST), | 4 | 159.986 | 44.725 | 151.947 |
| Primary | | | | |
| Small Intestine, Normal | 97 | 61.454 | 24.241 | 60.23 |
| Stomach, Adenocarcinoma (Excluding Signet Ring Cell | 27 | 186.598 | 113.859 | 146.447 |
| Type), Primary | | | | |
| Stomach, Adenocarcinoma, Signet Ring Cell Type, Primary | 9 | 164.955 | 74.288 | 170.523 |
| Stomach, Gastrointestinal Stromal Tumor (GIST), Primary | 9 | 99.259 | 43.37 | 104.269 |
| Stomach, Normal | 52 | 93.083 | 52.839 | 79.504 |
| Thyroid Gland, Follicular Carcinoma, Primary | 3 | 129.16 | 95.772 | 83.155 |
| Thyroid Gland, Normal | 24 | 60.847 | 26.391 | 63.367 |
| Thyroid Gland, Papillary Carcinoma, Primary; All Variants | 29 | 65.447 | 25.161 | 58.688 |
| Urinary Bladder, Normal | 9 | 56.905 | 21.981 | 48.891 |
| Urinary Bladder, Transitional Cell Carcinoma, Primary | 4 | 278.795 | 125.176 | 271.553 |
| Uterine Cervix, Adenocarcinoma, Primary | 3 | 293.178 | 270.738 | 213.411 |
| Uterine Cervix, Normal | 115 | 78.201 | 72.59 | 69.419 |
| Vulva, Normal | 4 | 82.187 | 33.953 | 72.273 |
| Vulva, Squamous Cell Carcinoma, Primary | 5 | 201.097 | 75.24 | 216.477 |

Method of Treatment with PARP Inhibitors

PARP inhibitors have potential therapeutic benefit when used independently in the treatment of various diseases such as, myocardial ischemia, stroke, head trauma, and neurodegenerative disease, and as an adjunct therapy with other agents including chemotherapeutic agents, radiation, oligonucleotides, or antibodies in cancer therapy. Without limiting the scope of the present embodiments, it shall be understood that various PARP inhibitors are known in the art and are all within the scope of the present embodiments. Some of the examples of PARP inhibitors are disclosed herein but they are not in any way limiting to the scope of the present description.

A great preponderance of PARP inhibitors have been designed as analogs of benzamides, which bind competitively with the natural substrate NAD in the catalytic site of PARP. The PARP inhibitors include, but are not limited to, benzamides, cyclic benzamides, quinolones and isoquinolones and benzopyrones (U.S. Pat. No. 5,464,871, U.S. Pat. No. 5,670,518, U.S. Pat. No. 6,004,978, U.S. Pat. No. 6,169,104, U.S. Pat. No. 5,922,775, U.S. Pat. No. 6,017,958, U.S. Pat. No. 5,736,576, and U.S. Pat. No. 5,484,951, all incorporated herein in their entirety). The PARP inhibitors include a variety of cyclic benzamide analogs (i.e. lactams) which are potent inhibitors at the NAD site. Other PARP inhibitors include, but are not limited to, benzimidazoles and indoles (EP 841924, EP 1127052, U.S. Pat. No. 6,100,283, U.S. Pat. No. 6,310,082, US 2002/156050, US 2005/054631, WO 05/012305, WO 99/11628, and US 2002/028815). A number of low-molecular-weight inhibitors of PARP have been used to elucidate the functional role of poly ADP-ribosylation in DNA repair. In cells treated with alkylating agents, the inhibition of PARP leads to a marked increase in DNA-strand breakage and cell killing (Durkacz et al, 1980, Nature 283: 593-596; and Berger, N. A., 1985, Radiation Research, 101: 4-14). Subsequently, such inhibitors have been shown to enhance the effects of radiation response by suppressing the repair of potentially lethal damage (Ben-Hur et al, 1984, British Journal of Cancer, 49 (Suppl. VI): 34-42; and Schlicker et al, 1999, Int. J. Radiat. Bioi., 75: 91-100). PARP inhibitors have been reported to be effective in radio sensitizing hypoxic tumor cells (U.S. Pat. Nos. 5,032,617, 5,215,738 and 5,041,653). Furthermore, PARP knockout (PARP −/−) animals exhibit genomic instability in response to alkylating agents and γ-irradiation (Wang et al, 1995, Genes Dev., 9: 509-520; and Menissier de Murcia et al, 1997, Proc. Natl. Acad. Sci. USA, 94: 7303-7307).

Oxygen radical DNA damage that leads to strand breaks in DNA, which are subsequently recognized by PARP, is a major contributing factor to such disease states as shown by PARP inhibitor studies (Cosi et al, 1994, J. Neurosci. Res., 39: 38-46; and Said et al, 1996, Proc. Natl. Acad. Sci. U.S.A., 93: 4688-4692). It has also been demonstrated that efficient retroviral infection of mammalian cells is blocked by the inhibition of PARP activity. Such inhibition of recombinant retroviral vector infections was shown to occur in various different cell types (Gaken et al, 1996, J. Virology, 70(6): 3992-4000). Inhibitors of PARP have thus been developed for the use in anti-viral therapies and in cancer treatment (WO91/18591). Moreover, PARP inhibition has been speculated to delay the onset of aging characteristics in human fibroblasts (Rattan and Clark, 1994, Biochem. Biophys. Res. Comm., 201 (2): 665-672). This may be related to the role that PARP plays in controlling telomere function (d'Adda di Fagagna et al, 1999, Nature Gen., 23(1): 76-80).

PARP inhibitors may possess the following structural characteristics: 1) amide or lactam functionality; 2) an NH proton of this amide or lactam functionality could be conserved for effective bonding; 3) an amide group attached to an aromatic ring or a lactam group fused to an aromatic ring; 4) optimal cis-configuration of the amide in the aromatic plane; and 5) constraining mono-aryl carboxamide into heteropolycyclic lactams (Costantino et al., 2001, J Med. Chem., 44:3786-3794). Virag et al., 2002, Pharmacol Rev., 54:375-429, 2002 summarizes various PARP inhibitors. Some of the examples of PARP inhibitors include, but are not limited to, isoquinolinone and dihydrolisoquinolinone (for example, U.S. Pat. No. 6,664,269, and WO 99/11624), nicotinamide, 3-aminobenzamide, monoaryl amides and bi-, tri-, or tetracyclic lactams, phenanthridinones (Perkins et al., 2001, Cancer Res., 61:4175-4183), 3,4-dihydro-5-methyl-isoquinolin-1(2H)-one and benzoxazole-4-carboxamide (Griffin et al., 1995, Anticancer Drug Des, 10:507-514; Griffin et al., 1998, J Med Chem, 41:5247-5256; and Griffin et al., 1996, Pharm Sci, 2:43-48), dihydroisoquinolin-1(2H)-nones, 1,6-naphthyridine-5(6H)-ones, quinazolin-4(3H)-ones, thieno[3,4-c]pyridin-4(5H)ones and thieno[3,4-d]pyrimidin-4(3H)ones, 1,5-dihydroxyisoquinoline, and 2-methyl-quinazolin-4[3H]-one (Yoshida et al., 1991, J Antibiot (Tokyo) 44:111-112; Watson et al., 1998, Bioorg Med. Chem., 6:721-734; and White et al., 2000, J Med. Chem., 43:4084-4097), 1,8-Napthalimide derivatives and (5H)phenanthridin-6-ones (Banasik et al., 1992, J Biol Chem, 267:1569-1575; Watson et al., 1998, Bioorg Med Chem., 6:721-734; Soriano et al., 2001, Nat Med., 7:108-113; Li et al., 2001, Bioorg Med Chem Lett., 11:1687-1690; and Jagtap et al., 2002, Crit Care Med., 30:1071-1082), tetracyclic lactams, 1,11b-dihydro-[2H]benzopyrano[4,3,2-de]isoquinolin-3-one, 1-methyl-4-phenyl-1,2,3,6-tetrahydropyridine (MPTP) (Zhang et al., 2000, Biochem Biophys Res Commun., 278:590-598; and Mazzon et al., 2001, Eur J Pharmacol, 415:85-94). Other examples of PARP inhibitors include, but are not limited to, those detailed in the patents: U.S. Pat. No. 5,719,151, U.S. Pat. No. 5,756,510, U.S. Pat. No. 6,015,827, U.S. Pat. No. 6,100,283, U.S. Pat. No. 6,156,739, U.S. Pat. No. 6,310,082, U.S. Pat. No. 6,316,455, U.S. Pat. No. 6,121,278, U.S. Pat. No. 6,201,020, U.S. Pat. Nos. 6,235,748, 6,306,889, U.S. Pat. No. 6,346,536, U.S. Pat. No. 6,380,193, U.S. Pat. No. 6,387,902, U.S. Pat. No. 6,395,749, U.S. Pat. No. 6,426,415, U.S. Pat. No. 6,514,983, U.S. Pat. No. 6,723,733, U.S. Pat. No. 6,448,271, U.S. Pat. No. 6,495,541, U.S. Pat. No. 6,548,494, U.S. Pat. No. 6,500,823, U.S. Pat. No. 6,664,269, U.S. Pat. No. 6,677,333, U.S. Pat. No. 6,903,098, U.S. Pat. No. 6,924,284, U.S. Pat. No. 6,989,388, U.S. Pat. No. 6,277,990, U.S. Pat. No. 6,476,048, and U.S. Pat. No. 6,531,464. Additional examples of PARP inhibitors include, but are not limited to, those detailed in the patent application publications: US 2004198693A1, US 2004034078A1, US 2004248879A1, US 2004249841A1, US 2006074073A1, US 2006100198A1, US 2004077667A1, US 2005080096A1, US 2005171101A1, US 2005054631A1, WO 05054201A1, WO 05054209A1, WO 05054210A1, WO 05058843A1, WO 06003146A1, WO 06003147A1, WO 06003148A1, WO 06003150A1, and WO 05097750A1.

In one embodiment, the PARP inhibitors are compounds of Formula (Ia)

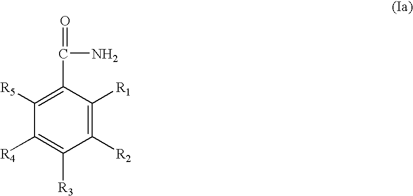

wherein R₁, R₂, R₃, R₄, and R₅ are, independently selected from the group consisting of hydrogen, hydroxy, amino, nitro, iodo, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents are always nitro, and at least one substituent positioned adjacent to a nitro is always iodo, and pharmaceutically acceptable salts, solvates, isomers, tautomers, metabolites, analogs, or prodrugs thereof. R₁, R₂, R₃, R₄, and R₅ can also be a halide such as chloro, fluoro, or bromo. Further details regarding compounds of formula Ia are provided in U.S. Pat. No. 5,464,871.

One compound of formula Ia is a compound according to the formula Ia

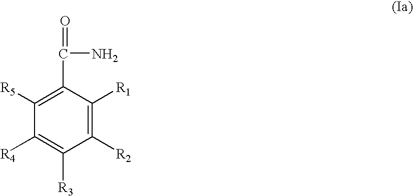

wherein R₂, R₃, R₄, and R₅ are, independent of one another, selected from the group consisting of hydrogen, hydroxy, amino, nitro, iodo, (C₁-C₆) alkyl, (C₁-C₆) alkoxy, (C₃-C₇) cycloalkyl, and phenyl and pharmaceutically acceptable salts thereof, wherein at least two of the five R₁, R₂, R₃, R₄, and R₅ substituents are always hydrogen and at least one of the five substituents are always nitro.

Another compound of formula Ia is

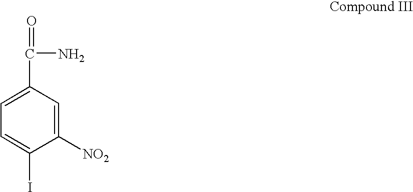

In some embodiments, metabolites to formula I or Ia are used in the methods described herein. Some metabolites useful in the present methods are of the Formula (Ib):

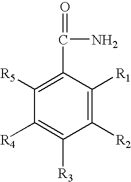

wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, or R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent a R₁, R₂, R₃, R₄ or R₅ group that is a nitroso, hydroxyamino, hydroxy or amino group. In some embodiments, the compounds of (2) are such that the iodo the iodo group is always adjacent a R₁, R₂, R₃, R₄ or R₅ group that is a nitroso, hydroxyamino, or amino group.

The following compositions are metabolite compounds, each represented by a chemical formula:

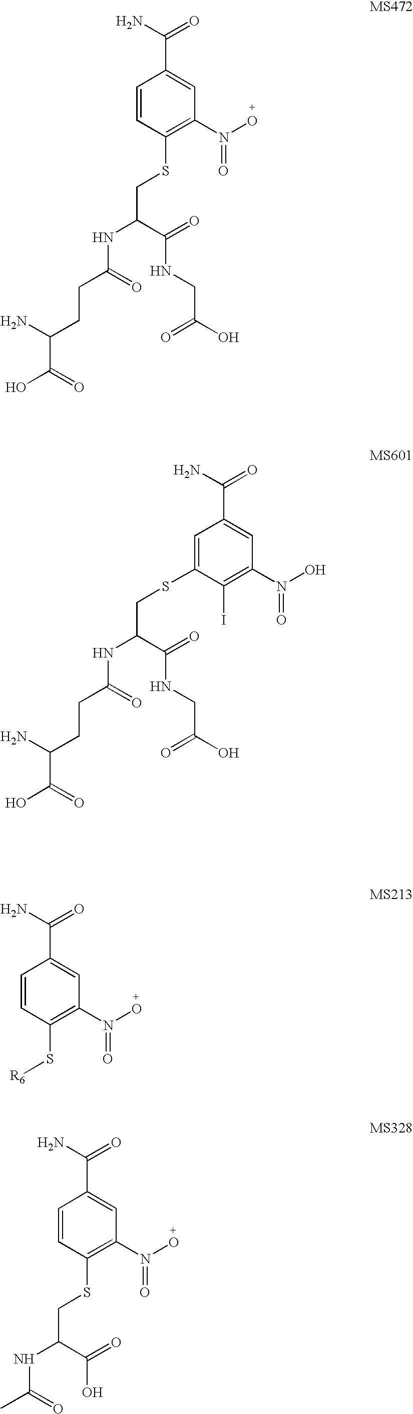

R₆ is selected from a group consisting of hydrogen, alkyl(C₁-C₈), alkoxy(C₁-C₈), isoquinolinones, indoles, thiazole, oxazole, oxadiazole, thiphene, or phenyl.

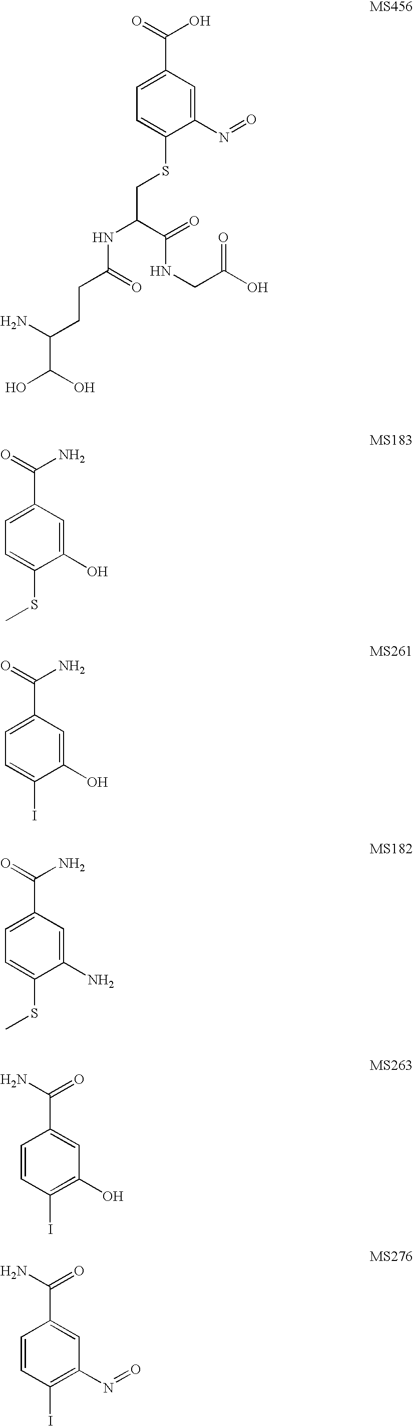

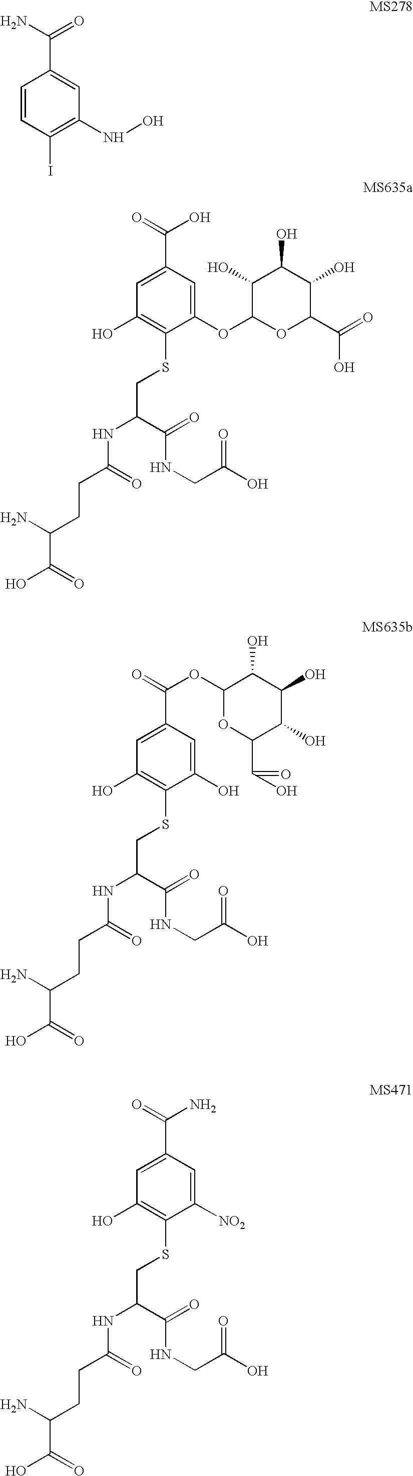

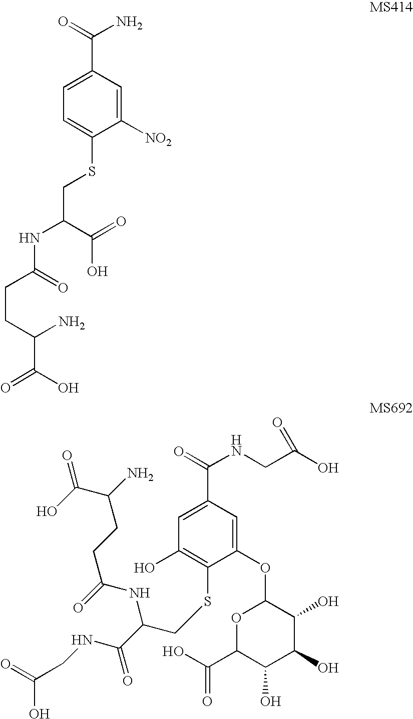

While not being limited to any one particular mechanism, the following provides an example for MS292 metabolism via a nitroreductase or glutathione conjugation mechanism:

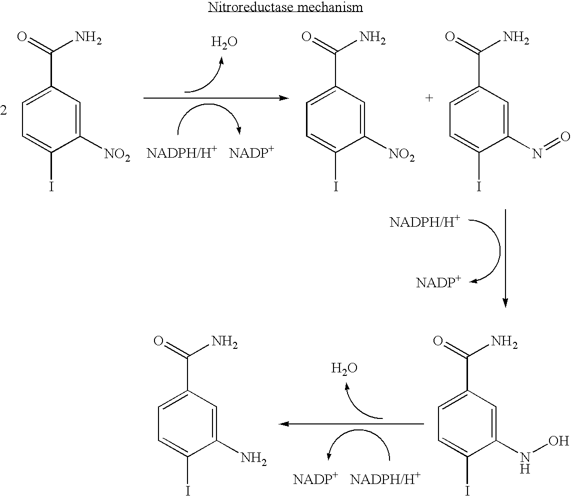

Compound III glutathione conjugation and metabolism:

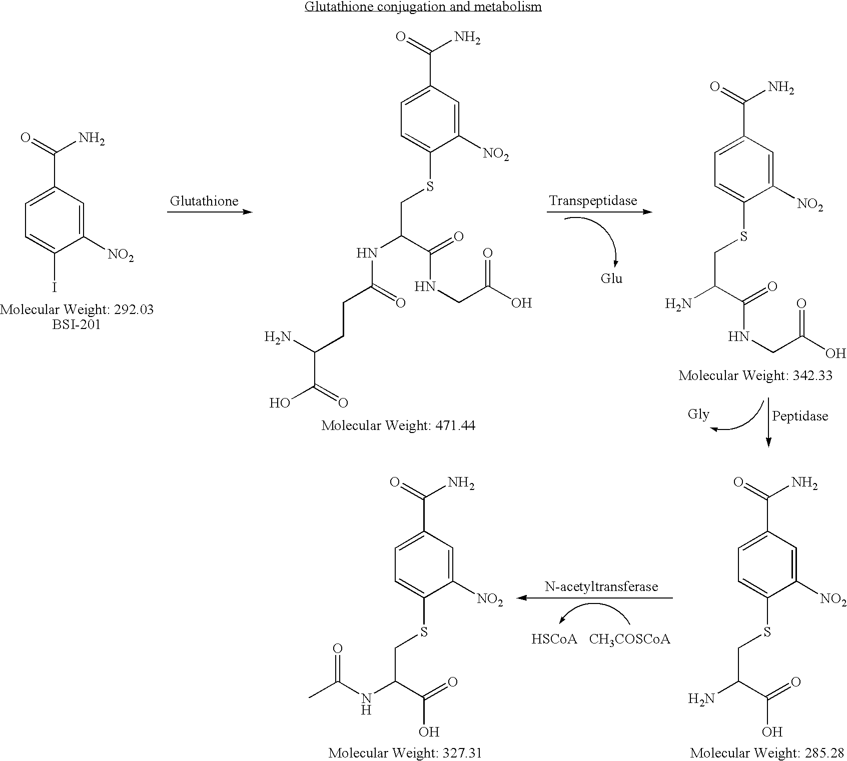

In some embodiments, benzopyrone compounds of formula II are used in the methods described herein. The benzopyrone compounds of formula II are,

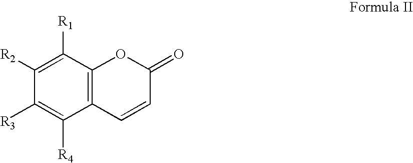

wherein R₁, R₂, R₃ and R₄ are independently selected from the group consisting of H, halogen, optionally substituted hydroxy, optionally substituted amine, optionally substituted lower alkyl, optionally substituted phenyl, optionally substituted C₄-C₁₀ heteroaryl and optionally substituted C₃-C₈ cycloalkyl or a salt, solvate, isomer, tautomers, metabolite, or prodrug thereof (U.S. Pat. No. 5,484,951 is incorporated herein by reference in its entirety).

Some embodiments employ a compound having the chemical formula:

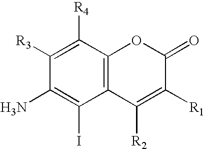

wherein R₁, R₂, R₃, or R₄ are each independently selected from the group consisting of hydrogen, hydroxy, amino, (C₁-C₆) alkyl, (C₁-C₆) alkoxy, (C₃-C₇) cycloalkyl, halo and phenyl and pharmaceutically acceptable salts thereof, wherein at least three of the four R₁, R₂, R₃, or R₄ substituents are always hydrogen.

Some embodiments employ a compound having the chemical formula:

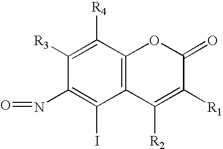

wherein R₁, R₂, R₃, or R₄ are each independently selected from the group consisting of hydrogen, hydroxy, amino, (C₁-C₆) alkyl, (C₁-C₆) alkoxy, (C₃-C₇) cycloalkyl, halo and phenyl and pharmaceutically acceptable salts thereof, wherein at least three of the four R₁, R₂, R₃, or R₄ substituents are always hydrogen.

Some embodiments employ a compound of the chemical formula:

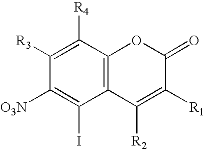

wherein R₁, R₂, R₃, or R₄, are each independently selected from the group consisting of hydrogen, hydroxy, amino, (C₁-C₆) alkyl, (C₁-C₆) alkoxy, (C₃-C₇) cycloalkyl, halo and phenyl, wherein at least three of the four R₁, R₂, R₃, or R₄ substituents are always hydrogen.

One embodiment relates to the following benzopyrone compound of formula II

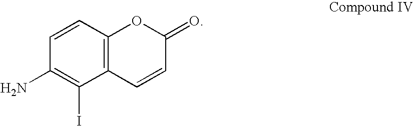

In yet another embodiment, the compound used in the methods described herein is

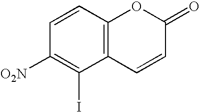

Further details regarding the benzopyrone compounds are in U.S. Pat. No. 5,484,951, which is herein incorporated by reference in its entirety.

It is likely that the most potent and effective PARP inhibitors (i.e., the likely candidates for drug development) are not yet available in the scientific literature but rather are undergoing clinical trials or may ultimately emerge in the various databases of published patents and pending patent applications. All such PARP inhibitors are within the scope of the present embodiments. In addition to selective, potent enzymatic inhibition of PARP, several additional approaches may be employed to inhibit the cellular activity of PARP in cells or in experimental animals. The inhibition of intracellular calcium mobilization protects against oxidant-induced PARP activation, NAD+depletion, and cell necrosis, as demonstrated in thymocytes (Virag et al., 1999, Mol. Pharmacol., 56:824-833) and in intestinal epithelial cells (Karczewski et al., 1999, Biochem Pharmacol., 57:19-26). Similar to calcium chelators, intracellular zinc chelators have been shown to protect against oxidant-mediated PARP activation and cell necrosis (Virag et al., 1999, Br J Pharmacol., 126:769-777). Intracellular purines (inosine, hypoxanthine), in addition to a variety of effects, may also exert biological actions as inhibitors of PARP (Virag et al., 2001, FASEB J., 15:99-107).

The methods provided may comprise the administration of PARP inhibitors by itself or in combination with other therapies. The choice of therapy that can be co-administered with the compositions described herein will depend, in part, on the condition being treated. For example, for treating acute myeloid leukemia, compounds described herein can be used in combination with radiation therapy, monoclonal antibody therapy, chemotherapy, bone marrow transplantation, or a combination thereof.

An effective therapeutic amount of the PARP inhibitors as disclosed herein is administered to a patient, (e.g., a mammal such as a human), to affect a pharmacological activity involving inhibition of a PARP enzyme or PARP activity. As such, PARP inhibitors may be useful in treating or preventing a variety of diseases and illnesses including neural tissue damage resulting from cell damage or death due to necrosis or apoptosis, cerebral ischemia and reperfusion injury or neurodegenerative diseases in an animal. In addition, compounds can also be used to treat a cardiovascular disorder in an animal, by administering an effective amount of the PARP inhibitor to the animal. Further still, the compounds can be used to treat cancer and to radiosensitize or chemosensitize tumor cells.

In some embodiments, the PARP inhibitors can be used to modulate damaged neurons, promote neuronal regeneration, prevent neurodegeneration and/or treat a neurological disorder. The PARP inhibitors inhibit PARP activity and, thus, are useful for treating neural tissue damage, particularly damage resulting from cancer, cardiovascular disease, cerebral ischemia and reperfusion injury or neurodegenerative diseases in animals. The PARP inhibitors are useful for treating cardiac tissue damage, particularly damage resulting from cardiac ischemia or caused by reperfusion injury in a patient. The compounds are useful for treating cardiovascular disorders selected from the group consisting of: coronary artery disease, such as atherosclerosis; angina pectoris; myocardial infarction; myocardial ischemia and cardiac arrest; cardiac bypass; and cardiogenic shock.

In another aspect, the PARP inhibitors can be used to treat cancer, or in combination with chemotherapeutics, radiotherapeutics, or radiation. The PARP inhibitors described herein can be “anti-cancer agents,” which term also encompasses “anti-tumor cell growth agents” and “anti-neoplastic agents.” For example, the PARP inhibitors are useful for treating cancers, and radiosensitizing and/or chemosensitizing tumor cells in cancers.

Radiosensitizers are known to increase the sensitivity of cancerous cells to the toxic effects of electromagnetic radiation. Many cancer treatment protocols currently employ radiosensitizers activated by the electromagnetic radiation of x-rays. Examples of x-ray activated radiosensitizers include, but are not limited to, the following: metronidazole, misonidazole, desmethylmisonidazole, pimonidazole, etanidazole, nimorazole, mitomycin C, RSU 1069, SR 4233, EO9, RB 6145, nicotinamide, 5-bromodeoxyuridine (BUdR), 5-iododeoxyuridine (IUdR), bromodeoxycytidine, fluorodeoxyuridine (FudR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, photofrin, benzoporphyrin derivatives, NPe6, tin etioporphyrin SnET2, pheoborbide-α, bacteriochlorophyll-α, naphthalocyanines, phthalocyanines, zinc phthalocyanine, and therapeutically effective analogs and derivatives of the same.

Radiosensitizers can be administered in conjunction with a therapeutically effective amount of one or more other PARP inhibitors, including but not limited to: PARP inhibitors which promote the incorporation of radiosensitizers to the target cells; PARP inhibitors which control the flow of therapeutics, to nutrients, and/or oxygen to the target calls. Similarly, chemosensitizers are also known to increase the sensitivity of cancerous cells to the toxic effects of chemotherapeutic compounds. Exemplary chemotherapeutic agents that can be used in conjunction with PARP inhibitors include, but are not limited to, adriamycin, camptothecin, dacarbazine, carboplatin, cisplatin, daunorubicin, docetaxel, doxorubicin, interferon (alpha, beta, gamma), interleukin 2, innotecan, paclitaxel, streptozotocin, temozolomide, topotecan, and therapeutically effective analogs and derivatives of the same. In addition, other therapeutic agents which can be used in conjunction with a PARP inhibitors include, but are not limited to, 5-fluorouracil, leucovorin, 5′-amino-5′-deoxythymidine, oxygen, carbogen, red cell transfusions, perfluorocarbons (e.g., Fluosol-DA), 2,3-DPG, BW12C, calcium channel blockers, pentoxyfylline, antiangiogenesis compounds, hydralazine, and L-BSO.

In some embodiments, the therapeutic agents for the treatment include antibodies or reagents that bind to PARP, and thereby lower the level of PARP in a subject. In other embodiments, cellular expression can be modulated in order to affect the level of PARP and/or PARP activity in a subject. Therapeutic and/or prophylactic polynucleotide molecules can be delivered using gene transfer and gene therapy technologies. Still other agents include small molecules that bind to or interact with the PARP and thereby affect the function thereof, and small molecules that bind to or interact with nucleic acid sequences encoding PARP, and thereby affect the level of PARP. These agents may be administered alone or in combination with other types of treatments known and available to those skilled in the art for treating diseases. In some embodiment, the PARP inhibitors for the treatment can be used either therapeutically, prophylactically, or both. The PARP inhibitors may either directly act on PARP or modulate other cellular constituents which then have an effect on the level of PARP. In some embodiments, the PARP inhibitors inhibit the activity of PARP.

The methods of treatment as disclosed herein can be via oral administration, transmucosal administration, buccal administration, nasal administration, inhalation, parental administration, intravenous, subcutaneous, intramuscular, sublingual, transdermal administration, ocular administration, and rectal administration.

Pharmaceutical compositions of PARP inhibitors suitable for use in treatment following the identification of a disease treatable by PARP inhibitors in a subject, include compositions wherein the active ingredient is contained in a therapeutically or prophylactically effective amount, i.e., in an amount effective to achieve therapeutic or prophylactic benefit. The actual amount effective for a particular application will depend, inter alia, on the condition being treated and the route of administration. Determination of an effective amount is well within the capabilities of those skilled in the art. The pharmaceutical compositions comprise the PARP inhibitors, one or more pharmaceutically acceptable carriers, diluents or excipients, and optionally additional therapeutic agents. The compositions can be formulated for sustained or delayed release.

The compositions can be administered by injection, topically, orally, transdermally, rectally, or via inhalation. The oral form in which the therapeutic agent is administered can include powder, tablet, capsule, solution, or emulsion. The effective amount can be administered in a single dose or in a series of doses separated by appropriate time intervals, such as hours. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Suitable techniques for preparing pharmaceutical compositions of the therapeutic agents are well known in the art.

A preferred dose for Compound III is 4 mg/kg IV over one hour twice weekly beginning on day 1 (doses of Compound III are preferably separated by at least 2 days). Compound III treatment is preferably given twice weekly as an IV infusion for three consecutive weeks in each 28-day cycle. Other preferred doses include 0.5, 1.0, 1.4, 2.8 and 4 mg/kg either as a monotherapy or a combination therapy.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular PARP inhibitor, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

### IGF1 Receptor/IGF Pathway and Modulators

As above, IGF1 receptor, IGF-1 or IGF-2 modulators, including inhibitors, may also be administered as disclosed herein. Picropodophyllin dosing, PPP, BMS554417, BMS536924, AG1024, NVP-AEW541, NVP-ADW742, and antibodies directed to IGF1 receptor or its ligands are examples of compounds that may be used in conjunction with the present methods. In one non-limiting embodiment, Picropodophyllin may be administered at a dose of 0.01-50 μM. In one non-limiting embodiment, Picropodophyllin may be administered at about 7 mg/kg/day or about 28 mg/kg/day. Other compounds that inhibit IFR-1 receptor or its ligands are also expressly contemplated herein. Provided herein is a method of treating triple negative breast cancer with a PARP inhibitor in combination with at least one anti-tumor agent. In one embodiment, the at least one anti-tumor agent is Picropodophyllin. Also described herein is a method of treating ER-negative, PR-negative, HER-2 negative metastatic breast cancer in a patient in need of such treatment, comprising administering to said patient a PARP inhibitor and Picropodophyllin.

### EGFR Pathways and Modulators

Similarly, EGFR modulators or inhibitors may also be administered as above, including Ceuximab, panitunmumam, matuzuman, MDX-446, nimutozumab, mAb 806, erbitux (IMC-C2225), IRESSA® (ZD1839), erlotinib, gefitinib, EKB-569, lapatinib (GW572016), PKI-166 and canertinib (Rocha-Lima et al., 2007, Cancer Control, 14:295-304). In one non-limiting embodiment, IRESSA® may be administered at a dose of 250 mg daily, 500 mg daily, 750 mg daily, or 1250 mg daily. Other compounds that inhibit EGFR, including nucleic acid expression or activity, or compounds that inhibit other targets in the erbB tyrosine kinase receptor family, are also contemplated herein. Provided herein is a method of treating lung cancer with a PARP inhibitor in combination with at least one anti-tumor agent. In one embodiment, the at least one anti-tumor agent is IRESSA®. Also described herein is a method of treating lung adenocarcinoma, small cell carcinoma, non-small cell carcinomas, squamous cell carcinoma or large cell carcinoma in a patient in need of such treatment, comprising administering to said patient a PARP inhibitor and IRESSA®.

### Standard of Care for Cancer Sites

In another aspect, PARP inhibitors are used in combination with the primary standards of treatment for the cancer being treated. Described herein is the standard of care for certain types of cancers. In some embodiments, the modulators and inhibitors disclosed herein are used in combination with the standard of care described herein.

Endometrial: There are four primary standards of care for treating endometrial cancers including surgery (total hysterectomy, bilateral salpingo-oophorectomy, and radical hysterectomy), radiation, chemotherapy, and hormone therapy. Adjuvant therapies involving said therapies are administered in some cases.

Breast: Breast cancer treatments currently involve breast-conserving surgery and radiation therapy with or without tamoxifen, total mastectomy with or without tamoxifen, breast-conserving surgery without radiation therapy, bilateral prophylactic total mastectomy without axillary node dissection, delivering tamoxifen to decrease the incidence of subsequent breast cancers, and adjuvant therapies involving said therapies.

Ovary: If the tumor is well- or moderately well-differentiated, total abdominal hysterectomy and bilateral salpingo-oophorectomy with omentectomy is adequate for patients with early stage disease. Patients diagnosed with stage III and stage IV disease are treated with surgery and chemotherapy.

Cervix: Methods to treat ectocervical lesions include loop electrosurgical excision procedure (LEEP), laser therapy, conization, and cryotherapy. For stage I and stage II tumors, treatment options include: total hysterectomy, conization, radical hysterectomy, and intracavitary radiation therapy alone, bilateral pelvic lymphadenectomy, postoperative total pelvic radiation therapy plus chemotherapy, and radiation therapy plus chemotherapy with cisplatin or cisplatin/5-FU. For stage III and stage IV tumors, the standard of treatment of cervical cancer is radiation and/or chemotherapy with drugs including cisplatin, ifosfamide, ifosfamide-cisplatin, paclitaxel, irinotecan, paclitaxel/cisplatin, and cisplatin/gemcitabine.

Testes: The standards of treatment of seminoma are radical inguinal orchiectomy with or without by single-dose carboplatin adjuvant therapy, removal of the testicle via radical inguinal orchiectomy followed by radiation therapy, and radical inguinal orchiectomy followed by combination chemotherapy or by radiation therapy to the abdominal and pelvic lymph nodes. For nonseminoma patients treatments include removal of the testicle through the groin followed by retroperitoneal lymph node dissection, radical inguinal orchiectomy with or without removal of retroperitoneal lymph nodes with or without fertility-preserving retroperitoneal lymph node dissection with or without chemotherapy.

Lung: In non-small cell lung cancer (NSCLC), results of standard treatment are poor except for the most localized cancers. All newly diagnosed patients with NSCLC are potential candidates for studies evaluating new forms of treatment. Surgery is the most potentially curative therapeutic option for this disease; radiation therapy can produce a cure in a small number of patients and can provide palliation in most patients. Adjuvant chemotherapy may provide an additional benefit to patients with resected NSCLC. In advanced-stage disease, chemotherapy is used.

Skin: The traditional methods of basal cell carcinoma treatment involve the use of cryosurgery, radiation therapy, electrodesiccation and curettage, and simple excision. Localized squamous cell carcinoma of the skin is a highly curable disease. The traditional methods of treatment involve the use of cryosurgery, radiation therapy, electrodesiccation and curettage, and simple excision.

Liver: Hepatocellular carcinoma is potentially curable by surgical resection, but surgery is the treatment of choice for only the small fraction of patients with localized disease. Other treatments remain in the clinical study phase including systemic or infusional chemotherapy, hepatic artery ligation or embolization, percutaneous ethanol injection, radiofrequency ablation, cryotherapy, and radiolabeled antibodies, often in conjunction with surgical resection and/or radiation therapy.

Thyroid: Standard treatment options of thyroid cancers include total thyroidectomy, lobectomy, and combinations of said surgeries with 1131 ablation, external-beam radiation therapy, thyroid-stimulating hormone suppression with thyroxine, and chemotherapy.

Esophagus: Primary treatment modalities include surgery alone or chemotherapy with radiation therapy. Effective palliation may be obtained in individual cases with various combinations of surgery, chemotherapy, radiation therapy, stents, photodynamic therapy, and endoscopic therapy with Nd: YAG laser.

Kidney: Surgical resection is the mainstay of treatment of this disease. Even in patients with disseminated tumor, locoregional forms of therapy may play an important role in palliating symptoms of the primary tumor or of ectopic hormone production. Systemic therapy has demonstrated only limited effectiveness.

In one embodiment, PARP inhibitors are combined with other chemotherapeutics such as, irinotecan, topotecan, cisplatin, or temozolomide to improve the treatment of a number of cancers such as colorectal and gastric cancers, and melanoma and glioma, respectively. In another embodiment, PARP inhibitors are combined with irinotecan to treat advanced colorectal cancer or with temozolomide to treat malignant melanoma.

In cancer patients, in one embodiment PARP inhibition is used to increase the therapeutic benefits of radiation and chemotherapy. In another embodiment, targeting PARP is used to prevent tumor cells from repairing DNA themselves and developing drug resistance, which may make them more sensitive to cancer therapies. In yet another embodiment, PARP inhibitors are used to increase the effect of various chemotherapeutic agents (e.g. methylating agents, DNA topoisomerase inhibitors, cisplatin etc.), as well as radiation, against a broad spectrum of tumors (e.g. glioma, melanoma, lymphoma, colorectal cancer, head and neck tumors).

### Kits

In yet another aspect, kits are provided for identifying a disease in a subject treatable by PARP modulators, wherein the kits can be used to detect the level of PARP in a sample obtained from a subject. For example, the kits can be used to identify the level and/or activity of PARP in normal and diseased tissue as described herein, where PARP level is differentially present in samples of a diseased patient and normal subjects. In one embodiment, a kit comprises a substrate comprising an adsorbent thereon, wherein the adsorbent is suitable for binding PARP and/or RNA, and instructions to identify PARP and/or level of PARP and/or PAR (monoribose and polyribose) by contacting a sample with the adsorbent and detecting PARP retained by the adsorbent. In another embodiment, a kit comprises (a) a reagent that specifically binds to or interacts with PARP; and (b) a detection reagent. In some embodiments, the kit may further comprise instructions for suitable operation parameters in the form of a label or a separate insert. Optionally, the kit may further comprise a standard or control information so that the test sample can be compared with the control information standard to determine if the test amount of PARP detected in a sample is a diagnostic amount.

The container means of the kits will generally include at least one vial, test tube, flask, bottle, syringe and/or other container means, into which the at least one polypeptide can be placed, and/or preferably, suitably aliquoted. The kits can include a means for containing at least one fusion protein, detectable moiety, reporter molecule, and/or any other reagent containers in close confinement for commercial sale. Such containers may include injection and/or blow-molded plastic containers in which the desired vials are stored. Kits can also include printed material for use of the materials in the kit.

Packages and kits can additionally include a buffering agent, a preservative and/or a stabilizing agent in a pharmaceutical formulation. Each component of the kit can be enclosed within an individual container and all of the various containers can be within a single package. Kits can be designed for cold storage or room temperature storage.

Additionally, the preparations can contain stabilizers (such as bovine serum albumin (BSA)) to increase the shelf-life of the kits. Where the compositions are lyophilized, the kit can contain further preparations of solutions to reconstitute the lyophilized preparations. Acceptable reconstitution solutions are well known in the art and include, for example, pharmaceutically acceptable phosphate buffered saline (PBS).

In some embodiments, the therapeutic agent can also be provided as separate compositions in separate containers within the kit for the treatment. Suitable packaging and additional articles for use (e.g., measuring cup for liquid preparations, foil wrapping to minimize exposure to air, and the like) are known in the art and may be included in the kit.

Packages and kits can further include a label specifying, for example, a product description, mode of administration and/or indication of treatment. Packages provided herein can include any of the compositions as described herein for treatment of any of the indications described herein.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions. Kits, therefore, can additionally include labels or instructions for using the kit components in any method described herein. A kit can include a compound in a pack, or dispenser together with instructions for administering the compound in a method described herein.

The kits may also include instructions teaching the use of the kit according to the various methods and approaches described herein. Such kits optionally include information, such as scientific literature references, package insert materials, clinical trial results, and/or summaries of these and the like, which indicate or establish the activities and/or advantages of the composition, and/or which describe dosing, administration, side effects, drug interactions, disease state for which the composition is to be administered, or other information useful to the health care provider. Such information may be based on the results of various studies, for example, studies using experimental animals involving in vivo models and studies based on human clinical trials. In various embodiments, the kits described herein can be provided, marketed and/or promoted to health providers, including physicians, nurses, pharmacists, formulary officials, and the like. Kits may, in some embodiments, be marketed directly to the consumer. In certain embodiments, the packaging material further comprises a container for housing the composition and optionally a label affixed to the container. The kit optionally comprises additional components, such as but not limited to syringes for administration of the composition.

Instructions can include instructions for practicing any of the methods described herein including treatment methods. Instructions can additionally include indications of a satisfactory clinical endpoint or any adverse symptoms that may occur, or additional information required by regulatory agencies such as the Food and Drug Administration for use on a human subject.

The instructions may be on “printed matter,” e.g., on paper or cardboard within or affixed to the kit, or on a label affixed to the kit or packaging material, or attached to a vial or tube containing a component of the kit. Instructions may additionally be included on a computer readable medium, such as a disk (floppy diskette or hard disk), optical CD such as CD- or DVD-ROM/RAM, magnetic tape, electrical storage media such as RAM and ROM, IC tip and hybrids of these such as magnetic/optical storage media.

In some embodiments, a kit may comprise reagents for the detection of DNA, RNA or protein expression levels in a sample of tumor cells from a patient to be treated.

Kits can, in some aspects, contain reagents and materials to conduct any of the assays described herein.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art; it should be understood that various alternatives to the embodiments described herein may be employed in practicing the methods described herein.

### Example 1

GeneChip arrays have been widely used for monitoring mRNA expression in many areas of biomedical research. The high-density oligonucleotide array technology allows researchers to monitor tens of thousands of genes in a single hybridization experiment as they are expressed differently in tissues and cells. The expression profile of a mRNA molecule of a gene is obtained by the combined intensity information from probes in a probe set, which consists of 11-20 probe pairs of oligonucleotides of 25 bp in length, interrogating a different part of the sequence of a gene.

The gene expressions were assessed using the Affymetrix human genome genechips (45,000 gene transcripts covering 28,473 UniGene clusters). Approximately 5 μg total RNA from each sample were labeled using high yield transcript labeling kit and labeled RNAs were hybridized, washed, and scanned according to manufacturer's specifications (Affymetrix, Inc., Santa Clara, Calif.). Affymetrix Microarray Suite 5.0 software (MAS5) was used to estimate transcript signal levels from scanned images (Affymetrix). The signals on each array were normalized to a trimmed mean value of 500, excluding lowest 2% and highest 2% of the signals. An Affymetrix probe set representing a unique GenBank sequence is referred as a probe or gene hereafter for convenience. To verify any errors in the expressions caused by image defects, the correlation coefficient of each array to an idealized distribution was determined where the idealized distribution is mean of all arrays. The genes are filtered from the remaining arrays using detection P value reported by MAS5. The genes having P>0.065 in 95% of the arrays are eliminated and all other signals are included for statistical comparisons of classes.

### Example 2

### Up-Regulation of PARP1 mRNA in Normal and Tumor Tissues

### Study Design and Materials and Methods

Tissue samples: Normal and carcinoma tissue samples were collected in the United States or United Kingdom. Specimens were harvested as part of a normal surgical procedure and flash frozen within 30 minutes of resection. Samples were shipped at −80° C. and stored in the vapor phase of liquid nitrogen at −170 to −196° C. until processed. Internal pathology review and confirmation were performed on samples subjected to analysis. H&E-stained glass slides generated from an adjacent portion of tissue were reviewed in conjunction with original diagnostic reports and samples were classified into diagnostic categories. A visual estimate of the percent of tissue involvement by tumor was recorded during slide review by the pathologist and indicates the fraction of malignant nucleated cells. Adjuvant studies such as ER/PR and Her-2/neu expression studies were performed by methodologies including immunohistochemistry and fluorescence in situ hybridization. These results as well as attendant pathology and clinical data were annotated within a sample inventory and management databases (Ascenta, BioExpress databases; Gene Logic, Gaithersburg, Md.).

RNA extraction, quality control, and expression profiling: RNA was extracted from samples by homogenization in Trizol® Reagent (Invitrogen, Carlsbad, Calif.) followed by isolation with a RNeasy kit (Qiagen, Valencia, Calif.) as recommended by the manufacturer. RNA was evaluated for quality and integrity (Agilent 2100 Bioanalyzer derived 28s/18s ratio and RNA integrity number), purity (via absorbance ratio at A260/A280), and quantity (via absorbance at A260 or alternative assay). Gene expression levels were assessed using Affymetrix human genome U133A and B GeneChips (45,000 probesets representing more than 39,000 transcripts derived from approximately 33,000 well-substantiated human genes). Two micrograms (2 μg) of total RNA was used to prepare cRNA using Superscript II™ (Invitrogen, Carlsbad, Calif.) and a T7 oligo dT primer for cDNA synthesis and an Affymetrix GeneChip® IVT Labeling Kit (Affymetrix, Santa Clara, Calif.). Quantity and purity of cRNA synthesis product was assessed using UV absorbance. Quality of cRNA synthesis was assessed using either the Agilent Bioanalyzer or a MOPS agarose gel. The labeled cRNA was subsequently fragmented, and 10 μg was hybridized to each array at 45° C. over 16-24 hours. Arrays were washed and stained according to manufacturer recommendations and scanned on Affymetrix GeneChip Scanners. Array data quality was evaluated using a proprietary high throughput application which assesses the data against multiple objective standards including 5′/3′ GAPDH ratio, signal/noise ratio, and background as well as other additional metrics (e.g. outlier, vertical variance) which must be passed prior to inclusion for analysis. GeneChip analysis was performed with Microarray Analysis Suite version 5.0, Data Mining Tool 2.0, and Microarray database software (www.affymetrix.com). All of the genes represented on the GeneChip were globally normalized and scaled to a signal intensity of 100.

Quality Control: RNA is evaluated for quality and integrity via Agilent Bioanalyzer derived 28s/28s ratio and RNA integrity number (RIN)), purity (via absorbance ratio at A260/A280), and quantity (via absorbance at A260 or alternative assay (i.e. ribogreen)). Quantity and purity of cRNA synthesis product is assessed using UV absorbance. Quality of cRNA synthesis is assessed using either the Agilent Bioanalyzer or a MOPS agarose gel. Array quality is evaluated using a proprietary high throughput application by which arrays are evaluated against several strict objective standards such as 5′/3′ GAPDH ratio, signal/noise ratio. and background as well as over thirty additional metrics (e.g. outlier, vertical variance). Data generated throughout the process is managed within the quality system to ensure data integrity of the data.

Statistical Analysis: The mean and 90%, 95%, 99%, and 99.9% upper confidence limits for an individual predicted value (UCLs) were calculated. Because we are assessing the likelihood that individual samples external to the normal set are within the baseline distribution, the prediction interval, rather than the confidence interval for the mean, was selected to estimate the expected range for future individual measurements. The prediction interval is defined by the formula, X̅±AS\sqrt{1+(1/n)}, where X̅ is the mean of the normal breast samples, S is the standard deviation of the normal samples, n is the sample size of the normal samples, and A is the 100(1−(p/2))th percentile of the Student's t-distribution with n−1 degrees of freedom. Prior knowledge of the PARP1 gene's elevated expression in oncology samples indicated a primary interest in up-regulation relative to the baseline. Therefore, lower confidence limits were not calculated. The samples were grouped into various subcategories according to well-accepted characteristics including tumor stage, smoking status, or age. Some samples were members of more than one subcategory and some were not members of any subcategory beyond the primary cancer type. Each carcinoma sample was identified as being above the 90%, 95%, 99%, or 99.9% UCLs. Pearson's correlations were calculated for 44,759 probe sets on the Affyrmetrix HG-U133 A/B array set as compared to PARP1. Correlations were based on the set of carcinoma samples tested.

All analysis was performed using SAS v8.2 for Windows (www.sas.com) and utilized MAS 5 expression intensities as calculated from the Affymetrix GeneChip® Operating System (www.affymetrix.com). The PARP1 gene is represented on the HG-U133A array by a single probe set with the identifier “208644_at”. All results were generated based on the MAS5 expression signal intensities for this probe set.

Individual normal and cancerous samples from breast, ovarian endometrium, lung, and prostate tissues were selected. Any cancerous sample may be represented in more than one subtype grouping.

Breast Cancer Results: The expression of PARP1 in infiltrating duct carcinoma (IDC) is significantly elevated compared to normals where approximately about 70% of IDC may have PARP1 expression above the 95% upper confidence limit of the normal population, supporting findings previously observed by BiPar. As observed in the analysis, Further analysis into various subgroups of IDC samples reveals that the percentage of IDC observed to have elevated PARP1 expression increases to 88% to 89% if their ER status is negative or if their Her2-neu status is negative. The percentage of PR negative samples above the Normal 95% UCL, 79%, is less pronounced but still elevated. In addition, PARP1 expression tends to be slightly higher in the ER(−), PR(−), and Her2-neu(−) breast IDC (infiltrating duct carcinoma) classes as compared to their respective (+) classes. This finding is not observed in the p53 classes or in the tumor stage classes. The fact that individual samples are contributing to multiple categories in this analysis could be influencing this conclusion. A review of the supplementary dataset reveals that the highest PARP1 expresser in the ER(−) group is the same high expressor in the PR(−) and Her2-neu(−) groups. The same is true for the lowest expressor in the (+) groups. This suggests that any therapies targeting over expression of PARP1 may be more effective in cases where the ER, PR, or Her2-neu tests are negative.

Ovarian Results: Normal ovary and cancerous ovary samples were selected from the BioExpress® System that were members of sample sets defined for the ASCENTA® System. All of the ovarian cancers expressed higher mean PARP1 than normal ovary. Clear cell adenocarcinoma and mucinous cystadenocarcinoma samples expressed considerably lower PARP1 than did the other subtypes, and the variance in expression was also lower. In individual sample assessments, most pathologic subtypes of ovarian cancer showed a majority of samples above the 95% UCL: (a) Papillary serous, serous cystadenocarcinoma, granulosa cell tumor and Mullerian mixed tumor all had a similar high incidence of samples above the 95% UCL; (b) In endometrioid adenocarcinoma about half of the samples were above the 95% UCL; and (c) In clear cell adenocarcinoma and mucinous cystadenocarcinoma one-third or less of the samples were above the 95% UCL.

In addition, clinical sub-class comparisons of PARP1 expression in ovarian samples revealed: (a) Papillary serous stage I was similar to papillary serous stage III; and (B) Papillary serous elevated CA125 was similar to papillary serous.

Accordingly, the expression of PARP1 in ovarian cancer samples is elevated compared to normals. In addition, despite this finding, not all ovarian cancer samples exhibit this overexpression. This wider distribution and shift towards higher expression in the ovarian cancer groups indicate that ˜75% of ovarian cancers have PARP1 expression above the 95% upper confidence limit of normal ovary expression. Further analysis into various subgroups of ovarian cancer samples reveals that the percentage of ovarian cancer samples observed to have elevated PARP1 expression increases to ˜90% if they are of the subtypes papillary serous adenocarcinoma, serous cystadenocarcinoma, Mullerian mixed tumor, or granulosa cell tumor. Clear cell adenocarcinoma and mucinous cystadenocarcinoma did demonstrated elevated PARP1 in one-third or less of the samples assessed.

Endometrial Results: The expression of PARP1 in endometrial cancer was generally elevated compared to normals. Moreover, all of the endometrial cancers expressed higher mean PARP1 signal intensities than normal endometrium. The Mullerian Mixed Tumor samples expressed considerably higher PARP1 than did the other subtypes. PARP1 expression was above the 95% upper confidence limit of the normal population (“over-expression”) in about one-quarter of all endometrial, about three-quarters of all lung, and about one-eighth of all prostate cancer samples. The Mullerian mixed tumors and the lung squamous cell carcinomas exhibited the highest incidences of elevated PARP1 expression.

Individual samples from the all endometrial cancer subtypes were also individually tested relative to the normal endometrium sample distribution. Each was defined as exceeding the 90%, 95%, 99%, and 99.9% upper confidence limits of the normal set. The elevated expression of PARP1 in cancerous endometrium samples is apparent relative to normal endometrium samples. The cancerous endometrium sample expression of PARP1 exhibits a much higher degree of variation (i.e., greater spread) than that of the normal endometrium samples. No outliers were observed within the normal endometrium sample set with respect to PARP1 expression. Most pathologic subtypes of endometrium cancer showed a majority of samples above the 90% UCL. Of particular note, Mullerian Mixed Tumor had the highest incidence (85.7%) of samples above the 95% UCL and remained high (71.4%) at the 99.9% UCL

Lung Results: In normal and malignant lung sample classes, all of the lung cancers expressed higher mean PARP1 signal intensities than normal lung. Individual samples from the all lung cancer subtypes were individually tested relative to the normal lung sample distribution. The elevated expression of PARP1 in cancerous lung samples is apparent relative to normal lung samples. The cancerous lung sample expression of PARP1 exhibits a higher degree of variation (i.e., greater spread) than that of the normal lung samples.

Prostate Results: Although the prostate cancer group expressed a somewhat higher mean PARP1 signal intensity than the normal prostate group, PARP1 expression was only slightly elevated in cancerous prostate samples relative to normal prostate samples. The cancerous prostate sample expression of PARP1 exhibits a similar degree of variation (i.e., equivalent spread) than that of the normal prostate samples.

### Example 3

### Co-Expression of PARP1 mRNA and Other Targets in Normal and Carcinoma Tissues

The PARP1 gene is represented on the HG-U133A array by a single probe set with the identifier “208644_at”. Other genes, such as BRCA1, BRCA2, RAD51, MRE11, p53, PARP2 and MUCIN 16, are represented on the HG-U133A/B array set by respective informative probe sets. The list of probe sets mapped to each of the seven genes in the ovary sample analysis is listed in Table XXXIII.

TABLE XXXIII
Comparison Genes and their Corresponding HG-U133A/B Probe Set IDs

| | | Fragment |
|---|---|---|
| Gene Symbol | Title | Name(s) |
| BRCA1 | Breast cancer 1, early onset | 204531_s_at |
| BRCA2 | Breast cancer 2, early onset | 214727_at |
| MRE11A | MRE11 meiotic recombination 11 | 205395_s_at, |
| | homolog A (S. cerevisiae) | 242456_at |
| MUC16 | Mucin 16, cell surface associated | 220196_at |
| PARP2 | Poly (ADP-ribose) polymerase family, | 204752_x_at, |
| | member 2 | 214086_s_at, |
| | | 215773_x_at |
| RAD51 | RAD51 homolog (RecA homolog, | 205024_s_at |
| | E. coli) (S. cerevisiae) | |
| TP53 | Tumor protein p53 (Li-Fraumeni | 201746_at, |
| | syndrome) | 211300_s_at |

Comparison of PARP1 to Selected Genes in Ovary Samples: PARP1 expression was correlated to the expression of other genes as measured on the HG-U133A/B array set. Correlations were based on the full set of 194 samples selected for this analysis. Table XXXIV summarizes the results of this analysis. For MRE11A, PARP2, and TP53, more than one probe set is tiled on the HG-U133A/B array set.

TABLE XXXIV
Pearson correlations of PARP1 expression to selected probe sets

| | | Correlation with |
|---|---|---|
| Gene Symbol | Fragment | 208644_at (PARP1) |
| MRE11A | 205395_s_at | 0.327 |
| | 242456_at | 0.058 |
| MUC16 | 220196_at | 0.398 |
| PARP2 | 204752_x_at | 0.048 |
| | 214086_s_at | 0.052 |
| | 215773_x_at | 0.071 |
| RAD51 | 205024_s_at | 0.488 |
| TP53 | 201746_at | 0.214 |
| | 211300_s_at | 0.311 |

In no case was a negative correlation found. Positive correlations indicate that the probe sets are changing in the same direction as PARP1. When PARP1 has low expression, such as in normal samples, the expression of these correlated genes is also expected to be low. When PARP1 has elevated expression, such as in the malignant samples, the expression of these correlated genes is expected to be elevated. All of these genes, with the exception of PARP2, appear to be markers of malignancy in ovarian cancers and respond in a similar manner to PARP2.

Other genes that are co-regulated with PARP1 in ovarian cancer are included in Table XXXV below:

TABLE XXXV
Genes and their pathways that are co-regulated with PARP1 in ovarian
cancer

| Name | Description |
|---|---|
| PTGS2 | prostaglandin-endoperoxide synthase 2 |
| PARP1 | poly (ADP-ribose) polymerase family, member 1 |
| NGFB | nerve growth factor, beta |
| MKI67 | antigen identified by monoclonal antibody Ki 67 |
| IL4 | interleukin 4 |
| IGF1R | insulin-like growth factor I receptor |
| IGF1 | insulin-like growth factor 1 |
| HGF | hepatocyte growth factor |
| FOXM1 | forkhead box M1 |
| FOS | FBJ osteosarcoma oncogene |
| ESR1 | estrogen receptor 1 (alpha) |
| ERBB2 | v-erb-b2 erythroblastic leukemia viral oncogene homolog 2, |
| | neuro/glioblastoma derived oncogene homolog (avian) |
| EGR1 | early growth response 1 |
| EGFR | epidermal growth factor receptor |
| CCND1 | cyclin D1 |
| CALR | calreticulin |
| BCL2 | B-cell leukemia/lymphoma 2 |

Correlation of PARP1 expression to the genes BRCA1, BRCA2, RAD51, MRE11, p53, PARP2 and MUCIN 16 indicated significant correlation to all except PARP2. RAD51 had the highest correlation.

Correlation of PARP1 expression to genes expressed in endometrial, lung and prostate tissue samples was also tested. Correlation of PARP1 to all other genes identified genes with correlations to PARP1 as high as 80%. Among the endometrium and lung samples, a common set of genes associated with cell proliferation were identified that correlated highly (i.e. in the top 40) in both tissues.

Comparison of PARP1 to Selected Genes—Endometrium Results: PARP1 expression was correlated to all other probe sets as measured on the HG-U133A/B array set. Where available, the gene symbol and gene name have been provided for each probe set analyzed. Correlations were based on the full set of 80 samples selected for this analysis. Table XXXVI summarizes the 40 most highly correlated probe sets when compared to PARP1.

TABLE XXXVI
Pearson Correlations of PARP1 Expression to Selected Probe Sets.

| | | | Correlation to |
|---|---|---|---|
| Probe Set | Gene Symbol | Gene Name | PARP1 |
| 218585_s_at | DTL | denticleless homolog (Drosophila) | 0.765 |
| 207828_s_at | CENPF | centromere protein F, 350/400ka (mitosin) | 0.753 |
| 204444_at | KIF11 | kinesin family member 11 | 0.739 |
| 218107_at | WDR26 | WD repeat domain 26 | 0.736 |
| 211609_x_at | PSMD4, | proteasome (prosome, macropain) 26S subunit, non- | 0.727 |
| | PSMD4P2 | ATPase, 4, proteasome (prosome, macropain) 26S | |
| | | subunit, non-ATPase, 4, pseudogene 2 | |
| 218252_at | CKAP2 | cytoskeleton associated protein 2 | 0.719 |
| 210460_s_at | PSMD4, | proteasome (prosome, macropain) 26S subunit, non- | 0.714 |
| | PSMD4P2 | ATPase, 4, proteasome (prosome, macropain) 26S | |
| | | subunit, non-ATPase, 4, pseudogene 2 | |
| 210052_s_at | TPX2 | TPX2, microtubule-associated, homolog (Xenopus | 0.709 |
| | | laevis) | |
| 206364_at | KIF14 | kinesin family member 14 | 0.708 |
| 200910_at | CCT3 | chaperonin containing TCP1, subunit 3 (gamma) | 0.707 |
| 200896_x_at | HDGF | hepatoma-derived growth factor (high-mobility group | 0.704 |
| | | protein 1-like) | |
| 218605_at | TFB2M | transcription factor B2, mitochondrial | 0.703 |
| 202107_s_at | MCM2 | MCM2 minichromosome maintenance deficient 2, | 0.701 |
| | | mitotin (S. cerevisiae) | |
| 201292_at | TOP2A | topoisomerase (DNA) II alpha 170 kDa | 0.699 |
| 236641_at | KIF14 | kinesin family member 14 | 0.698 |
| 204822_at | TTK | TTK protein kinase | 0.695 |
| 223381_at | CDCA1 | cell division cycle associated 1 | 0.692 |
| 201664_at | SMC4 | structural maintenance of chromosomes 4 | 0.691 |
| 202954_at | UBE2C | ubiquitin-conjugating enzyme E2C | 0.690 |
| 226242_at | C1orf131 | chromosome 1 open reading frame 131 | 0.686 |
| 201663_s_at | SMC4 | structural maintenance of chromosomes 4 | 0.685 |
| 228273_at | | | 0.685 |
| 225766_s_at | TNPO1 | transportin 1 | 0.685 |
| 223530_at | TDRKH | tudor and KH domain containing | 0.685 |
| 203145_at | SPAG5 | sperm associated antigen 5 | 0.684 |
| 222680_s_at | DTL | denticleless homolog (Drosophila) | 0.682 |
| 212023_s_at | MKI67 | antigen identified by monoclonal antibody Ki-67 | 0.676 |
| 222433_at | ENAH | enabled homolog (Drosophila) | 0.670 |
| 209172_s_at | CENPF | centromere protein F, 350/400ka (mitosin) | 0.670 |
| 219918_s_at | ASPM | asp (abnormal spindle)-like, microcephaly associated | 0.669 |
| | | (Drosophila) | |
| 200594_x_at | HNRPU | Heterogeneous nuclear ribonucleoprotein U (scaffold | 0.666 |
| | | attachment factor A) | |
| 222752_s_at | C1orf75 | chromosome 1 open reading frame 75 | 0.663 |
| 201478_s_at | DKC1 | dyskeratosis congenital 1, dyskerin | 0.663 |
| 208938_at | PRCC | papillary renal cell carcinoma (translocation-associated) | 0.663 |
| 201381_x_at | CACYBP | calcyclin binding protein | 0.662 |
| 202580_x_at | FOXM1 | forkhead box M1 | 0.661 |
| 201479_at | DKC1 | dyskeratosis congenital 1, dyskerin | 0.661 |
| 201774_s_at | CNAP1 | chromosome condensation-related SMC-associated | 0.657 |
| | | protein 1 | |
| 211762_s_at | KPNA2, | hypothetical protein MGC40489, karyopherin alpha 2 | 0.656 |
| | LOC643995, | (RAG cohort 1, importin alpha 1), region containing | |
| | LOC645625, | similar to pleckstrin homology domain containing, family | |
| | LOC650526, | M (with RUN domain) member 1; adapter protein 162; | |
| | MGC40489 | hypothetical protein MGC40489, similar to Importin | |
| | | alpha-2 subunit (Karyopherin alpha-2 subunit) (SRP1- | |
| | | alpha) (RAG cohort protein 1) | |

The gene that correlates best with PARP1 expression is DTL with a Pearson correlation of 0.765. The top 40 probe sets all had positive correlations to PARP1. Positive correlations represent cases where the change in expression in PARP1 and the positively correlated probe sets is the same. Negatively correlated probe sets were also seen, but none of those negative correlations ranked in the top 40 on the absolute scale. The highest negatively correlated probe set mapped to the HOM-TES-103 gene (Hypothetical Protein LOC25900, isoform 3) with a correlation of −0.636.

Comparison of PARP1 to Selected Genes—Lung Results: PARP1 expression was correlated to all other probe sets as measured on the HG-U133A/B array set. Where available, the gene symbol and gene name have been provided for each probe set analyzed. Correlations were based on the set of 347 samples, after removal of four outlier normal samples, selected for this analysis. Table XXXVII summarizes the 40 most highly correlated probe sets when compared to PARP1.

TABLE XXXVII
Pearson Correlations of PARP1 Expression to Selected Probe Sets.

| | | | Correlation to |
|---|---|---|---|
| Probe Set | Gene Symbol | Gene Name | PARP1 |
| 223229_at | UBE2T | ubiquitin-conjugating enzyme E2T (putative) | 0.815 |
| 204641_at | NEK2 | NIMA (never in mitosis gene a)-related kinase 2 | 0.785 |
| 206550_s_at | NUP155 | nucleoporin 155 kDa | 0.749 |
| 225082_at | CPSF3 | cleavage and polyadenylation specific factor 3, 73 kDa | 0.745 |
| 204962_s_at | CENPA | centromere protein A | 0.740 |
| 207828_s_at | CENPF | centromere protein F, 350/400ka (mitosin) | 0.728 |
| 222640_at | DNMT3A | DNA (cytosine-5-)-methyltransferase 3 alpha | 0.717 |
| 209642_at | BUB1 | BUB1 budding uninhibited by benzimidazoles 1 | 0.712 |
| | | homolog (yeast) | |
| 203316_s_at | LOC645472, | similar to small nuclear ribonucleoprotein E, small | 0.711 |
| | LOC648527, | nuclear ribonucleoprotein polypeptide E, small nuclear | |
| | LOC651086, | ribonucleoprotein polypeptide E-like 1 | |
| | SNRPE, | | |
| | SNRPEL1 | | |
| 209971_x_at | JTV1 | JTV1 gene | 0.710 |
| 216952_s_at | LMNB2 | lamin B2 | 0.709 |
| 202580_x_at | FOXM1 | forkhead box M1 | 0.708 |
| 204033_at | TRIP13 | thyroid hormone receptor interactor 13 | 0.707 |
| 221436_s_at | CDCA3 | cell division cycle associated 3 | 0.706 |
| 222958_s_at | DEPDC1 | DEP domain containing 1 | 0.704 |
| 209408_at | KIF2C | kinesin family member 2C | 0.700 |
| 210052_s_at | TPX2 | TPX2, microtubule-associated, homolog (Xenopus | 0.699 |
| | | laevis) | |
| 203145_at | SPAG5 | sperm associated antigen 5 | 0.697 |
| 208079_s_at | AURKA | aurora kinase A | 0.696 |
| 202705_at | CCNB2 | cyclin B2 | 0.695 |
| 201897_s_at | CKS1B | CDC28 protein kinase regulatory subunit 1B | 0.695 |
| 220147_s_at | FAM60A | family with sequence similarity 60, member A | 0.694 |
| 219918_s_at | ASPM | asp (abnormal spindle)-like, microcephaly associated | 0.694 |
| | | (Drosophila) | |
| 208696_at | CCT5 | chaperonin containing TCP1, subunit 5 (epsilon) | 0.692 |
| 201263_at | TARS | threonyl-tRNA synthetase | 0.692 |
| 218252_at | CKAP2 | cytoskeleton associated protein 2 | 0.692 |
| 202870_s_at | CDC20 | CDC20 cell division cycle 20 homolog (S. cerevisiae) | 0.692 |
| 218512_at | WDR12 | WD repeat domain 12 | 0.690 |
| 225244_at | C1orf142 | chromosome 1 open reading frame 142 | 0.688 |
| 201013_s_at | PAICS | phosphoribosylaminoimidazole carboxylase, | 0.687 |
| | | phosphoribosylaminoimidazole succinocarboxamide | |
| | | synthetase | |
| 202613_at | CTPS | CTP synthase | 0.686 |
| 212694_s_at | PCCB | propionyl Coenzyme A carboxylase, beta polypeptide | 0.684 |
| 203432_at | TMPO | Thymopoietin | 0.684 |
| 214710_s_at | CCNB1 | cyclin B1 | 0.684 |
| 218355_at | KIF4A | kinesin family member 4A | 0.680 |
| 201698_s_at | SFRS9 | splicing factor, arginine/serine-rich 9 | 0.679 |
| 202095_s_at | BIRC5 | baculoviral IAP repeat-containing 5 (survivin) | 0.679 |
| 202690_s_at | SNRPD1 | small nuclear ribonucleoprotein D1 polypeptide 16 kDa | 0.677 |
| 204444_at | KIF11 | kinesin family member 11 | 0.677 |

The gene that correlates best with PARP1 expression is UBE2T with a Pearson correlation of 0.815. The top 40 probe sets all had positive correlations to PARP1. Positive correlations represent cases where the change in expression in PARP1 and the positively correlated probe sets is the same. Negatively correlated probe sets were also seen, but none of those negative correlations ranked in the top 40 on the absolute scale. The highest negatively correlated probe set mapped to the TGFBR2 gene (Transforming Growth Factor, beta receptor II) with a correlation of −0.670.

Comparison of PARP1 to Selected Genes—Prostate Results: PARP1 expression was correlated to all other probe sets as measured on the HG-U133A/B array set. Some probe sets map to the same gene while other probe sets have no known gene annotation available. Where available, the gene symbol and gene name have been provided for each probe set analyzed. Correlations were based on the set of 114 samples selected for this analysis. Table XXXVIII summarizes the 40 most highly correlated probe sets when compared to PARP1.

TABLE XXXVIII
Pearson Correlations of PARP1 Expression to Selected Probe Sets.

| | | | Correlation to |
|---|---|---|---|
| Probe Set | Gene Symbol | Gene Name | PARP1 |
| 212871_at | MAPKAPK5 | mitogen-activated protein kinase-activated protein | 0.522 |
| | | kinase 5 | |
| 221761_at | ADSS | adenylosuccinate synthase | 0.517 |
| 226470_at | GGTL3 | gamma-glutamyltransferase-like 3 | −0.515 |
| 201376_s_at | HNRPF | heterogeneous nuclear ribonucleoprotein F | 0.476 |
| 200764_s_at | CTNNA1 | catenin (cadherin-associated protein), alpha 1, | 0.471 |
| | | 102 kDa | |
| 203992_s_at | UTX | ubiquitously transcribed tetratricopeptide repeat, X | 0.468 |
| | | chromosome | |
| 217791_s_at | ALDH18A1 | aldehyde dehydrogenase 18 family, member A1 | 0.466 |
| 210027_s_at | APEX1 | APEX nuclease (multifunctional DNA repair | 0.466 |
| | | enzyme) 1 | |
| 201209_at | HDAC1 | histone deacetylase 1 | 0.465 |
| 217970_s_at | CNOT6 | CCR4-NOT transcription complex, subunit 6 | 0.462 |
| 217748_at | ADIPOR1 | adiponectin receptor 1 | 0.459 |
| 201829_at | NET1 | neuroepithelial cell transforming gene 1 | 0.458 |
| 210250_x_at | ADSL | adenylosuccinate lyase | 0.458 |
| 203739_at | ZNF217 | zinc finger protein 217 | 0.453 |
| 203222_s_at | TLE1 | transducin-like enhancer of split 1 (E(sp1) homolog, | 0.452 |
| | | Drosophila) | |
| 222777_s_at | WHSC1 | Wolf-Hirschhorn syndrome candidate 1 | 0.449 |
| 204005_s_at | PAWR | PRKC, apoptosis, WT1, regulator | 0.448 |
| 204667_at | FOXA1 | forkhead box A1 | 0.448 |
| 204400_at | EFS | embryonal Fyn-associated substrate | −0.447 |
| 205925_s_at | RAB3B | RAB3B, member RAS oncogene family | 0.446 |
| 215714_s_at | SMARCA4 | SWI/SNF related, matrix associated, actin dependent | 0.444 |
| | | regulator of chromatin, subfamily a, member 4 | |
| 212602_at | WDFY3 | WD repeat and FYVE domain containing 3 | 0.444 |
| 219281_at | MSRA | methionine sulfoxide reductase A | −0.444 |
| 211938_at | EIF4B | eukaryotic translation initiation factor 4B | 0.442 |
| 200644_at | MARCKSL1 | MARCKS-like 1 | 0.442 |
| 213541_s_at | ERG | v-ets erythroblastosis virus E26 oncogene like | 0.439 |
| | | (avian) | |
| 203932_at | HLA-DMB | major histocompatibility complex, class II, DM beta | 0.439 |
| 203593_at | CD2AP | CD2-associated protein | 0.439 |
| 37005_at | NBL1 | neuroblastoma, suppression of tumorigenicity 1 | −0.437 |
| 223566_s_at | BCOR | BCL6 co-repressor | 0.437 |
| 208778_s_at | TCP1 | t-complex 1 | 0.435 |
| 204363_at | F3 | coagulation factor III (thromboplastin, tissue factor) | 0.435 |
| 203769_s_at | STS | steroid sulfatase (microsomal), arylsulfatase C, | −0.435 |
| | | isozyme S | |
| 201830_s_at | NET1 | neuroepithelial cell transforming gene 1 | 0.435 |
| 207627_s_at | TFCP2 | transcription factor CP2 | 0.434 |
| 212836_at | POLD3 | polymerase (DNA-directed), delta 3, accessory | −0.434 |
| | | subunit | |
| 210291_s_at | ZNF174 | zinc finger protein 174 | 0.434 |
| 201118_at | PGD | phosphogluconate dehydrogenase | 0.430 |
| 200751_s_at | HNRPC, | heterogeneous nuclear ribonucleoprotein C (C1/C2), | 0.430 |
| | LOC653447 | similar to heterogeneous nuclear ribonucleoprotein C | |

The gene that correlates best with PARP1 expression is MAPKAPK5 with a pearson correlation of 0.522. The top 40 probe sets had a mix of positive and negative correlations to PARP1. Positive correlations represent cases where the change in expression in PARP1 and the positively correlated probe set is in the same direction. Negatively correlated probe sets represent cases where the expression change is in the opposite direction as PARP1. The highest negatively correlated probe set mapped to the GGTL3 gene (Gamma-glutamyltransferase-like 3) with a correlation of −0.515.

Correlation of PARP1 expression to the other genes on the HG-U133 A/B array set identified genes with correlations as high as 70% to 80% in endometrium and lung. Although the best correlating gene in each tissue was not the same, there were some concordant probe sets among the top 40 lists. Table XXXIX lists the 7 probe sets that were ranked in the top 40 for both endometrium and lung and displays linked Gene Ontology Biological Process terms. The genes represented are associated with cell proliferation. None of these probes sets rank in the top 5000 for the prostate samples selected for this analysis.

TABLE XXXIX
Concordant Probe Sets Between 40 Best Correlated Probe Sets in Lung and Endometrium.

| | | | | | Lung | | | |
|---|---|---|---|---|---|---|---|---|
| | | | | Endometrium | Correlation | | | |
| Probe | Gene | | Biological Process | Correlation | to | Endometrium | Lung | Average |
| set | Symbol | Gene name | (GO Terms) | to PARP1 | PARP1 | Rank | Rank | Rank |
| 207828_s_at | CENPF | Centromere | G2 phase of mitotic | 0.75314 | 0.72803 | 2 | 6 | 4 |
| | | protein F, | cell cycle, cell | | | | | |
| | | 350/400ka | division, cell | | | | | |
| | | (mitosin) | proliferation, | | | | | |
| | | | kinetochore | | | | | |
| | | | assembly, | | | | | |
| | | | metaphase plate | | | | | |
| | | | congression, | | | | | |
| | | | mitosis, mitotic | | | | | |
| | | | spindle checkpoint, | | | | | |
| | | | negative regulation | | | | | |
| | | | of transcription, | | | | | |
| | | | regulation of striated | | | | | |
| | | | muscle | | | | | |
| | | | development, | | | | | |
| | | | response to drug | | | | | |
| 202580_x_at | FOXM1 | Forkhead | Regulation of | 0.66143 | 0.70839 | 36 | 12 | 24 |
| | | box M1 | transcription, DNA- | | | | | |
| | | | dependent, | | | | | |
| | | | transcription | | | | | |
| 210052_s_at | TPX2 | TPX2, | Cell proliferation, | 0.70885 | 0.69871 | 8 | 17 | 12.5 |
| | | microtubule- | mitosis | | | | | |
| | | associated, | | | | | | |
| | | homolog | | | | | | |
| | | (Xenopus | | | | | | |
| | | laevis) | | | | | | |
| 203145_at | SPAG5 | Sperm | Cell cycle, cell | 0.68365 | 0.69731 | 25 | 18 | 21.5 |
| | | associated | division, mitosis, | | | | | |
| | | antigen 5 | phosphoinositide- | | | | | |
| | | | mediated signaling, | | | | | |
| | | | spindle organization | | | | | |
| | | | and biogenesis | | | | | |
| 219918_s_at | ASPM | Asp | Cell cycle, cell | 0.66859 | 0.69381 | 30 | 23 | 26.5 |
| | | (abnormal | division, mitosis | | | | | |
| | | spindle)- | | | | | | |
| | | like | | | | | | |
| | | microcephaly | | | | | | |
| | | associated | | | | | | |
| | | (Drosophila) | | | | | | |
| 218252_at | CKAP2 | Cytoskeleton | Not assigned | 0.71875 | 0.69163 | 6 | 26 | 16 |
| | | associated | | | | | | |
| | | protein 2 | | | | | | |
| 204444_at | KIF11 | Kinesin | Cell cycle, cell | 0.73886 | 0.67701 | 3 | 39 | 21 |
| | | family | division, | | | | | |
| | | member 11 | microtubule-based | | | | | |
| | | | movement, mitosis, | | | | | |
| | | | mitotic spindle | | | | | |
| | | | organization and | | | | | |
| | | | biogenesis | | | | | |

PARP1 is involved in base excision repair following DNA damage and appears as an obligatory step in a detection/signaling pathway leading to the repair of DNA strand breaks. It is therefore noteworthy that PARP1 is co-regulated with other genes that are essential for cell cycle, chromosome separation, cell division and mitosis. The best correlating probe set in prostate has a notably lower correlation than the best correlating probe sets in either endometrium or lung. If PARP1 is relatively unchanged in normal prostate versus prostate adenocarcinoma, age 60 and over, it is not surprising that the PARP1 expression in prostate would have lower correlations to the other probe sets on the array set. Because of the lack of statistical significance in the cancer group, the best correlating prostate gene list was not compared to the other tissues.

Conclusions: The expression of PARP1 in endometrial and lung cancer samples is generally elevated compared to normals. Similar signal elevation was not seen the in the prostate cancer samples evaluated. The figures show that, despite this finding, not all endometrial and lung cancer samples exhibit this overexpression. This wider distribution and shift towards higher expression in the endometrial and lung cancer groups indicate that ˜37% of endometrial and ˜77% of lung cancers have PARP1 expression above the 95% upper confidence limit of their respective normal expression. Further analysis into various subgroups of endometrial cancer samples reveals that the percentage of cancer samples observed to have elevated PARP1 expression increases to ˜86% if they are of the Mullerian mixed tumor subtype. Clear Cell Adenocarcinoma and Mucinous Cystadenocarcinoma did demonstrated elevated PARP1 in one-third or less of the samples assessed and may represent less sensitive cancer types. These findings should be further investigated and confirmed. In summary, (1) PARP1 expression is higher in endometrial and lung cancer than in their respective normal tissue; (2) certain subtypes of endometrial and lung cancer appear to exhibit higher expression levels than other subtypes. Specifically, Mullerian mixed tumor, and lung squamous cell carcinoma samples showed higher percentages of samples above the Normal UCL's than the other classes; and (3) 7 genes were ranked in both the endometrial and lung top 40 probe sets that correlate best with PARP1. These genes are associated with cell proliferation and mitosis.

### Example 4

### Monitoring PARP Expression in Tissue Samples

Assay Description and Methods: XP™-PCR is a multiplex RT-PCR methodology that allows for the expression analysis of multiple genes in a single reaction (Quin-Rong Chen et al.: Diagnosis of the Small Round Blue Cell Tumors Using Mutliplex Polymerase Chain Reaction. J. Mol. Diagnostics, Vol. 9. No. 1, February 2007). A defined combination of gene specific and universal primers used in the reaction results in a series of fluorescently labeled PCR products whose size and quantity are measured using the capillary electrophoresis instrument GeXP.

Sample Treatments: Briefly, freshly purified tissue samples will be plated in 24-well plates at 6×10⁶ cells per well. One half of the samples will be lysed immediately and the others will be quickly frozen in a dry ice and ethanol bath and stored at −80° C. for 24 hours. Total RNA from each sample will be isolated following Althea Technologies, Inc. SOP

Total RNA Isolation Using Promega SV96 Kit (Cat. No. Z3505). The concentration of the RNA obtained from each sample will be obtained using 03-XP-008, RNA Quantitation Using the Quant-it Ribogreen RNA Assay Kit (Cat. No. R-11490). A portion of RNA from each sample will be adjusted to 5 ng/μL and then subjected to XP™-PCR.

XP™-PCR: Multiplex RT-PCR will be performed using 25 ng of total RNA of each sample using a previously described protocol (Quin-Rong Chen et al.: Diagnosis of the Small Round Blue Cell Tumors Using Mutliplex Polymerase Chain Reaction. J. Mol. Diagnostics, Vol. 9. No. 1, February 2007). The RT reactions will be carried out as described in SOP 11-XP-002, cDNA Production from RNA with the Applied Biosystems 9700. PCR reactions will be carried out on each cDNA according to SOP 11-XP-003, XP™-PCR with the Applied Biosystems 9700. To monitor efficiency of the RT and PCR reactions 0.24 attamoles of Kanamycin RNA will be spiked into each RT reaction. Two types of positive control RNA will be used. Other assay controls include ‘No Template Controls’ (NTC) where water instead of RNA will be added to separate reactions and ‘Reverse Transcriptase minus’ (RT−) controls where sample RNA will be subjected to the procedure without reverse transcriptase.

Expression Analysis and Calculations: PCR reactions will be analyzed by capillary electrophoresis. The fluorescently labeled PCR reactions will be diluted, combined with Genome Lab size standard-400 (Beckman-Coulter, Part Number 608098), denatured, and loaded onto the Beckman Coulter using SOP 11-XP-004, Operation and Maintenance of the CEQ 8800 Genetic Analysis System. The data obtained from the 8800 will be analyzed with expression analysis software to generate relative expression values for each gene. The expression of each target gene relative to the expression of either cyclophilin A, GAPDH, or β-actin within the same reaction is reported as the mean of the replicate. The standard deviation and percent coefficient of variance (% CV) associated with these values will also be reported when appropriate.

Statistical Analysis Method: The mathematical form of the ANOVA model to be used in this analysis is:

Yᵢⱼₖₗ=μ+αᵢ+βⱼ+γₖ+ωₗ₍ᵢⱼₖ₎+εᵢⱼₖₗ i=1 . . . 5 j=1 . . . 4 k=1 . . . 3 l=1 . . . 3

Coν(Yᵢⱼₖₗ, Yᵢⱼₖₗ)=σ²_{ω}+σ²_{τ} Coν(Yᵢⱼₖₗ,Y_{ijkl′})=σ²_{ω} Coν(Yᵢⱼₖₗ,Y_{ijk′l})=0 (1)

Here Yᵢⱼₖₗ is the normalized Rfu ratio obtained in the i^{th} sample under the j^{th} dosing concentration at the k^{th} time point from the l^{th} replicate. The model parameter μ is the overall mean normalized Rfu ratio, an unknown constant, αᵢ is a fixed effect due to sample i, βⱼ is a fixed effect due to dosing concentration j, γₖ is a fixed effect due to time point k, and ωₗ₍ᵢⱼₖ₎ is a random effect due to the l^{th} replicate in the i^{th} sample under j^{th} dosing concentration at k^{th} time point, which is assumed Normally distributed with mean 0 and variance σ²_{ω}. εᵢⱼₖₗ is a random error term associated with the normalized Rfu ratio from the i^{th} sample under the j^{th} dosing concentration at the k^{th} time point from the l^{th} replicate, assumed Normally distributed with mean 0 and variance σ_{ε}².

lme function in nlme package in R will be used to analyze the data with respect to the model above. The overall dosing effect (H₀: β₁=β₂=β₃=β₄=β₅=0 versus H₁: At least one βᵢ is different) will be tested in F-test for each gene.

### Example 5

### PARP Expression in Syngeneic Samples Using Q-RT-PCR

Assay Description and Methods: XP™-PCR is a multiplex RT-PCR methodology that allows for the expression analysis of multiple genes in a single reaction (Kahn et al., 2007). A defined combination of gene specific and universal primers used in the reaction results in a series of fluorescently labeled PCR products whose size and quantity are measured using the capillary electrophoresis instrument GeXP.

XP™-PCR: Multiplex RT-PCR was performed using 25 ng of total RNA of each sample using a previously described protocol (Khan et al., 2007). The RT reactions were carried out as described in SOP 11-XP-002, cDNA Production from RNA with the Applied Biosystems 9700. PCR reactions were carried out on each cDNA according to SOP 11-XP-003, XP™-PCR with the Applied Biosystems 9700. To monitor efficiency of the RT and PCR reactions 0.24 attamoles of Kanamycin RNA was spiked into each RT reaction. A positive control RNA was used and is detailed below in the Assay Discussion section. Other assay controls included ‘No Template Controls’ (NTC) where water instead of RNA was added to separate reactions and ‘Reverse Transcriptase minus’ (RT−) controls where sample RNA was subjected to the procedure without reverse transcriptase.

Expression Analysis and Calculations: PCR reactions were analyzed by capillary electrophoresis. The fluorescently labeled PCR reactions were diluted, combined with Genome Lab size standard-400 (Beckman-Coulter, Part Number 608098), denatured, and loaded onto the Beckman Coulter using SOP 11-XP-004, Operation and Maintenance of the CEQ 8800 Genetic Analysis System. The data obtained from the 8800 was analyzed with our proprietary expression analysis software to generate relative expression values for each gene. The expression of each target gene relative to the expression of glucuronidase beta (GUSB) within the same reaction is reported as the mean of the replicate. The standard deviation and percent coefficient of variance (% CV) associated with these values are also reported when appropriate.

Sample Description: Frozen human breast and lung tissues were obtain during surgery as a syngeneic pair on dry ice. They consisted of a tumor sample and a normal sample from each of studied individuals.

Sample RNA Extraction: RNA was extracted from each sample using a RiboPure™ RNA isolation kit from Ambion Cat. # 1924). To insure that the samples would be thawed only under RNase denaturing conditions, each frozen sample was placed on a new sample collection tray on top of dry ice. Using a new razor blade for each sample, an approximately 100 mg piece of lung tissue and 200 mg piece of breast tissue was cut and immediately placed into a labeled tube containing the TRI Reagent and two ceramic beads. The samples were then homogenized using a Qiagen Laboratory Vibration Mill Type MM300 for 2 minutes at 20 MHz. The orientation of the mixer mill sample block was then reversed and the samples were homogenized for another 2 minutes. The RNA was then isolated from the homogenate following the RiboPure™ protocol supplied with the kit.

Following isolation, each sample of RNA was subjected to a DNase reaction following SOP 3-XP-001 DNase I treatment of RNA to remove any residual sample DNA.

Immediately following the DNase heat inactivation step of the DNase reaction, the ribonuclease inhibitor SUPERase-In (Ambion, Cat. No. AM2696) was added to each sample at a final concentration of 1 U/μL.

RNA Quantitation: The concentration of the RNA was determined using the RiboGreen RNA Quantitation Kit (Invitrogen, Cat. No. R11490) and by following SOP 3-EQ-031 Wallac Victor2 1420 Multilabel Counter.

Sample RNA Quality: A sample of RNA from each sample was analyzed on an Agilent Bioanalyzer following Althea Technology's SOP 11-XP-001 Operation of Agilent 2100 Bioanalyzer.

Sample Requirements: Samples were processed according to the following protocols: Triplicate definition (each sample of RNA was assayed in three separate XP™-PCR reactions) and RT-PCR Reaction Sample Requirements (25 ng of total RNA was utilized in each reaction).

XP™-PCR: RT-PCR Controls are as follows: (1) The reverse transcription controls for the presence of DNA contamination in the RNA (RT minus) were negative; and (2) The PCR controls for DNA contamination in the reagents (no template control) were negative. Positive Control: The human positive control RNA that was used in the assay was Ambion Human Reference RNA (HUR), (Ambion, custom order).

### Pathway Analysis of PARP1-Activated Tumors

Data sources: Gene expression dataset received from BiPar Sciences were analyzed using Reset 5.0 molecular interaction database (Yuryev et al., 2006, Bioinformatics, 7:171). The release database was enhanced with 2344 biological process pathways automatically build, 249 cellular component networks and 129 metabolic pathways from KEGG (Daraselia et al., 2007, Bioinformatics, 8:243).

Identification of samples with PARP1 differential expression: The analysis of PARP1-activated tumors was performed using the expression data provided by BiPar Sciences Inc. The samples from four tumor tissues were analyzed: breast, endometrium, ovary and lung. The MAS5 normalized samples from every tissue were separated into two classes: tumors with low PARP1 expression and tumors with high PARP1 expression. The minimum difference in PARP1 expression between any pair of samples from two classes was 2-fold change. The results of finding samples with differential PARP1 expression are shown in Table XL.

TABLE XL
Results of selecting samples with PARP1 differential expression

| | | | | No. of | |
|---|---|---|---|---|---|
| | | | No. of | samples | |
| | | File with | samples with | with high | |
| | | selected | low PARP1 | PARP1 | |
| Tissue | Original BiPar files | samples | expression | expression | Chip |
| Breast | 10766_BIPAR_2nd_48_MAS5_HU133.txt, | Breast DE | 17 | 17 | HG-U133 |
| | 10766_BIPAR_1st_48_MAS5_HU133.txt | samples with | | | |
| | | PARP1 | | | |
| | | correlation.txt | | | |
| Endometrium | 10766_BIPAR_2nd_48_MAS5_HU133.txt, | Endometrium | 8 | 8 | HG-U133 |
| | 10766_BIPAR_1st_48_MAS5_HU133.txt | DE samples with | | | |
| | | PARP1 | | | |
| | | correlation.txt | | | |
| Ovary | 10727_BIPAR_MAS5_HG- | Ovary DE | 3 | 6 | HG-U133 |
| | U133A_with_gene_annotations.txt, | samples with | | | |
| | 10727_BIPAR_MAS5_HG- | PARP1 | | | |
| | U133B_with_gene_annotations.txt | correlation.txt | | | |
| Lung | JA00567.xls | Lung syngeneic | 1 | 1 | HG-U133 |
| | | DE samples with | | | Plus 2.0 |
| | | PARP1 | | | |
| | | correlation.txt | | | |

All files with selected samples have the following columns:
Columns with gene identifiers from the original microarray file;
Correlation mode—absolute value of the gene profile correlation with PARP1 gene;
Correlation—gene profile correlation with PARP1 gene;
high/low log ratio—log 2 ratio of the average expression of a gene in PARP1 high-expressing tumors to average expression of a gene in PARP1 low-expressing tumors;
Samples with low PARP1 expression; and
Samples with high PARP1 expression.

Identification of significant genes: The fold of expression change for every gene was calculated as the log ratio between average normalized signal intensity among samples with low PARP1 levels and corresponding average among tumors with high PARP1 expression. For lung samples where the data about normal tissues was available the ratio was calculated as the difference between the fold change expression in PARP1 over-expressing tumors relative to normal tissues and the fold change expression in PARP1 low-expressing tumors relative to normal tissues.

The p-values indicating the confidence of the differential expression was calculated using unpaired t-test for breast, endometrium and ovarian samples. It was impossible to calculate p-value for lung samples because they had only one sample for each class of tumors.

TABLE XLI
Identification of significant genes. The table contains actual gene
count, duplicate probes were removed, probes that could not mapped
onto proteins in ResNet5 were not counted

| | | | >2 fold + p- | |
|---|---|---|---|---|
| | >2 fold change | >0.01 p-value | value change | |
| Tissue | cutoff | cutoff | cutoff | File with p-value calculated |
| Breast | 2169 | 2824 | 416 | Breast DE samples with |
| | | | | PARP1 correlation p-value.txt |
| Endometrium | 3936 | 1030 | 409 | Endometrium DE samples with |
| | | | | PARP1 correlation p-value.txt |
| Ovary | 4614 | 344 | 189 | Ovary DE samples with |
| | | | | PARP1 correlation p-value.txt |
| Lung | 4923 | N/A | N/A | Lung syngeneic DE samples |
| | | | | with PARP1 correlation.txt |

All files with selected samples have the following columns:
Columns with gene identifiers from the original microarray file;
Correlation mode—absolute value of the gene profile correlation with PARP1 gene;
Correlation—gene profile correlation with PARP1 gene;
high/low log ratio—log 2 ratio of the average expression of a gene in PARP1 high-expressing tumors to average expression of a gene in PARP1 low-expressing tumors;
p-value of differential expression calculated by unpaired t-test;
average expression value in PARP1 low-expressing tumors;
average expression value in PARP1 high-expressing tumors;
Samples with low PARP1 expression; and
Samples with high PARP1 expression.

Comparative analysis of significant genes: For each of three statistical cutoffs described in Table XLI the following comparative analysis was performed on three levels: (1) Direct comparison of differentially expressed genes to find significant genes common for three or four tissues; (2) Comparative Gene Ontology analysis to find GO groups differentially expressed and common for three or four tissues; and (3) Comparative pathway analysis to find pathways differentially expressed/co-regulated and common for three or four tumor types (breast, ovarian, endometrium and lung).

The common significant genes, GO groups and pathways were first identified between three tissues: breast, endometrium and ovary. Separately the common significant genes were identified between all four tissues. This was done intentionally due to the small number of samples from lung tissue that could skew the comparative analysis.

The identification of common GO groups and pathways was performed using “Find groups” and “Find pathway” option in Pathway Studio for each tissue. “Find groups” and “Find pathway” options identify significant groups and pathway by comparing differentially expressed genes with groups and pathway in the Pathway Studio database using Fisher Exact test.

The groups/pathway common for three or four tissues were found by calculating the intersection between lists of GO groups or lists of pathways. Only groups/pathways with Fisher Exact test p-value smaller than 0.001 were considered for finding groups/pathways common among all tissues.

The results of comparative analysis for each of three statistical cutoffs are: 2 fold cutoff; p-value 0.01 cutoff; and 2-fold +p-value 0.01 cutoff.

The results of comparative Gene Ontology and pathway analysis depict the list of GO groups and pathways with significant overrepresentation of differentially expressed genes for every tissue as well as the GO groups and pathways over represented in all four tissues.

Ontology analysis of significant genes: Gene Ontology analysis of significant genes was performed using Fisher Exact test as described in previous section. The results of the analysis are available as follows: 2 fold cutoff; p-value 0.01 cutoff; and 2-fold +p-value 0.01 cutoff.

Network analysis: Physical networks were built from significant genes identified for each tissue using Build pathway tool option “Find direct interactions between selected entities” with filter settings to include Binding interaction only. The networks were built for each tissue as well as for significant genes common for all three tissues.

The expression regulatory network was built using Build pathway tool option “Find direct interactions between selected entities” with filter settings to include Expression and Promoter Binding regulatory relations.

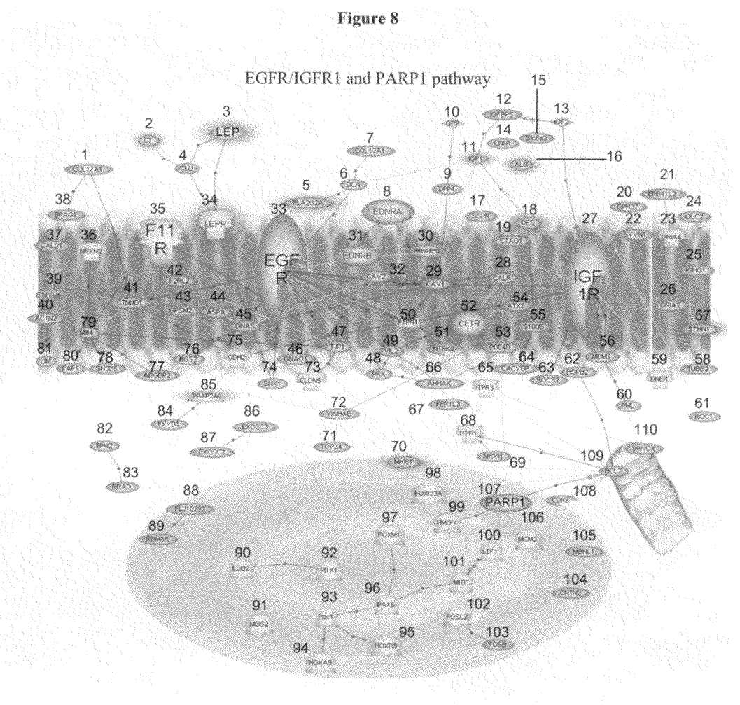

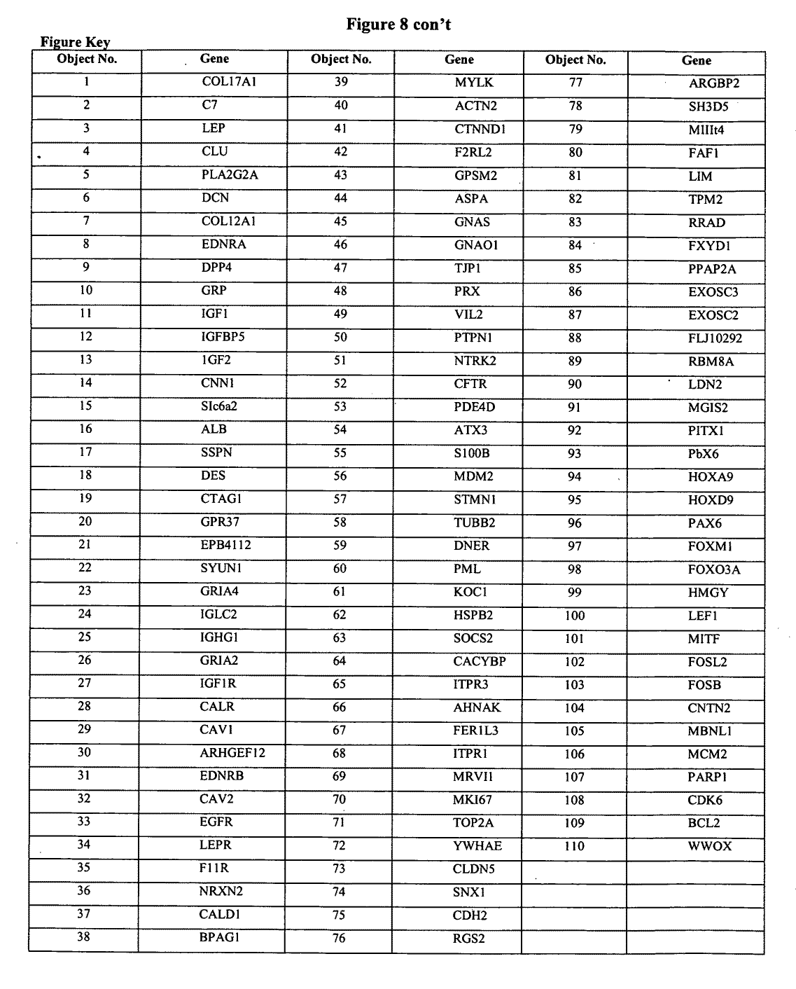

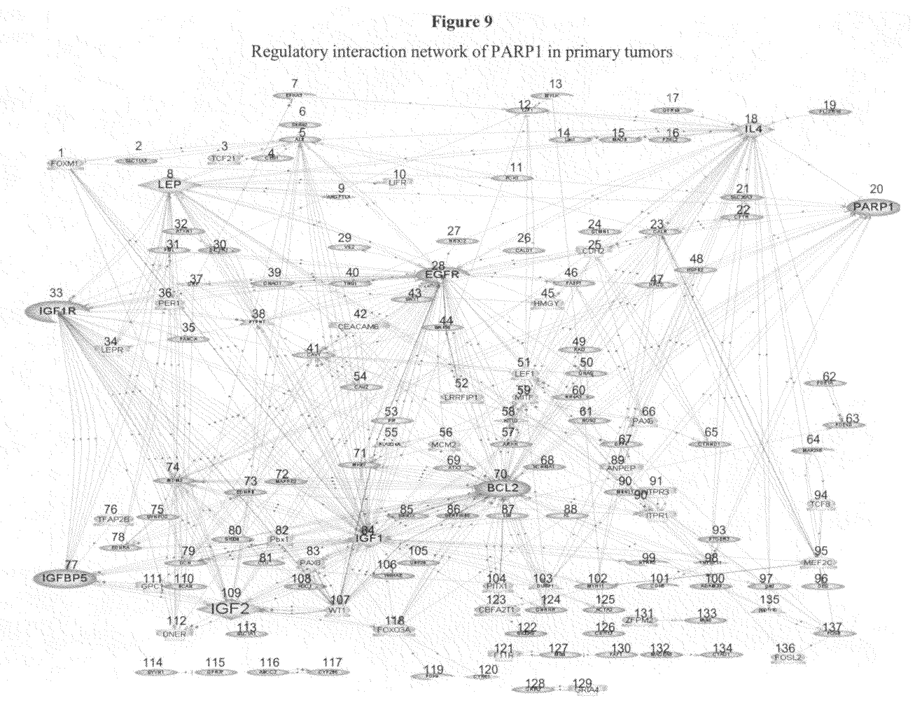

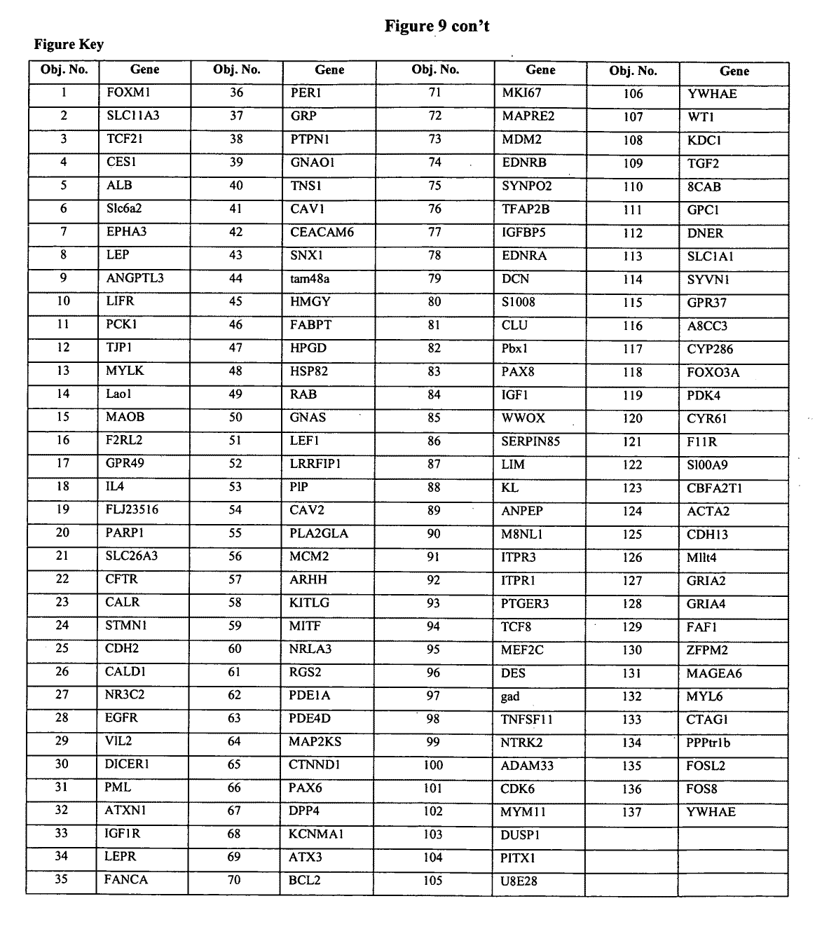

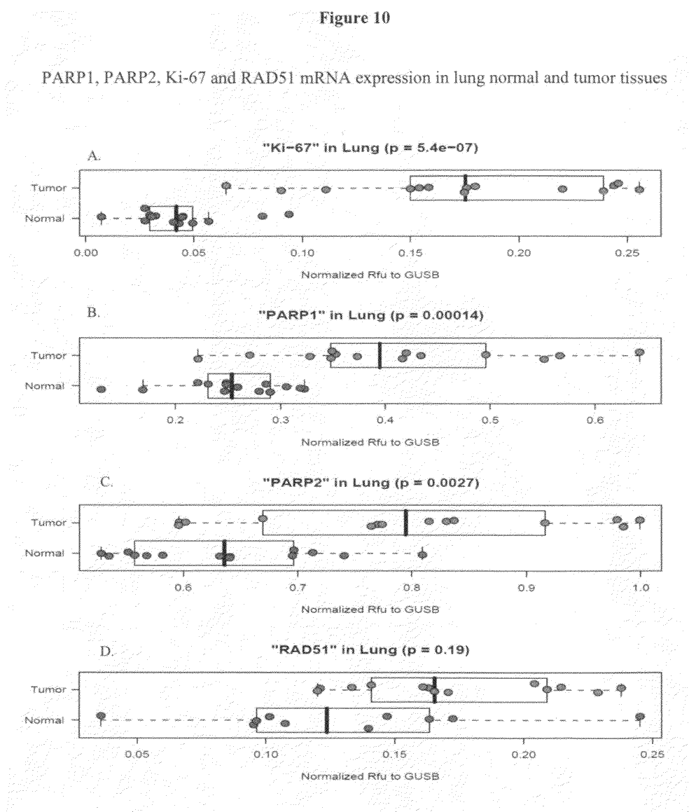

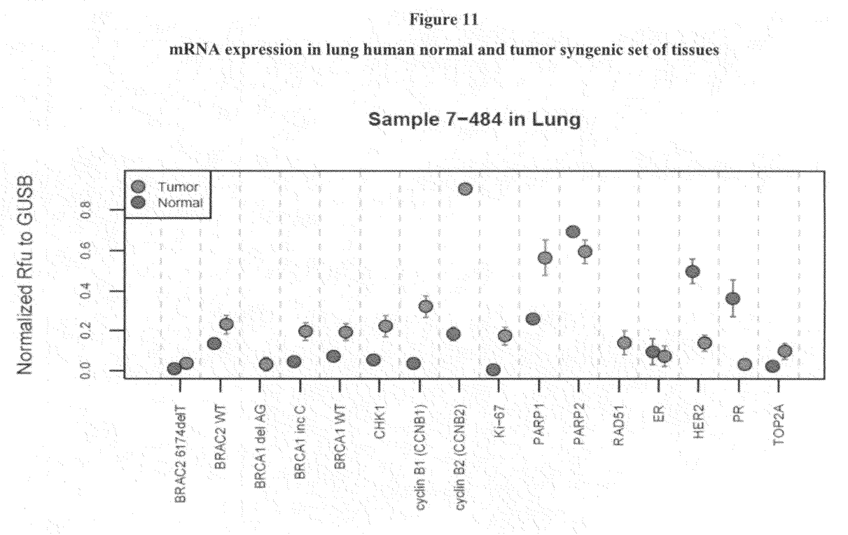

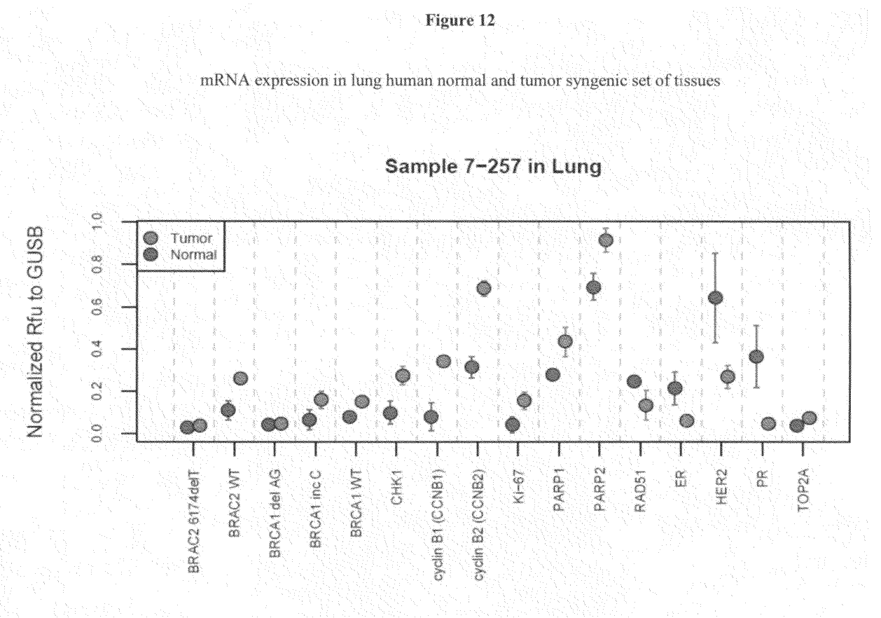

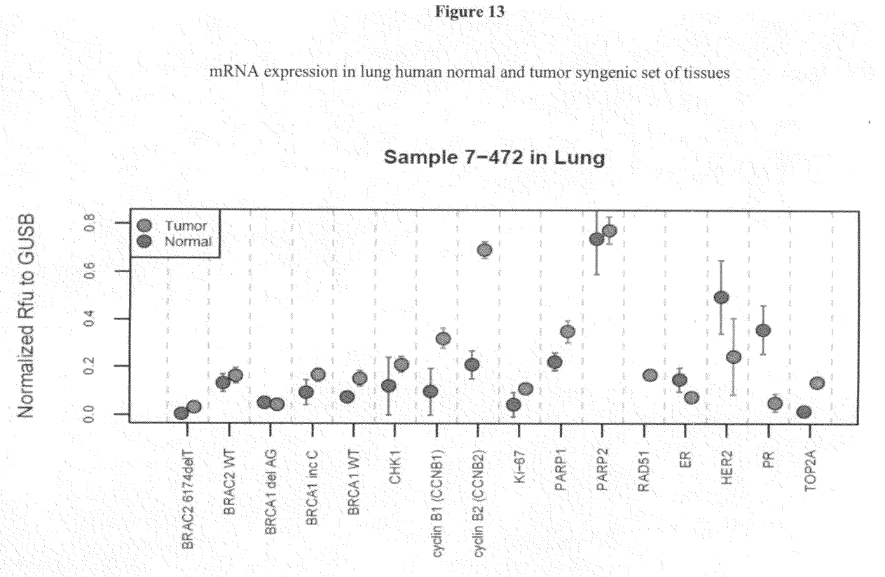

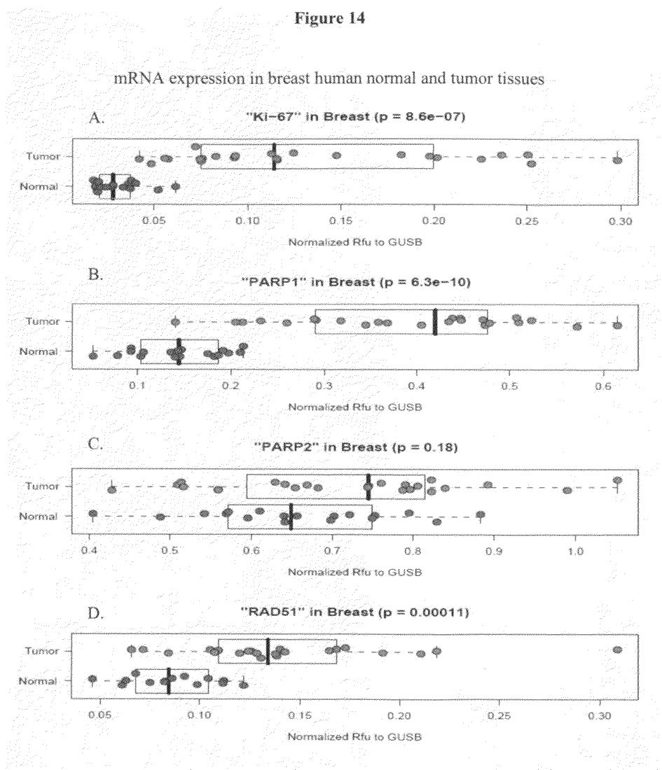

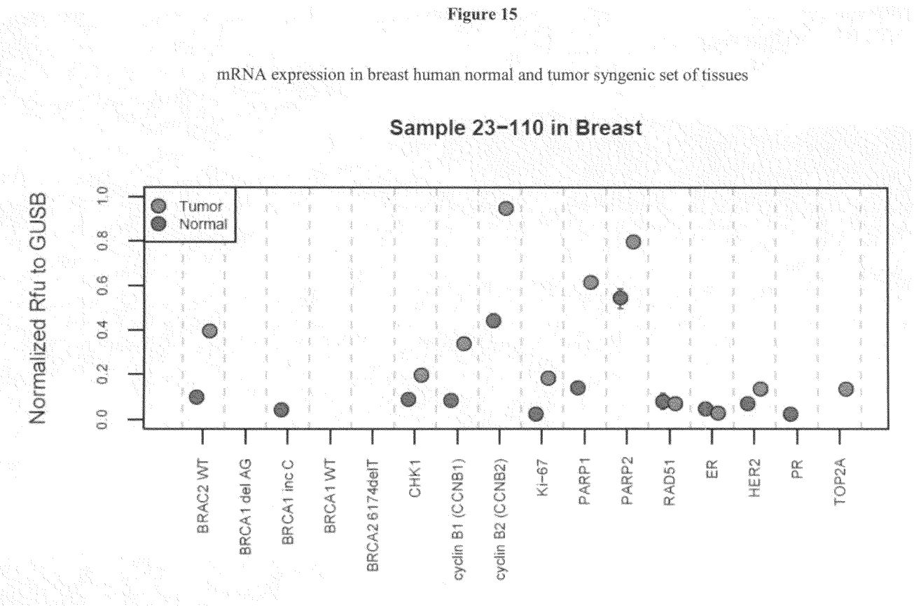

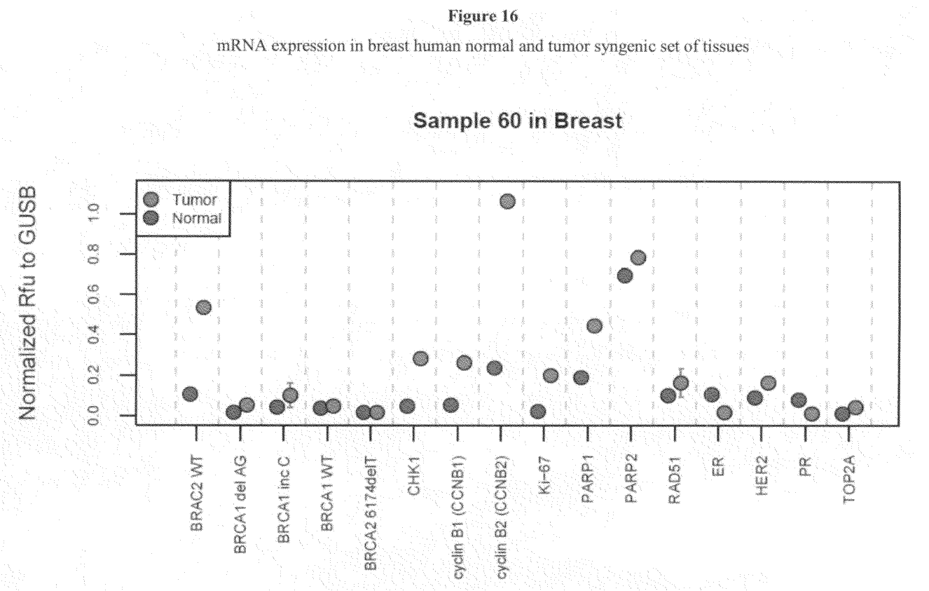

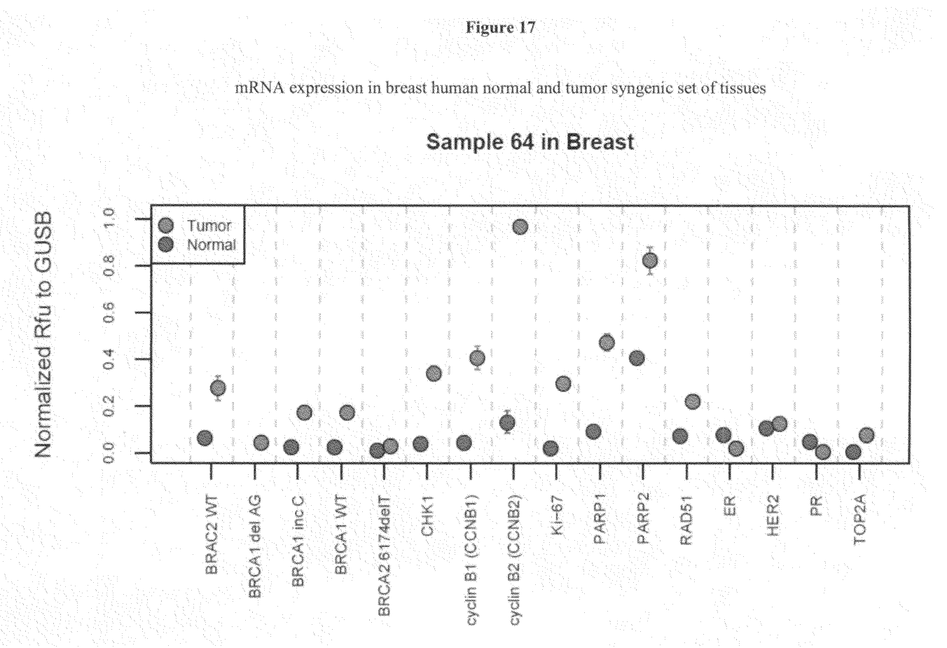

The networks were built from each group of significant genes as well as for significant genes common between each pair of tissues and common between 3 tissues and 4 tissues. Two examples of networks are also shown on FIGS. 8 and 9.

The networks were compared using PathwayStudio (Ariadne Genomics) to find proteins that appear on the networks from significant genes selected with cutoff 2-fold. The results of comparison are available from Network analysis folder. The list of proteins present in both physical and regulatory networks in all three tissues is available. The proteins having the biggest connectivity in all networks were EGFR, BCL2, IGF1, CAV1, LEP, IGF1R, ALB, MDM2, IGF2, FOXM1, CALR, PAX6, WT1 and PARP1. See (Yuryev et al., 2006, BMC Bioinformatics, 7:171; Daraselia et al., 2007, BMC Bioinformatics 8:243; Sivachenko et al., 2007, J. Bioinform. Comput. Biol. 5(2B):429-56). Accordingly, the results demonstrate that along with upregulation of PARP1 expression in breast, endometrium, ovary and lung cancers, EGFR, BCL2, IGF1, CAV1, LEP, IGF1R, ALB, MDM2, IGF2, FOXM1, CALR, PAX6 and WT1 are co-regulated in all four tumor tissues.

The presence of PARP1 in all networks indicates that PARP1 is an important regulatory target in PARP1-activated tumors and showed the presence of regulatory network aimed on PARP1 activation. Other proteins in the networks can be used as biomarkers for selecting PARP1-activated tumors for PARP1 inhibitor therapy or as targets in combinational therapy with PARP1 inhibitors.

WT1, FOXM1, CALR and PAX6 are transcription factors probably responsible for activation of the PARP1 expression regulatory network. FOXM1 was also found significant in the network enrichment analysis below.

The fact that IGF1, IGF2, and IGF1R are present in all networks indicates that PARP1-activated tumors should be IGF sensitive. There was no consistent correlation between IGF pathway genes and PARP1 across all tissues. The correlation or absence of correlation between these two functional modules must be accessed by more sensitive technique than microarray. Currently available data suggest that there are no direct causative relationships between PARP1 and IGF pathway. It is more likely that that they are under control of common set of transcription factors which combinatorial effects manifest differently in different tissue context.

Network enrichment analysis: The log ratios between gene expression in low-PARP1 and PARP1-overexpressing tumors was calculated as log ratio between average expression values in samples with PARP1 differentially expression. The calculated log ratios were imported into Pathway Studio Enterprise to perform Network enrichment analysis algorithm (Sivachenko et al., 2007, J. Bioinform. Comput. Biol. 5(2B):429-56) using “Find significant regulators” command. The top 500 significant regulators for each tissue in Expression or Promoter Binding networks are available. WT1 was found to be significant regulator in Promoter Binding network in all three tissues, FOXM1 was found to be a significant regulator in Expression network in all three tissues.

### Example 6

To further investigate the correlation of co-regulated genes and PARP upregulation in tumors, IGF1R, IGF2, EGFR, TYMS, DHFR, VEGF, MMP9, VEGFR, VEGFR2, IRAK1, ERBB3, AURKA, BCL2, UBE2S mRNA levels were measured and compared to expression levels in normal tissues as described above. The results are shown in Tables XIX to XXXI.

### Materials and Methods

Tissue samples: Normal and carcinoma tissue samples were collected in the United States or United Kingdom. Specimens were harvested as part of a normal surgical procedure and flash frozen within 30 minutes of resection. Samples were shipped at −80° C. and stored in the vapor phase of liquid nitrogen at −170 to −196° C. until processed. Internal pathology review and confirmation were performed on samples subjected to analysis. H&E-stained glass slides generated from an adjacent portion of tissue were reviewed in conjunction with original diagnostic reports and samples were classified into diagnostic categories. A visual estimate of the percent of tissue involvement by tumor was recorded during slide review by the pathologist and indicates the fraction of malignant nucleated cells. Adjuvant studies such as ER/PR and Her-2/neu expression studies were performed by methodologies including immunohistochemistry and fluorescence in situ hybridization. These results as well as attendant pathology and clinical data were annotated within a sample inventory and management databases (Ascenta, BioExpress databases; Gene Logic, Gaithersburg, Md.).

RNA extraction, quality control, and expression profiling: RNA was extracted from samples by homogenization in Trizol® Reagent (Invitrogen, Carlsbad, Calif.) followed by isolation with a RNeasy kit (Qiagen, Valencia, Calif.) as recommended by the manufacturer. RNA was evaluated for quality and integrity (Agilent 2100 Bioanalyzer derived 28s/18s ratio and RNA integrity number), purity (via absorbance ratio at A260/A280), and quantity (via absorbance at A260 or alternative assay). Gene expression levels were assessed using Affymetrix human genome U133A and B GeneChips (45,000 probesets representing more than 39,000 transcripts derived from approximately 33,000 well-substantiated human genes). Two micrograms (2 μg) of total RNA was used to prepare cRNA using Superscript II™ (Invitrogen, Carlsbad, Calif.) and a T7 oligo dT primer for cDNA synthesis and an Affymetrix GeneChip® IVT Labeling Kit (Affymetrix, Santa Clara, Calif.). Quantity and purity of cRNA synthesis product was assessed using UV absorbance. Quality of cRNA synthesis was assessed using either the Agilent Bioanalyzer or a MOPS agarose gel. The labeled cRNA was subsequently fragmented, and 10 μg was hybridized to each array at 45° C. over 16-24 hours. Arrays were washed and stained according to manufacturer recommendations and scanned on Affymetrix GeneChip Scanners. Array data quality was evaluated using a proprietary high throughput application which assesses the data against multiple objective standards including 5′/3′ GAPDH ratio, signal/noise ratio, and background as well as other additional metrics (e.g. outlier, vertical variance) which must be passed prior to inclusion for analysis. GeneChip analysis was performed with Microarray Analysis Suite version 5.0, Data Mining Tool 2.0, and Microarray database software (http://www.affymetrix.com). All of the genes represented on the GeneChip were globally normalized and scaled to a signal intensity of 100.

Quality Control: RNA is evaluated for quality and integrity via Agilent Bioanalyzer derived 28s/28s ratio and RNA integrity number (RIN)), purity (via absorbance ratio at A260/A280), and quantity (via absorbance at A260 or alternative assay (i.e. ribogreen)). Quantity and purity of cRNA synthesis product is assessed using UV absorbance. Quality of cRNA synthesis is assessed using either the Agilent Bioanalyzer or a MOPS agarose gel. Array quality is evaluated using a proprietary high throughput application by which arrays are evaluated against several strict objective standards such as 5′/3′ GAPDH ratio, signal/noise ratio. and background as well as over thirty additional metrics (e.g. outlier, vertical variance). Data generated throughout the process is managed within the quality system to ensure data integrity of the data.

### Example 8

Cytotoxicity Studies: To investigate the effects of treatment of PARP and co-regulated gene modulators on cancer growth and progression, cytotoxicity studies may be performed.

Different types of cancer cell lines of different origin or primary cells may be seeded on 48 or 96 wells plate. The cells may be cultured in the appropriate medium. Cultures can be maintained in a 37° C. incubator in a humidified atmosphere of 95% O₂/5% CO₂. After the cells are seeded (24 hours), medium is removed and replaced with culture medium in the presence of various concentrations of PARP1 and IGF1R and/or EGFR inhibitors, for example Compound III with the small molecule IGF1R kinase inhibitor NVP-AEW541 and/or Erbitux®, a monoclonal antibody to EGFR. After 6 days of incubation at 37° C., cell viability is measured using the Cell Titer-Blue, Cell Viability Assay (Promega) (see O'Brien et al., 2000, Eur. J. Biochem., 267:5421-5426; Gonzalez and Tarloff, 2001). This assay incorporates a fluorometric/colorimetric growth indicator based on detection by vital dye reduction. Cytotoxicity is measured by growth inhibition.

Cytotoxicity may also be assessed by counting the number of viable cells. Cells are harvested by washing the monolayer with PBS, followed by a brief incubation in 0.25% trypsin and 0.02% EDTA. The cells are then collected, washed twice by centrifugation and resuspended in PBS. Cell number and viability is then determined by staining a small volume of cell suspension with a 0.2% typan blue saline solution and examining the cells in a hemocytometer. Cell number and viability can be assayed by staining cells with Annexin-FITC or/and with propidium iodide and analyzed by flow cytometry

### Example 9

Cell Proliferation Studies: To investigate the effects of treatment of PARP and co-regulated gene modulators on cancer growth and progression, cell proliferation studies may be performed.

Cultured cells may be incubated in the presence of various concentrations of the test substance, for example Compound III with the small molecule IGF1R kinase inhibitor NVP-AEW541 and/or Erbitux®, a monoclonal antibody to EGFR. The cultured cells are plated in a black 96-well MultiPlate (tissue culture grade; flat, clear bottom) at a final volume of 100 ul/well in a humidified atmosphere at 37° C. 10 ul/well BrdU labeling solution is added to the cells (final concentration of BrdU: 10 uM) and the cells are reincubated for an additional 2 to 25 hours at 37° C. The MP is centrifuged at 300×g for 10 min and the labeling medium is removed with suction using a canula. The cells are dried using a hair-dryer for about 15 min., or alternatively, at 60° C. for 1 h. 200 ul/well FixDenat is added to the cells and incubated for 30 min. at 15-25° C. FixDenat solution is removed thoroughly by flicking off and tapping. 100 ul/well Anti-BrdU-POD working solution is added and incubated for approx. 90 min. at 15-25° C. Antibody conjugate is removed by flicking off and wells rinsed three times with 200-300 ul/well washing solution. Washing solution is removed by tapping. Then 100 ul/well substrate solution is added to each well. The light emission of the samples can be measured in a microplate luminometer with photomultiplier.

### Example 10

Xenograft cancer models can be employed to measure the effects of treatment of PARP and co-regulated gene modulators on cancer growth and progression.

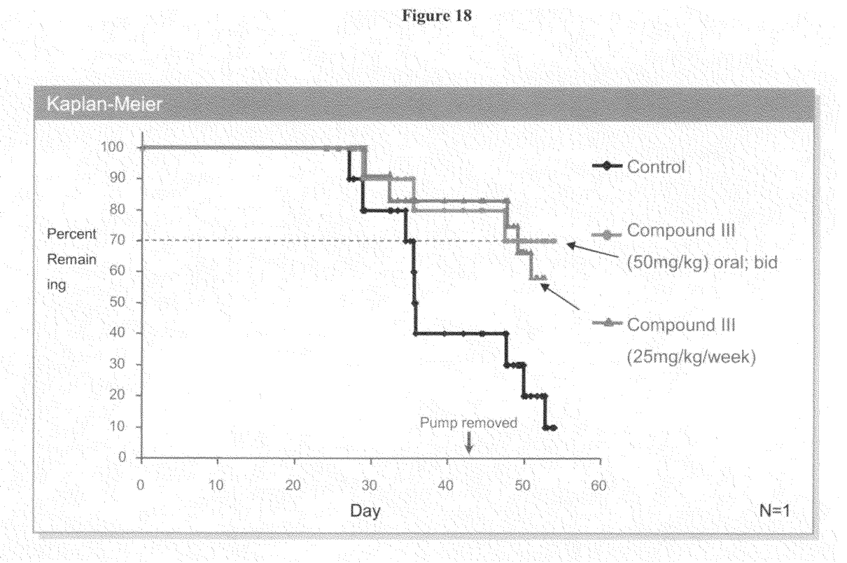

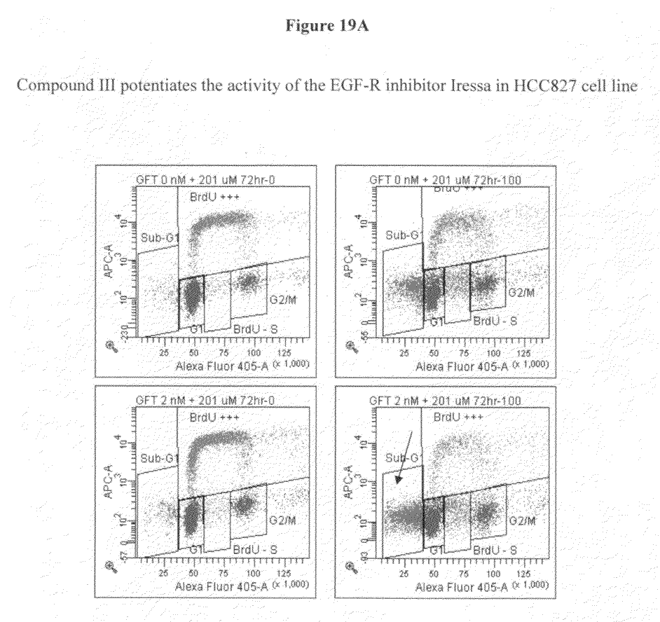

For example, PARP1 inhibition by Compound III has been shown in the human ovarian adenocarcinoma OVCAR-3 xenograft model to inhibit tumor growth and improve survival of mice. See FIG. 18. Moreover, ovarian adenocarcinoma OVCAR-3 cells produce IGF-I and IGF-II, and express IGF1R, supporting the existence of an autocrine loop. Previous studies have shown that treatment with NVP-AEW541, a small molecular weight inhibitor of the IGF-1R kinase, can inhibit growth of OVCAR-3 tumor (Gotlieb et al., 2006, Gynecol Oncol. 100(2):389-96). Importantly, neither treatment with Compound III nor NVP-AEW541 fully inhibits tumor growth. Accordingly, from this data it is expected that the combination of a PARP inhibitor, e.g. Compound III, and an IGF1R inhibitor, e.g. NVP-AEW541, would inhibit tumor growth in mice even further.

### Example 11

The effect of a combination of PARP1 and IGF1 receptor inhibitors in treatment of IDC breast cancer with chemotherapeutic agents can be determined.

A multi-center, open-label, randomized study to demonstrate the therapeutic effectiveness in the treatment of IDC breast cancer with a PARP1 inhibitor (Compound III), IGF1R (NVP-AEW541) inhibitor and chemotherapeutic agent (e.g. gemcitabine, carboplatin, cisplatin) will be conducted. The therapeutic efficacy of this combination therapy will be compared to the therapeutic efficacy of the chemotherapeutic agent alone.

Study Design: An open label, 2-arm randomized, safety and efficacy study in which up to 90 patients (45 in each arm) will be randomized to either: Study Arm 1: Chemotherapeutic agent alone, for example gemcitabine (1000 mg/m²; 30 min IV infusion) or carboplatin (AUC 2; 60 min IV infusion) on days 1 and 8 of a 21-day cycle; or Study Arm 2: Chemotherapeutic agent+IGF1R and PARP1 inhibitor, for example gemcitabine (1000 mg/m²; 30 min IV infusion) or Carboplatin (AUC 2; 60 min IV infusion) on days 1 and 8 of a 21-day cycle with Compound III (4 mg/kg 1 hour IV infusion) and NVP-AEW541 (25 mg/kg; bid) on days 1, 4, 8 and 11 of each 21-day cycle.

Assessment: Tumors will be assessed by standard methods (e.g., CT) at baseline and then approximately every 6-8 weeks thereafter in the absence of clinically evident progression of disease.

### Example 12

The effects of Compound III and its nitroso metabolite on the cell cycle in cancer cell lines in combination with second agents were determined.

Compound III and Compound III-1 compounds were tested in the presence of the second agent according the schedule indicated in the Table below.

| Agent | Compound III | Cell line |
|---|---|---|
| IGF1R inhibitor | +/− | MDA-MB- |
| | | 468 |
| EGFR inhibitor | +/− | HCC827 |

### Material and Methods

Cell Culture: Triple negative MDA-MB-468 human breast carcinoma, U251 human glioblastoma and lung adenocarcinoma HCC827 cells were cultured in Dulbecco Modified Eagle Medium with 10% fetal bovine serum. Cells were plated at a seeding density 10⁵ per P100 or at 10⁴ per P60 in growth media and incubated 12-18 h at 37° C., 5% CO₂. Compounds with and without secondary agent (see Table 1) were added as a single dose for 72 hours. DMSO was used as a control. Following treatment, cells were analyzed with BrdU ELISA Assay (Roche Applied Science), FACS based cell cycle assay or TUNEL.

Compounds: Compound III was dissolved directly from dry powder in DMSO (cat #472301, Sigma-Aldrich) for each separate experiment, then the entire volume of the stock solution was used to prepare 111 nM, 313 nM and 1 μM working concentrations in cell culture medium to avoid any possibility of precipitation and the corresponding loss of compound. Control experiments were carried out with the matching volume/concentration of the vehicle (DMSO); in these controls, the cells showed no changes in their growth or cell cycle distribution.

PI Exclusion, Cell Cycle and TUNEL Assays (FACS): After the addition of drugs and incubation, cells were taken for counting and PI (Propidium Iodide) exclusion assay. One part of the cells was centrifuged and resuspended in 0.5 ml ice-cold PBS containing 5 μg/ml of PI. The other part of the cells was fixed in ice-cold 70% ethanol and stored in a freezer overnight. For cell cycle analysis, cells were stained with propidium iodide (PI) using standard procedures. Cellular DNA content was determined by flow cytometry using BD LSRII FACS, and the percentages of cells in G1, S or G2/M were determined using ModFit software.

To detect apoptosis, the cells were labeled with the “In Situ Cell Death Detection Kit, Fluorescein” (Roche Diagnostics Corporation, Roche Applied Science, Indianapolis, Ind.). Briefly, fixed cells were centrifuged and washed once in phosphate-buffered saline (PBS) containing 1% bovine serum albumin (BSA), then resuspended in 2 ml permeabilization buffer (0.1% Triton X-100 and 0.1% sodium citrate in PBS) for 25 min at room temperature and washed twice in 0.2 ml PBS/1% BSA. The cells were resuspended in 50 μl TUNEL reaction mixture (TdT enzyme and labeling solution) and incubated for 60 min at 37° C. in a humidified dark atmosphere in an incubator. The labeled cells were washed once in PBS/1% BSA, then resuspended in 0.5 ml ice-cold PBS containing 1 μg/ml 4′,6-diamidino-2-phenylindole (DAPI) for at least 30 min. All cell samples were analyzed with a BD LSR II (BD Biosciences, San Jose, Calif.). All flow cytometry analyses were carried out using triplicate samples containing at least 30,000 cells each (typical results of independent experiments are shown). The coefficient of variance in all the experiments was equal or less than 0.01.

Bromodeoxyuridine (BrdU) labeling assay and FACS-based cell cycle analysis: 50 μl of BrdU (Sigma Chemical Co., St. Louis, Mo.) stock solution (1 mM) was added to achieve final concentration of 10 μM BrdU. Then cells were incubated for 30 min at 37° C. and fixed in ice-cold 70% ethanol and stored at 4° C. overnight. Fixed cells were centrifuged and washed once in 2 ml PBS, then re-suspended in 0.7 ml of denaturation solution (0.2 mg/ml pepsin in 2 N HCl) for 15 min at 37° C. in the dark, then 1.04 ml 1M Tris buffer (Trizma base, Sigma Chemical Co.) was added to terminate the hydrolysis. Cells were washed in 2 ml PBS and resuspended in 100-μl (1:100 dilution) of anti-BrdU antibody (DakoCytomation, Carpinteria, Calif.) in TBFP permeable buffer (0.5% Tween-20, 1% bovine serum albumin and 1% fetal bovine serum in PBS), incubated for 25 min at room temperature in the dark and washed in 2 ml PBS. The primary antibody-labeled cells were resuspended in 100 μl ALEXA FLUOR® F(ab′)₂ fragment of goat anti-mouse IgG (H+L) (1:200 dilution, 2 mg/mL, Molecular Probes, Eugene, Oreg.) in TBFP buffer and incubated for 25 min at room temperature in the dark and washed in 2 ml PBS, then re-suspended in 0.5 ml ice-cold PBS containing 1 μg/ml 4′,6-diamidino-2-phenylindole (DAPI) for at least 30 min. All cell samples were analyzed with a BD LSR II (BD Biosciences, San Jose, Calif.). All flow cytometry analyses were carried out using triplicate samples containing at least 30,000 cells each (typical results of independent experiments are shown). The coefficient of variance in all the experiments was equal or less than 0.01.

### Results

Compounds were dissolved at the start of the experiment in 100% DMSO to 10 mM stock solution.

MDA-MB-468 human breast carcinoma cells and lung cancer adenocarcinoma cell line HCC827 cells were tested for suitability to FACS-based cell cycle analysis.

FACS analysis based on DNA content and BrdU assay.

Two different dose concentrations of Compound III were selected based on preliminary results of the proliferation and survival analysis.

The active dose combinations were tested for their effects on cell survival, cell cycle distribution and BrdU incorporation by FACS analysis.

Concentration Verification and Stability. Triplicate samples of cells were taken within 5 min and within 15 min after dosing, collected by centrifugation, washed by PBS and stored at −70° C. The samples were shipped to the sponsor's designee for further analysis (Alta Analytical Laboratory).

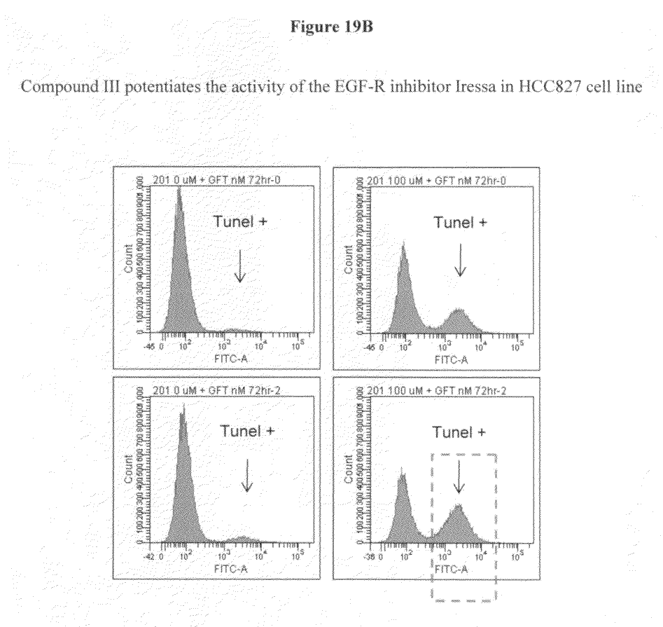

Representative results are presented in the Table below and in FIG. 19.

Response of triple negative breast cancer cells MDA-MB-468
to the combinations of Compound III with IGF-R inhibitor
Picropodophyllin (PPP)

| | Sub- | | | | | | Vital |
|---|---|---|---|---|---|---|---|
| | G1 | G1 | S | G2/M | TUNEL(+) | BrdU(−) S | Cell |
| PPP | | | | | | | |
| 0 nM + | | | | | | | |
| 201 uM | | | | | | | |
| 0 | 0.81 | 50.96 | 30.37 | 16.04 | 0.7 | 1.82 | 100 |
| 50 | 1.01 | 50.20 | 31.34 | 15.21 | 0.9 | 2.23 | 82 |
| 100 | 1.12 | 40.63 | 34.52 | 20.16 | 1.6 | 3.56 | 61 |
| PPP | | | | | | | |
| 200 nM + | | | | | | | |
| 201 uM | | | | | | | |
| 0 | 1.22 | 51.42 | 30.22 | 15.01 | 0.9 | 2.13 | 89 |
| 50 | 1.32 | 49.75 | 31.41 | 15.10 | 2.7 | 2.43 | 77 |
| 100 | 1.63 | 37.51 | 35.58 | 21.30 | 2.1 | 3.98 | 59 |
| PPP | | | | | | | |
| 400 nM + | | | | | | | |
| 201 uM | | | | | | | |
| 0 | 7.77 | 37.29 | 25.32 | 20.17 | 4.1 | 9.45 | 60 |
| 50 | 7.25 | 32.88 | 28.47 | 22.37 | 4.2 | 9.03 | 42 |
| 100 | 5.93 | 23.62 | 31.78 | 29.98 | 6.9 | 8.69 | 32 |

Compound III was shown to potentiate the activity of the EGF-R inhibitor IRESSA® in HCC827 cell line (See FIGS. 19A and 19B).

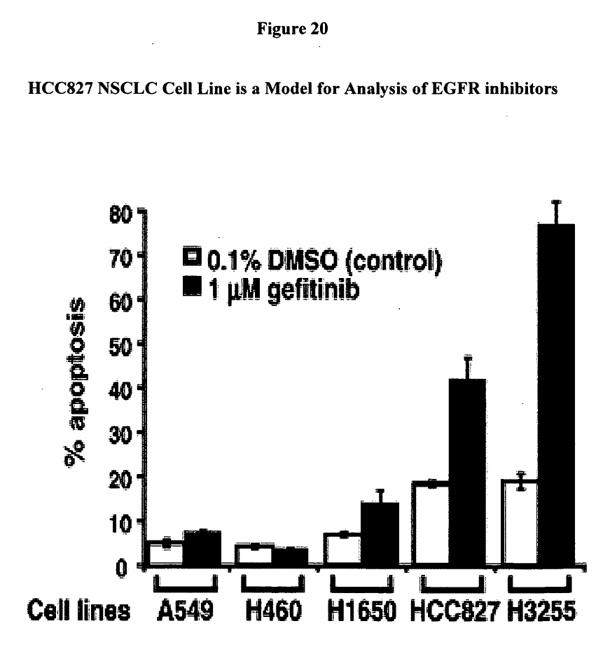

The HCC827 non-small cell lung cancer (NSCLC) cell line has been established as a model for analysis of EGFR inhibitors. See also FIG. 20.

| | | Gefitinib |
|---|---|---|
| | Mutation status of | sensitivity |
| Cell line | EGFR, KRAS | (IC₅₀, μM) |
| H358 | KRAS: G12V | ≈10 |
| H1650 | EGFR: E746-A750del | >10 |
| H1666 | EGFR: wt; KRAS: wt | ≈4 |
| H1734 | KRAS: G13C | >10 |
| H1975 | EGFR: L858R, T790M | >10 |
| HCC827 | EGFR: E746_A750del | <0.1 |
| H3255 | EGFR: L858R | <0.1 |

A summary of the response of lung cancer cells HCC827 to the combination of compound III with IRESSA® is shown in the following tables:

| | G1 | S | G2/M | Viable Cell | Sub-G1 | TUNEL(+) |
|---|---|---|---|---|---|---|
| GFT 0 nM + | | | | | | |
| 201 μM | | | | | | |
| 0 | 65.9 | 24.3 | 6.3 | 100.0 | 1.9 | 3.2 |
| 50 | 46.3 | 40.6 | 8.8 | 65.0 | 2.4 | 5.7 |
| 100 | 43.8 | 19.9 | 12.5 | 25.0 | 15.8 | 32.8 |
| GFT 2 nM + | | | | | | |
| 201 μM | | | | | | |
| 0 | 51.0 | 34.4 | 9.2 | 56.0 | 3.4 | 6.2 |
| 50 | 52.3 | 28.9 | 9.4 | 37.0 | 6.6 | 13.3 |
| 100 | 38.9 | 12.6 | 12.3 | 15.0 | 27.4 | 46.5 |

### Example 13

To further investigate co-regulated genes and PARP upregulation in tumors, IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, CDK1, CKD2, CDK9, farnesyl transferase, UBE2A, UBE2D2, UBE2G1, USP28, UBE2S, or a combination thereof, mRNA levels are measured and compared to expression levels in normal tissues as described above.

### Materials and Methods

Tissue samples: Normal and cancerous tissue samples are collected in the United States or United Kingdom. Specimens are harvested as part of a normal surgical procedure and flash frozen within 30 minutes of resection. Samples are shipped at −80° C. and stored in the vapor phase of liquid nitrogen at −170 to −196° C. until processed. Internal pathology review and confirmation are performed on samples subjected to analysis. H&E-stained glass slides generated from an adjacent portion of tissue are reviewed in conjunction with original diagnostic reports and samples are classified into diagnostic categories. A visual estimate of the percent of tissue involvement by tumor is recorded during slide review by the pathologist and indicates the fraction of malignant nucleated cells. Adjuvant studies such as ER/PR and Her-2/neu expression studies are performed by methodologies including immunohistochemistry and fluorescence in situ hybridization. These results as well as attendant pathology and clinical data are annotated within a sample inventory and management databases (Ascenta, BioExpress databases; Gene Logic, Gaithersburg, Md.).

RNA extraction, quality control, and expression profiling: RNA is extracted from samples by homogenization in Trizol® Reagent (Invitrogen, Carlsbad, Calif.) followed by isolation with a RNeasy kit (Qiagen, Valencia, Calif.) as recommended by the manufacturer. RNA is evaluated for quality and integrity (Agilent 2100 Bioanalyzer derived 28s/18s ratio and RNA integrity number), purity (via absorbance ratio at A260/A280), and quantity (via absorbance at A260 or alternative assay). Gene expression levels are assessed using Affymetrix human genome U133A and B GeneChips (45,000 probesets representing more than 39,000 transcripts derived from approximately 33,000 well-substantiated human genes). Two micrograms (2 μg) of total RNA is used to prepare cRNA using Superscript II™ (Invitrogen, Carlsbad, Calif.) and a T7 oligo dT primer for cDNA synthesis and an Affymetrix GeneChip® IVT Labeling Kit (Affymetrix, Santa Clara, Calif.). Quantity and purity of cRNA synthesis product is assessed using UV absorbance. Quality of cRNA synthesis is assessed using either the Agilent Bioanalyzer or a MOPS agarose gel. The labeled cRNA is subsequently fragmented, and 10 μg is hybridized to each array at 45° C. over 16-24 hours. Arrays are washed and stained according to manufacturer recommendations and scanned on Affymetrix GeneChip Scanners. Array data quality is evaluated using a proprietary high throughput application which assesses the data against multiple objective standards including 5′/3′ GAPDH ratio, signal/noise ratio, and background as well as other additional metrics (e.g. outlier, vertical variance) which must be passed prior to inclusion for analysis. GeneChip analysis is performed with Microarray Analysis Suite version 5.0, Data Mining Tool 2.0, and Microarray database software (www.affymetrix.com). All of the genes represented on the GeneChip are globally normalized and scaled to a signal intensity of 100.

Quality Control: RNA is evaluated for quality and integrity via Agilent Bioanalyzer derived 28s/28s ratio and RNA integrity number (RIN)), purity (via absorbance ratio at A260/A280), and quantity (via absorbance at A260 or alternative assay (i.e. ribogreen)). Quantity and purity of cRNA synthesis product is assessed using UV absorbance. Quality of cRNA synthesis is assessed using either the Agilent Bioanalyzer or a MOPS agarose gel. Array quality is evaluated using a proprietary high throughput application by which arrays are evaluated against several strict objective standards such as 5′/3′ GAPDH ratio, signal/noise ratio. and background as well as over thirty additional metrics (e.g. outlier, vertical variance). Data generated throughout the process is managed within the quality system to ensure data integrity of the data.

PARP1 inhibitors and inhibitors of co-regulated genes may be administered to the patient as in Example 11.

### Example 14

To further investigate co-regulated genes and PARP upregulation in breast tumors, BRCA1, BRCA2, or a combination thereof, mRNA levels are measured and compared to expression levels in normal tissues as described above.

### Materials and Methods

Tissue samples: Normal and cancerous breast tissue samples are collected in the United States or United Kingdom. Specimens are harvested as part of a normal surgical procedure and flash frozen within 30 minutes of resection. Samples are shipped at −80° C. and stored in the vapor phase of liquid nitrogen at −170 to −196° C. until processed. Internal pathology review and confirmation are performed on samples subjected to analysis. H&E-stained glass slides generated from an adjacent portion of tissue are reviewed in conjunction with original diagnostic reports and samples are classified into diagnostic categories. A visual estimate of the percent of tissue involvement by tumor is recorded during slide review by the pathologist and indicates the fraction of malignant nucleated cells. Adjuvant studies such as protein expression studies are performed by methodologies including immunohistochemistry and fluorescence in situ hybridization. These results as well as attendant pathology and clinical data are annotated within a sample inventory and management databases (Ascenta, BioExpress databases; Gene Logic, Gaithersburg, Md.).

RNA extraction, quality control, and expression profiling: RNA is extracted from samples by homogenization in Trizol® Reagent (Invitrogen, Carlsbad, Calif.) followed by isolation with a RNeasy kit (Qiagen, Valencia, Calif.) as recommended by the manufacturer. RNA is evaluated for quality and integrity (Agilent 2100 Bioanalyzer derived 28s/18s ratio and RNA integrity number), purity (via absorbance ratio at A260/A280), and quantity (via absorbance at A260 or alternative assay). Gene expression levels are assessed using Affymetrix human genome U133A and B GeneChips (45,000 probesets representing more than 39,000 transcripts derived from approximately 33,000 well-substantiated human genes). Two micrograms (2 μg) of total RNA is used to prepare cRNA using Superscript II™ (Invitrogen, Carlsbad, Calif.) and a T7 oligo dT primer for cDNA synthesis and an Affymetrix GeneChip® IVT Labeling Kit (Affymetrix, Santa Clara, Calif.). Quantity and purity of cRNA synthesis product is assessed using UV absorbance. Quality of cRNA synthesis is assessed using either the Agilent Bioanalyzer or a MOPS agarose gel. The labeled cRNA is subsequently fragmented, and 10 μg is hybridized to each array at 45° C. over 16-24 hours. Arrays are washed and stained according to manufacturer recommendations and scanned on Affymetrix GeneChip Scanners. Array data quality is evaluated using a proprietary high throughput application which assesses the data against multiple objective standards including 5′/3′ GAPDH ratio, signal/noise ratio, and background as well as other additional metrics (e.g. outlier, vertical variance) which must be passed prior to inclusion for analysis. GeneChip analysis is performed with Microarray Analysis Suite version 5.0, Data Mining Tool 2.0, and Microarray database software (www.affymetrix.com). All of the genes represented on the GeneChip are globally normalized and scaled to a signal intensity of 100.

Quality Control: RNA is evaluated for quality and integrity via Agilent Bioanalyzer derived 28s/28s ratio and RNA integrity number (RIN)), purity (via absorbance ratio at A260/A280), and quantity (via absorbance at A260 or alternative assay (i.e. ribogreen)). Quantity and purity of cRNA synthesis product is assessed using UV absorbance. Quality of cRNA synthesis is assessed using either the Agilent Bioanalyzer or a MOPS agarose gel. Array quality is evaluated using a proprietary high throughput application by which arrays are evaluated against several strict objective standards such as 5′/3′ GAPDH ratio, signal/noise ratio. and background as well as over thirty additional metrics (e.g. outlier, vertical variance). Data generated throughout the process is managed within the quality system to ensure data integrity of the data.

BRCA1, BRCA2 and PARP levels are determined and assessed in normal versus cancerous breast tissue.

PARP1 inhibitors and inhibitors of co-regulated genes may be administered as in Example 11.

While embodiments have been shown and described herein, it will be obvious to those skilled in the art that such embodiments are provided by way of example only. Numerous variations, changes, and substitutions will now occur to those skilled in the art without departing from the embodiments described herein. It should be understood that various alternatives to the embodiments described herein may be employed in practicing the embodiments described. It is intended that the following claims define the scope of the embodiments and that methods and structures within the scope of these claims and their equivalents be covered thereby.

What is claimed is:

1. A method of identifying genes useful in the treatment of a patient with a disease susceptible to PARP modulator treatment, the method comprising:
a. identifying a disease treatable with at least one PARP modulator, wherein the expression level of PARP in a plurality of samples from a population is regulated in comparison to a control sample;
b. determining the expression level of a panel of genes in the plurality of samples; and
c. identifying genes that are co-regulated with said PARP regulation, wherein the expression level of said co-regulated genes in the plurality of samples are increased or decreased in comparison to a control sample;
wherein modulation of said genes that are co-regulated with PARP regulation is useful in the treatment of a disease susceptible to PARP modulator treatment.

2. The method of claim 1 wherein said co-regulated genes include genes expressed in the PARP, IGF1 receptor, or EGFR pathways.

3. The method of claim 1 wherein said PARP modulator is a PARP inhibitor.

4. The method of claim 1 wherein said PARP inhibitor is selected from the group consisting of benzamide, quinolone, isoquinolone, benzopyrone, cyclic benzamide, benzimidazole, indole and pharmaceutically salts, solvates, isomers, tautomers, metabolites, analogs, or prodrugs thereof.

5. The method of claim 4 wherein said PARP inhibitor is 4-iodo, 3-nitro benzamide or a metabolite thereof.

6. The method of claim 1 wherein said co-regulated genes include IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, CDK1, CDK2, CDK9, farnesyl transferase, UBE2S, ABCC1, ABCC5, ABCD4, ACADM, ACLSL1, ACSL3, ACY1L2, ADM, ADRM1, AGPAT5, AHCY, AK3L1, AK3L2, AKIIP, AKR1B1, AKR1C1, AKR1C2, AKR1C3, ALDH18A1, ALDOA, ALOX5, ALPL, ANP32E, AOF1, APG5L, ARFGEF1, ARL5, ARPP-19, ASPH, ATF5, ATF7IP, ATIC, ATP11A, ATP11C, ATP1A1, ATP1B1, ATP2A2, ATP5G3, ATP5J2, ATP6V0B, B3GNT1, B4GALT2, BACE2, BACH, BAG2, BASP1, BCAT1, BCL2L1, BCL6, BGN, BPNT1, C1QBP, CACNB3, CAMK2D, CAP2, CCAR1, CD109, CD24, CD44, CD47, CD58, CD74, CD83, CD9, CDC14B, CDC42EP4, CDC5L, CDK4, CDK6, CDS1, CDW92, CEACAM6, CELSR2, CFLAR, CGI-90, CHST6, CHSY1, CKLFSF4, CKLFSF6, CKS1B, CMKOR1, CNDP2, CPD, CPE, CPSF3, CPSF5, CPSF6, CPT1B, CRR9, CSH2, CSK, CSNK2A1, CSPG2, CTPSCTSB, CTSD, CXADR, CXCR4, CXXC5, CXXC6, DAAM1, DCK, DDAH1, DDIT4, DDR1, DDX21, DDX39, DHTKD1, DLAT, DNAJA1, DNAJB11, DNAJC1, DNAJC10, DNAJC9, DNAJD1, DUSP10, DUSP24, DUSP6, DVL3, ELOVL6, EME1, ENO1, ENPP4, EPS8, ETNK1, ETV6, F11R, FA2H, FABP5, FADS2, FAS, FBXO45, FBXO7, FLJ23091, FTL, FTLL1, FZD6, G1P2, GALNT2, GALNT4, GALNT7, GANAB, GART, GBAS, GCHFR, GCLC, GCLM, GCNT1, GFPT1, GGA2, GGH, GLUL, GMNN, GMPS, GPI, GPR56, GPR89, GPX1, GRB10, GRHPR, GSPT1, GSR, GTPBP4, HDAC1, HDGF, HIG2, HMGB3, HPRT1, HPS5, HRMT1L2, HS2ST1, HSPA4, HSPA8, HSPB1, HSPCA, HSPCAL3, HSPCB, HSPD1, HSPE1, HSPH1, HTATIP2, HYOU1, ICMT, IDE, IDH2, IFI27, IGFBP3, IGSF4, ILF2, INPP5F, INSIG1, KHSRP, KLF4, KMO, KPNA2, KTN1, LAP3, LASS2, LDHA, LDHB, LGR4, LPGAT1, LTB4DH, LYN, MAD2L1, MADP-1, MAGED1, MAK3, MALAT1, MAP2K3, MAP2K6, MAP3K13, MAP4K4, MAPK13, MARCKS, MBTPS2, MCM4, MCTS1, MDH1, MDH2, ME1, ME2, METAP2, METTL2, MGAT4B, MKNK2. MLPH, MOBK1B, MOBKL1A, MSH2, MTHFD2, MUC1, MX1, MYCBP, NAJD1, NAT1, NBS1, NDFIP2, NEK6, NET1, NME1, NNT, NQO1, NRAS, NSE2, NUCKS, NUSAP1, NY-REN-41, ODC1, OLR1, P4HB, PAFAH1B1, PAICS, PANK1, PCIA1, PCNA, PCTK1, PDAP1, PDIA4, PDIA6, PDXK, PERP, PFKP, PFTK1, PGD, PGK1, PGM2L1, PHCA, PKIG, PKM2, PKP4, PLA2G4A, PLCB1, PLCG2, PLD3, PLOD1, PLOD2, PMS2L3, PNK1, PNPT1, PON2, PP, PPIF, PPP1CA, PPP2R4, PPP3CA, PRCC, PRKD3, PRKDC, PRPSAP2, PSAT1, PSENEN, PSMA2, PSMA5, PSMA7, PSMB3, PSMB4, PSMD14, PSMD2, PSMD3, PSMD4, PSMD8, PTGFRN, PTGS1, PTK9, PTPN12, PTPN18, PTS, PYGB, RAB10, RAB11FIP1, RAB14, RAB31, RAB3IP, RACGAP1, RAN, RANBP1, RAP2B, RBBP4, RBBP7, RBBP8, RDH10, RFC3, RFC4, RFC5, RGS19IP1, RHOBTB3, RNASEH2A, RNGTT, RNPEP, ROBO1, RRAS2, SART2, SAT, SCAP2, SCD4, SDC2, SDC4, SEMA3F, SERPINE2, SFI1, SGPL1, SGPP1, SGPP2, SH3GLB2, SHC1, SMARCC1, SMC4L1, SMC4L1, SMS, SNRPD1, SORD, SORL1, SPP1, SQLE, SRD5A1, SRD5A2L, SRM, SRPK1, SS18, SSBP1, SSR3, ST3GAL5, ST6GAL1, ST6GALNAC2, STX18, SULF2, SWAP70, TA-KRP, TALA, TBL1XR1, TFRC, TIAM1, TKT, TMPO, TNFAIP2, TNFSF9, TOX, TPD52, TPI1, TPP1, TRA1, TRIP13, TRPS1, TSPAN13, TSTA3, TXN, TXNL2, TXNL5, TXNRD1, UBAP2L, UBE2A, UBE2D2, UBE2G1, UBE2V1, UCHL5, UGDH, UNC5CL, USP28, USP47, UTP14A, VDAC1, WIG1, YWHAB, YWHAE, YWHAZ, or a combination thereof.

7. The method of claim 1 wherein the mRNA level of each co-regulated gene is measured.

8. The method of claim 1 wherein said tissue sample is selected from the group consisting of tumor sample, hair, blood, cell, tissue, organ, brain tissue, blood, serum, sputum, saliva, plasma, nipple aspirant, synovial fluid, cerebrospinal fluid, sweat, urine, fecal matter, pancreatic fluid, trabecular fluid, cerebrospinal fluid, tears, bronchial lavage, swabbing, bronchial aspirant, semen, prostatic fluid, precervicular fluid, vaginal fluids, and pre-ejaculate.

9. The method of claim 1 wherein said disease is selected from the group consisting of cancer, inflammation, metabolic disease, CVS disease, CNS disease, disorder of hematolymphoid system, disorder of endocrine and neuroendocrine, viral infection, disorder of urinary tract, disorder of respiratory system, disorder of female genital system, and disorder of male genital system.

10. The method of claim 9, wherein the disease is breast cancer, lung cancer, endometrial cancer, ovarian cancer, bone osteosarcoma or Ewing's sarcoma.

11. The method of claim 10, wherein the breast cancer is triple-negative breast cancer.

12. The method of claim 1 wherein said method further comprises providing a conclusion regarding said disease to a patient, a health care provider or a health care manager, said conclusion being based on said decision.

13. A method of treating a patient with a disease susceptible to PARP modulator treatment, the method comprising:
a. identifying a disease treatable with at least one PARP modulator, wherein the expression level of PARP in a sample from a patient with said disease is regulated in comparison to a reference sample;
b. identifying at least one co-regulated gene in said sample in comparison to a reference sample;
c. treating said patient with modulators to PARP and the co-regulated gene.

14. The method of claim 13, wherein said co-regulated gene includes a gene expressed in the PARP, IGF1 receptor, or EGFR pathways.

15. The method of claim 13, wherein said co-regulated gene is IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, UBE2S, CDK1, CDK2, CDK9, farnesyl transferase, ABCC1, ABCC5, ABCD4, ACADM, ACLSL1, ACSL3, ACY1L2, ADM, ADRM1, AGPAT5, AHCY, AK3L1, AK3L2, AKIIP, AKR1B1, AKR1C1, AKR1C2, AKR1C3, ALDH18A1, ALDOA, ALOX5, ALPL, ANP32E, AOF1, APG5L, ARFGEF1, ARL5, ARPP-19, ASPH, ATF5, ATF7IP, ATIC, ATP11A, ATP11C, ATP1A1, ATP1B1, ATP2A2, ATP5G3, ATP5J2, ATP6V0B, B3GNT1, B4GALT2, BACE2, BACH, BAG2, BASP1, BCAT1, BCL2L1, BCL6, BGN, BPNT1, C1QBP, CACNB3, CAMK2D, CAP2, CCAR1, CD109, CD24, CD44, CD47, CD58, CD74, CD83, CD9, CDC14B, CDC42EP4, CDC5L, CDK4, CDK6, CDS1, CDW92, CEACAM6, CELSR2, CFLAR, CGI-90, CHST6, CHSY1, CKLFSF4, CKLFSF6, CKS1B, CMKOR1, CNDP2, CPD, CPE, CPSF3, CPSF5, CPSF6, CPT1B, CRR9, CSH2, CSK, CSNK2A1, CSPG2, CTPSCTSB, CTSD, CXADR, CXCR4, CXXC5, CXXC6, DAAM1, DCK, DDAH1, DDIT4, DDR1, DDX21, DDX39, DHTKD1, DLAT, DNAJA1, DNAJB11, DNAJC1, DNAJC10, DNAJC9, DNAJD1, DUSP10, DUSP24, DUSP6, DVL3, ELOVL6, EME1, ENO1, ENPP4, EPS8, ETNK1, ETV6, F11R, FA2H, FABP5, FADS2, FAS, FBXO45, FBXO7, FLJ23091, FTL, FTLL1, FZD6, G1P2, GALNT2, GALNT4, GALNT7, GANAB, GART, GBAS, GCHFR, GCLC, GCLM, GCNT1, GFPT1, GGA2, GGH, GLUL, GMNN, GMPS, GPI, GPR56, GPR89, GPX1, GRB10, GRHPR, GSPT1, GSR, GTPBP4, HDAC1, HDGF, HIG2, HMGB3, HPRT1, HPS5, HRMT1L2, HS2ST1, HSPA4, HSPA8, HSPB1, HSPCA, HSPCAL3, HSPCB, HSPD1, HSPE1, HSPH1, HTATIP2, HYOU1, ICMT, IDE, IDH2, IFI27, IGFBP3, IGSF4, ILF2, INPP5F, INSIG1, KHSRP, KLF4, KMO, KPNA2, KTN1, LAP3, LASS2, LDHA, LDHB, LGR4, LPGAT1, LTB4DH, LYN, MAD2L1, MADP-1, MAGED1, MAK3, MALAT1, MAP2K3, MAP2K6, MAP3K13, MAP4K4, MAPK13, MARCKS, MBTPS2, MCM4, MCTS1, MDH1, MDH2, ME1, ME2, METAP2, METTL2, MGAT4B, MKNK2. MLPH, MOBK1B, MOBKL1A, MSH2, MTHFD2, MUC1, MX1, MYCBP, NAJD1, NAT1, NBS1, NDFIP2, NEK6, NET1, NME1, NNT, NQO1, NRAS, NSE2, NUCKS, NUSAP1, NY-REN-41, ODC1, OLR1, P4HB, PAFAH1B1, PAICS, PANK1, PCIA1, PCNA, PCTK1, PDAP1, PDIA4, PDIA6, PDXK, PERP, PFKP, PFTK1, PGD, PGK1, PGM2L1, PHCA, PKIG, PKM2, PKP4, PLA2G4A, PLCB1, PLCG2, PLD3, PLOD1, PLOD2, PMS2L3, PNK1, PNPT1, PON2, PP, PPIF, PPP1CA, PPP2R4, PPP3CA, PRCC, PRKD3, PRKDC, PRPSAP2, PSAT1, PSENEN, PSMA2, PSMA5, PSMA7, PSMB3, PSMB4, PSMD14, PSMD2, PSMD3, PSMD4, PSMD8, PTGFRN, PTGS1, PTK9, PTPN12, PTPN18, PTS, PYGB, RAB10, RAB11FIP1, RAB14, RAB31, RAB3IP, RACGAP1, RAN, RANBP1, RAP2B, RBBP4, RBBP7, RBBP8, RDH10, RFC3, RFC4, RFC5, RGS19IP1, RHOBTB3, RNASEH2A, RNGTT, RNPEP, ROBO1, RRAS2, SART2, SAT, SCAP2, SCD4, SDC2, SDC4, SEMA3F, SERPINE2, SFI1, SGPL1, SGPP1, SGPP2, SH3GLB2, SHC1, SMARCC1, SMC4L1, SMC4L1, SMS, SNRPD1, SORD, SORL1, SPP1, SQLE, SRD5A1, SRD5A2L, SRM, SRPK1, SS18, SSBP1, SSR3, ST3GAL5, ST6GAL1, ST6GALNAC2, STX18, SULF2, SWAP70, TA-KRP, TALA, TBL1XR1, TFRC, TIAM1, TKT, TMPO, TNFAIP2, TNFSF9, TOX, TPD52, TPI1, TPP1, TRA1, TRIP13, TRPS1, TSPAN13, TSTA3, TXN, TXNL2, TXNL5, TXNRD1, UBAP2L, UBE2A, UBE2D2, UBE2G1, UBE2V1, UCHL5, UGDH, UNC5CL, USP28, USP47, UTP14A, VDAC1, WIG1, YWHAB, YWHAE, YWHAZ, or a combination thereof.

16. The method of claim 13, wherein said disease is a cancer.

17. The method of claim 16, wherein said cancer is selected from the group consisting of colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, intraductal carcinoma, mucinous carcinoma, phyllodes tumor, Ewing's sarcoma, ovarian adenocarcinoma, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung adenocarcinoma, kidney carcinoma, urinary bladder carcinoma, Wilm's tumor, and lymphoma.

18. The method of claim 13, wherein said expression level of PARP and said co-regulated genes are up-regulated and the treatment decision is to treat said disease with inhibitors to PARP and said co-regulated genes.

19. The method of claim 13, wherein said expression level of PARP and said co-regulated genes are down-regulated and the treatment decision is a decision to not treat said disease with inhibitors to PARP and said co-regulated genes.

20. The method of claim 13, wherein said PARP modulator is a PARP inhibitor.

21. The method of claim 20, wherein said PARP inhibitor is selected from the group consisting of benzamide, quinolone, isoquinolone, benzopyrone, cyclic benzamide, benzimidazole, indole, and pharmaceutically salts, solvates, isomers, tautomers, metabolites, analogs, or prodrugs thereof.

22. The method of claim 21, wherein said PARP inhibitor is 4-iodo, 3-nitro benzamide or a metabolite thereof.

23. A method of treating a disease susceptible to PARP modulator treatment, the method comprising:
a. identifying a disease treatable with at least one PARP modulator, wherein the expression level of PARP in a plurality of samples is regulated in comparison to a reference sample;
b. identifying at least one co-regulated gene in said plurality of samples in comparison to a reference sample;
c. treating a patient with said disease with modulators to PARP and the co-regulated gene.

24. The method of claim 23, wherein said co-regulated gene includes a gene expressed in the PARP, IGF1 receptor, or EGFR pathways.

25. The method of claim 23, wherein said co-regulated gene includes IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, CDK1, CDK2, CDK9, farnesyl transferase, UBE2S, ABCC1, ABCC5, ABCD4, ACADM, ACLSL1, ACSL3, ACY1L2, ADM, ADRM1, AGPAT5, AHCY, AK3L1, AK3L2, AKIIP, AKR1B1, AKR1C1, AKR1C2, AKR1C3, ALDH18A1, ALDOA, ALOX5, ALPL, ANP32E, AOF1, APG5L, ARFGEF1, ARL5, ARPP-19, ASPH, ATF5, ATF7IP, ATIC, ATP11 A, ATP11C, ATP1A1, ATP1B1, ATP2A2, ATP5G3, ATP5J2, ATP6V0B, B3GNT1, B4GALT2, BACE2, BACH, BAG2, BASP1, BCAT1, BCL2L1, BCL6, BGN, BPNT1, C1QBP, CACNB3, CAMK2D, CAP2, CCAR1, CD109, CD24, CD44, CD47, CD58, CD74, CD83, CD9, CDC14B, CDC42EP4, CDC5L, CDK4, CDK6, CDS1, CDW92, CEACAM6, CELSR2, CFLAR, CGI-90, CHST6, CHSY1, CKLFSF4, CKLFSF6, CKS1B, CMKOR1, CNDP2, CPD, CPE, CPSF3, CPSF5, CPSF6, CPT1B, CRR9, CSH2, CSK, CSNK2A1, CSPG2, CTPSCTSB, CTSD, CXADR, CXCR4, CXXC5, CXXC6, DAAM1, DCK, DDAH1, DDIT4, DDR1, DDX21, DDX39, DHTKD1, DLAT, DNAJA1, DNAJB11, DNAJC1, DNAJC10, DNAJC9, DNAJD1, DUSP10, DUSP24, DUSP6, DVL3, ELOVL6, EME1, ENO1, ENPP4, EPS8, ETNK1, ETV6, F11R, FA2H, FABP5, FADS2, FAS, FBXO45, FBXO7, FLJ23091, FTL, FTLL1, FZD6, G1P2, GALNT2, GALNT4, GALNT7, GANAB, GART, GBAS, GCHFR, GCLC, GCLM, GCNT1, GFPT1, GGA2, GGH, GLUL, GMNN, GMPS, GPI, GPR56, GPR89, GPX1, GRB10, GRHPR, GSPT1, GSR, GTPBP4, HDAC1, HDGF, HIG2, HMGB3, HPRT1, HPS5, HRMT1L2, HS2ST1, HSPA4, HSPA8, HSPB1, HSPCA, HSPCAL3, HSPCB, HSPD1, HSPE1, HSPH1, HTATIP2, HYOU1, ICMT, IDE, IDH2, IFI27, IGFBP3, IGSF4, ILF2, INPP5F, INSIG1, KHSRP, KLF4, KMO, KPNA2, KTN1, LAP3, LASS2, LDHA, LDHB, LGR4, LPGAT1, LTB4DH, LYN, MAD2L1, MADP-1, MAGED1, MAK3, MALAT1, MAP2K3, MAP2K6, MAP3K13, MAP4K4, MAPK13, MARCKS, MBTPS2, MCM4, MCTS1, MDH11, MDH2, ME1, ME2, METAP2, METTL2, MGAT4B, MKNK2. MLPH, MOBK1B, MOBKL1A, MSH2, MTHFD2, MUC1, MX1, MYCBP, NAJD1, NAT1, NBS1, NDFIP2, NEK6, NET1, NME1, NNT, NQO1, NRAS, NSE2, NUCKS, NUSAP1, NY-REN-41, ODC1, OLR1, P4HB, PAFAH1B1, PAICS, PANK1, PCIA1, PCNA, PCTK1, PDAP1, PDIA4, PDIA6, PDXK, PERP, PFKP, PFTK1, PGD, PGK1, PGM2L1, PHCA, PKIG, PKM2, PKP4, PLA2G4A, PLCB1, PLCG2, PLD3, PLOD1, PLOD2, PMS2L3, PNK1, PNPT1, PON2, PP, PPIF, PPP1CA, PPP2R4, PPP3CA, PRCC, PRKD3, PRKDC, PRPSAP2, PSAT1, PSENEN, PSMA2, PSMA5, PSMA7, PSMB3, PSMB4, PSMD14, PSMD2, PSMD3, PSMD4, PSMD8, PTGFRN, PTGS1, PTK9, PTPN12, PTPN18, PTS, PYGB, RAB10, RAB11FIP1, RAB14, RAB31, RAB3IP, RACGAP1, RAN, RANBP1, RAP2B, RBBP4, RBBP7, RBBP8, RDH10, RFC3, RFC4, RFC5, RGS19IP1, RHOBTB3, RNASEH2A, RNGTT, RNPEP, ROBO1, RRAS2, SART2, SAT, SCAP2, SCD4, SDC2, SDC4, SEMA3F, SERPINE2, SFI1, SGPL1, SGPP1, SGPP2, SH3GLB2, SHC1, SMARCC1, SMC4L1, SMC4L1, SMS, SNRPD1, SORD, SORL1, SPP1, SQLE, SRD5A1, SRD5A2L, SRM, SRPK1, SS18, SSBP1, SSR3, ST3GAL5, ST6GAL1, ST6GALNAC2, STX18, SULF2, SWAP70, TA-KRP, TALA, TBL1XR1, TFRC, TIAM1, TKT, TMPO, TNFAIP2, TNFSF9, TOX, TPD52, TPI1, TPP1, TRA1, TRIP13, TRPS1, TSPAN13, TSTA3, TXN, TXNL2, TXNL5, TXNRD1, UBAP2L, UBE2A, UBE2D2, UBE2G1, UBE2V1, UCHL5, UGDH, UNC5CL, USP28, USP47, UTP14A, VDAC1, WIG1, YWHAB, YWHAE, YWHAZ, or a combination thereof.

26. The method of claim 23, wherein said disease is a cancer.

27. The method of claim 26, wherein said cancer is selected from the group consisting of breast cancer, lung cancer, endometrial cancer, ovarian cancer, bone osteosarcoma and Ewing's sarcoma.

28. The method of claim 27, wherein said breast cancer is triple negative cancer.

29. The method of claim 23, wherein said expression level of PARP and said co-regulated genes are up-regulated and the treatment decision is to treat said disease with inhibitors to PARP and said co-regulated genes.

30. The method of claim 23, wherein said expression level of PARP and said co-regulated genes are down-regulated and the treatment decision is a decision to not treat said disease with inhibitors to PARP and said co-regulated genes.

31. The method of claim 23, wherein said PARP modulator is a PARP inhibitor.

32. The method of claim 31, wherein said PARP inhibitor is selected from the group consisting of benzamide, quinolone, isoquinolone, benzopyrone, tautomers, metabolites, analogs, or prodrugs thereof.

33. The method of claim 32, wherein said PARP inhibitor is 4-iodo, 3-nitro benzamide or a metabolite thereof.

34. A method of treating a cancer susceptible to PARP inhibitor treatment, the method comprising:
a. identifying a cancer treatable with at least one PARP inhibitor, wherein the expression level of PARP in a plurality of cancer samples is up-regulated;
b. identifying at least one co-upregulated gene in said plurality of samples;
c. treating a patient with said cancer with inhibitors to PARP and the co-regulated gene.

35. The method of claim 34, wherein said co-regulated gene includes a gene expressed in the PARP, IGF1 receptor, or EGFR pathways.

36. The method of claim 34, wherein said co-regulated gene includes IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, CDK1, CDK2, CDK9, farnesyl transferase, UBE2S, ABCC1, ABCC5, ABCD4, ACADM, ACLSL1, ACSL3, ACY1L2, ADM, ADRM1, AGPAT5, AHCY, AK3L1, AK3L2, AKIIP, AKR1B1, AKR1C1, AKR1C2, AKR1C3, ALDH18A1, ALDOA, ALOX5, ALPL, ANP32E, AOF1, APG5L, ARFGEF1, ARL5, ARPP-19, ASPH, ATF5, ATF7IP, ATIC, ATP11A, ATP11C, ATP1A1, ATP1B1, ATP2A2, ATP5G3, ATP5J2, ATP6V0B, B3GNT1, B4GALT2, BACE2, BACH, BAG2, BASP1, BCAT1, BCL2L1, BCL6, BGN, BPNT1, C1QBP, CACNB3, CAMK2D, CAP2, CCAR1, CD109, CD24, CD44, CD47, CD58, CD74, CD83, CD9, CDC14B, CDC42EP4, CDC5L, CDK4, CDK6, CDS1, CDW92, CEACAM6, CELSR2, CFLAR, CGI-90, CHST6, CHSY1, CKLFSF4, CKLFSF6, CKS1B, CMKOR1, CNDP2, CPD, CPE, CPSF3, CPSF5, CPSF6, CPT1B, CRR9, CSH2, CSK, CSNK2A1, CSPG2, CTPSCTSB, CTSD, CXADR, CXCR4, CXXC5, CXXC6, DAAM1, DCK, DDAH1, DDIT4, DDR1, DDX21, DDX39, DHTKD1, DLAT, DNAJA1, DNAJB11, DNAJC1, DNAJC10, DNAJC9, DNAJD1, DUSP10, DUSP24, DUSP6, DVL3, ELOVL6, EME1, ENO1, ENPP4, EPS8, ETNK1, ETV6, F11R, FA2H, FABP5, FADS2, FAS, FBXO45, FBXO7, FLJ23091, FTL, FTLL1, FZD6, G1P2, GALNT2, GALNT4, GALNT7, GANAB, GART, GBAS, GCHFR, GCLC, GCLM, GCNT1, GFPT1, GGA2, GGH, GLUL, GMNN, GMPS, GPI, GPR56, GPR89, GPX1, GRB10, GRHPR, GSPT1, GSR, GTPBP4, HDAC1, HDGF, HIG2, HMGB3, HPRT1, HPS5, HRMT1L2, HS2ST1, HSPA4, HSPA8, HSPB1, HSPCA, HSPCAL3, HSPCB, HSPD1, HSPE1, HSPH1, HTATIP2, HYOU1, ICMT, IDE, IDH2, IFI27, IGFBP3, IGSF4, ILF2, INPP5F, INSIG1, KHSRP, KLF4, KMO, KPNA2, KTN1, LAP3, LASS2, LDHA, LDHB, LGR4, LPGAT1, LTB4DH, LYN, MAD2L1, MADP-1, MAGED1, MAK3, MALAT1, MAP2K3, MAP2K6, MAP3K13, MAP4K4, MAPK13, MARCKS, MBTPS2, MCM4, MCTS1, MDH1, MDH2, ME1, ME2, METAP2, METTL2, MGAT4B, MKNK2. MLPH, MOBK1B, MOBKL1A, MSH2, MTHFD2, MUC1, MX1, MYCBP, NAJD1, NAT1, NBS1, NDFIP2, NEK6, NET1, NME1, NNT, NQO1, NRAS, NSE2, NUCKS, NUSAP1, NY-REN-41, ODC1, OLR1, P4HB, PAFAH1B1, PAICS, PANK1, PCIA1, PCNA, PCTK1, PDAP1, PDIA4, PDIA6, PDXK, PERP, PFKP, PFTK1, PGD, PGK1, PGM2L1, PHCA, PKIG, PKM2, PKP4, PLA2G4A, PLCB1, PLCG2, PLD3, PLOD1, PLOD2, PMS2L3, PNK1, PNPT1, PON2, PP, PPIF, PPP1CA, PPP2R4, PPP3CA, PRCC, PRKD3, PRKDC, PRPSAP2, PSAT1, PSENEN, PSMA2, PSMA5, PSMA7, PSMB3, PSMB4, PSMD14, PSMD2, PSMD3, PSMD4, PSMD8, PTGFRN, PTGS1, PTK9, PTPN12, PTPN18, PTS, PYGB, RAB10, RAB11FIP1, RAB14, RAB31, RAB3IP, RACGAP1, RAN, RANBP1, RAP2B, RBBP4, RBBP7, RBBP8, RDH10, RFC3, RFC4, RFC5, RGS19IP1, RHOBTB3, RNASEH2A, RNGTT, RNPEP, ROBO1, RRAS2, SART2, SAT, SCAP2, SCD4, SDC2, SDC4, SEMA3F, SERPINE2, SFI1, SGPL1, SGPP1, SGPP2, SH3GLB2, SHC1, SMARCC1, SMC4L1, SMC4L1, SMS, SNRPD1, SORD, SORL1, SPP1, SQLE, SRD5A1, SRD5A2L, SRM, SRPK1, SS18, SSBP1, SSR3, ST3GAL5, ST6GAL1, ST6GALNAC2, STX18, SULF2, SWAP70, TA-KRP, TALA, TBL1XR1, TFRC, TIAM1, TKT, TMPO, TNFAIP2, TNFSF9, TOX, TPD52, TPI1, TPP1, TRA1, TRIP13, TRPS1, TSPAN13, TSTA3, TXN, TXNL2, TXNL5, TXNRD1, UBAP2L, UBE2A, UBE2D2, UBE2G1, UBE2V1, UCHL5, UGDH, UNC5CL, USP28, USP47, UTP14A, VDAC1, WIG1, YWHAB, YWHAE, YWHAZ, or a combination thereof.

37. The method of claim 34, wherein said cancer is selected from the group consisting of colon adenocarcinoma, esophageal adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, lung cancer, endometrial cancer, ovarian cancer, ductal carcinoma, lobular carcinoma, intraductal carcinoma, mucinous carcinoma, phyllodes tumor, Ewing's sarcoma, ovarian adenocarcinoma, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung adenocarcinoma, kidney carcinoma, urinary bladder carcinoma, Wilm's tumor and lymphoma.

38. The method of claim 34, wherein said PARP inhibitor is selected from the group consisting of benzamide, quinolone, isoquinolone, benzopyrone, cyclic benzamide, benzimidazole, indole, and pharmaceutically salts, solvates, isomers, tautomers, metabolites, analogs, or prodrugs thereof.

39. The method of claim 38, wherein said PARP inhibitor is 4-iodo, 3-nitro benzamide or a metabolite thereof.

40. A method of treating a breast cancer susceptible to PARP inhibitor treatment, the method comprising
a. identifying a breast cancer treatable with at least one PARP inhibitor, wherein the expression level of PARP in a plurality of breast cancer samples is up-regulated;
b. identifying at least one co-upregulated gene in said plurality of samples;
c. treating a patient with said breast cancer with inhibitors to PARP and the co-regulated gene.

41. The method of claim 40, wherein said co-regulated gene includes a gene expressed in the PARP, IGF1 receptor, or EGFR pathways.

42. The method of claim 40, wherein said co-regulated gene includes IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, CDK1, CDK2, CDK9, farnesyl transferase, UBE2S, ABCC1, ABCC5, ABCD4, ACADM, ACLSL1, ACSL3, ACY1L2, ADM, ADRM1, AGPAT5, AHCY, AK3L1, AK3L2, AKIIP, AKR1B1, AKR1C1, AKR1C2, AKR1C3, ALDH18A1, ALDOA, ALOX5, ALPL, ANP32E, AOF1, APG5L, ARFGEF1, ARL5, ARPP-19, ASPH, ATF5, ATF7IP, ATIC, ATP11A, ATP11C, ATP1A1, ATP1B1, ATP2A2, ATP5G3, ATP5J2, ATP6V0B, B3GNT1, B4GALT2, BACE2, BACH, BAG2, BASP1, BCAT1, BCL2L1, BCL6, BGN, BPNT1, C1QBP, CACNB3, CAMK2D, CAP2, CCAR1, CD109, CD24, CD44, CD47, CD58, CD74, CD83, CD9, CDC14B, CDC42EP4, CDC5L, CDK4, CDK6, CDS1, CDW92, CEACAM6, CELSR2, CFLAR, CGI-90, CHST6, CHSY1, CKLFSF4, CKLFSF6, CKS1B, CMKOR1, CNDP2, CPD, CPE, CPSF3, CPSF5, CPSF6, CPT1B, CRR9, CSH2, CSK, CSNK2A1, CSPG2, CTPSCTSB, CTSD, CXADR, CXCR4, CXXC5, CXXC6, DAAM1, DCK, DDAH1, DDIT4, DDR1, DDX21, DDX39, DHTKD1, DLAT, DNAJA1, DNAJB11, DNAJC1, DNAJC10, DNAJC9, DNAJD1, DUSP10, DUSP24, DUSP6, DVL3, ELOVL6, EME1, ENO1, ENPP4, EPS8, ETNK1, ETV6, F11R, FA2H, FABP5, FADS2, FAS, FBXO45, FBXO7, FLJ23091, FTL, FTLL1, FZD6, G1P2, GALNT2, GALNT4, GALNT7, GANAB, GART, GBAS, GCHFR, GCLC, GCLM, GCNT1, GFPT1, GGA2, GGH, GLUL, GMNN, GMPS, GPI, GPR56, GPR89, GPX1, GRB10, GRHPR, GSPT1, GSR, GTPBP4, HDAC1, HDGF, HIG2, HMGB3, HPRT1, HPS5, HRMT1L2, HS2ST1, HSPA4, HSPA8, HSPB1, HSPCA, HSPCAL3, HSPCB, HSPD1, HSPE1, HSPH1, HTATIP2, HYOU1, ICMT, IDE, IDH2, IFI27, IGFBP3, IGSF4, ILF2, INPP5F, INSIG1, KHSRP, KLF4, KMO, KPNA2, KTN1, LAP3, LASS2, LDHA, LDHB, LGR4, LPGAT1, LTB4DH, LYN, MAD2L1, MADP-1, MAGED1, MAK3, MALAT1, MAP2K3, MAP2K6, MAP3K13, MAP4K4, MAPK13, MARCKS, MBTPS2, MCM4, MCTS1, MDH1, MDH2, ME1, ME2, METAP2, METTL2, MGAT4B, MKNK2. MLPH, MOBK1B, MOBKL1A, MSH2, MTHFD2, MUC1, MX1, MYCBP, NAJD1, NAT1, NBS1, NDFIP2, NEK6, NET1, NME1, NNT, NQO1, NRAS, NSE2, NUCKS, NUSAP1, NY-REN-41, ODC1, OLR1, P4HB, PAFAH1B1, PAICS, PANK1, PCIA1, PCNA, PCTK1, PDAP1, PDIA4, PDIA6, PDXK, PERP, PFKP, PFTK1, PGD, PGK1, PGM2L1, PHCA, PKIG, PKM2, PKP4, PLA2G4A, PLCB1, PLCG2, PLD3, PLOD1, PLOD2, PMS2L3, PNK1, PNPT1, PON2, PP, PPIF, PPP1CA, PPP2R4, PPP3CA, PRCC, PRKD3, PRKDC, PRPSAP2, PSAT1, PSENEN, PSMA2, PSMA5, PSMA7, PSMB3, PSMB4, PSMD14, PSMD2, PSMD3, PSMD4, PSMD8, PTGFRN, PTGS1, PTK9, PTPN12, PTPN18, PTS, PYGB, RAB10, RAB11FIP1, RAB14, RAB31, RAB3IP, RACGAP1, RAN, RANBP1, RAP2B, RBBP4, RBBP7, RBBP8, RDH10, RFC3, RFC4, RFC5, RGS19IP1, RHOBTB3, RNASEH2A, RNGTT, RNPEP, ROBO1, RRAS2, SART2, SAT, SCAP2, SCD4, SDC2, SDC4, SEMA3F, SERPINE2, SFI1, SGPL1, SGPP1, SGPP2, SH3GLB2, SHC1, SMARCC1, SMC4L1, SMC4L1, SMS, SNRPD1, SORD, SORL1, SPP1, SQLE, SRD5A1, SRD5A2L, SRM, SRPK1, SS18, SSBP1, SSR3, ST3GAL5, ST6GAL1, ST6GALNAC2, STX18, SULF2, SWAP70, TA-KRP, TALA, TBL1XR1, TFRC, TIAM1, TKT, TMPO, TNFAIP2, TNFSF9, TOX, TPD52, TPI1, TPP1, TRA1, TRIP13, TRPS1, TSPAN13, TSTA3, TXN, TXNL2, TXNL5, TXNRD1, UBAP2L, UBE2A, UBE2D2, UBE2G1, UBE2V1, UCHL5, UGDH, UNC5CL, USP28, USP47, UTP14A, VDAC1, WIG1, YWHAB, YWHAE, YWHAZ, or a combination thereof.

43. The method of claim 40, wherein said breast cancer is selected from the group consisting of lymphomas, carcinomas, hormone-dependent tumors, small cell carcinoma, ductal carcinoma, infiltrating ductal carcinoma, infiltrating breast lobular carcinoma, infiltrating carcinoma of mixed ductal and lobular type and metastatic infiltrating ductal carcinoma.

44. The method of claim 40, wherein said breast cancer is triple negative breast cancer.

45. The method of claim 40, wherein said PARP inhibitor is selected from the group consisting of benzamide, quinolone, isoquinolone, benzopyrone, cyclic benzamide, benzimidazole, indole, and pharmaceutically salts, solvates, isomers, tautomers, metabolites, analogs, or prodrugs thereof.

46. The method of claim 45, wherein said PARP inhibitor is 4-iodo, 3-nitro benzamide or a metabolite thereof.

47. A method of treating a lung cancer susceptible to PARP inhibitor treatment, the method comprising:
a. identifying a lung cancer treatable with at least one PARP inhibitor, wherein the expression level of PARP in a plurality of lung cancer samples is up-regulated;
b. identifying at least one co-upregulated gene in said plurality of samples;
c. treating a patient with said lung cancer with inhibitors to PARP and the co-regulated gene.

48. The method of claim 47, wherein said co-regulated gene includes a gene expressed in the PARP, IGF1 receptor, or EGFR pathways.

49. The method of claim 47, wherein said co-regulated gene includes IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, CDK1, CDK2, CDK9, farnesyl transferase, UBE2S, ABCC1, ABCC5, ABCD4, ACADM, ACLSL1, ACSL3, ACY1L2, ADM, ADRM1, AGPAT5, AHCY, AK3L1, AK3L2, AKIIP, AKR1B1, AKR1C1, AKR1C2, AKR1C3, ALDH18A1, ALDOA, ALOX5, ALPL, ANP32E, AOF1, APG5L, ARFGEF1, ARL5, ARPP-19, ASPH, ATF5, ATF7IP, ATIC, ATP11A, ATP11C, ATP1A1, ATP1B1, ATP2A2, ATP5G3, ATP5J2, ATP6V0B, B3GNT1, B4GALT2, BACE2, BACH, BAG2, BASP1, BCAT1, BCL2L1, BCL6, BGN, BPNT1, C1QBP, CACNB3, CAMK2D, CAP2, CCAR1, CD109, CD24, CD44, CD47, CD58, CD74, CD83, CD9, CDC14B, CDC42EP4, CDC5L, CDK4, CDK6, CDS1, CDW92, CEACAM6, CELSR2, CFLAR, CGI-90, CHST6, CHSY1, CKLFSF4, CKLFSF6, CKS1B, CMKOR1, CNDP2, CPD, CPE, CPSF3, CPSF5, CPSF6, CPT1B, CRR9, CSH2, CSK, CSNK2A1, CSPG2, CTPSCTSB, CTSD, CXADR, CXCR4, CXXC5, CXXC6, DAAM1, DCK, DDAH1, DDIT4, DDR1, DDX21, DDX39, DHTKD1, DLAT, DNAJA1, DNAJB11, DNAJC1, DNAJC10, DNAJC9, DNAJD1, DUSP10, DUSP24, DUSP6, DVL3, ELOVL6, EME1, ENO1, ENPP4, EPS8, ETNK1, ETV6, F11R, FA2H, FABP5, FADS2, FAS, FBXO45, FBXO7, FLJ23091, FTL, FTLL1, FZD6, G1P2, GALNT2, GALNT4, GALNT7, GANAB, GART, GBAS, GCHFR, GCLC, GCLM, GCNT1, GFPT1, GGA2, GGH, GLUL, GMNN, GMPS, GPI, GPR56, GPR89, GPX1, GRB10, GRHPR, GSPT1, GSR, GTPBP4, HDAC1, HDGF, HIG2, HMGB3, HPRT1, HPS5, HRMT1L2, HS2ST1, HSPA4, HSPA8, HSPB1, HSPCA, HSPCAL3, HSPCB, HSPD1, HSPE1, HSPH1, HTATIP2, HYOU1, ICMT, IDE, IDH2, IFI27, IGFBP3, IGSF4, ILF2, INPP5F, INSIG1, KHSRP, KLF4, KMO, KPNA2, KTN1, LAP3, LASS2, LDHA, LDHB, LGR4, LPGAT1, LTB4DH, LYN, MAD2L1, MADP-1, MAGED1, MAK3, MALAT1, MAP2K3, MAP2K6, MAP3K13, MAP4K4, MAPK13, MARCKS, MBTPS2, MCM4, MCTS1, MDH1, MDH2, ME1, ME2, METAP2, METTL2, MGAT4B, MKNK2. MLPH, MOBK1B, MOBKL1A, MSH2, MTHFD2, MUC1, MX1, MYCBP, NAJD1, NAT1, NBS1, NDFIP2, NEK6, NET1, NME1, NNT, NQO1, NRAS, NSE2, NUCKS, NUSAP1, NY-REN-41, ODC1, OLR1, P4HB, PAFAH1B1, PAICS, PANK1, PCIA1, PCNA, PCTK1, PDAP1, PDIA4, PDIA6, PDXK, PERP, PFKP, PFTK1, PGD, PGK1, PGM2L1, PHCA, PKIG, PKM2, PKP4, PLA2G4A, PLCB1, PLCG2, PLD3, PLOD1, PLOD2, PMS2L3, PNK1, PNPT1, PON2, PP, PPIF, PPP1CA, PPP2R4, PPP3CA, PRCC, PRKD3, PRKDC, PRPSAP2, PSAT1, PSENEN, PSMA2, PSMA5, PSMA7, PSMB3, PSMB4, PSMD14, PSMD2, PSMD3, PSMD4, PSMD8, PTGFRN, PTGS1, PTK9, PTPN12, PTPN18, PTS, PYGB, RAB10, RAB11FIP1, RAB14, RAB31, RAB3IP, RACGAP1, RAN, RANBP1, RAP2B, RBBP4, RBBP7, RBBP8, RDH10, RFC3, RFC4, RFC5, RGS19IP1, RHOBTB3, RNASEH2A, RNGTT, RNPEP, ROBO1, RRAS2, SART2, SAT, SCAP2, SCD4, SDC2, SDC4, SEMA3F, SERPINE2, SFI1, SGPL1, SGPP1, SGPP2, SH3GLB2, SHC1, SMARCC1, SMC4L1, SMC4L1, SMS, SNRPD1, SORD, SORL1, SPP1, SQLE, SRD5A1, SRD5A2L, SRM, SRPK1, SS18, SSBP1, SSR3, ST3GAL5, ST6GAL1, ST6GALNAC2, STX18, SULF2, SWAP70, TA-KRP, TALA, TBL1XR1, TFRC, TIAM1, TKT, TMPO, TNFAIP2, TNFSF9, TOX, TPD52, TPI1, TPP1, TRA1, TRIP13, TRPS1, TSPAN13, TSTA3, TXN, TXNL2, TXNL5, TXNRD1, UBAP2L, UBE2A, UBE2D2, UBE2G1, UBE2V1, UCHL5, UGDH, UNC5CL, USP28, USP47, UTP14A, VDAC1, WIG1, YWHAB, YWHAE, YWHAZ, or a combination thereof.

50. The method of claim 47, wherein said lung cancer is selected from the group consisting of lung adenocarcinoma, small cell carcinoma, non-small cell carcinomas, squamous cell carcinoma and large cell carcinoma.

51. The method of claim 47, wherein said PARP inhibitor is selected from the group consisting of benzamide, quinolone, isoquinolone, benzopyrone, cyclic benzamide, benzimidazole, indole, and pharmaceutically salts, solvates, isomers, tautomers, metabolites, analogs, or prodrugs thereof.

52. The method of claim 51, wherein said PARP inhibitor is 4-iodo, 3-nitro benzamide or a metabolite thereof.

53. A method of treating an endometrial cancer susceptible to PARP inhibitor treatment, the method comprising:
a. identifying an endometrial cancer treatable with at least one PARP inhibitor, wherein the expression level of PARP in a plurality of endometrial cancer samples is up-regulated;
b. identifying at least one co-upregulated gene in said plurality of samples;
c. treating said patient with inhibitors to PARP and the co-regulated gene.

54. The method of claim 53, wherein said co-regulated gene includes a gene expressed in the PARP, IGF1 receptor, or EGFR pathways.

55. The method of claim 53, wherein said co-regulated gene includes IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, CDK1, CDK2, CDK9, farnesyl transferase, UBE2S, ABCC1, ABCC5, ABCD4, ACADM, ACLSL1, ACSL3, ACY1L2, ADM, ADRM1, AGPAT5, AHCY, AK3L1, AK3L2, AKIIP, AKR1B1, AKR1C1, AKR1C2, AKR1C3, ALDH18A1, ALDOA, ALOX5, ALPL, ANP32E, AOF1, APG5L, ARFGEF1, ARL5, ARPP-19, ASPH, ATF5, ATF7IP, ATIC, ATP11A, ATP11C, ATP1A1, ATP1B1, ATP2A2, ATP5G3, ATP5J2, ATP6V0B, B3GNT1, B4GALT2, BACE2, BACH, BAG2, BASP1, BCAT1, BCL2L1, BCL6, BGN, BPNT1, C1QBP, CACNB3, CAMK2D, CAP2, CCAR1, CD109, CD24, CD44, CD47, CD58, CD74, CD83, CD9, CDC14B, CDC42EP4, CDC5L, CDK4, CDK6, CDS1, CDW92, CEACAM6, CELSR2, CFLAR, CGI-90, CHST6, CHSY1, CKLFSF4, CKLFSF6, CKS1B, CMKOR1, CNDP2, CPD, CPE, CPSF3, CPSF5, CPSF6, CPT1B, CRR9, CSH2, CSK, CSNK2A1, CSPG2, CTPSCTSB, CTSD, CXADR, CXCR4, CXXC5, CXXC6, DAAM1, DCK, DDAH1, DDIT4, DDR1, DDX21, DDX39, DHTKD1, DLAT, DNAJA1, DNAJB11, DNAJC1, DNAJC10, DNAJC9, DNAJD1, DUSP10, DUSP24, DUSP6, DVL3, ELOVL6, EME1, ENO1, ENPP4, EPS8, ETNK1, ETV6, F11R, FA2H, FABP5, FADS2, FAS, FBXO45, FBXO7, FLJ23091, FTL, FTLL1, FZD6, G1P2, GALNT2, GALNT4, GALNT7, GANAB, GART, GBAS, GCHFR, GCLC, GCLM, GCNT1, GFPT1, GGA2, GGH, GLUL, GMNN, GMPS, GPI, GPR56, GPR89, GPX1, GRB10, GRHPR, GSPT1, GSR, GTPBP4, HDAC1, HDGF, HIG2, HMGB3, HPRT1, HPS5, HRMT1L2, HS2ST1, HSPA4, HSPA8, HSPB1, HSPCA, HSPCAL3, HSPCB, HSPD1, HSPE1, HSPH1, HTATIP2, HYOU1, ICMT, IDE, IDH2, IFI27, IGFBP3, IGSF4, ILF2, INPP5F, INSIG1, KHSRP, KLF4, KMO, KPNA2, KTN1, LAP3, LASS2, LDHA, LDHB, LGR4, LPGAT1, LTB4DH, LYN, MAD2L1, MADP-1, MAGED1, MAK3, MALAT1, MAP2K3, MAP2K6, MAP3K13, MAP4K4, MAPK13, MARCKS, MBTPS2, MCM4, MCTS1, MDH1, MDH2, ME1, ME2, METAP2, METTL2, MGAT4B, MKNK2. MLPH, MOBK1B, MOBKL1A, MSH2, MTHFD2, MUC1, MX1, MYCBP, NAJD1, NAT1, NBS1, NDFIP2, NEK6, NET1, NME1, NNT, NQO1, NRAS, NSE2, NUCKS, NUSAP1, NY-REN-41, ODC1, OLR1, P4HB, PAFAH1B1, PAICS, PANK1, PCIA1, PCNA, PCTK1, PDAP1, PDIA4, PDIA6, PDXK, PERP, PFKP, PFTK1, PGD, PGK1, PGM2L1, PHCA, PKIG, PKM2, PKP4, PLA2G4A, PLCB1, PLCG2, PLD3, PLOD1, PLOD2, PMS2L3, PNK1, PNPT1, PON2, PP, PPIF, PPP1CA, PPP2R4, PPP3CA, PRCC, PRKD3, PRKDC, PRPSAP2, PSAT1, PSENEN, PSMA2, PSMA5, PSMA7, PSMB3, PSMB4, PSMD14, PSMD2, PSMD3, PSMD4, PSMD8, PTGFRN, PTGS1, PTK9, PTPN12, PTPN18, PTS, PYGB, RAB10, RAB11FIP1, RAB14, RAB31, RAB3IP, RACGAP1, RAN, RANBP1, RAP2B, RBBP4, RBBP7, RBBP8, RDH10, RFC3, RFC4, RFC5, RGS19IP1, RHOBTB3, RNASEH2A, RNGTT, RNPEP, ROBO1, RRAS2, SART2, SAT, SCAP2, SCD4, SDC2, SDC4, SEMA3F, SERPINE2, SFI1, SGPL1, SGPP1, SGPP2, SH3GLB2, SHC1, SMARCC1, SMC4L1, SMC4L1, SMS, SNRPD1, SORD, SORL1, SPP1, SQLE, SRD5A1, SRD5A2L, SRM, SRPK1, SS18, SSBP1, SSR3, ST3GAL5, ST6GAL1, ST6GALNAC2, STX18, SULF2, SWAP70, TA-KRP, TALA, TBL1XR1, TFRC, TIAM1, TKT, TMPO, TNFAIP2, TNFSF9, TOX, TPD52, TPI1, TPP1, TRA1, TRIP13, TRPS1, TSPAN13, TSTA3, TXN, TXNL2, TXNL5, TXNRD1, UBAP2L, UBE2A, UBE2D2, UBE2G1, UBE2V1, UCHL5, UGDH, UNC5CL, USP28, USP47, UTP14A, VDAC1, WIG1, YWHAB, YWHAE, YWHAZ, or a combination thereof.

56. The method of claim 53, wherein said endometrial cancer is selected from the group consisting of endometrium adenocarcinoma, cervix adenocarcinoma, vulva squamous cell carcinoma, basal cell carcinoma, uterine cancers, carcinomas and lymphomas.

57. The method of claim 53, wherein said PARP inhibitor is selected from the group consisting of benzamide, quinolone, isoquinolone, benzopyrone, cyclic benzamide, benzimidazole, indole, and pharmaceutically salts, solvates, isomers, tautomers, metabolites, analogs, or prodrugs thereof.

58. The method of claim 57, wherein said PARP inhibitor is 4-iodo, 3-nitro benzamide or a metabolite thereof.

59. A method of treating an ovarian cancer susceptible to PARP inhibitor treatment, the method comprising:
a. identifying an ovarian cancer treatable with at least one PARP inhibitor, wherein the expression level of PARP in a plurality of ovarian cancer samples is up-regulated;
b. identifying at least one co-upregulated gene in said plurality of samples;
c. treating said patient with inhibitors to PARP and the co-regulated gene.

60. The method of claim 59, wherein said co-regulated gene includes a gene expressed in the PARP, IGF1 receptor, or EGFR pathways.

61. The method of claim 59, wherein said co-regulated gene includes IGF1, IGF2, IGFR, EGFR, mdm2, Bcl2, ETS1, MMP-1, MMP-3, MMP-9, uPA, DHFR, TYMS, NFKB, IKK, REL, RELA, RELB, IRAK1, VAV3, AURKA, ERBB3, MIF, VEGF, VEGFR, VEGFR2, CDK1, CDK2, CDK9, farnesyl transferase, UBE2S, ABCC1, ABCC5, ABCD4, ACADM, ACLSL1, ACSL3, ACY1L2, ADM, ADRM1, AGPAT5, AHCY, AK3L1, AK3L2, AKIIP, AKR1B1, AKR1C1, AKR1C2, AKR1C3, ALDH18A1, ALDOA, ALOX5, ALPL, ANP32E, AOF1, APG5L, ARFGEF1, ARL5, ARPP-19, ASPH, ATF5, ATF7IP, ATIC, ATP11A, ATP11C, ATP1A1, ATP1B1, ATP2A2, ATP5G3, ATP5J2, ATP6V0B, B3GNT1, B4GALT2, BACE2, BACH, BAG2, BASP1, BCAT1, BCL2L1, BCL6, BGN, BPNT1, C1QBP, CACNB3, CAMK2D, CAP2, CCAR1, CD109, CD24, CD44, CD47, CD58, CD74, CD83, CD9, CDC14B, CDC42EP4, CDC5L, CDK4, CDK6, CDS1, CDW92, CEACAM6, CELSR2, CFLAR, CGI-90, CHST6, CHSY1, CKLFSF4, CKLFSF6, CKS1B, CMKOR1, CNDP2, CPD, CPE, CPSF3, CPSF5, CPSF6, CPT1B, CRR9, CSH2, CSK, CSNK2A1, CSPG2, CTPSCTSB, CTSD, CXADR, CXCR4, CXXC5, CXXC6, DAAM1, DCK, DDAH1, DDIT4, DDR1, DDX21, DDX39, DHTKD1, DLAT, DNAJA1, DNAJB11, DNAJC1, DNAJC10, DNAJC9, DNAJD1, DUSP10, DUSP24, DUSP6, DVL3, ELOVL6, EME1, ENO1, ENPP4, EPS8, ETNK1, ETV6, F11R, FA2H, FABP5, FADS2, FAS, FBXO45, FBXO7, FLJ23091, FTL, FTLL1, FZD6, G1P2, GALNT2, GALNT4, GALNT7, GANAB, GART, GBAS, GCHFR, GCLC, GCLM, GCNT1, GFPT1, GGA2, GGH, GLUL, GMNN, GMPS, GPI, GPR56, GPR89, GPX1, GRB10, GRHPR, GSPT1, GSR, GTPBP4, HDAC1, HDGF, HIG2, HMGB3, HPRT1, HPS5, HRMT1L2, HS2ST1, HSPA4, HSPA8, HSPB1, HSPCA, HSPCAL3, HSPCB, HSPD1, HSPE1, HSPH1, HTATIP2, HYOU1, ICMT, IDE, IDH2, IFI27, IGFBP3, IGSF4, ILF2, INPP5F, INSIG1, KHSRP, KLF4, KMO, KPNA2, KTN1, LAP3, LASS2, LDHA, LDHB, LGR4, LPGAT1, LTB4DH, LYN, MAD2L1, MADP-1, MAGED1, MAK3, MALAT1, MAP2K3, MAP2K6, MAP3K13, MAP4K4, MAPK13, MARCKS, MBTPS2, MCM4, MCTS1, MDH1, MDH2, ME1, ME2, METAP2, METTL2, MGAT4B, MKNK2. MLPH, MOBK1B, MOBKL1A, MSH2, MTHFD2, MUC1, MX1, MYCBP, NAJD1, NAT1, NBS1, NDFIP2, NEK6, NET1, NME1, NNT, NQO1, NRAS, NSE2, NUCKS, NUSAP1, NY-REN-41, ODC1, OLR1, P4HB, PAFAH1B1, PAICS, PANK1, PCIA1, PCNA, PCTK1, PDAP1, PDIA4, PDIA6, PDXK, PERP, PFKP, PFTK1, PGD, PGK1, PGM2L1, PHCA, PKIG, PKM2, PKP4, PLA2G4A, PLCB1, PLCG2, PLD3, PLOD1, PLOD2, PMS2L3, PNK1, PNPT1, PON2, PP, PPIF, PPP1CA, PPP2R4, PPP3CA, PRCC, PRKD3, PRKDC, PRPSAP2, PSAT1, PSENEN, PSMA2, PSMA5, PSMA7, PSMB3, PSMB4, PSMD14, PSMD2, PSMD3, PSMD4, PSMD8, PTGFRN, PTGS1, PTK9, PTPN12, PTPN18, PTS, PYGB, RAB10, RAB11FIP1, RAB14, RAB31, RAB3IP, RACGAP1, RAN, RANBP1, RAP2B, RBBP4, RBBP7, RBBP8, RDH10, RFC3, RFC4, RFC5, RGS19IP1, RHOBTB3, RNASEH2A, RNGTT, RNPEP, ROBO1, RRAS2, SART2, SAT, SCAP2, SCD4, SDC2, SDC4, SEMA3F, SERPINE2, SFI1, SGPL1, SGPP1, SGPP2, SH3GLB2, SHC1, SMARCC1, SMC4L1, SMC4L1, SMS, SNRPD1, SORD, SORL1, SPP1, SQLE; SRD5A1, SRD5A2L, SRM, SRPK1, SS18, SSBP1, SSR3, ST3GAL5, ST6GAL1, ST6GALNAC2, STX18, SULF2, SWAP70, TA-KRP, TALA, TBL1XR1, TFRC, TIAM1, TKT, TMPO, TNFAIP2, TNFSF9, TOX, TPD52, TPI1, TPP1, TRA1, TRIP13, TRPS1, TSPAN13, TSTA3, TXN, TXNL2, TXNL5, TXNRD1, UBAP2L, UBE2A, UBE2D2, UBE2G1, UBE2V1, UCHL5, UGDH, UNC5CL, USP28, USP47, UTP14A, VDAC1, WIG1, YWHAB, YWHAE, YWHAZ, or a combination thereof.

62. The method of claim 59, wherein said ovarian cancer is selected from the group consisting of lymphomas, carcinomas, hormone-dependent tumors, follicular carcinoma, ovarian adenocarcinoma, ovarian carcinoma, and solid tumors of the ovarian follicle.

63. The method of claim 59, wherein said PARP inhibitor is selected from the group consisting of benzamide, quinolone, isoquinolone, benzopyrone, cyclic benzamide, benzimidazole, indole, and pharmaceutically salts, solvates, isomers, tautomers, metabolites, analogs, or prodrugs thereof.

64. The method of claim 63, wherein said PARP inhibitor is 4-iodo, 3-nitro benzamide or a metabolite thereof.

65. A kit for diagnosing or staging a disease, the kit comprising:
a. means for measuring expression level of PARP in a tissue sample;
b. means for measuring expression level of genes previously identified as co-regulated with PARP; and
c. comparing said expression levels of PARP and co-regulated genes to a reference sample, wherein the level of expression as compared to the reference sample is indicative of the presence of disease or the disease stage.

66. The kit of claim 65, wherein the up-regulation of PARP and at least one co-regulated gene is indicative of the presence of disease.

67. The kit of claim 65, wherein the tissue sample is sample is selected from the group consisting of tumor sample, hair, blood, cell, tissue, organ, brain tissue, blood, serum, sputum, saliva, plasma, nipple aspirant, synovial fluid, cerebrospinal fluid, sweat, urine, fecal matter, pancreatic fluid, trabecular fluid, cerebrospinal fluid, tears, bronchial lavage, swabbing, bronchial aspirant, semen, prostatic fluid, precervicular fluid, vaginal fluids, and pre-ejaculate.

68. The kit of claim 65, wherein the mRNA level of each co-regulated gene is measured.

69. The kit of claim 68, wherein the mRNA level is measured using a polymerase chain reaction assay.

70. A kit for treatment of a disease susceptible to a PARP inhibitor, the kit comprising:
a. means for measuring expression level of PARP in a tissue sample, wherein an increase in expression level of PARP in comparison to a reference sample is indicative of a disease susceptible to a PARP inhibitor;
b. means for measuring expression level of genes previously identified as co-regulated with PARP, wherein an increase in the expression of said co-regulated genes is indicative of a use of an inhibitor to said co-regulated gene in the treatment of said disease; and
c. inhibitors to PARP and said co-regulated genes for treatment of said disease.

71. The kit of claim 70 wherein the tissue sample is sample is selected from the group consisting of tumor sample, hair, blood, cell, tissue, organ, brain tissue, blood, serum, sputum, saliva, plasma, nipple aspirant, synovial fluid, cerebrospinal fluid, sweat, urine, fecal matter, pancreatic fluid, trabecular fluid, cerebrospinal fluid, tears, bronchial lavage, swabbing, bronchial aspirant, semen, prostatic fluid, precervicular fluid, vaginal fluids, and pre-ejaculate.

72. The kit of claim 70, wherein the mRNA level of each co-regulated gene is measured.

73. The kit of claim 72, wherein the mRNA level is measured using a polymerase chain reaction assay.

74. The kit of claim 70, wherein said PARP inhibitor is selected from the group consisting of benzamide, quinolone, isoquinolone, benzopyrone, cyclic benzamide, benzimidazole, indole, and pharmaceutically salts, solvates, isomers, tautomers, metabolites, analogs, or prodrugs thereof.

75. The kit of claim 74, wherein said PARP inhibitor is 4-iodo, 3-nitro benzamide or a metabolite thereof.
